(19) United States
(12) Patent Application Publication
(10) Pub. No.: US 2024/0398771 A1
(43) Pub. Date: Dec. 5, 2024

(54) TREATMENT OF SCLERODERMA AND RAYNAUD'S SYNDROME

(71) Applicant: Aisa Pharma, Inc., Boston, MA (US)

(72) Inventor: Andrew Sternlicht, Boston, MA (US)

(21) Appl. No.: 18/226,333

(22) Filed: Jul. 26, 2023

Related U.S. Application Data
(60) Provisional application No. 63/470,601, filed on Jun. 2, 2023.

(51) Int. Cl. A61K 31/4418; A61K 31/4985; A61P 25/00

(52) U.S. Cl. CPC A61K 31/4418; A61K 31/4985; A61P 25/00

(57) ABSTRACT

Disclosed herein are methods of treating, e.g., Raynaud's syndrome and/or scleroderma in a subject in need thereof, using cilnidipine optionally in combination with tadalafil.

### CROSS-REFERENCE TO RELATED APPLICATIONS

This application claim priority to and benefit of U.S. Provisional Application No. 63/470,601, filed on Jun. 2, 2023, the contents of which are incorporated by reference in its entirety.

### BACKGROUND

Scleroderma is a disease resulting from microvascular pathology, immune and inflammatory activation with excessive fibrosis that causes hardening and tightening of skin and connective tissues. A particularly devastating consequence of the illness is over-production of collagen in the skin and organ tissues. Scleroderma manifests as a variety of symptoms that decrease quality of life, such as hardening and tightening of patches of skin, shiny skin, restricted movement due to hardness in skin, hair loss, white lumps under the skin due to calcium deposition, exaggerated responses to cold temperatures and emotional stress, numbness in finger and/or toe, pain in finger and/or toe, changes in color in finger and/or toe, digestive system, acid reflux, restricted movement of food through the digestive tract, malnutrition, fatigue, and anxiety. Over time, Scleroderma patients often present with other comorbidities, such as Raynaud's syndrome (e.g., secondary Raynaud's syndrome), endothelial dysfunction, gastrointestinal disorders, dyspnea (i.e., shortness of breath), calcinosis, cardiac dysfunction, and kidney dysfunction. Limited disease modifying treatments are available for scleroderma, and it is considered an orphan indication with approximately 150,000 patients in the United States. Raynaud's syndrome (also referred to herein as “Raynaud's disease”, “primary Raynaud's disease”, or “Raynaud's phenomenon” is a medical condition that is characterized by episodic and periodic reduction of blood flow to, e.g., the extremities, causing pain, numbness, discoloration, burning sensation, and neuropathic pain. Discoloration of the skin whose blood supply is reduced can accompany these symptoms. Tissue ischemia from reduced blood flow, as well as reperfusion when vasoconstriction ceases, produces, painful burning sensations which can be experienced by the subject during the ischemic attack as well as when blood flow is reestablished. Symptoms of Raynaud's phenomenon can be experienced after, e.g., changes in temperature (cold or hot) in body tissues and/or the experience of strong emotions (e.g., stress) by the subject. In some severe cases, symptoms can progress to digital ulceration and/or gangrene. It is estimated that about 6% of the population is afflicted with Raynaud's syndrome.

### SUMMARY

Described herein are methods and compositions that include the use of cilnidipine, a dual N-type and L-type calcium channel blocker selective for the N-type calcium channel, and optionally tadalafil, a phosphodiesterase type 5 inhibitor, for the treatment of scleroderma, Raynaud's syndrome, and related symptoms in a subject.

Scleroderma and Raynaud's syndrome are notoriously difficult to treat, with existing drugs (such as calcium channel blockers which are directed at improving only the Raynaud's symptoms and attacks that most patients experience) having the potential for serious side effects that are, without wishing to be bound by theory, believed to be at least in part the result of unselective (e.g., non-discriminate or low selectivity) calcium channel inhibition.

Based on these considerations, N-selective dual N- and L-type calcium channel inhibition can be useful to treat diseases and disorders that are associated with dysregulation of blood flow and sympathetic nervous system overactivity, including those featuring symptoms of neuropathic pain and vasoconstriction, such as scleroderma. Cilnidipine can also, in some embodiments, possess selective sodium channel blocker activity (e.g., Nav 1.7 sodium channel blocker activity), transient receptor potential vanilloid-1 ion channel (TRP-v1) blocker activity, reduce the release of Interleukin-1 (IL-1), decrease P2X7R activity, decrease serum uric acid levels, decrease thrombospondin, and decrease TGF-□ for the treatment of scleroderma in a subject. This can be useful in the treatment of scleroderma, Raynaud's syndrome, and many comorbidities associated with scleroderma, such as endothelial dysfunction, gastrointestinal disorders, and calcinosis.

It is understood that, in contrast to L-type calcium channel blockade, dual N-type and L-type calcium channel blockade selective for the N-type calcium channel can decrease sympathetic activity, as well as dilate both arterioles and the venous system, resulting in less adverse events than patients treated with dual L and N-calcium channel antagonists with lower levels of N type calcium channel selectivity (such as, e.g., a lack of peripheral edema caused by lesser dilation of the venous system). It is therefore postulated that this may enable dosing of a cilnidipine at higher levels than non-N selective calcium channel blockers, thus increasing potency at producing disease modifying effects. In addition, dual L-channel type and N-channel type calcium channel blockade selective for the N-type calcium channel is believed to stimulate the release of NO and endothelial nitric oxide synthase (eNOS) expression, which are critical for normal endothelial function. Further, N-type calcium channels are more widely located in the body than L-type channels, and have been identified in the nervous system, heart, kidney, venules, and the endothelium. In the spine, N-type channels are located pre-synaptically and regulate sympathetic nerve activity; these channels may involve suppression of both arteriole and venule constriction in the fingers and toes that occur in secondary Raynaud's syndrome in subjects having scleroderma.

A beneficial effect of L-type calcium channel inhibition is the dilation of the arteries in smooth muscle, causing an increase in arterial diameter, referred to as vasodilation. However, L-type calcium channel inhibition induces a homeostatic reflex mechanism in which norepinephrine is produced. The norepinephrine induces vasoconstriction, as well as elevating heart rate, and thus partially offsets the vasodilating effects of the L-type calcium channel inhibition. A useful complementary effect of N-type calcium channel inhibition is the decrease of norepinephrine release and sympathetic outflow presynaptically in the spinal cord at the level of the dorsal root ganglion, which can counteract the homeostasis mechanism triggered by blockade of the L-type calcium channel. It is believed that dual N-type and L-type calcium channel blockers selective for the N-type calcium channel are able to achieve an optimal balance of N- vs. L-type calcium channel inhibition to realize these effects. Cilnidipine (cilnidipine, 1,4-dihydro-2,6-dimethyl,4-(3-nitro phenyl)-3,5-pyridinedicarboxylic acid 2-methoxyethyl (2 E)-3-phenyl-2-propenyl ester) is a dihydropyridine CCB that dilates blood vessels, increases blood flow, inhibits sympathetic nervous system activity, and improves endothelial structure and function.

Additional advantages include an increase in bone density in certain subjects (e.g., subjects afflicted with osteoporosis), beneficial renal effects, and improvement in vascular modeling which has been shown to reduce the progression of arterial disease with long term use. The beneficial renal effects are understood to be an effect of reduced renal constriction and improved blood flow in the kidney. Cilnidipine may have analgesic and anti-fibrotic effects. As a result of its pharmacodynamics, in hypertensive patients cilnidipine has been shown to have improved efficacy compared to other hypertensive treatments including other CCBs, while exhibiting a better safety profile with less tachycardia, headaches, flushing, and reduced pedal edema reported.

Cilnidipine is understood to exert an optimum balance of selective N- vs. L-type calcium channel inhibition (which can have a 5 fold to 50-fold to 100-fold selectivity for N-type calcium channel over L-type calcium channel), which can make it surprisingly effective at treating neuropathic pain and vasoconstriction, particularly when they occur concurrently, such as in scleroderma and/or Raynaud's syndrome. The potential role of cilnidipine and dual N-type and L-type calcium channel blockers selective for the N-type calcium channels in treating scleroderma and Raynaud's syndrome has not been recognized. Cilnidipine also has activity against the Nav 1.7 sodium channel, which may further contribute to its efficacy in the treatment of scleroderma and Raynaud's syndrome. See, e.g., WO 2021/178903 and U.S. Provisional Application No. 63/470,053, filed on May 31, 2023, each of which is incorporated by reference herein in its entirety.

Phosphodiesterase inhibitors have been explored for treatment in patients having, e.g., neuropathic pain and/or vasoconstriction; however, at the doses that have been explored in these studies, common side effects occur such as headache, flushing, and dyspepsia and occasionally hypotension when used concurrently with nitrates.

Based on these considerations, cilnidipine and optionally tadalafil (a phosphodiesterase type 5 inhibitor) can be surprisingly useful to treat scleroderma and Raynaud's disease (e.g., secondary Raynaud's disease (e.g., secondary Raynaud's disease in subjects having scleroderma)). It is believed that the selective inhibition of the N-type calcium channel by cilnidipine enables the use of a lower dose of tadalafil than would otherwise be required, therefore improving tolerability in the subject. Furthermore, the combination of cilnidipine and tadalafil is particularly advantageous due, e.g., to the similar time it takes for each drug to achieve maximal plasma concentrations (2 hr_{Tmax} for cilnidipine and 2 hr_{Tmax} for tadalafil. This combination reaches maximal plasma concentrations faster than the commonly used non-N-selective calcium channel blockers amlodipine (10 hours) or extended-release nifedipine (3 hours), which is an additional benefit of using both cilnidipine and tadalafil for treating scleroderma patients that have secondary Raynaud's disease compared to other non-N-selective calcium channel blockers. Further, both drugs can be dosed at similar intervals (e.g., once daily), providing further basis for their complementarity.

Additional advantages of cilnidipine include, for example:
Improvement in endothelial function and endothelial concentrations of nitric oxide by improving blood flow, reducing pain that is, e.g., a consequence of reduced blood flow.
Improvement in cardiac and left ventricle functioning resulting in reduction of pain due, e.g., to ischemia.
Improvement in the incidence and severity of atherosclerosis including reducing pain caused, e.g., by a reduction in blood flow, and reducing the overall incidence of atherosclerotic-related events.
Decrease in overall sympathetic nervous system activity and plasma concentration of norepinephrine, which can decrease pain due to net arteriole dilation and decrease in sympathetically mediated pain syndromes.
Improvement in overall autonomic functioning, which may improve gut function in patients whose gut function (e.g., competency of the lower esophageal sphincter and peristalsis and gastric emptying), is compromised due to impaired autonomic function as occurs in certain disease states (e.g., scleroderma).
Decrease in oxidative stress and reduction in reactive oxygen species.

Based on these considerations, a cilnidipine, optionally combined with tadalafil, can be surprisingly useful to treat scleroderma and/or Raynaud's syndrome.

Disclosed herein is a method of treating one or more symptoms associated with a comorbidity of secondary Raynaud's syndrome in a subject in need thereof, comprising:
determining that the subject has (i) secondary Raynaud's syndrome, and (ii) a comorbidity of secondary Raynaud's syndrome;
orally administering about 30 mg or about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject;
wherein the subject is cilnidipine-naïve; and
wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human.

As used herein, the phrase “fixed dosage form” refers to the simultaneous administration of two or more therapeutic agents to a subject in the form of a single composition or dosage.

As used herein, the term “comorbidity of secondary Raynaud's syndrome” refers to a disease or medical condition that is present in a subject identified or diagnosed as having secondary Raynaud's syndrome, and wherein the disease or medical condition is associated with (e.g., directly or indirectly causes) the secondary Raynaud's syndrome. In some embodiments, a comorbidity of secondary Raynaud's syndrome is systemic sclerosis, lupus, rheumatoid arthritis, or any combination thereof.

As used herein, the term “non-N-selective calcium channel blocker” refers to an agent that blocks one or more calcium channels, but either (1) does not block the N-type calcium channel, or (2) blocks the N-type calcium channel, but not selectively over the L-type calcium channel (e.g., selectively blocks the L-type calcium channel over the N-type calcium channel). Examples of non-N-selective calcium channel blockers include, but are not limited to, nifedipine, nicardipine, amlodipine, Z-944, nimodipine, verapamil, diltiazem, felodipine, isradipine, nisoldipine, nitrendipine, and pharmaceutical salts thereof.

As used herein, the term “vasoconstriction” refers to the reduction in diameter of a blood vessel (e.g., an artery, vein, or capillary) resulting in reduced blood flow to the tissue the vasoconstricted blood vessels circulate blood to and from.

The term “therapeutically effective amount,” as used herein, refers to a sufficient amount of a chemical entity being administered which will relieve to an extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. When a combination of two or more chemical entities is administered (e.g., cilnidipine and tadalafil), a therapeutically effective amount of each component of the combination is understood to be the therapeutically effective amount of each component when used in conjunction with the other component, which can be different (e.g., lower) than the therapeutically effective amount of each component when administered alone.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism.

The terms “systemic sclerosis”, “scleroderma”, and “SSc” are used interchangeably herein.

For purposes of clarification, when a parameter, score, state, condition, or statistic in a subject is increased, decreased, or improved after administration of the cilnidipine or a pharmaceutically acceptable salt thereof and optionally tadalafil or a pharmaceutically acceptable salt thereof, the increase, decrease, or improvement is, for example, measured, assessed, or obtained in relation to the parameter, score, state, condition, or statistic measured, assessed, or obtained before administration of the cilnidipine and tadalafil, unless otherwise specified herein. The parameter, score, state, condition, or statistic can be a single measurement, score, or assessment, an average of a plurality of measurements, scores, or assessments, or a daily average of a plurality of measurements, scores, or assessments. Unless otherwise specified herein, measurements, scores, or assessments are typically taken within 1 month (e.g., within 3 weeks, 2 weeks, 10 days, 9 days, 8 days, 7 days, 6 days, 5 days, 4 days, 3 days, 2 days, 1 day, 18 hours, 12 hours, or 6 hours) of the administration of the cilnidipine and tadalafil.

The details of one or more embodiments of the invention are set forth in the description below. Other features and advantages of the invention will be apparent from the description and drawings, and from the claims.

### DESCRIPTION OF DRAWINGS

FIG. 1 shows a schematic of a study to assess the safety and efficacy of cilnidipine alone and in combination with tadalafil in subjects having secondary Raynaud's disease.

FIG. 2 shows a schedule of assessments in the double-blind parallel group.

FIG. 3A and FIG. 3B show a schedule of assessments in the double-blind 4-way crossover group.

FIG. 4A and FIG. 4B show a schedule of assessments in the double-blind 2-way crossover group.

### DETAILED DESCRIPTION

Disclosed herein is a method of reducing the frequency, severity, and/or duration of one or more symptoms associated with a comorbidity of secondary Raynaud's syndrome in a subject. In some embodiments, the subject is previously determined to have (i) secondary Raynaud's syndrome, and (ii) a comorbidity of secondary Raynaud's syndrome. In some embodiments, the method comprises orally administering about 20 mg or about 30 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject. In some embodiments, the method comprises orally administering about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject. In some embodiments, the method comprises orally administering about 30 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject.

Disclosed herein is a method of reducing the frequency, severity, and/or duration of one or more symptoms associated with a comorbidity of secondary Raynaud's syndrome in a subject previously determined to have (i) secondary Raynaud's syndrome, and (ii) a comorbidity of secondary Raynaud's syndrome, comprising:
orally administering about 30 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject.

Disclosed herein is a method of reducing the frequency, severity, and/or duration of one or more symptoms associated with a comorbidity of secondary Raynaud's syndrome in a subject previously determined to have (i) secondary Raynaud's syndrome, and (ii) a comorbidity of secondary Raynaud's syndrome, comprising:
orally administering about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject.

In some embodiments, the method comprises administering cilnidipine or a pharmaceutically acceptable salt thereof. In some embodiments, the method comprises administering cilnidipine or a pharmaceutically acceptable salt thereof and tadalafil or a pharmaceutically acceptable salt thereof.

In some embodiments, the subject is naïve to calcium channel blockers. In some embodiments, the subject is cilnidipine-naïve.

In some embodiments, determining that the subject has secondary Raynaud's syndrome comprises determining that the subject has a Raynaud's Condition Score (RCS) of at least 10 (e.g., at least 15, at least 20, at least 25, at least 30, at least 35, at least 40, at least 45, at least 50, at least 55, at least 60, at least 65, at least 70, at least 75, at least 80, at least 85, or at least 90). In some embodiments, determining that the subject has secondary Raynaud's syndrome comprises determining that the subject has a Raynaud's Condition Score (RCS) of at least 40.

In some embodiments, determining that the subject has secondary Raynaud's syndrome comprises performing a nailfold capillaroscopy on the subject. In some embodiments, performing the nailfold capillaroscopy on the subject comprises determining capillary abnormalities in the subject. In some embodiments, determining capillary abnormalities in the subject comprises determining mega capillaries in the subject.

In some embodiments, determining that the subject has secondary Raynaud's syndrome comprises exposing a digit (e.g., a finger) of the subject to a temperature of lesser than 36° C. (e.g., lesser than 30° C., lesser than 25° C., lesser than 20° C., lesser than 15° C., lesser than 10° C., lesser than 5° C., or lesser than 0° C.), then determining that the color of the digit of the subject became more white or more blue.

In some embodiments, the comorbidity of secondary Raynaud's syndrome is systemic sclerosis, lupus, rheumatoid arthritis, or any combination thereof. In some embodiments, the comorbidity of secondary Raynaud's syndrome is systemic sclerosis.

In some embodiments, the comorbidity of secondary Raynaud's syndrome is lupus. In some embodiments, the comorbidity of secondary Raynaud's syndrome is rheumatoid arthritis.

In some embodiments, determining that the subject has systemic sclerosis comprises determining one or more (e.g., 2 or more, 3 or more, 4 or more, 2, 3, 4, or 5) of the following:
calcium deposits underneath the skin of the subject;
determining that the color of a digit of the subject became more white or more blue after exposure of the digit to a temperature of lesser than 36° C. (e.g., lesser than 30° C., lesser than 25° C., lesser than 20° C., lesser than 15° C., lesser than 10° C., lesser than 5° C., or lesser than 0° C.);
abnormal movement of the esophagus as determined by a medical professional (e.g., a physician or a nurse practitioner);
sclerodatyly; or
telangiectasias.

In some embodiments, determining that the subject has systemic sclerosis comprises determining that the subject has an erythrocyte sedimentation rate of at least 13 mm/hr (e.g., at least 15 mm/hr, at least 20 mm/hr, at least 22 mm/hr, at least 25 mm/hr, at least 29 mm/hr, at least 30 mm/hr, at least 35 mm/hr, or at least 40 mm/hr). In some embodiments, determining that the subject has systemic sclerosis comprises determining that the subject has an erythrocyte sedimentation rate of at least 22 mm/hr. In some embodiments, determining that the subject has systemic sclerosis comprises determining that the subject has an erythrocyte sedimentation rate of at least 29 mm/hr.

In some embodiments, determining that the subject has lupus comprises determining that the subject has an erythrocyte sedimentation rate of at least 13 mm/hr (e.g., at least 15 mm/hr, at least 20 mm/hr, at least 22 mm/hr, at least 25 mm/hr, at least 29 mm/hr, at least 30 mm/hr, at least 35 mm/hr, or at least 40 mm/hr). In some embodiments, determining that the subject has lupus comprises determining that the subject has an erythrocyte sedimentation rate of at least 22 mm/hr. In some embodiments, determining that the subject has lupus comprises determining that the subject has an erythrocyte sedimentation rate of at least 29 mm/hr.

In some embodiments, determining that the subject has lupus comprises determining a positive antinuclear antibody test. In some embodiments, determining that the subject has lupus comprises determining proteinuria and/or hematuria in the subject.

In some embodiments, determining that the subject has rheumatoid arthritis comprises determining that the subject has a C-reactive protein concentration of at least 0.8 mg/L (e.g., at least 1 mg/L, at least 3 mg/L, at least 7 mg/L, at least 10 mg/L, at least 12 mg/L, at least 15 mg/L, at least 20 mg/L).

In some embodiments, determining that the subject has rheumatoid arthritis comprises determining that a magnetic resonance image (MRI) of a joint of the subject shows an abnormally low joint space width as determined by a medical professional (e.g., a physician, a nurse practitioner, or a technician).

In some embodiments, determining that the subject has rheumatoid arthritis comprises determining that an X-ray of a joint of the subject shows an abnormally low joint space width as determined by a medical professional (e.g., a physician, a nurse practitioner, or a technician).

In some embodiments, determining that the subject has rheumatoid arthritis comprises determining that the subject has an erythrocyte sedimentation rate of at least 13 mm/hr (e.g., at least 15 mm/hr, at least 20 mm/hr, at least 22 mm/hr, at least 25 mm/hr, at least 29 mm/hr, at least 30 mm/hr, at least 35 mm/hr, or at least 40 mm/hr). In some embodiments, determining that the subject has rheumatoid arthritis comprises determining that the subject has an erythrocyte sedimentation rate of at least 22 mm/hr. In some embodiments, determining that the subject has rheumatoid arthritis comprises determining that the subject has an erythrocyte sedimentation rate of at least 29 mm/hr.

In some embodiments, the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.

Disclosed herein is a method of reducing the frequency, severity, and/or duration of one or more symptoms associated with a comorbidity of secondary Raynaud's syndrome in a cilnidipine-naïve subject previously determined to have (i) secondary Raynaud's syndrome, and (ii) a comorbidity of secondary Raynaud's syndrome, comprising:
orally administering about 30 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with a comorbidity of secondary Raynaud's syndrome are selected from the group consisting of: tooth pain, muscle pain, back pain, joint pain, headache, skin ulcer pain, neuropathic pain, gastrointestinal symptoms (e.g., gastrointestinal pain), or any combination thereof.

Disclosed herein is a method of reducing the frequency, severity, and/or duration of one or more symptoms associated with a comorbidity of secondary Raynaud's syndrome in a cilnidipine-naïve subject previously determined to have (i) secondary Raynaud's syndrome, and (ii) a comorbidity of secondary Raynaud's syndrome, comprising:
orally administering about 20 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with a comorbidity of secondary Raynaud's syndrome are selected from the group consisting of: tooth pain, muscle pain, back pain, joint pain, headache, skin ulcer pain, neuropathic pain, gastrointestinal symptoms (e.g., gastrointestinal pain), or any combination thereof.

In some embodiments, after a first administration of cilnidipine or a pharmaceutically acceptable salt thereof and tadalafil or a pharmaceutically acceptable salt thereof, the severity and/or duration of the one or more symptoms is reduced in the subject.

In some embodiments, the cilnidipine or a pharmaceutically acceptable salt thereof and the tadalafil or a pharmaceutically acceptable salt thereof are administered to the subject once daily. In some embodiments, the cilnidipine or a pharmaceutically acceptable salt thereof and the tadalafil or a pharmaceutically acceptable salt thereof are administered to the subject for at least five days. In some embodiments, the cilnidipine or a pharmaceutically acceptable salt thereof and the tadalafil or a pharmaceutically acceptable salt thereof are administered to the subject once daily for at least five days.

In some embodiments, the cilnidipine or a pharmaceutically acceptable salt thereof and the tadalafil or a pharmaceutically acceptable salt thereof are administered to the subject in the morning.

In some embodiments, the cilnidipine or a pharmaceutically acceptable salt thereof and tadalafil or a pharmaceutically acceptable salt thereof are administered as a fixed dosage form.

In some embodiments, the cilnidipine or a pharmaceutically acceptable salt thereof and tadalafil or a pharmaceutically acceptable salt thereof are formulated as a liquid suspension or as an oral thin film. In some embodiments, the cilnidipine or a pharmaceutically acceptable salt thereof and tadalafil or a pharmaceutically acceptable salt thereof are formulated as a liquid suspension. In some embodiments, the cilnidipine or a pharmaceutically acceptable salt thereof and tadalafil or a pharmaceutically acceptable salt thereof are formulated as an oral thin film.

In some embodiments, the frequency of the one or more symptoms associated with the comorbidity of Raynaud's syndrome in the subject is reduced by at least 25% after administering the cilnidipine or a pharmaceutically acceptable salt thereof and the tadalafil or a pharmaceutically acceptable salt thereof.

Disclosed herein is a method of reducing the frequency, severity, and/or duration of one or more symptoms associated with a comorbidity of secondary Raynaud's syndrome in a subject previously determined to have (i) secondary Raynaud's syndrome, and (ii) a comorbidity of secondary Raynaud's syndrome, comprising:
orally administering about 30 mg of cilnidipine or a pharmaceutically acceptable salt thereof on a free base basis of cilnidipine to the subject;
wherein the one or more symptoms associated with a comorbidity of secondary Raynaud's syndrome is selected from the group consisting of: tooth pain, muscle pain, back pain, joint pain, headache, skin ulcer pain, neuropathic pain, gastrointestinal symptoms (e.g., gastrointestinal pain), or any combination thereof.

Disclosed herein is a method of reducing the frequency, severity, and/or duration of one or more symptoms associated with a comorbidity of secondary Raynaud's syndrome in a subject previously determined to have (i) secondary Raynaud's syndrome, and (ii) a comorbidity of secondary Raynaud's syndrome, comprising:
orally administering about 20 mg of cilnidipine or a pharmaceutically acceptable salt thereof on a free base basis of cilnidipine to the subject;
wherein the one or more symptoms associated with a comorbidity of secondary Raynaud's syndrome is selected from the group consisting of: tooth pain, muscle pain, back pain, joint pain, headache, skin ulcer pain, neuropathic pain, gastrointestinal symptoms (e.g., gastrointestinal pain), or any combination thereof.

Disclosed herein is a method of treating one or more symptoms associated with a comorbidity of secondary Raynaud's syndrome in a subject in need thereof, comprising:
determining that the subject has (i) secondary Raynaud's syndrome, and (ii) a comorbidity of secondary Raynaud's syndrome;
orally administering about 30 mg or about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject;
wherein the subject is cilnidipine-naïve; and
wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.

In some embodiments, about 20 mg of cilnidipine or a pharmaceutically acceptable salt thereof on a free base basis of cilnidipine is administered to the subject. In some embodiments, about 30 mg of cilnidipine or a pharmaceutically acceptable salt thereof on a free base basis of cilnidipine is administered to the subject.

In some embodiments, the tadalafil is not administered to the subject. In some embodiments, the tadalafil is administered to the subject.

In some embodiments, after a first administration of cilnidipine or a pharmaceutically acceptable salt thereof, the severity and/or duration of the one or more symptoms is reduced in the subject.

In some embodiments, the cilnidipine or a pharmaceutically acceptable salt thereof is administered to the subject once daily. In some embodiments, the cilnidipine or a pharmaceutically acceptable salt thereof is administered to the subject for at least five days. In some embodiments, the cilnidipine or a pharmaceutically acceptable salt thereof is administered to the subject once daily for at least five days.

In some embodiments, the cilnidipine or a pharmaceutically acceptable salt thereof is administered to the subject in the morning.

In some embodiments, the comorbidity of secondary Raynaud's syndrome is systemic sclerosis, lupus, rheumatoid arthritis, or any combination thereof.

In some embodiments, the cilnidipine or a pharmaceutically acceptable salt thereof is formulated as a liquid suspension or as an oral thin film. In some embodiments, the cilnidipine or a pharmaceutically acceptable salt thereof is formulated as a liquid suspension. In some embodiments, the cilnidipine or a pharmaceutically acceptable salt thereof is formulated as an oral thin film.

In some embodiments, the frequency of the one or more symptoms associated with the comorbidity of Raynaud's syndrome in the subject is reduced by at least 25% after administering the cilnidipine or a pharmaceutically acceptable salt thereof and the tadalafil or a pharmaceutically acceptable salt thereof.

In some embodiments, reducing the frequency, severity, and/or duration of one or more symptoms comprises reducing the frequency and severity of the one or more symptoms. In some embodiments, reducing the frequency, severity, and/or duration of one or more symptoms comprises reducing the frequency of the one or more symptoms. In some embodiments, reducing the frequency, severity, and/or duration of one or more symptoms comprises reducing the severity of the one or more symptoms. In some embodiments, reducing the duration of one or more symptoms comprises reducing the severity of the one or more symptoms.

In some embodiments, the one or more symptoms comprises tooth pain.

In some embodiments, the one or more symptoms comprises muscle pain.

In some embodiments, the one or more symptoms comprises back pain.

In some embodiments, the one or more symptoms comprises joint pain.

In some embodiments, the one or more symptoms comprises headache.

In some embodiments, the one or more symptoms comprises skin ulcer pain.

In some embodiments, the one or more symptoms comprises neuropathic pain.

In some embodiments, the one or more symptoms comprises gastrointestinal symptoms.

In some embodiments, the one or more symptoms is tooth pain.

In some embodiments, the one or more symptoms is muscle pain.

In some embodiments, the one or more symptoms is back pain.

In some embodiments, the one or more symptoms is joint pain.

In some embodiments, the one or more symptoms is headache.

In some embodiments, the one or more symptoms is skin ulcer pain.

In some embodiments, the one or more symptoms is neuropathic pain.

In some embodiments, the one or more symptoms is gastrointestinal symptoms.

In some embodiments, the one or more symptoms of the comorbidity of Raynaud's syndrome are selected from the group consisting of: pain, gastrointestinal symptoms, vasoconstriction, anemia, fatigue, change in coloration of the skin, cyanosis, reperfusion, deoxygenation of the blood, digital ulcerations, reduced temperature in one or more parts of the body, changes in the endothelium of a blood vessel, swelling, impaired vision, or any combination thereof.

In some embodiments, the symptom is pain. In some embodiments, the pain is neuropathic pain. In some embodiments, the pain is tooth pain, muscle pain, back pain, joint pain, headache, skin ulcer pain, neuropathic pain, gastrointestinal pain, or any combination thereof.

In some embodiments, the one or more symptoms associated with a comorbidity of secondary Raynaud's syndrome are selected from the group consisting of: tooth pain, muscle pain, back pain, joint pain, headache, skin ulcer pain, neuropathic pain, gastrointestinal symptoms (e.g., gastrointestinal pain), or any combination thereof.

In some embodiments, the symptom is a gastrointestinal symptom.

In some embodiments, the symptom is vasoconstriction. In some embodiments, the vasoconstriction comprises vasoconstriction of a body part, and the temperature of the vasoconstricted body part is lower than the subject's body temperature. In some embodiments, the body part is a finger of the subject.

In some embodiments, the symptom is anemia.

In some embodiments, the symptom is fatigue.

In some embodiments, the symptom is change in coloration of the skin.

In some embodiments, the symptom is cyanosis.

In some embodiments, the symptom is reperfusion.

In some embodiments, the symptom is deoxygenation of the blood.

In some embodiments, the symptom is digital ulcerations.

In some embodiments, the symptom is reduced temperature in one or more parts of the body.

In some embodiments, the symptom is changes in the endothelium of a blood vessel.

In some embodiments, the symptom is swelling.

In some embodiments, the symptom is impaired vision.

In some embodiments, the subject has scleroderma. In some embodiments, the subject is diagnosed with scleroderma. In some embodiments, the subject is identified as having scleroderma. In some embodiments, the scleroderma is limited scleroderma. In some embodiments, the scleroderma is diffuse scleroderma.

Disclosed herein is a method of reducing the frequency, severity, and/or duration of one or more symptoms associated with systemic sclerosis in a subject previously determined to have systemic sclerosis, comprising:
orally administering about 30 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject.

Disclosed herein is a method of reducing the frequency, severity, and/or duration of one or more symptoms associated with systemic sclerosis in a subject previously determined to have Raynaud's syndrome, comprising:
orally administering about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject.

In some embodiments, the method comprises administering cilnidipine or a pharmaceutically acceptable salt thereof. In some embodiments, the method comprises administering cilnidipine or a pharmaceutically acceptable salt thereof and tadalafil or a pharmaceutically acceptable salt thereof.

In some embodiments, the subject is naïve to calcium channel blockers. In some embodiments, the subject is cilnidipine-naïve.

In some embodiments, the subject was previously determined to have Raynaud's syndrome (e.g., secondary Raynaud's syndrome).

In some embodiments, reducing the frequency, severity, and/or duration of one or more symptoms comprises reducing the frequency and severity of the one or more symptoms. In some embodiments, reducing the frequency, severity, and/or duration of one or more symptoms comprises reducing the frequency of the one or more symptoms.

In some embodiments, reducing the frequency, severity, and/or duration of one or more symptoms comprises reducing the severity of the one or more symptoms. In some embodiments, reducing the duration of one or more symptoms comprises reducing the severity of the one or more symptoms.

In some embodiments, one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of reducing the frequency, severity, and/or duration of one or more symptoms associated with systemic sclerosis in a cilnidipine-naïve subject previously determined to have systemic sclerosis, comprising:
orally administering about 30 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of reducing the frequency, severity, and/or duration of one or more symptoms associated with systemic sclerosis in a cilnidipine-naïve subject previously determined to have systemic sclerosis, comprising:
orally administering about 20 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of reducing the frequency and/or severity of one or more symptoms associated with systemic sclerosis in a cilnidipine-naïve subject previously determined to have systemic sclerosis, comprising:
orally administering about 30 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of reducing the frequency and/or severity of one or more symptoms associated with systemic sclerosis in a cilnidipine-naïve subject previously determined to have systemic sclerosis, comprising:
orally administering about 20 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of treating one or more symptoms associated with systemic sclerosis in a subject in need thereof, comprising:
determining that the subject has systemic sclerosis;
orally administering about 30 mg or about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject;
wherein the subject is cilnidipine-naïve;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof; and
wherein the cilnidipine or a pharmaceutically acceptable salt thereof and the tadalafil or a pharmaceutically acceptable salt thereof are administered to the subject once daily for at least five days.

In some embodiments, after a first administration of cilnidipine or a pharmaceutically acceptable salt thereof and tadalafil or a pharmaceutically acceptable salt thereof, the severity and/or duration of the one or more symptoms is reduced in the subject.

In some embodiments, the cilnidipine or a pharmaceutically acceptable salt thereof and the tadalafil or a pharmaceutically acceptable salt thereof are administered to the subject once daily for at least five days.

In some embodiments, the cilnidipine or a pharmaceutically acceptable salt thereof and the tadalafil or a pharmaceutically acceptable salt thereof are administered to the subject in the morning. In some embodiments, (when the tadalafil is optional), the cilnidipine or a pharmaceutically acceptable salt thereof and the optional tadalafil or a pharmaceutically acceptable salt thereof are administered to the subject in the morning.

In some embodiments, the cilnidipine or a pharmaceutically acceptable salt thereof and tadalafil or a pharmaceutically acceptable salt thereof are administered as a fixed dosage form.

In some embodiments, the cilnidipine or a pharmaceutically acceptable salt thereof and tadalafil or a pharmaceutically acceptable salt thereof are formulated as a liquid suspension or as an oral thin film.

In some embodiments, after administering the cilnidipine or a pharmaceutically acceptable salt thereof and the tadalafil or a pharmaceutically acceptable salt thereof to the subject, the frequency of emergent medical care, systemic sclerosis related medical facility visits, hospitalization, or any combination thereof of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof and the tadalafil or a pharmaceutically acceptable salt thereof to the subject, the frequency of emergent medical care of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof and the tadalafil or a pharmaceutically acceptable salt thereof to the subject, the frequency of systemic sclerosis related medical facility visits of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof and the tadalafil or a pharmaceutically acceptable salt thereof to the subject, the frequency of hospitalization of the subject are reduced in comparison to before administering the cilnidipine or a pharmaceutically acceptable salt thereof and the tadalafil or a pharmaceutically acceptable salt thereof to the subject.

In some embodiments, reducing the frequency and/or severity of one or more symptoms associated with systemic sclerosis comprises measuring or assessing an improvement in the Scleroderma Health Assessment Questionnaire (SHAQ®) in the subject after administering the cilnidipine or a pharmaceutically acceptable salt thereof and tadalafil or a pharmaceutically acceptable salt thereof to the subject.

In some embodiments, an improvement of at least 30% in overall systemic sclerosis disease severity is measured or assessed in the visual analog scale (VAS) of overall systemic sclerosis disease severity in the subject after administering the cilnidipine or a pharmaceutically acceptable salt thereof and tadalafil or a pharmaceutically acceptable salt thereof to the subject.

Disclosed herein is a method of reducing the frequency, severity, and/or duration of one or more symptoms associated with systemic sclerosis in a subject previously determined to have systemic sclerosis, comprising:
orally administering about 30 mg of cilnidipine or a pharmaceutically acceptable salt thereof on a free base basis of cilnidipine to the subject;
wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

Disclosed herein is a method of reducing the frequency, severity, and/or duration of one or more symptoms associated with systemic sclerosis in a subject previously determined to have (i) systemic sclerosis and (ii) systemic sclerosis, lupus, rheumatoid arthritis, or any combination thereof, comprising:
orally administering about 20 mg of cilnidipine or a pharmaceutically acceptable salt thereof on a free base basis of cilnidipine to the subject;
wherein the one or more symptoms associated with a comorbidity of secondary Raynaud's syndrome is selected from the group consisting of: tooth pain, muscle pain, back pain, joint pain, headache, skin ulcer pain, neuropathic pain, gastrointestinal symptoms (e.g., gastrointestinal pain), or any combination thereof.

In some embodiments, after a first administration of cilnidipine or a pharmaceutically acceptable salt thereof, the severity and/or duration of the one or more symptoms is reduced in the subject.

In some embodiments, the cilnidipine or a pharmaceutically acceptable salt thereof is administered to the subject once daily for at least five days.

In some embodiments, the cilnidipine or a pharmaceutically acceptable salt thereof is administered to the subject in the morning.

In some embodiments, the cilnidipine or a pharmaceutically acceptable salt thereof is formulated as a liquid suspension or as an oral thin film.

In some embodiments, after administering the cilnidipine or a pharmaceutically acceptable salt thereof, the frequency of emergent medical care, systemic sclerosis related medical facility visits, hospitalization, or any combination thereof of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof to the subject, the frequency of emergent medical care of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof to the subject, the frequency of systemic sclerosis related medical facility visits of the subject are reduced in comparison to before administering the cilnidipine or a pharmaceutically acceptable salt thereof and the tadalafil or a pharmaceutically acceptable salt thereof to the subject.

In some embodiments, after administering the cilnidipine or a pharmaceutically acceptable salt thereof to the subject, the frequency of hospitalization of the subject are reduced in comparison to before administering the cilnidipine or a pharmaceutically acceptable salt thereof and the tadalafil or a pharmaceutically acceptable salt thereof to the subject.

In some embodiments, reducing the frequency and/or severity of one or more symptoms associated with systemic sclerosis comprises measuring or assessing an improvement in the Scleroderma Health Assessment Questionnaire (SHAQ®) in the subject after administering the cilnidipine or a pharmaceutically acceptable salt thereof to the subject.

In some embodiments, an improvement of at least 30% in overall systemic sclerosis disease severity is measured or assessed in the visual analog scale (VAS) of overall systemic sclerosis disease severity in the subject after administering the cilnidipine or a pharmaceutically acceptable salt thereof to the subject.

In some embodiments, reducing the frequency and/or severity of one or more symptoms of systemic sclerosis comprises reducing the frequency of one or more symptoms of systemic sclerosis. In some embodiments, reducing the frequency and/or severity of one or more symptoms of systemic sclerosis comprises reducing the severity of one or more symptoms of systemic sclerosis. In some embodiments, reducing the frequency and/or severity of one or more symptoms of systemic sclerosis comprises reducing the frequency and severity of one or more symptoms of systemic sclerosis.

In some embodiments, the one or more symptoms of scleroderma are selected from the group consisting of: skin ulcers, kidney dysfunction, lung dysfunction, cardiac dysfunction, calcinosis, pain, fibrotic changes in the skin, hardening and/or tightening of regions of skin, shiny skin, restricted movement due to hardness in skin, hair loss, white lumps under the skin due to calcium deposition, exaggerated responses to cold temperatures and emotional stress, numbness in finger and/or toe, pain in finger and/or toe, changes in color in finger and/or toe, digestive system, acid reflux, restricted movement of food through the digestive tract, malnutrition, fatigue, anxiety, telangiectasia, and any combination thereof.

In some embodiments, the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof.

In some embodiments, the symptom is skin ulcers.

In some embodiments, the symptom is kidney dysfunction. In some embodiments, the kidney dysfunction is reduced glomerular filtration. In some embodiments, the method comprises measuring an increase in glomerular filtration in the subject after administering the cilnidipine or a pharmaceutically acceptable salt thereof and optionally the tadalafil or a pharmaceutically acceptable salt thereof.

In some embodiments, the symptom is lung dysfunction.

In some embodiments, the symptom is cardiac dysfunction.

In some embodiments, the symptom is calcinosis.

In some embodiments, the symptom is pain.

In some embodiments, the symptom is fibrotic changes in the skin.

In some embodiments, the symptom is hardening and/or tightening of regions of skin.

In some embodiments, the symptom is shiny skin.

In some embodiments, the symptom is restricted movement due to hardness in skin.

In some embodiments, the symptom is hair loss.

In some embodiments, the symptom is white lumps under the skin due to calcium deposition.

In some embodiments, the symptom is exaggerated responses to cold temperatures.

In some embodiments, the symptom is exaggerated responses to emotional stress.

In some embodiments, the symptom is numbness in finger and/or toe.

In some embodiments, the symptom is pain in finger and/or toe.

In some embodiments, the symptom is changes in color in finger and/or toe.

In some embodiments, the symptom is digestive system dysfunction. In some embodiments, the digestive system dysfunction comprises heartburn, difficulty swallowing, bloating, diarrhea, constipation, fecal incontinence, or any combination thereof.

In some embodiments, the symptom is acid reflux.

In some embodiments, the symptom is restricted movement of food through the digestive tract.

In some embodiments, the symptom is malnutrition.

In some embodiments, the symptom is fatigue.

In some embodiments, the symptom is anxiety.

In some embodiments, the symptom is telangiectasia.

In some embodiments, the method comprises reducing fibrosis in the subject. In some embodiments, reducing fibrosis comprises reducing formation of collagen and extracellular matrix proteins in the subject. In some embodiments, the collagen is formed by fibroblasts. In some embodiments, the fibrosis is renal fibrosis or myocardial fibrosis.

In some embodiments, the subject has skin ulcers. In some embodiments, the skin ulcers are digital ulcers. In some embodiments, the digital ulcers are finger ulcers. In some embodiments, after administering the cilnidipine or a pharmaceutically acceptable salt thereof and optionally tadalafil or a pharmaceutically acceptable salt thereof, the number and/or severity of the skin ulcers is reduced.

In some embodiments, the subject has (e.g., is identified or diagnosed as having) Raynaud's syndrome. In some embodiments, the Raynaud's syndrome is selected from the group consisting of: primary Raynaud's syndrome; secondary Raynaud's syndrome; Raynaud's syndrome of the nipple, nose, ear, penis, tongue, and/or any alar circulatory region. In some embodiments, the Raynaud's syndrome is primary Raynaud's syndrome. In some embodiments, the Raynaud's syndrome is secondary Raynaud's syndrome.

In some embodiments, vasoconstriction in the subject is reduced after administering cilnidipine or a pharmaceutically acceptable salt thereof and optionally tadalafil or a pharmaceutically acceptable salt thereof to the subject. In some embodiments, the vasoconstriction comprises vasoconstriction of a body part, and the temperature of the vasoconstricted body part is lower than the subject's body temperature. In some embodiments, the body part is a finger of the subject.

In some embodiments, before administering cilnidipine or a pharmaceutically acceptable salt thereof and optionally tadalafil or a pharmaceutically acceptable salt thereof to the subject, the subject is diagnosed with scleroderma. In some embodiments, before administering cilnidipine or a pharmaceutically acceptable salt thereof and optionally tadalafil or a pharmaceutically acceptable salt thereof to the subject, the subject is diagnosed with Raynaud's syndrome (e.g., secondary Raynaud's syndrome). In some embodiments, before administering cilnidipine or a pharmaceutically acceptable salt thereof and optionally tadalafil or a pharmaceutically acceptable salt thereof to the subject, the subject is diagnosed with Raynaud's syndrome (e.g., secondary Raynaud's syndrome) and scleroderma.

In some embodiments, the method comprises reducing fibrosis in the subject. In some embodiments, reducing fibrosis comprises reducing formation of collagen and extracellular matrix proteins in the subject. In some embodiments, the collagen is formed by fibroblasts. In some embodiments, the fibrosis is renal fibrosis or myocardial fibrosis.

In some embodiments, reducing the frequency, severity, and/or duration (e.g., the frequency and/or severity; or the severity and/or duration; or the frequency) of one or more symptoms comprises reducing the frequency, severity, and/or duration of the symptoms when compared to (1) the frequency, severity, and/or duration of the one or more symptoms in the subject before start of the treatment (e.g., before administration of the cilnidipine or a pharmaceutically acceptable salt thereof and optionally tadalafil or a pharmaceutically acceptable salt thereof, and wherein the frequency, severity, and/or duration of the one or more symptoms before administration of the cilnidipine or a pharmaceutically acceptable salt thereof and optionally tadalafil or a pharmaceutically acceptable salt thereof can, for example, be evaluated by a single measurement or assessment, or an average of a plurality of measurements or assessments taken, e.g., over the course of a 2 week period, a 7 day period, a 6 day period, a 5 day period, a 4 day period, a 3 day period, a 2 day period, or a 1 day period (e.g., a 7 day period)), wherein, for example, the reduction in frequency, severity, and/or duration of the symptoms is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 6 weeks, 2 months, 3 months, or 1 year of treatment); and/or (2) the frequency, severity, and/or duration of the one or more symptoms experienced by a subject after the subject was administered a placebo. In some embodiments, the reduction in frequency, severity, and/or duration of the symptoms is greatest within 2 days (e.g., within 1.5 days, within 1 day, within 20 hours, within 16 hours, within 12 hours, within 8 hours, within 6 hours, within 4 hours, within 2 hours, or within 1 hour after administration of the cilnidipine or a pharmaceutically acceptable salt thereof and optionally tadalafil or a pharmaceutically acceptable salt thereof. In some embodiments, the reduction in frequency, severity, and/or duration of the symptoms is greatest within 8 hours after administration of the cilnidipine or a pharmaceutically acceptable salt thereof and optionally tadalafil or a pharmaceutically acceptable salt thereof. In some embodiments, the reduction in the frequency, severity, and/or duration of one or more symptoms of Raynaud's syndrome and/or scleroderma can be measured by one or more patient assessments, examples of which are discussed herein.

In some embodiments, after administering the cilnidipine or a pharmaceutically acceptable salt thereof and optionally tadalafil or a pharmaceutically acceptable salt thereof, frequency (e.g., the mean weekly frequency) of one or more symptoms associated with the comorbidity of Raynaud's syndrome in the subject is reduced by at least 5%, for example, at least 10%, at least 20%, at least 25%, at least 30%, at least 35%, at least 40%, at least 45%, at least 50%, at least 55%, at least 60%, at least 70%, at least 80%, at least 90%, or at least 95% (e.g., at least 25%; e.g., at least 40%; e.g., at least 45%). In some embodiments, the frequency of one or more symptoms associated with the comorbidity of Raynaud's syndrome in the subject is reduced by at least 25%. In some embodiments, the subject experiences no symptoms after administering the cilnidipine or a pharmaceutically acceptable salt thereof and optionally tadalafil or a pharmaceutically acceptable salt thereof.

In some embodiments, reducing the frequency, severity, and/or duration of one or more symptoms of scleroderma comprises measuring or assessing an improvement in the Scleroderma Health Assessment Questionnaire (SHAQ®) in the subject after administering the cilnidipine or a pharmaceutically acceptable salt thereof and optionally tadalafil or a pharmaceutically acceptable salt thereof to the subject. In some embodiments, an improvement in the Scleroderma Health Assessment Questionnaire (SHAQ®) is measured in the subject after administering the cilnidipine or a pharmaceutically acceptable salt thereof and optionally tadalafil or a pharmaceutically acceptable salt thereof to the subject. In some embodiments, an improvement in the Scleroderma Health Assessment Questionnaire comprises an improvement in at least one (e.g., 1 to 8, 2 to 6, 2 to 4, 4 to 6, 6 to 8, 1, 2, 3, 4, 5, 6, 7, or 8) of 1) dressing and grooming, 2) arising, 3) eating, 4) walking, 5) hygiene, 6) reach, 7) grip, and 8) common daily activities in the subject. Information on the Scleroderma Health Assessment Questionnaire (SHAQ®) can be found in the Examples and at Steen V D, Medsger T A Jr. The value of the Health Assessment Questionnaire and special patient-generated scales to demonstrate change in systemic sclerosis patients over time. Arthritis Rheum. 1997 November; 40(11):1984-91 and Poole J L, Steen V D. The use of the Health Assessment Questionnaire (HAQ) to determine physical disability in systemic sclerosis. Arthritis Care Res. 1991 March; 4(1):27-31, each of which is incorporated by reference herein in its entirety. In some embodiments, reducing the severity of one or more symptoms of scleroderma comprises measuring or assessing an improvement in overall scleroderma disease severity. In some embodiments, an improvement in overall scleroderma disease severity is measured or assessed in the subject. In some embodiments, an improvement (e.g., at least 10%, at least 20%, at least 30%, at least 40%, at least 50%, at least 60%, or about 63% improvement) in overall scleroderma disease severity is measured or assessed in the visual analog scale of overall scleroderma disease severity in the subject. In some embodiments, an improvement of at least 30% in overall scleroderma disease severity is measured or assessed in the visual analog scale (VAS) of overall disease severity in the subject.

In some embodiments, reducing the severity of one or more symptoms of the comorbidity of Raynaud's syndrome comprises measuring or assessing an improvement (e.g., reduction) in the Raynaud's condition scale (RCS) in the subject after administering cilnidipine or a pharmaceutically acceptable salt thereof and optionally tadalafil or a pharmaceutically acceptable salt thereof to the subject. In some embodiments, an improvement (e.g., reduction) in the Raynaud's condition scale (RCS) is measured in the subject after administering cilnidipine or a pharmaceutically acceptable salt thereof and optionally tadalafil or a pharmaceutically acceptable salt thereof to the subject. In some embodiments, an improvement (e.g., reduction) in the Raynaud's condition scale (RCS) is a reduction in one score or the average (e.g., daily average) of a plurality of scores measured after administering the cilnidipine or a pharmaceutically acceptable salt thereof and optionally tadalafil or a pharmaceutically acceptable salt thereof to the subject relative to one score or the average (e.g., daily average) of a plurality of scores measured before administering the cilnidipine or a pharmaceutically acceptable salt thereof and optionally tadalafil or a pharmaceutically acceptable salt thereof to the subject. In some embodiments, when a plurality of scores is measured before and after administration, they are taken over the same period of time (e.g., the same number of days). In some embodiments, the period of time is 2 weeks, 7 days, 6 days, 5 days, 4 days, 3 days, 2 days, or 1 day (e.g., 7 day). In some embodiments, a daily average of a plurality of scores is the sum of the plurality of scores divided by the number of days during which the scores were obtained. In some embodiments, the reduction in the Raynaud's condition scale is at least about 2% (e.g., at least about 4%, 5%, 8%, 10%, 20%, 25%, 27%, 30%, 40%, 45%, 50%, 70%, 90%, 95%) after administering the cilnidipine or a pharmaceutically acceptable salt thereof and optionally tadalafil or a pharmaceutically acceptable salt thereof to the subject. In some embodiments, the reduction in the Raynaud's condition scale is at least about 20% after administering the cilnidipine or a pharmaceutically acceptable salt thereof and optionally tadalafil or a pharmaceutically acceptable salt thereof to the subject. In some embodiments, the reduction in the Raynaud's condition scale is at least about 25% after administering the cilnidipine or a pharmaceutically acceptable salt thereof and optionally tadalafil or a pharmaceutically acceptable salt thereof to the subject. In some embodiments, the reduction in the Raynaud's condition scale is at least about 27% after administering the cilnidipine or a pharmaceutically acceptable salt thereof and optionally tadalafil or a pharmaceutically acceptable salt thereof to the subject. In some embodiments, the reduction in the Raynaud's condition scale is at least about 45% after administering the cilnidipine or a pharmaceutically acceptable salt thereof and optionally tadalafil or a pharmaceutically acceptable salt thereof to the subject. Information on the Raynaud's condition scale can be found in the Examples and at Black C M, Halkier-Sorensen L, Belch J J et al. Oral iloprost in Raynaud's phenomenon secondary to systemic sclerosis: a multicentre, placebo-controlled, dose-comparison study. Br J Rheumatol 1998; 37:952-60, which is incorporated by reference herein in its entirety.

In some embodiments, the subject has skin ulcerations. In some embodiments, the skin ulcerations are digital ulcerations. In some embodiments, after administering cilnidipine or a pharmaceutically acceptable salt thereof and optionally tadalafil or a pharmaceutically acceptable salt thereof to the subject, the number and/or severity of the skin ulcerations (e.g., digital ulcerations) is reduced. In some embodiments, after administering cilnidipine or a pharmaceutically acceptable salt thereof and optionally tadalafil or a pharmaceutically acceptable salt thereof to the subject, one or more skin ulcerations (e.g., digital ulcerations) in the subject exhibits healing. In some embodiments, the subject exhibits an improvement in skin ulcer (e.g., digital ulcer) severity after administering cilnidipine or a pharmaceutically acceptable salt thereof and optionally tadalafil or a pharmaceutically acceptable salt thereof to the subject. In some embodiments, the improvement comprises a reduction in a score provided by the visual analog scale (VAS). In some embodiments, one score or an average of a plurality of scores is reduced by at least about 0.25% (e.g., at least about 0.5%, at least about fELF11%, at least about 2%, at least about 3%, at least about 4%, at least about 5%, at least about 8%, at least about 10%, at least about 15%, at least about 20%, at least about 25%, at least about 30%, at least about 40%, at least about 50%, at least about 60%, at least about 70%, at least about 80%, at least about 90%, or at least about 95%) after administering cilnidipine or a pharmaceutically acceptable salt thereof and optionally tadalafil or a pharmaceutically acceptable salt thereof relative to one score or an average of a plurality of scores taken before administering cilnidipine or a pharmaceutically acceptable salt thereof and optionally tadalafil or a pharmaceutically acceptable salt thereof to the subject. In some embodiments, an improvement (e.g., at least 5%, at least 10%, at least 15%, at least 20%, at least 25%, at least 30%, at least 32%, or about 35% improvement) in the digital ulcer visual analog scale (VAS) is measured or assessed in the subject. In some embodiments, an at least 15% improvement in the digital ulcer visual analog scale (VAS) is measured or assessed in the subject. In some embodiments, an at least 32% improvement in the digital ulcer visual analog scale (VAS) is measured or assessed in the subject. In some embodiments, when a plurality of scores are obtained before and after administration, they are taken over the same period of time (e.g., the same number of days). In some embodiments, after administering cilnidipine or a pharmaceutically acceptable salt thereof and optionally tadalafil or a pharmaceutically acceptable salt thereof to the subject, at least one (e.g., at least two, at least three, at least 5, at least 10, or at least 15) digital ulcers fully heal. Further information on the measurement of digital ulcer severity using VAS is in the Examples.

In some embodiments, an improvement (e.g., at least 5%, at least 10%, at least 15%, at least 20%, at least 25%, at least 30%, at least 35%, at least 40%, at least 45%, or about 49% improvement) in the Raynaud's visual analog scale (VAS) is measured or assessed in the subject. In some embodiments, an at least 30% improvement in the Raynaud's visual analog scale (VAS) is measured or assessed in the subject.

In some embodiments, an improvement in breathing is determined in the subject as measured by increased oxygen saturation in the blood of the subject. In some embodiments, an improvement in breathing (e.g., at least 10%, at least 20%, at least 30%, at least 40%, at least 50%, at least 60%, or about 67% improvement) is determined in the subject as measured by the breathing visual analog scale (VAS).

In some embodiments, an improvement in the Standard Disability Index (e.g., at least 10%, at least 20%, at least 25%, at least 30%, at least 40%, at least 50%, or about 50% improvement) is measured or assessed in the subject. Further information on the Standard Disability Index can be found in the Examples.

In some embodiments, an improvement in the Alternative Disability Index (e.g., at least 10%, at least 20%, at least 30%, at least 40%, or about 42% improvement) is measured or assessed in the subject. Further information on the Alternative Disability Index can be found in the Examples.

In some embodiments, a reduction in the Raynaud's severity visual analog scale (VAS) 0-10 cm is measured in the subject after administering the cilnidipine or a pharmaceutically acceptable salt thereof and optionally tadalafil or a pharmaceutically acceptable salt thereof to the subject. In some embodiments, a reduction in a score provided by the visual analog scale is measured in the subject. In some embodiments, the reduction in the score provided by the visual analog scale comprises a reduction in a single score or the average of a plurality of scores measured after administering the cilnidipine or a pharmaceutically acceptable salt thereof and optionally tadalafil or a pharmaceutically acceptable salt thereof to the subject relative to a single score or the average of a plurality of scores measured before administering the cilnidipine or a pharmaceutically acceptable salt thereof and optionally tadalafil or a pharmaceutically acceptable salt thereof to the subject. In some embodiments, when a plurality of scores is obtained before and after administration, they are taken over the same period of time (e.g., the same number of days). In some embodiments, the score is reduced by at least 5%, at least 10%, at least 15%, at least 20%, at least 25%, at least 30%, at least 35%, at least 40%, at least 45%, at least 50%, at least 60%, at least 70%, at least 80%, at least 90%, at least 95%, or at least 98%. In some embodiments, the score is reduced by at least 20%.

In some embodiments, the method comprises reducing a sensation of burning pain, paresthesia, dysesthesia, hypoesthesia, or hyperesthesia (e.g., burning pain) in the subject. A sensation of burning pain can be measured using one or more of the following: the Galer neuropathic pain scale, the ID pain Scale, NPQ, PainDETECT, LANS S, DN4 scales, and/or the Standardized Evaluation of Pain (StEP) tool. See, for example, Cruccu G, Truini A. Tools for assessing neuropathic pain. PLoS Med. 2009; 6(4):e1000045. doi:10.1371/journal.pmed.1000045, which is incorporated by reference herein in its entirety.

In some embodiments, reducing the severity of one or more symptoms of the comorbidity of Raynaud's syndrome comprises measuring a reduction in the Raynaud's severity scale (RSS) after administering the cilnidipine or a pharmaceutically acceptable salt thereof and optionally tadalafil or a pharmaceutically acceptable salt thereof to the subject. In some embodiments, a reduction in the Raynaud's severity scale (RSS) is measured in the subject after administering the cilnidipine or a pharmaceutically acceptable salt thereof and optionally tadalafil or a pharmaceutically acceptable salt thereof to the subject. In some embodiments, the reduction is a reduction in one measurement or the average of a plurality of measurements taken after administering the cilnidipine or a pharmaceutically acceptable salt thereof and optionally tadalafil or a pharmaceutically acceptable salt thereof to the subject relative to one measurement or the average of a plurality of measurements taken before administering the cilnidipine or a pharmaceutically acceptable salt thereof and optionally tadalafil or a pharmaceutically acceptable salt thereof to the subject. In some embodiments, when a plurality of measurements is taken before and after administration, they are taken over the same period of time (e.g., the same number of days). For example, a reduction of at least about 2% (e.g., at least about 4%, 5%, 8%, 10%, 20%, 30%, 40%, 50%, 70%, 90%, 95%) in the RSS is measured in the subject after administering the cilnidipine or a pharmaceutically acceptable salt thereof and optionally tadalafil or a pharmaceutically acceptable salt thereof to the subject. In some embodiments, a reduction of at least 10% is measured or assessed in the subject after administering the cilnidipine or a pharmaceutically acceptable salt thereof and optionally tadalafil or a pharmaceutically acceptable salt thereof to the subject. Information on the Raynaud's severity scale can be found at Wigley F M, Wise R A, Seibold J R et al. Intravenous iloprost infusion in patients with Raynaud phenomenon secondary to systemic sclerosis. A multicenter, placebo-controlled, double-blind study. Ann Intern Med 1994; 120:199-206, which is incorporated by reference herein in its entirety.

In some embodiments, a reduction in the average pain score (e.g., average weekly pain score) is measured in the subject after administering the cilnidipine or a pharmaceutically acceptable salt thereof and optionally tadalafil or a pharmaceutically acceptable salt thereof to the subject. For example, a reduction of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. In some embodiments, a reduction in the average pain score is about 10% in the subject after administering the cilnidipine or a pharmaceutically acceptable salt thereof and optionally tadalafil or a pharmaceutically acceptable salt thereof to the subject. In some embodiments, a reduction in the pain score is a reduction in one score or the average of a plurality of scores measured after administering the cilnidipine or a pharmaceutically acceptable salt thereof and optionally tadalafil or a pharmaceutically acceptable salt thereof to the subject relative to one score or the average of a plurality of scores measured before administering the cilnidipine or a pharmaceutically acceptable salt thereof and optionally tadalafil or a pharmaceutically acceptable salt thereof to the subject. In some embodiments, when a plurality of scores are obtained before and after administration, they are taken over the same period of time (e.g., the same number of days). In some embodiments, the time period is 2 weeks, 7 days, 6 days, 5 days, 4 days, 3 days, 2 days, or 1 day (e.g., 7 day). Various scales used to measure pain include the Galer neuropathic pain scale, the Likert pain score, the ID pain Scale, NPQ, PainDETECT, LANS S, DN4 scales, and/or the Standardized Evaluation of Pain (StEP) tool. See, for example, the Examples or Cruccu G, Truini A. Tools for assessing neuropathic pain. PLoS Med. 2009; 6(4):e1000045. doi:10.1371/journal.pmed.1000045, which is incorporated by reference herein in its entirety.

In some embodiments, an increase in the temperature of a body part is measured in the subject after administering the cilnidipine or a pharmaceutically acceptable salt thereof and optionally tadalafil or a pharmaceutically acceptable salt thereof to the subject. For example, an increase of at least about 0.5% (e.g., at least about 1%, 2%, 3%, 4%, 5%, 8%, 10%, 12%, 15%, or 20%) in the temperature of the body part is measured in the subject after administering the cilnidipine or a pharmaceutically acceptable salt thereof and optionally tadalafil or a pharmaceutically acceptable salt thereof to the subject. In some embodiments, the increase in the temperature of the body part is an increase in one measurement or the average of a plurality of measurements taken after administering the cilnidipine or a pharmaceutically acceptable salt thereof and optionally tadalafil or a pharmaceutically acceptable salt thereof relative to one measurement or the average of a plurality of measurements taken before administration. In some embodiments, when a plurality of measurements is taken before and after administration, they are taken over the same period of time (e.g., the same number of days). In some embodiments, the increase in temperature is measured by thermography. In some embodiments, the body part is a finger (e.g., an index finger, middle finger, or ring finger (e.g., an index finger)).

In some embodiments, an improvement in the SF-12 index of functional wellbeing is measured in the subject after administering the cilnidipine or a pharmaceutically acceptable salt thereof and optionally tadalafil or a pharmaceutically acceptable salt thereof to the subject. In some embodiments, the improvement is an improvement in one score or the average of a plurality of scores taken after administering cilnidipine or a pharmaceutically acceptable salt thereof and optionally tadalafil or a pharmaceutically acceptable salt thereof relative to one score or the average of a plurality of scores taken before administration. In some embodiments, when a plurality of scores are taken before and after administration, they are taken over the same period of time (e.g., the same number of days). In some embodiments, an improvement of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. Information on the SF-12 index of functional wellbeing can be found at https://www.physio-pedia.com/12-Item_Short_Form_Survey_(SF-12), which is incorporated by reference herein in its entirety.

In some embodiments, an improvement (i.e., an increase) in the Reactive Hyperemia Index (LnRHI) as measured by Endo PAT is measured in the subject after administering the cilnidipine or a pharmaceutically acceptable salt thereof and optionally tadalafil or a pharmaceutically acceptable salt thereof to the subject. In some embodiments, the improvement is an improvement in one score or the average of a plurality of scores taken after administering the cilnidipine or a pharmaceutically acceptable salt thereof and optionally tadalafil or a pharmaceutically acceptable salt thereof relative to one score or the average of a plurality of scores taken before administration. In some embodiments, when a plurality of scores are taken before and after administration, they are taken over the same period of time (e.g., the same number of days). For example, an increase of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. In some embodiments, an improvement in the Reactive Hyperemia Index (LnRHI) is an improvement in the average of a plurality of values measured after administering the cilnidipine or a pharmaceutically acceptable salt thereof and optionally tadalafil or a pharmaceutically acceptable salt thereof to the subject relative to the average of a plurality of values measured before administering the cilnidipine or a pharmaceutically acceptable salt thereof and optionally tadalafil or a pharmaceutically acceptable salt thereof to the subject taken over the same time period. In some embodiments, the time period is 2 weeks, 7 days, 6 days, 5 days, 4 days, 3 days, 2 days, or 1 day (e.g., 7 days). In some embodiments, the subject exhibits a reactive hyperemia index of less than 0.51 before administering the cilnidipine or a pharmaceutically acceptable salt thereof and optionally tadalafil or a pharmaceutically acceptable salt thereof to the subject and a reactive hyperemia index of at least 0.51 (e.g., 0.51 to 0.7; or at least 0.71) after administering the cilnidipine or a pharmaceutically acceptable salt thereof and optionally tadalafil or a pharmaceutically acceptable salt thereof to the subject. In some embodiments, the subject exhibits a reactive hyperemia index of 0.51 to 0.7 before administering the cilnidipine or a pharmaceutically acceptable salt thereof and optionally tadalafil or a pharmaceutically acceptable salt thereof to the subject and a reactive hyperemia index of at least 0.71 after administering the cilnidipine or a pharmaceutically acceptable salt thereof and optionally tadalafil or a pharmaceutically acceptable salt thereof to the subject.

In some embodiments, an improvement in endothelial function as measured by Endo PAT is measured in the subject after administering the cilnidipine or a pharmaceutically acceptable salt thereof and optionally tadalafil or a pharmaceutically acceptable salt thereof to the subject. In some embodiments, the improvement in endothelial function as measured by Endo PAT is an improvement in one measurement or an average of plurality of measurements taken after administering the cilnidipine or a pharmaceutically acceptable salt thereof and optionally tadalafil or a pharmaceutically acceptable salt thereof to the subject relative to one measurement or an average of plurality of measurements taken before administering the cilnidipine or a pharmaceutically acceptable salt thereof and optionally tadalafil or a pharmaceutically acceptable salt thereof to the subject. For example, an improvement of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. In some embodiments, nitric oxide levels in the endothelium increase after administering the cilnidipine or a pharmaceutically acceptable salt thereof and optionally tadalafil or a pharmaceutically acceptable salt thereof to the subject as measured by Endo PAT in the subject. In some embodiments, the increase in nitric oxide levels in the endothelium as measured by Endo PAT is an increase in one measurement or an average of plurality of measurements taken after administering the cilnidipine or a pharmaceutically acceptable salt thereof and optionally tadalafil or a pharmaceutically acceptable salt thereof to the subject relative to one measurement or an average of plurality of measurements taken before administering the cilnidipine or a pharmaceutically acceptable salt thereof and optionally tadalafil or a pharmaceutically acceptable salt thereof to the subject. For example, an increase of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. Information on the Reactive Hyperemia Index can be found at https://study.com/academy/lesson/reactive-hyperemia-definition-test.html, which is incorporated by reference herein in its entirety. Information on Endo PAT can be found at https://www.aimil.com/products/endopat#::text=The%20EndoPAT%E2%84%A2%20is%20the%2only%20FDA%20approved%20diagnostic,in%20peripheral%20arterioles%20in%20response%20to%20oxidative%20stress., which is incorporated by reference herein in its entirety.

In some embodiments, an improvement (i.e., a decrease) in the University of California Los Angeles Scleroderma Clinical Trials Consortium Gastrointestinal Tract 2.0 (UCLA SCTC GIT 2.0) questionnaire is observed in the subject after administering the cilnidipine or a pharmaceutically acceptable salt thereof and optionally tadalafil or a pharmaceutically acceptable salt thereof to the subject. In some embodiments, a decrease in the UCLA SCTC GIT 2.0 questionnaire is a decrease in one score or the average of a plurality of total scores measured after administering the cilnidipine or a pharmaceutically acceptable salt thereof and optionally tadalafil or a pharmaceutically acceptable salt thereof to the subject relative to one score or the average of a plurality of total scores measured before administering the cilnidipine or a pharmaceutically acceptable salt thereof and optionally tadalafil or a pharmaceutically acceptable salt thereof to the subject. In some embodiments, when a plurality of scores is taken before and after administration, they are taken over the same period of time (e.g., the same number of days). In some embodiments, the time period is 2 weeks, 7 days, 6 days, 5 days, 4 days, 3 days, 2 days, or 1 day (e.g., 7 day). In some embodiments, a decrease of at least about 2% (e.g., at least about 4%, 5%, 8%, 10%, 20%, 30%, 40%, 50%, 70%, 90%, 95%) is measured in the subject after administering the cilnidipine or a pharmaceutically acceptable salt thereof and optionally tadalafil or a pharmaceutically acceptable salt thereof to the subject. More information on the UCLA SCTC GIT 2.0 questionnaire, including how the total score is obtained, can be found in the Examples section.

In some embodiments, an improvement in at least one question (e.g., 2, 3, 4, 5, 6, 7, 8, 9, or 10 questions) in the Assessment of Systemic sclerosis-associated Raynaud's Phenomenon (ASRAP) is observed in the subject after administering the cilnidipine or a pharmaceutically acceptable salt thereof and optionally tadalafil or a pharmaceutically acceptable salt thereof to the subject relative to before administering the cilnidipine or a pharmaceutically acceptable salt thereof and optionally tadalafil or a pharmaceutically acceptable salt thereof to the subject. In some embodiments, the improvement in a question is an improvement of at least one degree. An improvement of a degree is defined as an improvement from “very much/a lot” to “quite a bit”, “quite a bit” to “somewhat”, “somewhat” to “a little bit”, or “a little bit” to “not at all”. In some embodiments, the improvement in a questions is an improvement of two degrees, three degrees, or four degrees. More information on the Assessment of Systemic sclerosis-associated Raynaud's Phenomenon (ASRAP), can be found in the Examples section and Pauling et. al. American College of Rheumatology Convergence 2021, Abstract Number 401 (https://acrabstracts.org/abstract/item-reduction-for-the-assessment-of-systemic-sclerosis-associated-raynauds-phenomenon-asrap-questionnaire-using-data-from-the-international-multicentre-asrap-validation-study/), which is incorporated by reference herein in its entirety.

In some embodiments, the cilnidipine or a pharmaceutically acceptable salt thereof is administered in the morning. In some embodiments, the tadalafil or a pharmaceutically acceptable salt thereof or a pharmaceutically acceptable salt thereof is administered in the morning. In some embodiments, the cilnidipine or a pharmaceutically acceptable salt thereof and optionally tadalafil or a pharmaceutically acceptable salt thereof or a pharmaceutically acceptable salt thereof are administered in the morning. In some embodiments, administering the cilnidipine or a pharmaceutically acceptable salt thereof and/or tadalafil or a pharmaceutically acceptable salt thereof or a pharmaceutically acceptable salt thereof in the morning comprises administering the cilnidipine or a pharmaceutically acceptable salt thereof and/or tadalafil or a pharmaceutically acceptable salt thereof or a pharmaceutically acceptable salt thereof between 5 AM and 11:59 AM (e.g., between 6 AM and 11 AM, between 6 AM and 9 AM, between 7 AM and 10 AM, between 8 AM and 11 AM, between 8 AM and 10 AM, or between 8 AM and 10 AM).

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the cilnidipine or a pharmaceutically acceptable salt thereof and optionally tadalafil or a pharmaceutically acceptable salt thereof are administered to the subject for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, at least 5 days, at least 1 week, at least 2 weeks, at least 3 weeks, at least 4 weeks, at least 3 months, at least 6 months, at least 1 year, at least 2 years, at least 5 years, at least 10 years, at least 15 years, at least 20 years, at least 30 years, or for the remainder of the lifespan of the subject.

In some embodiments, the cilnidipine or a pharmaceutically acceptable salt thereof and tadalafil or a pharmaceutically acceptable salt thereof are administered separately, sequentially, or simultaneously. In some embodiments, the cilnidipine or a pharmaceutically acceptable salt thereof and tadalafil or a pharmaceutically acceptable salt thereof are administered separately. In some embodiments, the cilnidipine or a pharmaceutically acceptable salt thereof and tadalafil or a pharmaceutically acceptable salt thereof are administered sequentially. In some embodiments, the cilnidipine or a pharmaceutically acceptable salt thereof and tadalafil or a pharmaceutically acceptable salt thereof are administered simultaneously. In some embodiments, the cilnidipine or a pharmaceutically acceptable salt thereof and tadalafil or a pharmaceutically acceptable salt thereof are administered simultaneously as a fixed dosage form.

In some embodiments, after administering cilnidipine or a pharmaceutically acceptable salt thereof and optionally tadalafil or a pharmaceutically acceptable salt thereof to the subject, the subject experiences gastrointestinal symptoms that are ameliorated by the consumption of food prior to administering the cilnidipine or a pharmaceutically acceptable salt thereof and optionally tadalafil or a pharmaceutically acceptable salt thereof to the subject. In some embodiments, the subject consumes food up to about 6 hours before administering the cilnidipine or a pharmaceutically acceptable salt thereof and optionally tadalafil or a pharmaceutically acceptable salt thereof to the subject. For example, the subject consumes food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the cilnidipine or a pharmaceutically acceptable salt thereof and optionally tadalafil or a pharmaceutically acceptable salt thereof to the subject. For example, the subject consumes food concurrently with administering the cilnidipine or a pharmaceutically acceptable salt thereof and optionally tadalafil or a pharmaceutically acceptable salt thereof to the subject.

It is understood that cilnidipine or a pharmaceutically acceptable salt thereof may decrease the blood pressure of subjects that are hypertensive. As such, it may be beneficial to administer an agent that increases blood pressure in combination with the cilnidipine or a pharmaceutically acceptable salt thereof and optionally tadalafil or a pharmaceutically acceptable salt thereof. In some embodiments of the methods disclosed herein, the method further comprises administering to the subject a therapeutically effective amount of an agent that increases blood pressure. In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines. In some embodiments, the blood pressure of the subject before and after administering the cilnidipine or a pharmaceutically acceptable salt thereof, tadalafil or a pharmaceutically acceptable salt thereof, and the agent that increases blood pressure is substantially the same.

In some embodiments, the method comprises administering at least one additional therapeutic agent to the subject. The at least one additional therapeutic agent can be administered simultaneously, separately, sequentially, or in combination with the cilnidipine or a pharmaceutically acceptable salt thereof and optionally tadalafil or a pharmaceutically acceptable salt thereof. Non-limiting examples of additional therapeutic agents include calcium channel blockers, sodium channel blockers (e.g., Nav 1.7 sodium channel blocker), agents that increase blood pressure, and therapeutic agents that relieve pain.

In some embodiments, the at least one additional therapeutic agent is selected from the group consisting of: riociguat, amlodipine, nifedipine, nicardipine, conotoxins, cadmium, caroverine, gabapentin, levetiracetam, lamotrigine, NP078585, pregabalin, TROX-1, and ziconotide.

In some embodiments, the at least one additional therapeutic agent is selected from the group consisting of: nifedipine, verapamil hydrochloride, diltiazem hydrochloride, cimetidine, famotidine, nizatidine, ranitidine, esomeprazole, lansoprazole, omeprazole, pantoprazole, rabeprazole, and sucralfate.

In some embodiments, the at least one additional therapeutic agent is selected from the group consisting of: oxycodone, tramadol, Dilaudid, OxyContin, Cymbalta, Fentanyl Transdermal System, acetaminophen/oxycodone, Roxicodone, Ultram, hydromorphone, Percocet, MS Contin, Butrans, morphine, methadone, buprenorphine, duloxetine, fentanyl, Duragesic, Endocet, Roxanol, Kadian, Roxicet, ConZip, Methadose, Oxyfast, Dazidox, Fentora, Irenka, Methadone Diskets, Oramorph SR, Roxicodone Intensol, Xtampza ER, Actiq, Belbuca, ETH-Oxydose, Infumorph, naloxone/pentazocine, Oxaydo, Oxydose, OxyIR, ziconotide, Abstral, Astramorph PF, Buprenex, Dolophine, Duramorph, Duramorph PF, Embeda, Lazanda, MorphaBond ER, morphine/naltrexone, Prialt, RMS, Roxanol-T, Sublimaze, Subsys, Talwin Nx, Magnacet, Nalocet, Narvox, Perloxx, Primlev, Xolox, and Prolate.

In some embodiments, the cilnidipine or a pharmaceutically acceptable salt thereof is formulated to maintain the plasma level of the cilnidipine in the subject at 10% or greater (e.g., 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater) of the peak cilnidipine plasma level for at least 6 hours (e.g., at least 8 hours, at least 12 hours, at least 16 hours, at least 20 hours, at least 24 hours, at least 36 hours, or at least 48 hours) after administering the cilnidipine or a pharmaceutically acceptable salt thereof and optionally tadalafil or a pharmaceutically acceptable salt thereof to the subject. It is understood that the peak cilnidipine plasma level is the highest plasma concentration of the cilnidipine observed in the subject after administering the cilnidipine to the subject.

In some embodiments, a combination of cilnidipine or a pharmaceutically acceptable salt thereof and is administered as a pharmaceutical composition that includes the cilnidipine or a pharmaceutically acceptable salt thereof and optionally tadalafil or a pharmaceutically acceptable salt thereof, and one or more pharmaceutically acceptable excipients, and optionally one or more additional therapeutic agents as described herein.

In some embodiments, the cilnidipine or a pharmaceutically acceptable salt thereof and optionally tadalafil or a pharmaceutically acceptable salt thereof can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, cilnidipine or a pharmaceutically acceptable salt thereof and optionally tadalafil or a pharmaceutically acceptable salt thereof, or a pharmaceutical composition thereof can be administered to a subject orally. In some embodiments, orally administering the cilnidipine or a pharmaceutically acceptable salt thereof and optionally tadalafil or a pharmaceutically acceptable salt thereof comprises sublingually administering the cilnidipine or a pharmaceutically acceptable salt thereof and optionally tadalafil or a pharmaceutically acceptable salt thereof. In some embodiments, the chemical entities described herein or a pharmaceutical composition thereof are suitable for local delivery to the digestive or GI tract by way of oral administration in the form of, e.g., solid, liquid, liquid suspension, an oral thin film, or liquid gel dosage forms. In some embodiments, the oral thin film is a rapidly dissolving thin film formulated for sublingual administration.

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In some embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In some embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### EXEMPLARY EMBODIMENTS

Embodiment 1. A method of reducing the frequency, severity, and/or duration of one or more symptoms associated with a comorbidity of secondary Raynaud's syndrome in a cilnidipine-naïve subject previously determined to have (i) secondary Raynaud's syndrome, and (ii) a comorbidity of secondary Raynaud's syndrome, comprising:
   a. orally administering about 30 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
   b. wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.
Embodiment 2. The method of embodiment 1, wherein after a first administration of cilnidipine or a pharmaceutically acceptable salt thereof and tadalafil or a pharmaceutically acceptable salt thereof, the severity and/or duration of the one or more symptoms is reduced in the subject.
Embodiment 3. The method of embodiment 1, wherein the cilnidipine or a pharmaceutically acceptable salt thereof and the tadalafil or a pharmaceutically acceptable salt thereof are administered to the subject once daily for at least five days.
Embodiment 4. The method of embodiment 1, wherein the comorbidity of secondary Raynaud's syndrome is systemic sclerosis, lupus, rheumatoid arthritis, or any combination thereof.
Embodiment 5. The method of embodiment 1, wherein the cilnidipine or a pharmaceutically acceptable salt thereof and tadalafil or a pharmaceutically acceptable salt thereof are administered as a fixed dosage form.
Embodiment 6. The method of embodiment 1, wherein the cilnidipine or a pharmaceutically acceptable salt thereof and tadalafil or a pharmaceutically acceptable salt thereof are formulated as a liquid suspension or as an oral thin film.
Embodiment 7. The method of embodiment 1, wherein the frequency of the one or more symptoms associated with Raynaud's syndrome in the subject is reduced by at least 25% after administering the cilnidipine or a pharmaceutically acceptable salt thereof and the tadalafil or a pharmaceutically acceptable salt thereof.
Embodiment 8. A method of reducing the frequency, severity, and/or duration of one or more symptoms associated with a comorbidity of secondary Raynaud's syndrome in a subject previously determined to have (i) secondary Raynaud's syndrome, and (ii) a comorbidity of secondary Raynaud's syndrome, comprising:
   a. orally administering about 30 mg of cilnidipine or a pharmaceutically acceptable salt thereof on a free base basis of cilnidipine to the subject;
   b. wherein the one or more symptoms associated with the underlying disease is selected from the group consisting of: tooth pain, muscle pain, back pain, joint pain, headache, skin ulcer pain, neuropathic pain, gastrointestinal symptoms, or any combination thereof.
Embodiment 9. The method of embodiment 8, wherein after a first administration of cilnidipine or a pharmaceutically acceptable salt thereof, the severity and/or duration of the one or more symptoms is reduced in the subject.
Embodiment 10. The method of embodiment 8, wherein the cilnidipine or a pharmaceutically acceptable salt thereof is administered to the subject once daily for at least five days.
Embodiment 11. The method of embodiment 8, wherein the comorbidity of secondary Raynaud's syndrome is systemic sclerosis, lupus, rheumatoid arthritis, or any combination thereof.
Embodiment 12. The method of embodiment 8, wherein the cilnidipine or a pharmaceutically acceptable salt thereof is formulated as a liquid suspension or as an oral thin film.
Embodiment 13. The method of embodiment 8, wherein the frequency of the one or more symptoms associated with Raynaud's syndrome in the subject is reduced by at least 25% after administering the cilnidipine or a pharmaceutically acceptable salt thereof and the tadalafil or a pharmaceutically acceptable salt thereof.
Embodiment 14. A method of reducing the frequency and/or severity of one or more symptoms associated with systemic sclerosis in a cilnidipine-naïve subject previously determined to have systemic sclerosis, comprising:
   a. orally administering about 30 mg of cilnidipine or a pharmaceutically acceptable salt thereof on a free base basis of cilnidipine and about 5 mg of tadalafil or a pharmaceutically acceptable salt thereof on a free base basis of tadalafil to the subject;
   b. wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof;
   c. wherein the cilnidipine or a pharmaceutically acceptable salt thereof and the tadalafil or a pharmaceutically acceptable salt thereof are administered to the subject once daily for at least five days.
Embodiment 15. The method of embodiment 14, wherein the cilnidipine or a pharmaceutically acceptable salt thereof and the tadalafil or a pharmaceutically acceptable salt thereof are administered to the subject in the morning.
Embodiment 16. The method of embodiment 14, wherein the cilnidipine or a pharmaceutically acceptable salt thereof and tadalafil or a pharmaceutically acceptable salt thereof are administered as a fixed dosage form.
Embodiment 17. The method of embodiment 14, wherein the cilnidipine or a pharmaceutically acceptable salt thereof and tadalafil or a pharmaceutically acceptable salt thereof are formulated as a liquid suspension or as an oral thin film.
Embodiment 18. The method of embodiment 14, wherein after administering the cilnidipine or a pharmaceutically acceptable salt thereof and the tadalafil or a pharmaceutically acceptable salt thereof to the subject, the frequency of emergent medical care, systemic sclerosis related medical facility visits, hospitalization, or any combination thereof of the subject are reduced in comparison to before administering the cilnidipine or a pharmaceutically acceptable salt thereof and the tadalafil or a pharmaceutically acceptable salt thereof to the subject.
Embodiment 19. The method of embodiment 14, wherein reducing the frequency and/or severity of one or more symptoms associated with systemic sclerosis comprises measuring or assessing an improvement in the Scleroderma Health Assessment Questionnaire (SHAQ®) in the subject after administering the cilnidipine or a pharmaceutically acceptable salt thereof and tadalafil or a pharmaceutically acceptable salt thereof to the subject.
Embodiment 20. The method of embodiment 14, wherein an improvement of at least 30% in overall systemic sclerosis disease severity is measured or assessed in the visual analog scale (VAS) of overall systemic sclerosis disease severity in the subject after administering the cilnidipine or a pharmaceutically acceptable salt thereof and tadalafil or a pharmaceutically acceptable salt thereof to the subject.
Embodiment 21. A method of treating one or more symptoms associated with a comorbidity of secondary Raynaud's syndrome in a subject in need thereof, comprising:
   determining that the subject has (i) secondary Raynaud's syndrome, and (ii) a comorbidity of secondary Raynaud's syndrome
   orally administering about 30 mg or about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject;
   wherein the subject is cilnidipine-naïve; and
   wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.
Embodiment 22. A method of treating one or more symptoms associated with systemic sclerosis in a subject in need thereof, comprising:
   determining that the subject has systemic sclerosis;
   orally administering about 30 mg or about 20 mg of cilnidipine or a pharmaceutically acceptable salt thereof on a free base basis of cilnidipine and optionally about 5 mg of tadalafil or a pharmaceutically acceptable salt thereof on a free base basis of tadalafil to the subject;
   wherein the subject is cilnidipine-naïve;
   wherein the one or more symptoms associated with systemic sclerosis are selected from the group consisting of: skin ulcers, kidney dysfunction, lung dysfunction, cardiac dysfunction, calcinosis, pain, fibrotic changes in the skin, or any combination thereof; and
   wherein the cilnidipine or a pharmaceutically acceptable salt thereof and the tadalafil or a pharmaceutically acceptable salt thereof are administered to the subject once daily for at least five days.
Embodiment 23.

### EXAMPLES

### Example 1. Clinical Trial Protocol

TABLE 1
Abbreviations.

| Abbreviation | Term |
|---|---|
| ACR | American College of Rheumatology |
| AE | Adverse Event |
| ARB | Angiotensin II receptor blocker |
| ATC | Anatomical Therapeutic Class |
| AUC_{0-inf} | Area under the plasma concentration time |
| | curve from time zero to infinity |
| BMI | Body Mass Index |
| BP | Blood pressure |
| Ca | Calcium |
| CCB | Calcium channel blocker |
| cGMP | Cyclic guanosine monophosphate |
| CKD | Chronic kidney disease |
| CL/F | Apparent total clearance of the drug |
| | from plasma after oral administration |
| CRF | Case Report Form |
| CRP | C-reactive protein |
| CSR | Clinical Study Report |
| CTN | Clinical Trials Notification |
| CV | Coefficient of Variation |
| DDD | Distal-dorsal difference |
| DSMB | Data and Safety Monitoring Board |
| eCRF | Electronic case report form |
| EOS | End of study |
| ESR | Erythrocyte sedimentation rate |
| EULAR | European League Against Rheumatism |
| FIH | First in human |
| GCP | Good Clinical Practice |
| GEE | Generalized Estimating Equation |
| HREC | Human Research Ethics Committee |
| IB | Investigator's Brochure |
| ICF | Informed Consent Form |
| ICH | International Council for Harmonization |
| IP | Investigational product |
| ITT | Intent-to-treat |
| IVRS | Interactive voice response system |
| kel | Elimination rate constant |
| LS | Least Squares |
| MedDRA | Medical Dictionary for Regulatory Activities |
| NHMRC | National Health and Medical Research Council |
| NSAID | Nonsteroidal anti-inflammatory drug |
| PAH | Pulmonary arterial hypertension |
| PDE5 | Phosphodiesterase type 5 |
| PI | Principal Investigator |
| PK | Pharmacokinetic |
| PP | Per-Protocol |
| PT | Preferred term |
| RCS | Raynaud's Condition Score |
| RHI | Reactive Hyperemia Index |
| RP | Raynaud's Phenomenon |
| SAE | Serious Adverse Event |
| SAP | Statistical Analysis Plan |
| SBP | Systolic blood pressure |
| SD | Standard deviation |
| SE | Standard Error |
| SHAQ | Scleroderma Health Assessment Questionnaire |
| SMC | Safety Monitoring Committee |
| SOA | Schedule of Assessments |
| SOC | System organ class |
| SSc | Systemic Sclerosis |
| SSc-RP | Raynaud's Phenomenon secondary mostly |
| | to systemic sclerosis |
| t½ | Elimination half-life |
| TEAE | Treatment-emergent Adverse Event |
| TGA | Therapeutic Goods Administration |
| UCLA SCTC | University of California at Los Angeles |
| GIT 2.0 | Scleroderma Clinical Trials Consortium |
| | Gastrointestinal Tract 2.0 |
| VAS | Visual analog scale |
| VMA | Vanylmandelic acids |
| Vz/F | Apparent volume of distribution during |
| | terminal phase after non-intravenous |
| | administration |
| WHO | World Health Organization |
| WOCBP | Woman of childbearing potential |

A randomized, placebo-controlled phase 2a study was performed and is in progress to assess the safety and efficacy of cilnidipine (10 mg and 20 mg) alone and in combination with 5 mg tadalafil, in participants with diagnosis of scleroderma and/or secondary Raynaud's disease.

Objectives:

Primary Efficacy:
To evaluate the effect of cilnidipine alone and in combination with tadalafil on the
frequency of weekly Raynaud's Phenomenon (RP) attacks compared with placebo in
participants with RP secondary mostly to systemic sclerosis (SSc-RP).
Secondary Efficacy:
To evaluate the effect of cilnidipine alone and in combination with tadalafil on the
clinical features of SSc-RP and the severity and burden of SSc-RP symptoms.
Safety:
To evaluate the safety of cilnidipine alone and in combination with tadalafil compared to
placebo in participants with SSc-RP.
Exploratory (Part A, Dose Selection)
To assess the endothelial function of participants with SSc-RP and impact of treatment on
sympathetic activity.

Number of Participants (Planned):
Overall, 76 participants have been and/or will be enrolled in this study: 36 in the parallel-
group dose selection phase (Part A) and 40 in the 4-way crossover phase (Part B). Dropouts
will not be replaced.
Diagnosis and Main Criteria for Inclusion:
Participants at least 18 years of age diagnosed with severe secondary Raynaud's disease
(Raynaud's Condition Score [RCS] ≥ 40 and at least a 2-phase color change in fingers of
pallor, cyanosis, and/or reactive hypermedia in response to cold exposure or emotion) mostly
resulting from SSc and exhibiting regular and frequent RP attacks (averaging at least one
attack per day) during the Screening period.
Investigational Product, Dosage and Mode of Administration:
Cilnidipine
For Part A, Cilnidipine 10 mg and cilnidipine 20 mg for oral administration, has been and
will be provided to the site in cartons containing 16 tablets sealed in blister packs.
For Part B, the cilnidipine dose selected in Part A will be provided to the site in cartons
containing 16 tablets sealed in blister packs.
Tadalafil
Tadalafil 5 mg, for oral administration has been and will be over encapsulated (and back filled
with inert capsule filler consisting of only maize starch and pre-gelatinized maize starch, so
that the internal tablet cannot be detected) and provided in bottles containing 16 capsules to
the site.
Medications have been and will be dispensed during the preceding in-clinic study visit for
self-administration by the participant once daily in the morning for a 12 (±2) day period. The
duration of the Dosing Period will be confirmed by the study staff when the in-clinic study
visit is scheduled.
Duration of Treatment:
Part A, Double-blind, Parallel-group, Dose Selection: Participants enrolled participated
and will participate for up to 29 (±5) days:
Screening: up to 10 days (−3 days)
Treatment: 12 (±2) days
Follow-up: 7 days (+3 days)
Part B, Double-blind, Placebo-controlled, 4-way crossover: Participants enrolled will
participate for up to 77 days (±14 days):
Screening: up to 10 days (−3 days)
Treatment: 60 days (four Dosing Periods each lasting for 12 (±2) days, separated by a 4
(±1) day washout period)
Follow-up: 7 days (+3 days)
The total duration of the study is expected to be approximately 6 months.
Reference Therapy, Dosage, and Mode of Administration:
Placebo
Placebo tablets (matching cilnidipine) and placebo capsules (matching tadalafil), for oral
administration, were and will be provided to the site.

Criteria for Evaluation:

Primary Efficacy Endpoint:
Percentage change from baseline in frequency of weekly RP attacks.
Secondary Efficacy Endpoints:
Change from baseline in frequency of weekly RP attacks
Change from Baseline in average duration of weekly RP attacks
Change from Baseline in average severity of weekly RP attacks
Change from Baseline in average daily RCS
Change from baseline in highest (most severe) pain score recorded during weekly RP attacks
Change from baseline in average pain score recorded during weekly RP attacks
Change from baseline in net digital ulcer burden
Change from baseline in distal-dorsal difference (DDD) of the affected index finger sites
Change from baseline in participant quality of life measured using the Scleroderma Health Assessment
Questionnaire (SHAQ)
Change from baseline in Raynaud-visual analog scale (VAS)
Change from baseline in physician assessment of disease
Measurement of cilnidipine drug levels taken 2 to 6 hours following the last dose
The time to reach maximum degree of efficacy (in days) compared to baseline
The time to return to baseline symptom severity after termination of dosing
Impact of daily ambient temperature on symptomatic RP attacks
Use of rescue medications for breakthrough symptoms.
Exploratory Efficacy Endpoints (Part A):
Change from baseline in endothelial function as measured by reactive hyperemia index (RHI) using
Endo-PAT
Impact of treatment on sympathetic activity as measured by plasma vanylmandelic acids (VMA)
Safety Endpoints:
Incidence of adverse events (AEs) and serious adverse events (SAEs), including clinically significant
vital signs from the time of randomization until 7 days following the last protocol dose.
Serious adverse events will be monitored by a DSMB on an ongoing basis throughout the study.
Adverse events were and will be coded using the Medical Dictionary for Regulatory Activities (MedDRA)
and summarized by System Organ Class (SOC), Preferred Term (PT), and treatment group.
Statistical Methods:
Part A data will be analyzed in exploratory fashion to support cilnidipine dose selection and treatment effect
check for sample size confirmation for Part B. For Part B data, mixed effects model will be used for
analysis of the continuous efficacy endpoints in the crossover design. For nominal data, Chi-square tests
will be applied. Generalized Estimating Equations method will be used, as appropriate, for adjusting for
potential confounding factors. Safety endpoints will be summarized by treatment group. No multiple
comparison adjustment will be used to control alpha for the multiple comparisons.
Statistical Analyses:
Data from Part A and Part B will be analyzed separately. The primary and secondary efficacy analyses were
and will be based on the Intent-To-Treat (ITT) population. Analyses based on the PP population for Part B
will be considered secondary and confirmatory. All safety analyses will be performed on the safety
population. Subgroup analyses will also be performed.
Additional exploratory analyses may be performed and will be documented in the statistical analysis plan.
Any deviation from planned analyses described in this protocol will also be documented in the statistical
analysis plan.
Data will be summarized by treatment group for treatment effect comparison according to crossover design;
participants receiving each treatment will be pooled from all periods. Baseline will be defined as Screening
assessments for change from baseline analyses for all periods.
Sample Size Calculation:
The sample size was calculated based on the confidence bound in literature report (Rirash 2017). Assuming
a 2-sided.05 alpha for treatment (cilnidipine or combination therapy or tadalafil) versus placebo and a
common standard deviation (SD) of 0.5 for a decrease of 25% attacks (moderate effect size of 0.5) and
without controlling alpha for multiple efficacy comparisons, a sample size of ten participants in each paired
comparison group (cilnidipine or combination therapy or tadalafil) is needed for 80% power in a four period
crossover design, assuming a 20% dropout rate.

### INTRODUCTION

This protocol describes a study that has been and will be performced to evaluate the effect of cilnidipine alone and in combination with tadalifil on the frequency of weekly Raynaud's attacks in participants with SSc-RP.

### Investigational Plan

### Overall Study Design

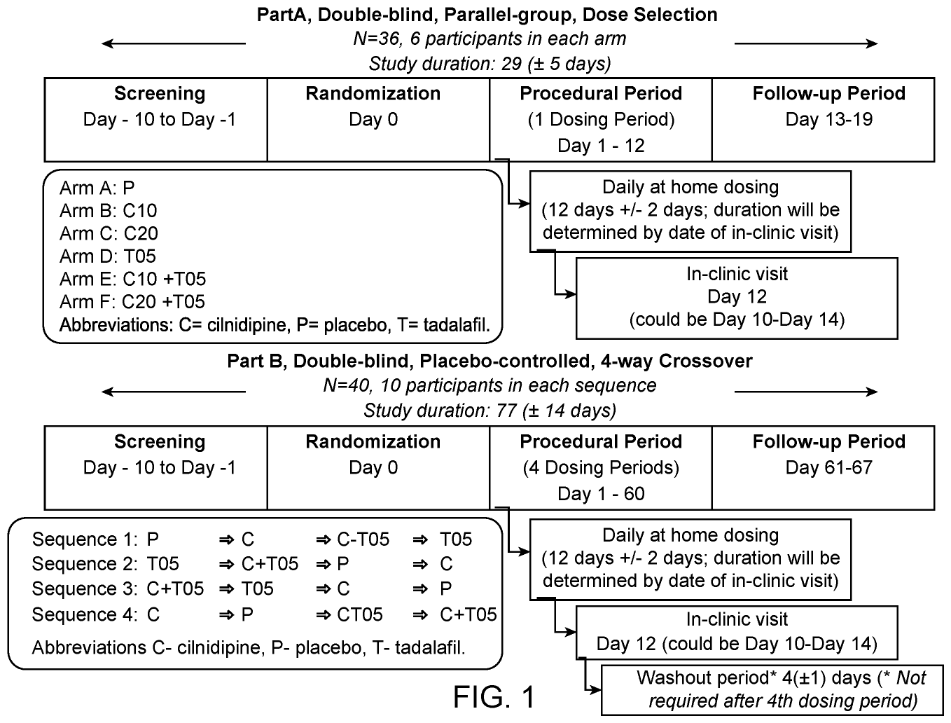

A schematic of the study design is provided in FIG. 1.

This was and is a randomized, placebo-controlled Phase 2a study to assess the safety and efficacy of cilnidipine alone and in combination with tadalafil, in participants who have frequent attacks of secondary RP mostly resulting from SSc. Oversight for the study will be provided by a DSMB.

Participants will underwent and/or will undergo a Screening period beginning up to 10 days prior to randomization. The initial screening and capacity was and will be conducted via phone at the start of the Screening period with eligibility finalized prior to randomization on Day 0. Participants were and will be required to provide informed consent in a 2-step process at Screening (upload of the E-Diary will be considered implied consent for the Screening period) and at Randomization (Day 0) before undertaking any study-specific procedures or assessments. Only participants who met/meet all of the inclusion and none of the exclusion criteria were and will be randomized.

The study consists of two parts.

Part A—Double-blind, Placebo-controlled, Parallel-group, Dose selection, will assess the safety and efficacy of two doses of cilnidipine (10 mg and 20 mg), alone and in combination with tadalafil. A total of 36 participants will be randomized to one of six pre-specified treatment arms. See Example 3 for data obtained in this phase of the study thus far, on 11 participants.

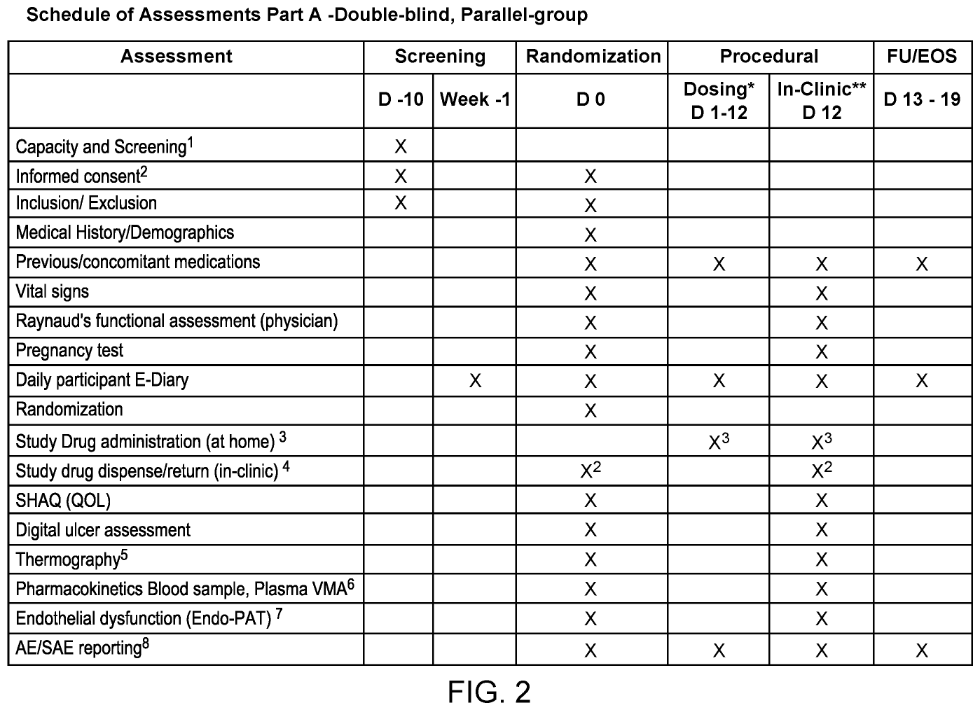

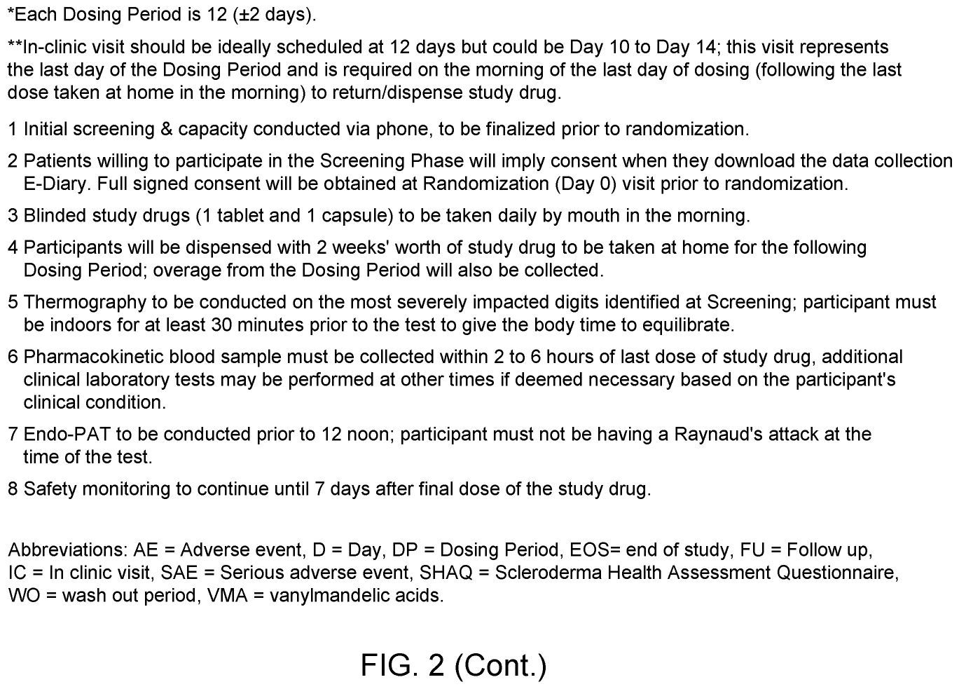

Please refer to FIG. 1 for treatment arms in Part A of the study. Dosing lasted or will last for 12 (±2) days in which participants self-administered or will self-administer daily doses of assigned treatment in the morning. Each participant took or will take one capsule and one tablet to blind the active therapy being received. Study visits and assessments occurred or will occur as delineated in the SoA presented in FIG. 2.

The data from Part A of the study will be reviewed by an unblinded DSMB prior to selecting the cilnidipine dose and confirming the sample size estimates for the randomized double-blind phase (Part B). The first review will occur after approximately 50% of participants have completed the study.

Part B—Double-blind, Placebo-controlled, 4-way Crossover will assess the safety and efficacy of cilnidipine (at the dose selected in Part A) alone and in combination with 5 mg tadalafil. A total of 40 participants (10 in each sequence) with a diagnosis of SSc-RP will be randomized into one of four pre-specified treatment sequences in a 4-way crossover design.

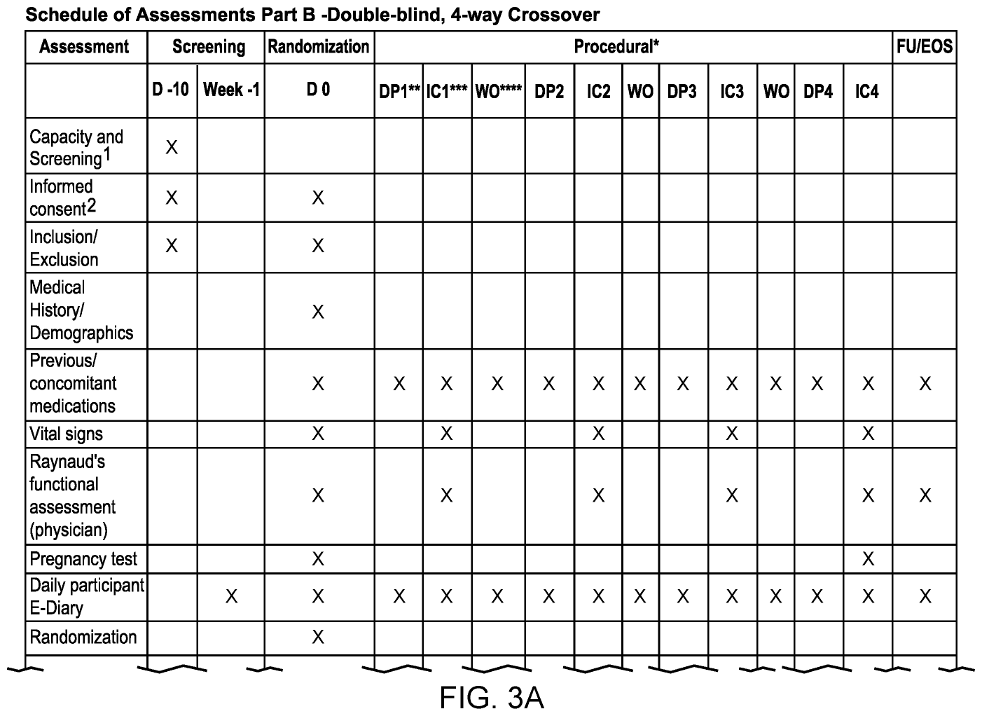

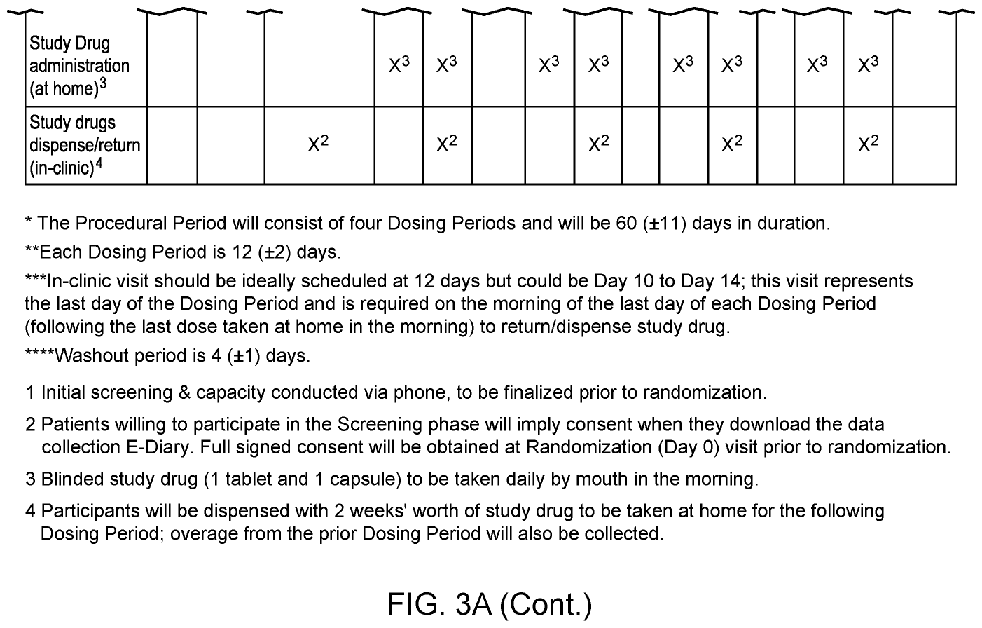

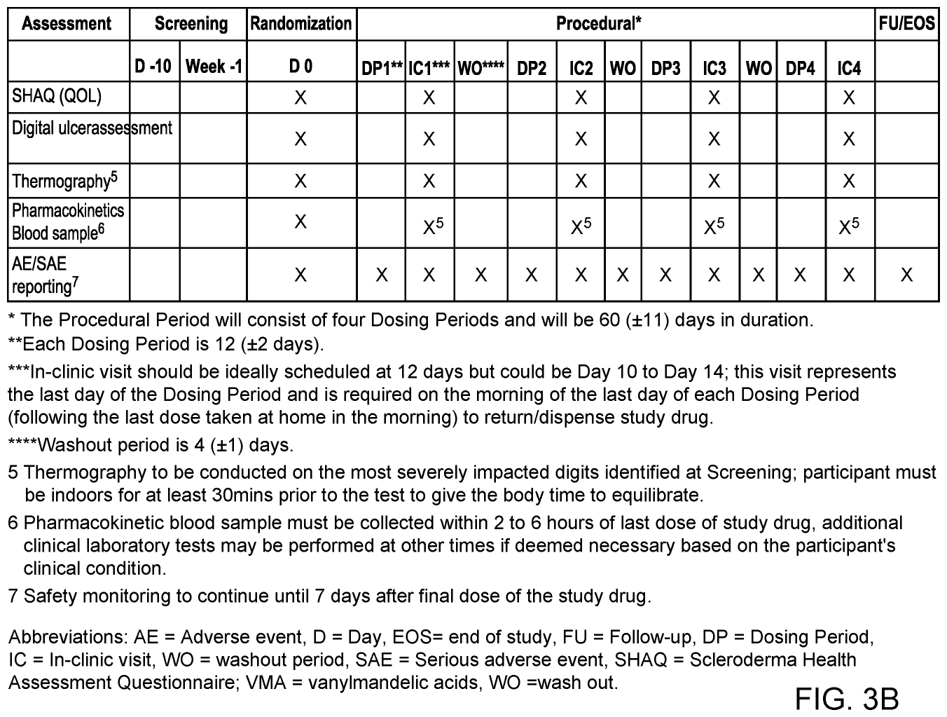

Please refer to FIG. 1 for treatment sequences in Part B of the study. Each participant will undergo four Dosing Periods in which they will receive a different treatment each Dosing Period followed by a 4 (±1) day washout period. Each Dosing Period will last for 12 (±2) days in which participants will self-administer daily doses of assigned treatment in the morning. At all Dosing Periods, each participant will take one capsule and one tablet to blind the active therapy being received. Study visits and assessments will occur as delineated in the SoA presented in FIG. 3A and/or FIG. 3B.

For both Part A and B of the study, participants were or are required to visit the clinic on last day of each Dosing Period (i.e., Day 10 to 14) to return/dispense study drug and conduct in person study assessments. Participants were or will be dispensed with 2 weeks' worth of study drug to be taken at home for the following Dosing Period; overage from the prior Dosing Period were or will also be collected.

Patients were or will be assessed for the occurrence of efficacy endpoints for each Dosing Period via the patient reported E-Diary and the in-clinic visit. Safety information was or will be collected from randomization until patient follow-up is complete (7 to 10) days after the last Dosing Period) and assessed for each Dosing Period.

### Dose Selection, Sample Size Confirmation and Safety Oversight

Safety oversight will be provided by a DSMB. The DSMB will conduct a review of the efficacy and safety data from Part A of the study. The first review occurred after data was available on the first 11 of the participants enrolled into the study. Subsequent reviews will occur as needed prior to commencement of Part B of the study to determine the optimal dose of cilnidipine to carry into Part 2 of the study and to assess the risk: benefit of cilnidipine (10 mg and 20 mg), alone and in combination with tadalafil.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.

Serious adverse events have been and will be monitored by the DSMB on an ongoing basis throughout the study.

### Safety Criteria for Stopping or Unblinding Treatment

Administration of study drug may be paused, and emergency unblinding of treatment conducted following consultation between the Investigator, the Medical Monitor, and the Sponsor representative under the following circumstances:
Symptoms of possible allergic phenomena: rash, hives, urticaria, changes in breathing or wheezing.
Systolic BP (SBP)<90 mmHg
   Reduction in BP (relative to baseline values) considered to be significant in the opinion of the Investigator on an absolute basis or consistent with symptoms (dizziness, light-headedness) suggestive of being related to reduction in BP.
   Two separate SBP measurements of <90 mmHg, taken at rest (where baseline SBP exceeded 110 mmHg), even in the absence of symptoms, shall be considered sufficient reason to unblind the study for that patient.
Dizziness, when going from a recumbent to standing position and/or if accompanied by a reduction in BP.
   Dizziness in the absence of a reduction in BP should be considered on an individual participant basis, as to its relative degree of severity, as to whether the participant continues in the study.
Unexpected adverse event or reaction.

### Study Termination

The study will be completed as planned unless:
New information or other evaluation regarding the safety of the study medication indicates a change in the known risk/benefit profile for the compound, such that the risk/benefit is no longer acceptable for participants participating in the study. This may be determined by the Sponsor, the Investigator, the HREC or regulatory authorities.
The study is terminated by the Sponsor for administrative reasons.

The Sponsor, Investigator, and the HREC reserve the right to terminate or suspend the study at any time; however, this should be discussed between the relevant parties beforehand and the reason for such decision recorded. Should this occur, all data available will also be recorded in the eCRFs. If the Sponsor, the HREC, or regulatory authority elects to terminate or suspend the study or the participation of the investigational site, a study-specific procedure for early termination or suspension will be provided by the Sponsor. The procedure will be followed by the investigational site during termination or study suspension.

The Investigator should notify the relevant HREC in writing of the study's completion or early discontinuation.

### Participant Population

The study has been and will be conducted in participants at least 18 years of age diagnosed with severe secondary Raynaud's disease (RCS≥40 and at least a 2-phase color change in fingers of pallor, cyanosis, and/or reactive hypermedia in response to cold exposure or emotion) mostly resulting from SSc (defined by consensus criteria 2013 American College of Rheumatology [ACR]) and exhibiting a frequency of attacks (at least one per day) during the Screening period.

Women of childbearing potential were or will be included and have been or are subject to contraceptive requirements during the study from Screening until study completion, including the follow-up period, and for at least 30 days after the last dose of study drug (see Section 4.2). Women of childbearing potential must demonstrate negative pregnancy testing at Screening. This is in line with regulatory Guidance on Nonclinical Safety Studies for the Conduct of Human Clinical Trials and Marketing Authorization for Pharmaceuticals (US FDA Guidance document, January 2010).

### Number of Participants

Overall, 76 participants have or will be enrolled in this study: 36 in the parallel-group dose selection phase (Part A) and 40 in the 4-way crossover phase (Part B). Dropouts will not be replaced.

### Participant Inclusion Criteria

To be eligible for this study, each participant has met or has to meet all of the following inclusion criteria:
1. Male or female participants, aged 18 to 80 years (inclusive at the time of informed consent).
Severe secondary Raynaud's disease (defined as RCS at baseline of ≥40) based on ACR criteria mostly resulting from SSc.
Regular and frequent Raynaud's attacks (averaging at least one attack per day) during the Screening week (in participants with at least a 2-phase color change in fingers of pallor, cyanosis, and/or reactive hypermedia in response to cold exposure or emotion).
Willingness to complete the daily diary entry's during the Screening period.
Participants must be willing and able to provide written informed consent after the nature of the study has been explained and prior to the commencement of any study procedures.
Willingness to forego other routine therapies for Raynaud's during study participation. Ongoing treatment with CCBs for SSc-RP is permitted if it is not clinically feasible to stop therapy and the participant has been on a stable dose for the last 2 months. Rescue therapy (with acetaminophen, nonsteroidal anti-inflammatory drug [NSAIDs], other codeine analgesics, fluoxetine, angiotensin II receptor blockers (ARBs) such as losartan) or CCBs will be allowed to manage breakthrough symptoms of SSc-RP but must be documented).
Participants who are on a stable dose (no change in the last 2 months) of a CCB for hypertension or sildenafil for pulmonary hypertension may continue these agents at this stable dose as long as they meet other inclusion criteria during Screening.
Have agreed not to use (or initiate treatment with) other investigational therapies or unapproved therapies during the course of the study outside of protocol allowances for rescue medication (such medications include but are not limited to: nitroglycerin, topical creams, fenoldopam, nimodipine, amlodipine, fluoxetine, pregabalin, gabapentin, verapamil, sildenafil, tadalafil, vardenafil).
Women of childbearing potential (WOCBP) who have agreed to use an effective method of contraception during the study and for 30 days after the last study dose. Acceptable methods of birth control in this study include oral contraceptive, barrier control, or implanted devices. A woman must agree not to donate eggs (ova; oocytes) for purposes of reproduction for at least 30 days after last dose of study drug.
Post-menopausal females, aged over 45 years who have not had a period for at least 12 months, and are not using hormonal contraception, or females documented as permanently sterile (e.g., bilateral tubal ligation; hysterectomy, bilateral salpingectomy).
Negative urine pregnancy test on Randomization day (WOCBP only).
All sexually active men (due to potential risk of drug exposure through the ejaculate) who agree to a barrier method of birth control during the study and for 30 days after the last dose of the study drug. All men must agree not to donate sperm for at least 30 days after receiving last dose of study drug.
Participants must have clinical laboratory values within normal range as specified by the testing laboratory at their most recent pre-screening sample, unless deemed not clinically significant by the Investigator or delegate. Lab sample may be within the last 2 months as long as there have been no significant clinical changes since the last labs and no new medications (e.g. Diuretics) have been introduced that are known to be associated with changes in clinical chemistry or hematology values (e.g. potassium with diuretics or cyclosporine associated with aplastic anemia).

### Participant Exclusion Criteria

A participant who has met or meets any of the following exclusion criteria must be excluded from the study:
1. Primary Raynaud's disease.
History of Raynaud's attacks of sufficient severity as to require in-patient hospitalization (within the last 6 months).
The SBP of <95 mm Hg during Randomization visit (Day 0).
Pulmonary hypertension requiring specific therapy for this condition.
Participants with an allergy to dihydropyridine CCBs that results in clinical findings such as profound hypotension, hives, rash, urticaria, wheezing and changes in breathing (Common treatment limiting adverse events [AEs] that occur with CCBs nifedipine and amlodipine such as edema, headache, heart rate changes, tachycardia, fatigue, constipation, flushing, drowsiness, dizziness should not limit enrollment into this study).
History of other chronic pain condition that could confound recording of pain scores during the study period.
Any prior or ongoing medical conditions, medical history, physical findings, or laboratory abnormality that in the opinion of the Investigator(s), would contraindicate administration of the study medication, or interfere with the study evaluations, or interfere with the participants ability to comply with the study protocol.
Cognitive or language difficulties that would impair completion of the study assessments.
Use of any investigational product (IP) or investigational medical device or participation in investigational drug studies within 30 days prior to enrollment in the study.
Those receiving nitrates, alpha blockers, PDE inhibitors (outside of stable administration for PAH), prostacyclins or endothelin antagonists.
History of orthostatic hypertension, dizziness or fainting spells, acute coronary or cerebrovascular event within 3 months of study enrollment.
History of major thoracic, abdominal, or vascular surgery within 6 months of study enrollment; History of sympathectomy.
Severe cardiomyopathy, severe valvular heart disease, chronic kidney disease (CKD) stage 3 or greater, evidence of malignancy, end stage lung disease.
Pregnant or lactating women.
Women of childbearing potential (WOCBP) unable to comply with contraceptive requirements during the study period.
Males with partners who are WOCBP and are unable to comply with the contraceptive requirements during the study.
History of drug or excess alcohol use that in the opinion of the Investigator(s) would affect the participant's ability to reliably participate in the study. N MIRC guidelines for regular alcohol consumption in healthy adults are no more than 10 standard drinks per week and no more than four standard drinks on any one day.
Use of tobacco products of any type in the preceding one month and for the duration of the study.

### Screen Failures

Screen failures are defined as participants who consent to participate in the clinical study but are not subsequently randomized in the study. A minimal set of screen failure information is required to ensure transparent reporting of screen failure participants to meet the Consolidated Standards of Reporting Trials publishing requirements and to respond to queries from regulatory authorities. Minimal information includes screen failure details and eligibility criteria. Participants who withdraw from the study, for any reason, prior to randomization will be considered screen failures. Individuals who do not meet the criteria for participation in this study (screen failure) may be re-screened following a one month waiting period. Re-screened participants should be assigned a new participant number.

### Participant Replacement

All participants who were or are randomized were or will be followed and included in the primary ITT analysis. Dropouts were not and will not be replaced.

### Participant Withdrawal Criteria

Participants may withdraw their consent to participate in the study at any time. If a participant withdraws consent, the date and reason for consent withdrawal should be documented. Participants will be encouraged to remain in the clinic to complete all necessary assessments and until the Investigator deems that it is safe to be discharged. Participant data will be included in the analysis up to the date of the withdrawal of consent.

Apart from withdrawal of consent, reasons for early termination of individual participants can include:
Protocol deviations or participant non-compliance (must be specified on the appropriate eCRF)
Serious or severe AEs
Administrative decision by the Investigator or the Sponsor
Death
Other (must be specified).

The primary reason for withdrawal will be identified and recorded on the appropriate eCRF, along with the date of withdrawal.

In accordance with applicable regulations, a participant has the right to withdraw from the study, at any time and for any reason, without prejudice to future medical care.

If a participant is withdrawn because of an AE, the Investigator must arrange for the participant to have appropriate follow-up care until the AE is resolved or has stabilized. Unresolved AEs will be followed until the last scheduled Follow-up/End of Study (EOS) visit or until the Investigator(s) determine that further follow-up is no longer indicated. In addition to AEs, other reasons for removal of participants from the study might include, but are not limited to, withdrawal of consent, administrative decision by the Investigator or the Sponsor, protocol deviation, or participant noncompliance.

If a participant asks or decides to withdraw from the study, all efforts will be made to complete and report the observations, especially those related to the listed primary and secondary objectives, as thoroughly as possible up to the date of withdrawal. Wherever possible, the tests and evaluations, including those listed for the EOS/follow-up visit, should be performed for all participants who discontinue prior to the completion of the study.

### Treatments

### Treatments Administered

### Investigational Product

### Cilnidipine

Cilnidipine is an orally administered dihydropyridine CCB that dilates blood vessels, increases blood flow, inhibits sympathetic nervous system activity, and improves endothelial structure and function. Please refer to IB for more information on composition of cilnidipine tablet (Profervia® Investigator's Brochure, 2020), which is incorporated by reference herein in its entirety.

Profervia® tablets are white film-coated tablets. Each tablet contains cilnidipine (10 or 20 mg) with microcrystalline cellulose, lactose, magnesium stearate, sodium starch glycollate, Opadry white, polyvinyl alcohol, titanium dioxide, macrogol, talc, and purified water.

Cilnidipine is commercially available and should be used only in accordance with this study protocol and IB.

Cilnidipine 10 mg and 20 mg oral tablets have been and will be provided to the site in cartons containing 16 tablets sealed in blister packs.

### Tadalafil

Tadalafil for oral administration belong to a class of medications called PDE5 inhibitors.

Tadalafil is commercially available and should be used only in accordance with this study protocol. Please refer to the pharmacy manual and product information sheet for more information on composition and storage information for tadalafil.

Tadalafil has been and will be over encapsulated (and back filled with inert capsule filler consisting only of maize starch and pre-gelatinized maize starch, so that the internal tablet cannot be detected) and provided in bottles containing 16 capsules to the site.

### Reference Products

Placebo tablets (matching cilnidipine) and placebo capsules (matching tadalafil) for oral administration have been and will be provided for this study.

### Dosage and Treatment Periods

At all Dosing Periods, each participant have taken or will take one capsule and one tablet to blind the active therapy being received. All medications for each Dosing Period (each Dosing Period will last for 12 days [±2 days]) have been or will be dispensed during the preceding in-clinic visit and then self-administered by the participant once daily, orally, in the morning.

If a participant accidentally misses a dose, they have been or should be advised to take the dose on the same day as soon as they realize. Only one tablet and capsule have been and should be taken each day. If more than one dose is lost, the participant should notify the study staff so that their in-clinic visit can be adjusted if needed.

Part A, Double-blind, Parallel-group, Dose Selection Study drug has been or will be self-administered daily for 12 (±2) days. Each participant has or will receive only one treatment. Please refer to FIG. 2 for more details.

Part B, Double-blind, Placebo-controlled, 4-way Crossover Study drug will be self-administered daily in four Dosing Periods separated by a four-day (±1) washout period. Each participant will receive a different treatment during each Dosing Period, with a total of four treatments received. Please refer to FIG. 3A and/or FIG. 3B for more details.

### Method of Assigning Participants to Treatment

### Randomization

A randomization list has been or will be prepared using a statistical software package by a Biostatistician.

Each participant has been or will be provided with a unique screening number post-documentation of informed consent. Once deemed eligible, the participant has been or will be assigned a sequential randomization number prior to first dosing. Participants who withdraw from the study or who fail eligibility, for any reason, prior to randomization will be considered screen failures.

### Concomitant Medications

All medications, including over the counter medications, vitamins, and herbal supplements, taken during the Screening period have been or will be reviewed by the Investigator to determine whether these medications render the participant as suitable for inclusion in the study.

Concomitant medications of interest have been or will be captured electronically from the start of the Screening period until study completion.

Treatment prior to enrollment with therapies for SSc-RP including but not limited to CCBs, nitroglycerin, topical creams fenoldopam, nimodipine, fluoxetine, pregabalin, gabapentin, sildenafil, tadalafil, vardenafil were or are permitted. In order to have been or be eligible, participants must be willing to forego these therapies for SSc-RP at the start of the Screening period and for the duration of the study. Participants who were or are on a stable dose (no change in dose in prior 2 months) of a CCB for hypertension or sildenafil for pulmonary hypertension may continue these agents at this stable dose as long as they meet other inclusion criteria during Screening. Similarly, participants who have been or are receiving CCBs to manage their symptoms of SSc-RP and are unwilling to stop treatment for the duration of the study would still be eligible if the participant's dose has been stable for the past 2 months. During the study participants were or will be able to decrease the dosage of their prior CCB for safety reasons only; no increase in dosage was or will be allowed during the study period.

The use of any other IP or investigational medical device within 30 days prior to Screening is prohibited.

Prior therapy or concomitant therapy (after study drug administration) with any medications, including both prescription and non-prescription drugs should be discussed with the Investigator and Sponsor's MM before study drug administration, except in the case of necessary treatment of AEs or where appropriate medical care necessitates that therapy should begin before the Investigator can consult with the Sponsor's MM.

### Rescue Medicine

Medications required as rescue therapy can be taken to manage breakthrough symptoms of SSc-RP but must be recorded in the participant E-Diary. First-line therapy may include acetaminophen, NSAIDs, or other codeine-based analgesics. These rescue medications may be taken for the duration of symptoms of a Raynaud's attack. For participants in whom first-line rescue therapy is not effective, additional rescue medication therapy may be started per Investigator discretion and could include fluoxetine, ARBs such as losartan or CCBs. Rescue therapy should continue as long as clinically needed during an acute attack, but then patients should return to the pre-rescue study medication regimen. All participants receiving rescue therapy should continue in the study undergoing subsequent Dosing Periods through study completion. If a participant requires regular rescue medicine during dosing through at least four sequential Dosing Periods, then the participant may drop out of the study at the discretion of the participant and the Investigator.

### Treatment Compliance

All doses have been or will be self-administered by participants remote from study sites (at home). For each Dosing Period, participants were or will be dispensed with two weeks' worth of study medication and will be asked to return the unused study medication on the last day of each Dosing Period at the time of in-clinic visit. The treatment compliance was or will be noted by the Investigator(s) during the in-clinic visit.

### Blinding

This study is double-blind. To maintain the blind, all study medication has been and will be provided to the site in a blinded fashion. Cilnidipine tablets and matching placebo was and will be supplied in cartons containing 16 tablets sealed in blister packs, identical in appearance. Tadalafil was and will be provided in an over encapsulated form. The capsules, tadalafil, and placebo were and will be identical in appearance and weight and will be supplied in bottles containing 16 capsules.

Each study drug was and will be labeled with a unique ID number. The interactive voice response system (IVRS) was or will have access to the treatment arm assignment for each individual ID number.

### In the Event of an Emergency: Unblinding a Code

It is recognized that, in the course of clinical practice, it may be necessary for the treating physician to have knowledge of the treatment assignment to ensure the safety of a study participant. This circumstance is extraordinary and will likely impact a minor fraction of the enrolled participants. Unblinding will be done via the IVRS. The treating physician is encouraged to contact the Sponsor MM in this circumstance. The Sponsor and DSMB will monitor all episodes of unblinding very carefully.

### Study Schedule

The SoAs for Part A and Part B of the study are provided in FIG. 2 and FIG. 3A and/or FIG. 3B.

Where possible, assessments have been and should be conducted in order of least invasive to most invasive.

This study consists of four periods:
Screening period (begins with initial participant contact through participant completion of the screening E-Diary)
Randomization period (from the time participant eligibility is confirmed and the participant randomized until immediately before the 1^{st} dose of study drug)
Procedural period (from the first dose of study drug in the first Dosing Period until the last day of dosing)
Follow-up period (from the end of the procedural period through 7 days).

### Part A—Double-Blind, Placebo-Controlled, Parallel-Group, Dose Selection

In Part A, the procedural period has required and will require only one Dosing Period i.e. participants have or will receive only one treatment during the procedural period. Within the procedural period for Part A there have been and will be two sub-periods:
Daily at home dosing (first ten to fourteen days of the Dosing Period)
In-clinic visit (the last day of the Dosing Period that occurs on Day 12 [±2 days]).

### Part B—Double-Blind, Placebo-Controlled, 4-Way Crossover

In Part B, the procedural period will require four Dosing Periods i.e. participants will receive four different treatments in a 4-way crossover design. Within the procedural period for Part B there will be three sub-periods associated with each Dosing Period/treatment received:
Daily at home dosing (first ten to fourteen days of the Dosing Period)
In-clinic visit (the last day of the Dosing Period that occurs on Day 12 [±2 days])
Washout period (four days of no dosing that occurs between each Dosing Period).

### Screening (Day −10 to Day −1)

Prior to enrolling in the study, and before performance of any procedures, potential participants have been or will be contacted via phone to discuss the details of the study and assess their eligibility and willingness to comply with all study procedures and duration. If the participant seemed or seems eligible and was or is interested in participating in the Screening period, then they were or will be asked to upload and start using an E-Diary to record the daily clinical features and symptoms of their SSc-RP for the next 7 to 10 days. Upload of the E-Diary was and will be considered implied consent for the Screening period, the data from which was and will be used to confirm eligibility and future baseline analyses assuming the participant is randomized.

During Screening, the E-Diary collected or will collect the following data to confirm eligibility and serve as the baseline measure for efficacy endpoints should the participant be randomized:
Number of daily Raynaud's attacks
Duration of each attack
Symptoms of each attack, including numbness, pain, tingling, color changes
Severity of each attack (all symptoms of the attack to be considered including tingling, numbness, pain, color changes)
Location of participant during each attack (inside/outside)
Pain score of each attack—using 11-point Likert scale, a validated pain scale which can be used to record intensity of pain
Daily RCS— a validated outcome measure used to assess the level of difficulty experienced due to RP each day
Rescue medications taken to manage breakthrough symptoms of SSc-RP including acetaminophen, NSAIDs, other codeine-based medicines, fluoxetine, ARBs such as losartan, CCBs
Other concomitant medications of interest including allergy medications, stable medications for pre-existing hypertension or pulmonary hypertension, and prohibited medications for SSc-RP (including topical creams, fenoldopam, nimodipine, pregabalin, gabapentin, sildenafil, tadalafil, vardenafil).

### Randomization Day (Day 0)

Participants were or will be scheduled to visit the clinic for Randomization (Day 0) assessments between days 7 to 10 of the Screening period. Only participants who seemed or seem eligible based on E-Diary compliance and frequency of RP attacks were or will be requested to visit clinic for randomization. The participant was or will also be provided with an Informed Consent Form (ICF). Prior to being asked to sign the consent form, participants were or will be given time to review study information and ask any questions.

After the consent form is signed and the following assessments will be carried out:
Medical history/demographics
Previous/concomitant medications
Vital signs
Raynaud's functional assessment (by physician)
Pregnancy test
Review of daily participant E-dairy
Scleroderma Health Assessment Questionnaire (SHAQ) which includes Raynaud's VAS to be completed by the participant with review by physician
Digital ulcer assessment
Thermography
Endo-PAT (Part A only)
Blood sample for PK
Plasma VMA (Part A only)
Inclusion/Exclusion
Randomization
AE/SAE reporting
Dispensing study medication

Note: The data collected for assessments that were and are performed first time on Randomization (Day 0) visit (vital signs, digital ulcer assessment, and Endo-PAT) served and will serve as baseline measure for efficacy endpoints for those assessments.

Routine hospital tests including hematology, biochemistry, inflammatory markers (C-reactive protein [CRP] and erythrocyte sedimentation rate [ESR]), antibody status (serum anti-topoisomerase [anti-Scl 70]) and nailfold capillaroscopy should be conducted as clinically indicated per standard of care but are not required per protocol. If conducted, results will be collected in the eCRF and used to describe the severity of disease in the baseline demographic and disease data.

### Dosing Periods

### Daily at Home Dosing (12 Days (+2 Days))

During the Dosing Period, participants were or will be required to self-administer the assigned study medication once daily in the morning at home. Participants were or will also be required to complete their E-Diary daily to capture the clinical features and symptoms of their SSc-RP, and report concomitant medications including rescue therapy (if any).

### In-Clinic Visit (Last Day of the Dosing Period)

Participants have been or will be required to visit the clinic following each Dosing Period—dosing Day 10 to Day 14. The day of the in-clinic visit is considered the last day of dosing in each Dosing Period.

After taking their last dose of study medication in the morning at home, the following assessments/procedures will take place during the in-clinic visit:

Following assessments/procedures have been and will take place on the day of the visit:
Vital signs
Blood sample for PK
Blood sample for Plasma VMA (Part A)
Daily participant E-dairy review for the most recent dosing sequence
Scleroderma Health Assessment Questionnaire (SHAQ) which includes Raynaud's VAS to be completed by the participant with review by physician.

Physician has or will assess the below at the in-clinic visit, details of which will be recorded in the eCRF:
Concomitant medications
Rating severity of participant's Raynaud's disease
Rating severity of participant's ulcers
Rating overall health of participant for past week
Physician's assessment of digital ulcers—how many ulcers, how many considered new, for each ulcer—location and diameter of the ulcer
Thermography (for two most symptomatic areas in terms of Raynaud's)
Endothelial dysfunction (Endo-PAT, Part A only)
AE/SAE reporting
Dispensing/returning study medication

Note: On the last clinic visit at the end of last Dosing Period (Dosing Period 1 for Part A and Dosing Period 4 for Part B), pregnancy test will be performed for WOCBP.

### Washout Period

In Part B, each Dosing Period will be separated by a 4-day (±1 day) washout period. The washout period will commence the day after the in-clinic visit during which participants will not take any study medication. During the washout period, participants will be required to complete the daily participant E-Diary, reporting their symptoms of SSc-RP, and use of any concomitant medications. Once the 4-day washout is completed the participant will commence the daily at home dosing for the study medication dispensed at the pervious in-clinic visit. No washout period is required after the fourth and final in-clinic visit. After this visit participants will proceed directly to follow-up.

### Follow-Up

Participants have been or will be followed for 7 days following completion of the final Dosing Period.

Participants have or will be requested to complete the E-Diary for 7 days in Follow-up period. Participants have or will also be requested to report use of any concomitant medications and any AEs/SAEs during the Follow-up period.

This visit marks the end of participation in this study.

### Early Termination (If Applicable)

Participants who withdraw early from the study will be encouraged to return to the clinic for an EOS assessment.

The following procedures will be conducted:
Participants will be requested to complete the E-Diary for 7 days following termination
Participants will be requested to report use of concomitant medications of interest for 7 days following termination
Participants will be requested to report any AEs/SAEs for 7 days following termination.

This visit marks the end of participation for participants that withdraw early from the study.

### Pharmacokinetic Assessments

### Blood Sample Collection

One 4 mL blood sample has or will be obtained during each in-clinic visit within 2 to 6 hours of last dose of study drug in that Dosing Period as delineated in the SoA (FIG. 2 and FIG. 3A and/or FIG. 3B). The level of cilnidipine in blood has or will be measured following last dose of the Dosing Period. Sample handling details have been or will be provided in the PK manual. The actual collection time of each sample must be recorded in the source data documentation, on the collection tube and in the eCRF.

### Efficacy Assessments

### Efficacy Parameters

Study procedures should be completed as delineated in the SoAs (FIG. 2 and FIG. 3A and/or FIG. 3B).

### E-Diary

The Sponsor-developed participant-informed E-Diary has been and will be used in this study to record data. Participants have been or will be required to keep and fill the E-Diary daily as delineated in the SoAs (FIG. 2 and FIG. 3A and/or FIG. 3B).

The relevant metrics measured by this tool daily are:
Study medication
SSc-RP symptoms (Reporting an attack including duration of attack)
Severity of the attack considering all symptoms of the attack e.g. tingling, numbness, pain, color changes (VAS 0-10 cm scale)
Participant's location at the time of the attack (inside home/outside home)
Selecting symptoms experienced during the attack (tingling, numbness, pain, color changes, other)
Pain rating during the attack (11-point Likert scale)
The RCS based on how much difficulty participants had with Raynaud's today, how many attacks the participant had, and how long they lasted. Participants will also be asked to consider how much pain, numbness, or other symptoms the Raynaud's caused in fingers (including painful sores), and how much the Raynaud's alone affected the use of hands today (VAS 0-10 cm scale).

The relevant metrics measured by this tool at the in-clinic visit (once in a Dosing Period) are:
Digital ulcer severity (VAS 0-10 cm scale)—Applicable only if participant has digital ulcers.
Scleroderma Health Assessment Questionnaire which includes Raynaud's VAS measuring participant quality of life over the past
   Note: Digital ulcer severity (VAS 0-10 cm scale) and SHAQ scale can also be done on paper in-clinic, if necessary. Also, external temperature will be a feed into the E-Diary database based on participant's location.

### Physician Assessments

The Physician has and will assess the below at the in-clinic visit, details of which will be recorded in the eCRF. In Part A, thermography and Endo-PAT will also be reported.
Rating severity of participant's Raynaud's disease
Rating severity of participant's ulcers
Rating overall health of participant for past week
Physician's assessment of digital ulcers—how many ulcers, how many considered new, for each ulcer: location and diameter of the ulcer
Recording temperature of the affected index finger sites at two symptomatic areas at baseline and every in-clinic visit using the Fluor thermographic camera
AE/SAE reporting.

Drug accountability, including dispensing and returning of the study medication will also be recorded at each visit.

### Scleroderma Health Assessment Questionnaire (SHAQ)

The standard, validated, patient reported outcome measures tool for SSc patients, the SHAQ, will be collected at the time points specified in the study schedules (FIG. 2 and FIG. 3A and/or FIG. 3B). To assess the participant's quality of life. The SHAQ includes a Raynaud's VAS that will also be reported separately.

### Thermography

Thermography assessments will be performed at the time points specified in the study schedules (FIG. 2 and FIG. 3A and/or FIG. 3B). Thermography will be conducted on the most severely impacted digits identified at Screening; the participant must be indoors for at least 30 minutes prior to the test to give the body time to equilibrate. Photos will be taken with the help of Fluor thermographic camera of these two areas at times specified in SoAs.

### Plasma Vanylmandelic Acids (VMA)

Plasma VMA is a metabolite of norepinephrine. One sample was and will be collected during each in-clinic visit in Part A of the study to assess if a difference in sympathetic activity with cilnidipine can be detected.

### Endothelial Dysfunction (Endo-PAT)

Assessments for endothelial dysfunction will be performed using Endo-PAT at timepoints specified in the Part A study schedule (FIG. 2); Endo-PAT assessment will not be performed in Part B of the study. The Endo-PAT is a diagnostic device used to assess endothelial vasodilator function in a rapid and non-invasive fashion.

The below points should be considered before assessment is started:
2. Prior to the study, the participant should fast for at least 4 hours, and should refrain from caffeine, vitamins or medications that might affect vascular tone for at least 8 hours. The participant must reconfirm their abstinence from tobacco and may use the restroom prior to the assessment.
The Endo-PAT assessment should be conducted in a quiet, dimly lit, temperature-controlled (25° C. for at least 30 minutes) exam room to reduce fluctuations in vascular tone.
Cell phones or paging devices should be silenced, and restrictive clothing that could interfere with blood flow to the arms should be removed. The participant should also remove watches, rings, or other jewelry on the hands or fingers.
Participant's fingers should be inspected for any deformities or injuries that could affect the study. The probes should not be placed on a finger that is cut or injured. Fingernails should not extend more than 5 mm or ⅕ of an inch beyond the tip of the finger tissue.
The index finger is recommended for the study; however, if this finger is unsuitable, a different digit (except the thumb) may be used, as long as the same finger is used on both hands.
The participant should be supine and comfortable for 15 minutes so as to attain a cardiovascular steady state.

### Safety Assessments

### Safety Parameters

Study procedures should be completed as delineated in the SoA (FIG. 2 and FIG. 3A and/or FIG. 3B). Any unscheduled procedures required for urgent evaluation of safety concerns must take precedence over all routine scheduled procedures

### Demographic/Medical History

Medical history (including alcohol and smoking status), date of birth, age (calculated), weight, sex, ethnicity, and race were and will be recorded at Randomization (Day 0) visit.

### Vital Signs

Vital signs (SBP, DBP, pulse rate, temperature) were measured and will be measured at the time points specified in the SoA (FIG. 2 and FIG. 3A and/or FIG. 3B) with participants resting for at least 5 minutes in a supine position. When the time of vital signs measurement coincides with a blood draw, the vital signs have been or will be taken before the scheduled blood draw where possible, ensuring the blood draw is within the window specified in the protocol.

Additional vital signs may be performed at other times if deemed necessary.

### Laboratory Assessments

Routine hospital laboratory tests including hematology, biochemistry, inflammatory markers (CRP and ESR), and antibody status (Scl-70) should be conducted as clinically indicated per standard of care but are not required per protocol. If conducted, results will be collected in the eCRF and used to describe the severity of disease in the baseline demographic and disease data.

Additional clinical laboratory tests may be performed at other times if deemed necessary, based on the participant's clinical condition.

### Pregnancy Testing

A urine pregnancy test will be performed at the Randomization (Day 0) visit and on the last clinic visit at the end of last Dosing Period for WOCBP only.

### Adverse and Serious Adverse Events

In this study, AEs and SAEs will be reported for all participants from the time of randomization until the completion of the Follow-up/EOS visit. Adverse events that are ongoing at the EOS visit will be marked as Not Recovered/Not resolved on the AE eCRF page.

The Investigator will do full AE review during in-clinic visit. All spontaneously volunteered and enquired for, as well as observed AEs, will be recorded in the participant's medical records and the eCRF.

### Complications of the Disease Under Study

Clinical features and symptoms of SSc-RP must be recorded as endpoints in the electronic data collection tools provided by the Sponsor, as well as in the source documents and should not be reported as AEs.

### Definition of Adverse Events

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.

Events meeting the definition of an AE include:
Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE).
Events that do not meet the definition of an AE include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen.

If there is evidence of an AE through report or observation, the Investigator or designee will evaluate further and record the following information:
Time of onset and resolution
Severity
Seriousness
Causality/relation to study treatment
Action taken regarding study drug
Action taken regarding AE
Outcome.

### Severity of an Adverse Event

Severity of AEs will be graded by the Investigator as one of:
Mild (Grade 1): A type of AE that is usually transient and may require only minimal treatment or therapeutic intervention. The event does not generally interfere with usual activities of daily living.
Moderate (Grade 2): A type of AE that is usually alleviated with additional specific therapeutic intervention. The event interferes with usual activities of daily living, causing discomfort but poses no significant or permanent risk of harm to the research participant.
Severe (Grade 3): A type of AE that interrupts usual activities of daily living, or significantly affects clinical status, or may require intensive therapeutic intervention.
Life-threatening (Grade 4): A type of AE that places the participant at immediate risk of death.
Death (Grade 5): Events that result in death.

### Causal Relationship of an Adverse Event

The Investigator will assess the relationship between study drug and the occurrence of each AE. The Investigator's assessment of the relationship of each AE to study drug will be recorded in the source documents and the eCRF. Alternative causes, such as medical history, concomitant therapy, other risk factors, and the temporal relationship of the event to the study drug should be considered and investigated, if appropriate. The following definitions are general guidelines to help assign grade of attribution:
Not related: The event is clearly related to other factors such as the participant's environment or clinical state, therapeutic interventions or concomitant drugs administered to the participant. This is especially so when an event occurs prior to the commencement of treatment with the study drug.
Unlikely: The temporal association, participant history, and/or circumstances are such that the study drug is not likely to have had an association with the observed event. Other conditions, including concurrent illness, progression, or expression of the disease state, or reaction to a concomitant drug administered appear to explain the event.
Possible: The event follows a reasonable temporal sequence from the time of study drug administration or follows a known response to the study drug but could have been produced by other factors such as the participant's clinical state, other therapeutic interventions, or concomitant drugs administered to the participant.
Probable: The event follows a reasonable temporal sequence from the time of study drug administration and follows a known response to the study drug and cannot be reasonably explained by other factors such as the participant's clinical state, other therapeutic interventions, or concomitant drugs administered to the participant.
Definite: The event follows a reasonable temporal sequence from the time of study drug administration or control abates upon discontinuation or cannot be explained by known characteristics of the participant's clinical state.
   Action Taken with Investigational Products

Should the Investigator need to alter the administration of the study drug from the procedure described in the protocol due to the wellbeing and safety of the participant then the action taken will be recorded on the AE eCRF page, as one of the following options:
Dose Reduced
Drug Interrupted
Drug Withdrawn
Not Applicable
Other.

### Outcome

Outcome of an AE will be recorded on the AE eCRF as follows:
Recovered/Resolved
Recovering/Resolving
Recovered/Resolved with Sequelae
Not Recovered/Not Resolving
Fatal
Unknown.

### Definition of Serious Adverse Event

An SAE is an AE occurring during any study phase (i.e. baseline, treatment, washout, or follow-up), and at any dose of the study drug (active or placebo), that fulfills one or more of the following:
Results in death
It is immediately life-threatening
It requires in participant hospitalization or prolongation of existing hospitalization
It results in persistent or significant disability or incapacity
Results in a congenital abnormality or birth defect
It is an important medical event that may jeopardize the participant or may require medical intervention to prevent one of the outcomes listed above.

Important medical events that may not be one of the above may be considered an SAE by the Investigator when, based upon appropriate medical judgment, they are considered clinically significant and may jeopardize the participant, or may require medical or surgical intervention to prevent one of the outcomes listed above.

An AE is considered “life-threatening” if, in the opinion of either the Investigator or the Sponsor, its occurrence places the participant at immediate risk of death. It does not include an AE that, had it occurred in a more severe form, might have caused death.

### Statistics

Statistical methods will be further outlined in the statistical analysis plan (SAP, see Example 2) and approved by the Sponsor. Procedures outlined in the SAP will supersede protocol specified statistical methods in the event of divergence.

Part A and Part B data will be analyzed separately. Analysis of Part A data was and will be mainly exploratory to support cilnidipine dose selection and treatment effect check for sample size confirmation for Part B.

In general, descriptive statistics (e.g. arithmetic mean, SD, median, minimum and maximum) will be calculated for continuous safety data by treatment and protocol specified time point, while frequency summary (e.g. number of observed and percentage of each categories) will be applied for categorical safety data by treatment and protocol specified time point.

### Sample Size

The sample size was calculated based on the CCB data in confidence bound in literature report (Rirash 2017). Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned.

After reviewing the results from Part A: Run-in phase, the sample size for Part B may be adjusted.

### Analysis Populations

Participant inclusion into each population has been or will be determined prior to the final analysis.

### Intent-To-Treat (ITT) Population

All participants who are entered into the study and complete screening, sign an informed consent for the study and randomized have been or will be included in the ITT population.

### Per Protocol Population

All participants who complete the study with all Dosing Periods (for Part B— at least 5 days of dosing within the last 7 days treatment for the first two periods, and 4 days of dosing within the last 7 days treatment for the second two periods) and meet all eligibility criteria, will be included in the PP Analysis Population.

### Pharmacokinetic Population

All participants who receive any amount of active study drug and have sufficiently evaluable concentration time profile to allow determination of at least one PK parameter will be included in the PK population. An evaluable PK profile will be determined at the discretion of the pharmacokineticist following examination of participants with dosing or protocol deviations that could potentially affect the PK profile. The PK population will be used for the summaries of all PK data.

### Safety Population

All randomized participants who received study drug have been or will be included in Safety population and will be classified according to the actual treatment received.

### Statistical Methods

### Participant Disposition

Participant disposition has been and will be analyzed using counts and percentages. The number and percentage of screened participants, enrolled participants, treated participants, participants discontinued from the study and study treatment, as well as the primary reason for discontinuation has been and will be analyzed and listed.

### Demographics, Medical History, and Baseline Characteristics

Demography and baseline characteristics data has been and will be analyzed using descriptive statistics. The following demographic variables will be summarized by dose level: race, gender, age, height and weight, concomitant diseases (Hypertension, peripheral vascular disease, diabetes, CKD and stage, osteoporosis, history and type of heart arrhythmia).

In addition, the following baseline characteristics of Raynauds Disease have been and will be analyzed will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

Medical history terms are coded using the MedDRA® Version 22.0 or higher. Medical history has been and will be analyzed using descriptive statistics by MedDRA® SOC and PT.

### Prior and Concomitant Medication

Prior and concomitant medications were or will be coded using the most current version of the WHO drug dictionary available at the start of the study. Prior and concomitant medications will be listed by participant and summarized by treatment using anatomical therapeutic chemical (ATC) and preferred name.

### Treatment Compliance and Exposure

Treatment compliance and exposure has been and will be summarized and listed by treatment for all participants in the Safety population.

### Analysis of Primary Endpoint

Percent change from baseline evaluation for frequency of weekly RP attacks is and will be the primary efficacy endpoint. Data collected in the last 7 days of each Dosing Period was and will be used for this analysis. Screening assessments were and will be used as baseline for the analysis of all periods. No multiple comparison adjustment will be used to control alpha for the multiple comparisons. Mixed effects model will be used for analysis of the primary endpoint according to the crossover design. Other efficacy endpoints of continuous variables will be analyzed using similar methodology. For nominal data, Chi-square tests will be applied. Generalized Estimating Equations method will be used, as appropriate, for adjusting for potential confounding factors. In addition, the final analysis will assess whether in this study of severe Raynaud's disease participants, the minimally important difference, previously concluded of 14-15 points on the 100 point RCS scale (Khanna, 2010) has been achieved in the cilnidipine dose group. It also will record the percentage of participants achieving a PASS (34 point difference from baseline on a 0-100 VAS) (Khanna, 2010) in each treatment group.

### Analysis of Secondary Endpoints

The secondary endpoints of change from baseline evaluation will be compared among treatment groups using mixed effects model. Kaplan-Meier method will be used to evaluate time to event endpoints. To evaluate the impact of daily ambient temperature on symptomatic Raynaud's attack, logistic regression will be used for temperature versus the occurrence of Raynaud's attack (Yes/No). The effect of temperature on the severity score of Raynaud's attacks and difference of using rescue medication between treatment groups will be evaluated by Chi-square test. The impact of therapy in sympathetic activity will be assessed by mixed model for repeated measures.

### Safety Analyses

All safety assessments, including AEs, laboratory evaluations, vital signs, and other safety assessments will be analyzed using the Safety population.

### Adverse Event

Adverse events will be coded using the most current version of the MedDRA® Version 22.0 or higher. The analysis of AEs will be based on the concept of treatment emergent AEs. Treatment emergent AEs will be tabulated by treatment group and will include the number of participants for whom the event occurred, the severity, and relationship to study drug. Treatment emergent AEs (TEAEs) leading to discontinuation and SAEs with onset after the start of study drug will also be summarized.

All AEs and SAEs (including those with onset or worsening before the start of study drug) through the end of the study will be listed.

### Laboratory Evaluations

Baseline laboratory evaluations will be listed and summarized by treatment.

### Vital Signs

Vital signs (BP [systolic and diastolic], pulse rate, and oral temperature) will be listed and summarized by treatment and protocol specified collection time point. Observed and change from baseline will be summarized at each protocol specified collection time point.

### Other Safety Assessments

The following assessments will be listed by participant:
Pregnancy Test
Raynaud's function assessment by physician.

### Pharmacokinetics

Plasma concentrations and actual blood sampling times will be listed by treatment and protocol specified time point and summarized using descriptive statistics number of measurements, arithmetic mean, SD, and % CV, geometric mean, minimum, median, and maximum—at each scheduled time point. Individual and mean plasma concentration-time profiles will also be presented graphically for each treatment.

Pharmacokinetic parameters will be computed from the individual plasma concentrations using a non-compartmental approach.

Value for elimination rate constant (kₑₗ), elimination half-life (t_{1/2}), Area under the plasma concentration-time curve from time zero to infinity (AUC_{0-inf}), apparent total clearance of the drug from plasma after oral administration (CL/F) or apparent volume of distribution during terminal phase after non-intravenous administration (Vz/F) will not be reported. Additional analyses will be performed as deemed necessary upon review of the data.

### REFERENCES

Khanna P P, Maranian P, Gregory J, Khanna D. The minimally important difference and patient acceptable symptom state for the Raynaud's condition score in patients with Raynaud's phenomenon in a large randomised controlled clinical trial. Ann Rheum Dis. 2010; 69(3):588-591. doi:10.1136/ard.2009.107706
Rirash F, Tingey P C, Harding S E, et al. Calcium channel blockers for primary and secondary Raynaud's phenomenon. The Cochrane database of systematic reviews, 2017; 12(12), CD000467.

### Example 2. Statistical Analysis Plan

### Introduction

The purpose of this statistical analysis plan (SAP) is to describe the procedures and the statistical methods that have been and will be used to analyze data and report results collected as described in Example 1.

### Study Objectives

This study is a placebo-controlled phase 2a study to test the efficacy and safety of cilnidipine alone and in combination with tadalafil in participants who have frequent attacks of secondary RP mostly resulting from SSc (e.g., SSc-RP). The study consists of two parts, Part A and Part B.

The primary purpose of Part A (a double-blind, placebo-controlled, parallel-group study testing 6 treatment combinations) was and is to generate efficacy and safety data that allows the DSMB to select the dose of cilnidipine (10 mg or 20 mg) to be studied in Part B and to confirm the sample size estimates for Part B of the study.

Part B will provide the primary evidence of efficacy and safety. Part B is a double-blind, placebo-controlled, 4-way crossover study, designed to assess the safety and efficacy of cilnidipine (at the dose selected in Part A) alone and in combination with 5 mg tadalafil. Participants will be randomized to one of four prespecified treatment sequences in a 4-way crossover design.

### Primary Efficacy Objective

The primary efficacy objective of the study was and is to evaluate the effect of cilnidipine alone and in combination with tadalafil on the frequency of weekly Raynaud's Phenomenon (RP) attacks compared with placebo in participants with Raynaud's Phenomenon secondary mostly to systemic sclerosis (SSc-RP).

### Secondary Efficacy Objective

The secondary efficacy objective of the study is to evaluate the effect of cilnidipine alone and in combination with tadalafil on the clinical, measured, and global features of SSc-RP and the severity and burden of these SSc-RP symptoms.

### Safety Objective

The safety objective of the study was and is to evaluate the safety of cilnidipine alone and in combination with tadalafil compared to placebo in participants with SSc-RP.

### Exploratory Objective (Part A, Dose Selection)

To assess the endothelial function of participants with SSc-RP and impact of treatment on sympathetic activity and vascular functioning (Part A).

### Study Endpoints

### Primary Efficacy Endpoint

The primary efficacy endpoint of this study is:
Percentage change from baseline in frequency of weekly RP attacks.

### Secondary Efficacy Endpoints

Secondary efficacy endpoints of the study include:
Change from baseline in frequency of weekly RP attacks
Change from baseline in average duration of weekly RP attacks
Change from baseline in average severity of weekly RP attacks
Change from baseline in average daily Raynaud's Condition Score (RCS)
Change from baseline in highest (most severe) pain score recorded during weekly RP attacks
Change from baseline in average pain score recorded during weekly RP attacks
Change from baseline in net digital ulcer burden
Change from baseline in Distal-dorsal difference (DDD) of the affected index finger sites
Change from baseline in participant quality of life measured using the Scleroderma Health Assessment Questionnaire (SHAQ)
Change from baseline in participant gastrointestinal symptoms (of sclerosis) as assessed with the UCLA SCTC GIT 2.0 questionnaire
Change from baseline in Raynaud-Visual analog scale (VAS)
Change from baseline in physician assessment of disease
Measurement of cilnidipine drug levels taken 2 to 6 hours following the last dose
The time to reach maximum degree of efficacy (in days) compared to baseline
The time to return to baseline symptom severity after termination of dosing
Impact of daily ambient temperature on symptomatic RP attacks
Use of rescue medications for breakthrough symptoms.

### Safety Endpoints

The safety endpoint of the study is:
Incidence of adverse events (AEs) and serious adverse events (SAEs), including clinically significant vital signs from the time of randomization until 7 days following the last protocol dose.

### Exploratory Endpoint (Part A Dose Selection)

Change from baseline in endothelial function as measured by Reactive Hyperemia Index (RHI) using Endothelial Dysfunction (Endo-PAT)

### Summary of the Study Design

### General Study Design and Plan

A schematic of the overall study design is provided in FIG. 1. Participants have or will undergo a screening period beginning up to 10 days prior to randomization. The initial screening and capacity was or will be conducted via phone at the start of the Screening period with eligibility finalized prior to randomization on Day 0. Participants were or will be required to provide informed consent in a 2-step process at Screening (by agreeing to complete the screening Diary) and at Randomization (Day 0) before undertaking any study-specific procedures or assessments. Only participants who met or will meet all of the inclusion and none of the exclusion criteria were or will be randomized.

### Part A—Double-Blind, Placebo-Controlled, Parallel-Group, Dose Selection

Assess the safety and efficacy of two doses of cilnidipine (10 mg and 20 mg), alone and in combination with tadalafil. A total of 36 participants were or will be randomized to one of six pre-specified parallel treatment arms.

Please refer to FIG. 1 for treatment arms in Part A of the study. Dosing lasted or will last for 12 (±2) days in which participants self-administered or will self-administer daily doses of assigned treatment in the morning. Each participant took or will take one capsule and one tablet to blind the active therapy being received.

The data from Part A of the study will be reviewed by a data and safety monitoring board (DSMB) including unblinded analysis results, to support selecting the cilnidipine dose and confirming the sample size estimates for the randomized, double-blind, crossover design, phase (Part B).

The first review by the DSMB will occur after 16 participants completed the study in Part A.

### Part B—Double-Blind, Placebo-Controlled, 4-Way Crossover

Assess the safety and efficacy of cilnidipine (at the dose selected in Part A) alone and in combination with 5 mg tadalafil. A total of 40 participants (10 in each sequence) with a diagnosis of SSc-RP will be randomized into one of four pre-specified treatment sequences in a 4-way crossover design. Part B is designed to provide the primary evidence for efficacy analyses.

FIG. 1 depicts the treatment sequences in Part B of the study. Each participant will undergo four dosing periods in which they will receive a different treatment each dosing period followed by a 4 (±1) day washout period. Each dosing period will last for 12 (±2) days in which participants will self-administer daily doses of assigned treatment in the morning. At all dosing periods, each participant will take one capsule and one tablet to blind the active therapy being received.

For both Part A and B of the study, participants were or are required to visit the clinic on last day of each dosing period (i.e., Day 10 to 14) to return/dispense study drug and conduct in person study assessments. Participants were or will be dispensed with 2 weeks' worth of study drug to be taken at home for the following dosing period; overage from the prior dosing period will also be collected.

Patients were and will be assessed for the occurrence of efficacy endpoints for each dosing period via the patient reported diary and the in-clinic visit. Safety information was and will be collected for each dosing period from randomization until patient follow-up is complete (7 to 10) days after the last dosing.

Patients completing Part A may also be enrolled in Part B, as long as they are eligible per the inclusion criteria of Example 1 is met.

### Randomization and Blinding

Each participant was or will be provided with a unique screening number post-documentation of informed consent. Once deemed eligible, the participant was or will be assigned a sequential randomization number prior to first dosing in each Part A and Part B as described below. Participants who withdraw from the study or who fail to meet inclusion criteria, for any reason, prior to randomization will be considered screen failures.

### Part A

A total of 36 participants were or will be randomized in a 1:1:1:1:1:1 ratio to receive one of six pre-specified, parallel treatment arms cilnidipine 10 mg, cilnidipine 20 mg, tadalafil 5 mg, cilnidipine 10 mg+tadalafil 5 mg, cilnidipine 20 mg+tadalafil 5 mg, or placebo.

### Part B

A total of 40 participants (10 in each sequence) will be randomized to 1 of 4 treatment sequences of 4 crossover periods, according to a 4×4 Williams square, as outlined in Table 1.

TABLE 1
Part B 4-way crossover

| | Dosing | Dosing | Dosing | Dosing |
|---|---|---|---|---|
| Sequence # | Period 1 | Period 2 | Period 3 | Period 4 |
| 1 | P | C | C + T05 | T05 |
| 2 | T05 | C + T05 | P | C |
| 3 | C + T05 | T05 | C | P |
| 4 | C | P | T05 | C + T05 |

Abbreviations: C = cilnidipine, P = placebo, T = tadalafil, N = number of participants, T05 = T 5 mg.
Dose of C, either 10 mg or 20 mg will be identified following completion of Part A.

Once a randomization number was or is assigned, it cannot be reassigned to any other participant. All participants who are randomized will be followed and included in the primary ITT analysis. Dropouts will not be replaced.

All randomization codes were or will be generated by the designated unblinded independent statistician prior to the start of the study. Sealed code break envelopes will be provided prior to start of the study.

### Sample Size and Statistical Power Considerations

The sample size was calculated based on the CCB data in confidence bound in literature report (Rirash 2017 referenced in Example 1). Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned for Part B.

After reviewing the results from Part A: Dose selection phase, the power assumptions will be reviewed and sample size for Part B may be adjusted.

### Statistical Considerations

### General Considerations

The statistical analysis will be conducted following the principles specified in the International Council for Harmonization (ICH) Topic E9 Statistical Principles for Clinical Trials. For more information, see https://www.ema.europa.eu/en/ich-e9-statistical-principles-clinical-trials, which is incorporated by reference herein in its entirety.

Unless otherwise noted, continuous variables will be summarized by number of subjects (n), mean, standard deviation (SD), first quartile (Q1), median, third quartile (Q3), minimum and maximum values. In addition, change from baseline values will be calculated at each time point and summarized descriptively. Categorical variables will be summarized by frequency count and the percentage of subjects in each category. Summaries will be generated for each treatment, where appropriate. Individual subject data will be presented in subject data listings.

The default significant level will be 5%; confidence intervals (CIs) will be 95% and all tests will be two-sided, unless otherwise specified in the description of the analyses. Min and max values will be rounded to the precision of the original value. Means, and medians will be rounded to one decimal place greater than the precision of the original value. SDs, SEs, and 95% CIs will be rounded to two decimal places greater than the precision of the original value. Percentages for summarizing categorical data will be rounded to one decimal place. P-values will be rounded to three decimal places. If a p-value is less than 0.001 it will be reported as “<0.001.” If a p-value is greater than 0.999 it will be reported as “>0.999.”

### Definitions of Analysis Populations

Participant inclusion into each analysis population will be determined prior to the final analysis.

### Intent-To-Treat (ITT) Population

All participants who enter or entered into the study and complete screening, sign or signed an informed consent for the study and randomized will be included in the ITT population. Treatment classification will be based on the randomized treatment for analysis.

### Per Protocol Population

All participants who complete the study with all Dosing Periods (for Part B— at least 5 days of dosing within the last 7 days treatment for the first two periods, and 4 days of doing within the last 7 days treatment for the second two periods), meet all eligibility criteria, and without any major/important protocol deviations, will be included in the per-protocol (PP) population. PP analysis population will be evaluated and finalized before database lock.

### Pharmacokinetic Population

All participants who receive any amount of active study drug and have sufficiently evaluable concentration time profile to allow determination of at least one pharmacokinetic (PK) parameter were or will be included in the PK population. An evaluable PK profile was or will be determined at the discretion of the pharmacokinetic specialist following examination of participants with dosing or protocol deviations that could potentially affect the PK profile. The PK population was or will be used for the summaries of all PK data.

### Safety Population

All randomized participants who received study drug were or will be included in Safety population and have been or will be classified according to the actual treatment received.

Data from Part A and Part B will be analyzed separately. The pooled analyses of Part A and Part B may be conducted as exploratory for participants with common treatment. The intent-to-treat (ITT) population will be used to summarize participant disposition. The primary and secondary efficacy analyses will be based on the ITT population. Analyses based on the PP population for Part B will be considered secondary and confirmatory. All safety analyses will be performed on the safety population.

### Definitions and Conventions for Data Handling

### Baseline Definition

In general, the non-missing measurements collected during the last 7 days prior to the date of randomization served and will serve as the data for calculation of baseline measurements for efficacy variables. The data collected for assessments that were or are performed first time on randomization (Day 0) visit (vital signs, digital ulcer assessment, and Endo-PAT) will serve as baseline measure for efficacy endpoints for those assessments. If there is no value on or prior to the date of randomization, then the baseline value will not be imputed, and will be set to missing.

### Study Day

Study day has been and will be calculated from the reference start date and was and will be used to show start/stop day of assessments and events.

Reference start date (Day 1) was or is defined as the date of first dose of study drug.
Study day=(date of event−reference start date+1) if the event date is on or later than reference start date
Study day=(date of event−reference start date) if the event date is earlier than reference start date
   In the situation where the event date is partial or missing, Study day, and any corresponding durations will appear partial or missing in the listings.

### Analysis Visit Windows

For all analyses for this study, the scheduled visit and/or time point from the case report form (CRF) (i.e., CRY visit) was or will be used as the analysis visit and/or time point.

Measurements collected from unscheduled visits will not be included in the by visit summary tables but will be included in the listings.

### Missing Data Handling Rules

Missing data as well as data from participants who drop out early was or will not be imputed.

### Examination of Subgroups

The following subgroup analysis will be performed for the primary efficacy analysis. Subgroup results need to be interpreted with caution if there are insufficient number of subjects in a subgroup.

### Part A/Part B:

Baseline RP attack frequency: >=13-<=20 attacks per week, >20-<=30 attacks per week, >30 attacks per week
Average baseline RCS score: Baseline RCS<35, >=35-<=75, >75
Time of RP attacks since daily study medication: <8 hours, >=8-<=16 hours, >16-<=24 hours
   Part B (additional analyses):
Age group (<45, >=45 and <=64, >=65)
Gender
Baseline Weight: <60 kg, >=60-<=100 kg, >100 kg
Baseline scleroderma diagnosis: Y/N
Smoking status: active, former, non-smoker
Duration of dosing: <10 days, >=10-<12 days, >=12-<=14 days
Stable use of concomitant CCBs: Y/N
Rescue medicines required: Y/N
Behavioral modifications required: Y/N

### Primary, Secondary and Other Variables

### Primary Efficacy Variable

Percent change from baseline evaluation for frequency of weekly RP attacks were or will be used as the primary efficacy variable. The sponsor-developed participant-informed diary will be used to record data. Frequency of weekly RP attacks is defined as the total number of RP attacks divided by the number of days with available diary data within the last 7 days of each dosing period then multiplied by 7. Total number of RP attacks during the last 7 days of screening period divided by the number of days with available data then multiplied by 7 will be used as baseline for the analysis of all periods.

The variable will be calculated as follows:

Percentage change={(weekly RP attacks for the last 7 days of each dosing period−Baseline weekly RP attacks)/Baseline weekly RP attacks}*100%.

### Secondary Efficacy Variables

Change from Baseline in Frequency of Weekly RP Attacks
The absolute change in frequency of weekly RP attacks from baseline to the end of each Dosing Period was and will be a secondary outcome variable and calculated as follows:

Absolute change=weekly RP attacks for the last 7 days of each dosing period−Baseline weekly RP attacks

Change from Baseline in Average Duration of Weekly RP Attacks

The absolute change in average duration of weekly RP attacks from baseline to the end of each dosing period was and will be a secondary outcome variable. The sponsor-developed participant-informed diary was and will be used to record data. Average duration of weekly RP attacks is defined as the total duration of RP attacks divided by total number of RP attacks within the last 7 days of each dosing period. Total duration of RP attacks during the last 7 days of screening period divided by the total number of RP attacks will be used as baseline for the analysis of all periods. The variable will be calculated similar to frequency data as before.

Change from Baseline in Average Severity of Weekly RP Attacks

The absolute change in average severity of weekly RP attacks (VAS 0-10 cm scale) from baseline to the end of each Dosing Period was and will be a secondary outcome variable. The sponsor-developed participant-informed diary will be used to record data. Average severity of weekly RP attacks is defined as the total severity scores of RP attacks divided by total number of RP attacks within the last 7 days of each dosing period. Total severity scores of RP attacks during the last 7 days of screening period divided by the total number of RP attacks will be used as baseline for the analysis of all periods. The variable will be calculated similar to frequency data as before.

Change from Baseline in Average Daily RCS

The RCS is based on how much difficulty participants had with Raynaud's today, how many attacks the participant had, and how long they lasted. In addition participants were and will be asked to consider how much pain, numbness, or other symptoms the Raynaud's caused in fingers (including painful sores), and how much the Raynaud's along affected the use of hand today (VAS 0-10 cm scale).

The absolute change in average daily RCS from baseline to the end of each dosing period was and will be a secondary outcome variable. The sponsor-developed participant-informed diary has been and will be used to record data. The average daily RCS is defined as the total RCS divided by the number of days with available diary data of each dosing period. Total RCS during screening period divided by the number of days with available data will be used as baseline for the analysis of all periods. If there are multiple daily RCS scores, the latest daily RCS will be used. The variable will be calculated similar to frequency data as before.

Change from Baseline in Highest (Most Severe) Pain Score Recorded During Weekly RP Attacks

The absolute change in highest pain score (11-point Likert scale) of weekly RP attacks from baseline to the end of each dosing period will be a secondary outcome variable. The sponsor-developed participant-informed diary will be used to record data. The highest pain score collected during the last 7 days of each dosing period will be used for this analysis. The highest pain score among the last 7 days of screening assessments will be used as baseline for the analysis of all periods. The variable will be calculated similar to frequency data as before.

Change from Baseline in Average Pain Score Recorded During Weekly RP Attacks

The absolute change in average pain score (11-point Likert scale) of weekly RP attacks from baseline to the end of each Dosing Period will be a secondary outcome variable. The sponsor-developed participant-informed diary will be used to record data. The average pain score of weekly RP attacks is defined as the total pain scores divided by total number of RP attacks within the last 7 days of each dosing period. Average of the last 7 days of screening assessments will be used as baseline for the analysis of all periods. The variable will be calculated similar to frequency data as before.

Change from Baseline in Net Digital Ulcer Burden

If participant has digital ulcers, the absolute change in digital ulcer severity (VAS 0-10 cm scale) from baseline to the end of each dosing period will be a secondary outcome variable. The digital ulcer will be assessed by physician at screening and the in-clinic visit. Screening assessments will be used as baseline for the analysis of all periods. The variable will be calculated similar to frequency data as before.

Change from Baseline in Distal Dorsal Difference (DDD) of the Affected Index Finger Sites

Thermography assessments will be performed by physician at the in-clinic visit. Thermography will be conducted on the ring, middle and index digits of both hands; the participant must be indoors for at least 30 minutes prior to the test to give the body time to equilibrate. Photos will be taken with the help of Fluor thermographic camera of these two areas at the in-clinic visit.

The absolute change in DDD measured by thermography from baseline to the end of each dosing period will be a secondary outcome variable. Each participant will have 4 DDD scores at each visit: PIP nailbed Left, DIP-PIP Left, PIP nailbed Right and DIP-PIP Right. The variable will be calculated similar to frequency data as before and the analysis will be summarized by hand and location.

Change from Baseline in Participant Quality of Life Measured Using the SHAQ

The standard, validated, patient reported outcome measures tool for SSc patients, the Scleroderma Health Assessment Questionnaire (SHAQ), will be used to assess the participant qualify of life. Details are attached in the Appendix. The participant quality of life will be scored, and a disability index calculated for each questionnaire completed by the patient at Screening and each in-clinic visit. The absolute change in participant quality of life from baseline to the end of each dosing period will be calculated similar to frequency data as before.

Change from Baseline in Participant Gastrointestinal Symptoms (of Sclerosis) as Assessed with the UCLA SCTC GIT 2.0 Questionnaire

The standard, validated, patient reported outcome measures tool for SSc patients, the University of California at Los Angeles Scleroderma Clinical Trials Consortium Gastrointestinal Tract 2.0 (UCLA SCTC GIT 2.0), will be used to assess the participant gastrointestinal symptoms (of sclerosis). Details are attached in the Appendix. The participant gastrointestinal symptoms will be assessed at Screening and each in-clinic visit. Participant responses to the questionnaire will be scored and used to calculate a total score indicating the impact of gastrointestinal symptoms on quality of life. The constipation score is not included in the calculation of the total score and will be reported separately. The absolute change in participant gastrointestinal symptoms from baseline to the end of each dosing period will be calculated similar to frequency data as before.

Change from Baseline in Raynaud-Visual Analog Scale (VAS)

The SHAQ includes a Raynaud's VAS, which will be reported at screening and each in-clinic visit. The absolute change in Raynaud-VAS, assessed by the participants response to the SHAQ question ‘In the past 7 days, how much have your Raynaud's interfered with your daily activities?’ at baseline and the end of each dosing period will be calculated similar to frequency data as before.

Change from Baseline in Physician Assessment of Disease

The Physician will rate severity of participant's Raynaud's disease at Screening and in-clinic visit. The absolute change in physician assessment of disease from baseline to the end of each dosing period will be calculated similar to frequency data as before.

### Measurement of Cilnidipine Drug Levels Taken 2 to 6 Hours Following the Last Dose

One 4 mL blood sample will be obtained during each in-clinic visit within 2 to 6 hours of the last dose of study drug in that dosing period. The level of cilnidipine in blood will be measured following last dose of the dosing period. The actual sampling times will be used in the PK parameter calculations.

Concentrations are used as supplied by the analytical laboratory for PK analysis. The units of concentration and resulting PK parameters, with amount or concentration in the unit, will be presented as they are received from the analytical laboratory. If values below LLOQ is noted, half of the LLOQ will be imputed for summary analysis, but below LLOQ will be left as is in listings.

### The Time to Reach Maximum Degree of Efficacy (in Days) Compared to Baseline

The maximum degree of efficacy is defined as the least daily frequency of PR attacks. If there are more than one day with the same least frequency of PR attacks, the time to the first maximum degree of efficacy will be used for this analysis.

The Time to Return to Baseline Symptom Severity after Termination of Dosing

During each washout period and follow-up period, the time to return to the worst baseline severity of weekly PR attacks will be calculated as the first time of return to the worst baseline severity—the previous in-clinic visit date. A participant with no improvement after baseline or never returns to baseline symptom severity will be regarded as censored.

### Safety Variables

### Extent of Exposure

Extent of exposure in days for each Dosing Period was and will be derived from the following formula:

$$Extent of exposure ( days ) = ( date of first dose of each Dosing Period ) - ( date of last dose of each Dosing Period ) + 1$$

All doses were and will be self-administered by participants remote from study sites (at home). For each dosing period, participants were or will be dispensed with two weeks' worth of study medication and will be asked to return the unused study medication on the last day of each Dosing Period at the time of in-clinic visit. The treatment compliance was and will be noted by the Investigator(s) during the in-clinic visit.

### Adverse Events

AEs will be coded by System Organ Class (SOC) and Preferred Term (PT) using the MedDRA® Version 22.0 or higher. The verbatim term will be included in the AE listings.

Treatment-emergent AEs (TEAEs) are defined as AEs that occur or worsen after the dose of study drug. If the timing of the start of an AE could not be determined unambiguously from the start or end dates provided, it will be assumed to be a TEAE. An AE is considered related if the relationship to either of the study drug has been indicated as possibly or probably or definite related by the investigator. An AE leading to study withdrawal is defined as an AE that cause a subject early terminated from the study. AEs will be identified as emerging in the following parts:
TEAEs for Part A will be those that started during Part A (i.e., from the first dose in Part A to end of follow up of Part A)
TEAEs for Part B will be those that started during Part B (i.e., from the first dose in Part B to end of follow-up of Part B).

The TEAEs by treatment will be presented according to the last treatment received prior to the AE start date for crossover periods in Part B.

All AEs will be listed by participant but only TEAEs will be summarized.

### Clinical Laboratory Variables

R outine hospital laboratory tests including hematology, biochemistry, inflammatory markers (CRP and ESR), and antibody status (Scl-70) will be conducted as clinically indicated per standard of care but are not required per protocol.

### Vital Signs

Vital signs including Systolic Blood Pressure (SBP), Diastolic Blood Pressure (DBP), pulse rate, and temperature were and will be measured at Screening and each in-clinic visit.

Changes in vital signs variables between baseline and each subsequent scheduled assessment will be calculated. Absolute values will be compared to the relevant reference ranges and classified as LNH (low (below range), normal (within range or on limits) or high (above range)). All values (absolute and change) falling outside the reference ranges (see Table 2) will be flagged.

TABLE 2
Vital signs reference ranges

| | Standard | Lower | Upper |
|---|---|---|---|
| Parameter | Units | Limit | Limit |
| Diastolic Blood | mmHg | 60 | 120 |
| Pressure (DBP) | | | |
| Systolic Blood | mmHg | 100 | 160 |
| Pressure (SBP) | | | |
| Pulse Rate | Beats/min | 40 | 120 |
| Body Temperature | Celsius | 36.5 | 38 |
| Weight | kg | 40 | 200 |

Body mass index (BMI) will be calculated from the height and weight as follows:

$$BMI ( kg / {m}^{2} ) = weight ( kg ) / {( height ( m ) )}^{2}$$

### Other Safety Variables

A urine pregnancy test will be performed at the randomization (Day 0) visit and on the last clinic visit at the end of last dosing period for Woman of childbearing potential (WOCBP) only.

Raynaud's function assessment will be conducted by Physician at Randomization (Day 0) visit and each in-clinic visit.

### Exploratory Variables

### Endothelial Function

Assessments for endothelial dysfunction will be performed using Endo-PAT at randomization (Day 0) visit and each in-clinic visit for Part A only; Endo-PAT assessment will not be performed in Part B of the study. The Endo-PAT is a diagnostic device used to assess endothelial vasodilator function in a rapid and non-invasive fashion. Endothelial function will be measured using the reactive hyperemia index (LnRHI): (normal LnRHI is ≥0.7; Grey zone 0.51-0.7; Abnormal ≤0.51). The absolute change from baseline will be calculated similar to frequency data as before.

### Analysis Methods

### Subject Information

### Disposition of Subjects

All screened participants will be included in the summaries of participant disposition. Separate summaries will be provided for Part A and Part B. The summaries will include the number of screened participants, the number of randomized participants, the number and percentage of treated participants, participants discontinued from the study and study treatment, and the primary reason for discontinuation.

The number and percentage of participants in each of the ITT population, PP population, PK population, and Safety population will also be summarized.

Listings of participant disposition will be provided by participant.

### Protocol Deviations

Prior to database lock, all Protocol Deviations (PDs) will be identified and documented based on a blinded review of potential PDs. The potential PDs will be reviewed by study team and classified as major or minor. All PDs will be listed by participant.

Major PDs will be summarized by classification and treatment sequence.

### Demographics and Baseline Characteristics

Demography and baseline characteristics data will be summarized using descriptive statistics. The following demographic variables will be summarized by treatment sequence: race, gender, age (summarized both as a continuous variable and as a categorical variable, with categories <45 years, >=45 to <=64 years, and >=65 years), height and weight, concomitant diseases (hypertension, peripheral vascular disease, diabetes, CKD and stage, osteoporosis, history and type of heart arrhythmia). Separate summaries will be provided for Part A and Part B.

In addition, the following baseline characteristics of Raynaud's Disease will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

Pregnancy test results will be listed but not summarized.

A listing of demographic and baseline characteristics will be provided by participant.

### Medical History

Medical history terms will be coded using the MedDRA® Version 22.0 or higher. Medical history will be summarized by MedDRA® SOC and PT. Medical history will be listed by participant.

### Prior and Concomitant Therapy

Medications will be coded using the most current version of the WHO drug dictionary available at the start of the study.

Those medications taken prior to first dose of randomized study drug will be denoted “Prior.” Those medications started at the same time or after the first dose of randomized study drug will be denoted “Concomitant.”

Medications will be presented according to whether they are “Prior” or “Concomitant,” as defined above. Note that a medication could be both prior and concomitant. If medication dates are incomplete and it is not clear whether the medication was concomitant, it will be assumed to be concomitant.

Prior and concomitant medications will be listed by participant and summarized by treatment using anatomical therapeutic chemical (ATC) and preferred name.

### Treatment Compliance and Exposure

Descriptive summary statistics will be provided for treatment exposure and compliance for each treatment sequence and all participants.

Mean, standard deviation, median, minimum, and maximum of amount of unused study medication returned will be provided. The cell frequencies and percentage of participants in each category (<50%, >=50% and <=75%, >75%) will be provided.

### Efficacy Analyses

### Primary Efficacy Analysis

### Primary Analysis

Percent change from baseline evaluation for frequency of weekly RP attacks was and will be the primary efficacy endpoint. Frequency of weekly RP attacks is defined as the total number of RP attacks divided by the number of days with available diary data within the last 7 days of each dosing period then multiplied by 7. Total number of RP attacks during the last 7 days of screening period divided by the number of days with available data then multiplied by 7 will be used as baseline for the analysis of all periods.

The absolute value, change and percent change from baseline for frequency of weekly RP attacks was or will be summarized via descriptive statistics by treatment.

Analysis will be performed in ITT population using a mixed model. The dependent variable is percent change from baseline in frequency of weekly RP attacks, and the independent variables include treatment, sequence, period, as fixed effects, and participant as a random effect. Kenward and Roger's method will be used to calculate the denominator degrees of freedom for the fixed effects (DDFM=KR). The lease square mean (95% CI) of percent change from baseline for each treatment and the least square difference between each treatment and placebo will be obtained from the LSMEANS statement.

Separate analyses were provided will be provided for Part A and Part B. The pooled analyses of Part A and Part B may be conducted as exploratory for participants with common treatment.

### Confirmatory Analysis

For the primary endpoint, a confirmatory analysis will be conducted in the same manner as the primary analysis in PP population.

### Subgroup Analysis

The following subgroups will be examined for the primary endpoint in the same manner as the primary analysis:

### Part A/Part B:

Baseline RP attack frequency: >=13-<=20 attacks per week, >20-<=30 attacks per week, >30 attacks per week
Average baseline RCS score: Baseline RCS<35, >=35-<=75, >75
Time of RP attacks since daily study medication: <8 hours, >=8-<=16 hours, >16
<=24 hours

### Part B (Additional Analyses):

Age group (<45, >=45 and <=64, >=65)
Gender
Baseline Weight: <60 kg, >=60-<=100 kg, >100 kg
Baseline scleroderma diagnosis: Y/N
Smoking status: active, former, non-smoker
Duration of dosing: <10 days, >=10-<12 days, >=12-<=14 days
Stable use of concomitant CCBs: Y/N
Rescue medicines required: Y/N
Behavioral modifications required: Y/N

### Secondary Efficacy Analyses

Change from Baseline Evaluation

The secondary endpoints of change from baseline evaluation including: frequency of weekly RP attacks, average duration of weekly RP attacks, average severity of weekly RP attacks, average daily RCS, highest pain score recorded during weekly RP attacks, average pain score recorded during weekly RP attacks, digital ulcer severity, Distal-dorsal difference (DDD) measured by thermography, quality of life measured by SHAQ, gastrointestinal symptoms assessed by UCLA SCTC GIT 2.0, Raynaud-VAS and physician assessment of disease, will be analyzed by treatment groups using mixed effect model in the same manner as the primary analysis.

### Time to Reach Maximum Degree of Efficacy

The mean time to reach maximum degree of efficacy will be summarized by treatment.

Time to reach maximum degree of efficacy (in days) will be evaluated using the Kaplan-Meier Survival Analysis approach. Descriptive summary, including time to event percent-tiles (25%, 50%, and 75%) and 95% confidence intervals and Kaplan-Meier mean (SE) will be estimated.

Time to Return to Baseline Symptom Severity after termination of dosing

The mean time to return to the worst baseline symptom severity will be summarized by treatment.

Time to return to the worst baseline symptom severity (in days) will be evaluated using the Kaplan-Meier Survival Analysis approach. If a participant doesn't return to baseline symptom severity during washout period or follow-up period, it will be regarded as censor for that Dosing Period. Descriptive summary, including number of participants (%) censored, time to event percent-tiles (25%, 50%, and 75%) and 95% confidence intervals and Kaplan-Meier mean (SE) will be estimated.

### Impact of Daily Ambient Temperature on Symptomatic RP Attacks

To examine the impact of daily ambient temperature on RP attacks, a Generalized Estimating Equation (GEE) with adjustment for the ambient temperature, treatment effect, period effect, and interaction between treatment and period will be conducted to investigate whether daily ambient temperature predicts the dichotomous dependent variable RP attacks (Yes/No). The outcome will be presented as odds ratio and 95% CI.

Mixed model will be adopted to test the effect of daily ambient temperature on the severity score of RP attacks.

### Use of Rescue Medications for Breakthrough Symptoms.

The difference of using rescue medication between treatment groups will be evaluated by Chi-square test.

### Exploratory Efficacy Analyses

Change from baseline in endothelial function as measured by LnRHI using Endo-PAT for Part A only will be analyzed by treatment groups using mixed effect model in the same manner as the primary analysis.

### Safety Analysis

All safety analyses will be performed on the Safety population. Safety data presented by treatment sequence will be summarized on an ‘as treated’ basis. Safety variables include treatment-emergent adverse events (TEAEs), laboratory evaluations, vital signs, and other safety assessments. Study Day 1 for all safety analyses is defined as the date of the first dose of study drug.

### Adverse Events

Adverse events will be coded using the most current version of the MedDRA® Version 22.0 or higher. Only those AEs that are treatment emergent will be included in summary tables. All AEs, treatment emergent or otherwise, will be presented in participant data listings. Separate summaries will be provided for Part A and Part B. as well as the pooled data. The pooled analyses of Part A and Part B may be conducted as exploratory for participants with common treatment.

An overview AE table, including number and percentage of participants with TEAEs, TEAEs by severity (mild, moderate, severe), TEAEs related to study drug, AEs leading to study withdrawal, AEs leading to study drug discontinuation, SAEs, SAEs related to study drug, and death will be provided.

In addition, number and percentage of subjects will be provided for the following summary tables:
TEAE by PT
TEAE by SOC and PT
TEAE by SOC, PT, and relationship (related, not related)
TEAE by SOC, PT, and maximum severity (mild, moderate, and severe)

A participant having the same AE (as determined by the coded MedDRA preferred term) more than once will be counted only once in the number and percentage of participants calculation for that AE. Similarly, if a participant had more than one AE in a SOC, the participant will be counted only once in the number of subjects with an AE for that SOC. If a participant has multiple AEs with the same preferred term, the maximum severity (severe>moderate>mild) recorded for the events will be presented in the AEs by severity table; if severity is missing, these TEAEs will not be included in the severity table. Similarly, if a participant has multiple AEs with the same preferred term, the worst relationship (related worse than not related) for the event will be presented in the AEs by relationship table; if relationship is missing for an AT it is assumed to be related.

### Laboratory Evaluations

Baseline laboratory evaluations will be summarized by treatment sequence and listed by participant.

### Vital Signs

Descriptive statistics for vital signs parameters (diastolic and systolic blood pressure, pulse rate, oral temperature, weight (if collected) and changes from baseline will be presented by visit and treatment sequence.

All vital signs will be listed by participant.

### Other Safety Analyses

Urine pregnancy test and Raynaud's function assessment will be listed by participant.

### Pharmacokinetics

Plasma concentrations and actual blood sampling times will be listed by treatment and protocol specified time point and summarized using descriptive statistics number of measurements, arithmetic mean, SD, and % CV, geometric mean, minimum, median, and maximum—at each scheduled time point. Individual and mean plasma concentration-time profiles will also be presented graphically for each treatment.

### Interim Analysis and Data Safety Monitoring Board

### Interim Analysis

No formal interim efficacy analyses are planned for this study.

### Data Safety Monitoring Board (DSMB)

Safety oversight was and will be provided by a DSMB, the details of which will be set out in a DSMB Charter. The DSMB plans on conducting a review of the efficacy and safety data from Part A of the study, when data is available on the first 16 to 25 patients that have completed the study. However an early review occurred after an initial 11 participants completed the study.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.

Serious adverse events will be monitored by the DSMB on an ongoing basis throughout the study.

### Summary of Major Changes in the Planned Analyses Appendices

### Scleroderma Health Assessment Questionnaire

The SHAQ is used to calculate a Disability Index to assess the participant qualify of life. The eight categories assessed by the Disability Index are 1) dressing and grooming, 2) arising, 3) eating, 4) walking, 5) hygiene, 6) reach, 7) grip, and 8) common daily activities. For each of these categories, patients report the amount of difficulty they have in performing two or three specific activities.

Ratings such as SOME, MUCH, or USUAL are deliberately not defined for the patients; patients are instructed to respond idiomatically, using their own frame of reference. For example, if a patient asks what “SOME” means, an appropriate response would be “Whatever you think ‘SOME’ means to you”.

### Scoring Conventions for the Disability Index

There are four possible responses for the Disability Index questions:
Without any difficulty=0
With some difficulty=1
With much difficulty=2
Unable to do=3
   The highest score reported by the patient for any component question of the eight categories determines the score for that category.
   If a component question is left blank or the response is too ambiguous to assign a score, then the score for that category is determined by the remaining completed question(s).
   If all component questions are blank or if more than one answer is given, then follow up with the respondent is required.
   If the respondent's mark is between the response columns, then move it to the closest one. If it's directly between the two, move it to the higher one.

Each of the disability items on the SHAQ has a companion aids/devices variable that is used to record what type(s) of assistance, if any, the participant uses for his/her usual activities. These variables (see below) are coded as follows:
0=No assistance is needed.
1=A special device is used by the patient in his/her usual activities.
2=The patient usually needs help from another person.
3=The patient usually needs BOTH a special device AND help from another person.

### Computed Variables:

The scoring variables and scoring rules permit the computation of two disability indices, the Standard Disability Index and the Alternative Disability Index. For either of these, a disability index cannot be computed if the patient does not have scores for at least six (6) categories.

1) The Standard Disability Index. “What is the Disability Level of this Person?”

This question results in a new set of category scores that are computed by adjusting the score for each category, if necessary, based on the patient's use of an aid or device or assistance for that category. If either devices and/or help from another person are checked for a category, the score is set to “2”, unless the score is already “3” (i.e., scores of “0” or “1” are increased to “2”). For example, if the highest score for the dressing category is “1”, and the patient says they use a device for dressing, the computed category score would be “2”. The sum of the computed categories scores is then calculated and divided by the number of categories answered. This gives a score in the 0 to 3 range.

2) The Alternative Disability Index. “What is the Disability Level of this Patient when Using Aids and Devices to Compensate for Disability?”

The aid and device variables are not used to calculate the alternative disability index; it is calculated by adding the scores for each of the categories and dividing by the number of categories answered. This gives a score in the 0 to 3 range.

### The UCLA SCTC GIT 2.0 Questionnaire

The UCLA SCTC GIT 2.0 Questionnaire contains 34 questions in 7 sections to ask about gastrointestinal symptoms and evaluate the Impact of life over the past 7 days. The 7 sections will obtain 7 scores: Reflux score (R), Distension/Bloating score (D/B), Faecal Soilage score (S), Diarrhoea score (D), Social functioning score (SF), Emotional wellbeing score (EWB) and Constipation score (C).

$$Total score = ( R + D / B + S + D + SF + EWB ) / 6.$$

Constipation score is not included in the calculation of total score.

### Partial Dates for Adverse Events and Prior/Concomitant Medication

Dates missing the day or both the day and month of the year will adhere to the following conventions in order to classify treatment-emergent AEs and to classify prior/concomitant medications:

### Adverse Events

The missing day of onset of an AE will be set to:
   First day of the month that the event occurred, if the onset YYYY-MM is after the YYYY-MM of first study treatment
   The day of the first study treatment, if the onset YYYY-MM is the same as YYYY-MM of the first study treatment
   The date of informed consent, if the onset YYYY-MM is before the YYYY-MM of the first treatment.
The missing day of resolution of an AE will be set to:
   The last day of the month of the occurrence. If the patient died in the same month, then set the imputed date as the death date.
If the onset date of an AE is missing both the day and month, the onset date will be set to:
   January 1 of the year of onset, if the onset year is after the year of the first study treatment
   The date of the first treatment, if the onset year is the same as the year of the first study treatment
   The date of informed consent, if the onset year is before the year of the first treatment
If the resolution date of an AE or end date of an IP is missing both the day and month, the date will be set to:
   December 31 of the year of occurrence. If the patient died in the same year, then set the imputed date as the death date.

### Prior/Concomitant Medication

The missing day of start date of a therapy will be set to the first day of the month that the event occurred.
The missing day of end date of a therapy will be set to the last day of the month of the occurrence.
If the start date of a therapy is missing both the day and month, the onset date will be set to January 1 of the year of onset.
If the end date of a therapy is missing both the day and month, the date will be set to December 31 of the year of occurrence.
If the start date of a therapy is null and the end date is not a complete date then the start date will be set to the earlier of the imputed partial end date and the date of the first study visit.
If the start date of a therapy is null and the end date is a complete date
   and the end date is after the date of the first study visit then the start date will be set to the date of the first study visit.
   otherwise the start date will be set to the end date of the therapy.
If the end date of a therapy is null and the start date is not a complete date then the end date will be set to the study end date.
If the end date of a therapy is null and the start date is a complete date
   and the start date is prior to the study end date then the end date will be set to the study end date.
   otherwise, the end date will be set to the start date of the therapy.

### Assessment of Systemic Sclerosis-Associated Raynaud's Phenomenon (ASRAP) Questionnaire

The Assessment of Systemic sclerosis-associated Raynaud's Phenomenon (ASRAP) questionnaire is a patient-reported outcome (PRO) instrument devised to assess the severity and impact of systemic sclerosis-associated Raynaud's Syndrome. See Pauling et. al. American College of Rheumatology Convergence 2021, Abstract Number 401 (https://acrabstracts.org/abstract/item-reduction-for-the-assessment-of-systemic-sclerosis-associated-raynauds-phenomenon-asrap-questionnaire-using-data-from-the-international-multicentre-asrap-validation-study/), which is incorporated by reference herein in its entirety.

### Example 3. Phase 2A Clinical Trial Data Collected

The following data was actually obtained in accordance with the protocol and plan delineated in Examples 1 and 2. The accompanying data involved 11 patients out of the planned 76 patient study. The study was done in patients with scleroderma (systemic sclerosis) who had relatively frequent Raynaud symptoms as they needed to average at least one attack per day during a screening period of up to two weeks. In this first phase of this double-blind placebo-controlled, prospective randomized study, patients were treated in parallel and after meeting study criteria were randomized to receive either placebo, cilnidipine 10 mg daily, cilnidipine 20 mg daily, tadalafil 5 mg daily, cilnidipine 10 mg plus 5 mg of tadalafil, or cilnidipine 20 mg plus 5 mg of tadalafil.

Tables A-1 to A-8 include data that relates to the frequency of symptomatic Raynaud's attacks.

TABLE A-1
Weekly Symptomatic RP Attacks Change and Percent Change from Baseline. ITT Population - Part A.
Frequency of weekly symptomatic RP attacks is defined as the total number of symptomatic RP attacks divided by the
number of days with available diary data within the last 7 days of the dosing period (excluding the last dosing day)
then multiplied by 7. Baseline is calculated as total number of symptomatic RP attacks during the last 7 days of
screening period (excluding the first dosing day) divided by the number of days with available data then multiplied
by 7. Only symptomatic RP attacks, defined as RP attacks with patient reported finger color changes associated with
at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean (SD) | 21.00 | 12.50 (7.778) | 8.00 (5.657) | 8.00 (1.414) | 14.25 (5.303) | 17.83 (8.721) |
| Median | 21.00 | 12.50 | 8.00 | 8.00 | 14.25 | 17.83 |
| Q1, Q3 | 21.00, 21.00 | 7.00, 18.00 | 4.00, 12.00 | 7.00, 9.00 | 10.50, 18.00 | 11.67, 24.00 |
| Min, Max | 21.0, 21.0 | 7.0, 18.0 | 4.0, 12.0 | 7.0, 9.0 | 10.5, 18.0 | 11.7, 24.0 |
| Weekly Symptomatic | | | | | | |
| RP Attacks | | | | | | |
| n | 1 | 2 | 2 | 2 | 2 | 2 |
| Mean (SD) | 14.00 | 8.00 (4.243) | 12.50 (7.778) | 4.50 (0.707) | 11.50 (2.121) | 8.83 (5.893) |
| Median | 14.00 | 8.00 | 12.50 | 4.50 | 11.50 | 8.83 |
| Q1, Q3 | 14.00, 14.00 | 5.00, 11.00 | 7.00, 18.00 | 4.00, 5.00 | 10.00, 13.00 | 4.67, 13.00 |
| Min, Max | 14.0, 14.0 | 5.0, 11.0 | 7.0, 18.0 | 4.0, 5.0 | 10.0, 13.0 | 4.7, 13.0 |
| Change from | | | | | | |
| Baseline | | | | | | |
| n | 1 | 2 | 2 | 2 | 2 | 2 |
| Mean (SD) | −7.00 | −4.50 (3.536) | 4.50 (2.121) | −3.50 (0.707) | −2.75 (3.182) | −9.00 (2.828) |
| Median | −7.00 | −4.50 | 4.50 | −3.50 | −2.75 | −9.00 |
| Q1, Q3 | −7.00, −7.00 | −7.00, −2.00 | 3.00, 6.00 | −4.00, −3.00 | −5.00, −0.50 | −11.00, −7.00 |
| Min, Max | −7.0, −7.0 | −7.0, −2.0 | 3.0, 6.0 | −4.0, −3.0 | −5.0, −0.5 | −11.0, −7.0 |
| Percent Change | | | | | | |
| from Baseline | | | | | | |
| n | 1 | 2 | 2 | 2 | 2 | 2 |
| Mean (SD) | −33.33 | −33.73 (7.296) | 62.50 (17.678) | −43.65 (1.122) | −16.27 (16.275) | −52.92 (10.017) |
| Median | −33.33 | −33.73 | 62.50 | −43.65 | −16.27 | −52.92 |
| Q1, Q3 | −33.33, −33.33 | −38.89, −28.57 | 50.00, 75.00 | −44.44, −42.86 | −27.78, −4.76 | −60.00, −45.83 |
| Min, Max | −33.3, −33.3 | −38.9, −28.6 | 50.0, 75.0 | −44.4, −42.9 | −27.8, −4.8 | −60.0, −45.8 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean (SD) | 15.33 (7.371) | 11.13 (4.802) | 12.33 (7.572) | 13.38 (5.528) | 12.92 (6.426) |
| Median | 18.00 | 9.75 | 9.00 | 14.25 | 11.67 |
| Q1, Q3 | 7.00, 21.00 | 8.00, 14.25 | 7.00, 21.00 | 8.75, 18.00 | 7.00, 18.00 |
| Min, Max | 7.0, 21.0 | 7.0, 18.0 | 7.0, 21.0 | 7.0, 18.0 | 4.0, 24.0 |
| Weekly Symptomatic | | | | | |
| RP Attacks | | | | | |
| n | 3 | 4 | 3 | 4 | 11 |
| Mean (SD) | 10.00 (4.583) | 8.00 (4.243) | 7.67 (5.508) | 9.75 (3.403) | 9.52 (4.689) |
| Median | 11.00 | 7.50 | 5.00 | 10.50 | 10.00 |
| Q1, Q3 | 5.00, 14.00 | 4.50, 11.50 | 4.00, 14.00 | 7.50, 12.00 | 5.00, 13.00 |
| Min, Max | 5.0, 14.0 | 4.0, 13.0 | 4.0, 14.0 | 5.0, 13.0 | 4.0, 18.0 |
| Change from | | | | | |
| Baseline | | | | | |
| n | 3 | 4 | 3 | 4 | 11 |
| Mean (SD) | −5.33 (2.887) | −3.13 (1.931) | −4.67 (2.082) | −3.63 (2.926) | −3.41 (4.893) |
| Median | −7.00 | −3.50 | −4.00 | −3.50 | −4.00 |
| Q1, Q3 | −7.00, −2.00 | −4.50, −1.75 | −7.00, −3.00 | −6.00, −1.25 | −7.00, −0.50 |
| Min, Max | −7.0, −2.0 | −5.0, −0.5 | −7.0, −3.0 | −7.0, −0.5 | −11.0, 6.0 |
| Percent Change | | | | | |
| from Baseline | | | | | |
| n | 3 | 4 | 3 | 4 | 11 |
| Mean (SD) | −33.60 (5.164) | −29.96 (18.401) | −40.21 (6.009) | −25.00 (14.410) | −18.32 (42.640) |
| Median | −33.33 | −35.32 | −42.86 | −28.17 | −33.33 |
| Q1, Q3 | −38.89, −28.57 | −43.65, −16.27 | −44.44, −33.33 | −33.73, −16.27 | −44.44, −4.76 |
| Min, Max | −38.9, −28.6 | −44.4, −4.8 | −44.4, −33.3 | −38.9, −4.8 | −60.0, 75.0 |

TABLE A-2
Weekly Symptomatic RP Attacks Change and Percent Change from Baseline (Excluding Outliers). ITT Population - Part A.
Frequency of weekly symptomatic RP attacks is defined as the total number of symptomatic RP attacks divided by the
number of days with available diary data within the last 7 days of the dosing period (excluding the last dosing day)
then multiplied by 7. Baseline is calculated as total number of symptomatic RP attacks during the last 7 days of
screening period (excluding the first dosing day) divided by the number of days with available data then multiplied
by 7. Only symptomatic RP attacks, defined as RP attacks with patient reported finger color changes associated with
at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean (SD) | 21.00 | 12.50 (7.778) | 12.00 | 8.00 (1.414) | 14.25 (5.303) | 17.83 (8.721) |
| Median | 21.00 | 12.50 | 12.00 | 8.00 | 14.25 | 17.83 |
| Q1, Q3 | 21.00, 21.00 | 7.00, 18.00 | 12.00, 12.00 | 7.00, 9.00 | 10.50, 18.00 | 11.67, 24.00 |
| Min, Max | 21.0, 21.0 | 7.0, 18.0 | 12.0, 12.0 | 7.0, 9.0 | 10.5, 18.0 | 11.7, 24.0 |
| Weekly Symptomatic | | | | | | |
| RP Attacks | | | | | | |
| n | 1 | 2 | 1 | 2 | 2 | 2 |
| Mean (SD) | 14.00 | 8.00 (4.243) | 18.00 | 4.50 (0.707) | 11.50 (2.121) | 8.83 (5.893) |
| Median | 14.00 | 8.00 | 18.00 | 4.50 | 11.50 | 8.83 |
| Q1, Q3 | 14.00, 14.00 | 5.00, 11.00 | 18.00, 18.00 | 4.00, 5.00 | 10.00, 13.00 | 4.67, 13.00 |
| Min, Max | 14.0, 14.0 | 5.0, 11.0 | 18.0, 18.0 | 4.0, 5.0 | 10.0, 13.0 | 4.7, 13.0 |
| Change from | | | | | | |
| Baseline | | | | | | |
| n | 1 | 2 | 1 | 2 | 2 | 2 |
| Mean (SD) | −7.00 | −4.50 (3.536) | 6.00 | −3.50 (0.707) | −2.75 (3.182) | −9.00 (2.828) |
| Median | −7.00 | −4.50 | 6.00 | −3.50 | −2.75 | −9.00 |
| Q1, Q3 | −7.00, −7.00 | −7.00, −2.00 | 6.00, 6.00 | −4.00, −3.00 | −5.00, −0.50 | −11.00, −7.00 |
| Min, Max | −7.0, −7.0 | −7.0, −2.0 | 6.0, 6.0 | −4.0, −3.0 | −5.0, −0.5 | −11.0, −7.0 |
| Percent Change | | | | | | |
| from Baseline | | | | | | |
| n | 1 | 2 | 1 | 2 | 2 | 2 |
| Mean (SD) | −33.33 | −33.73 (7.296) | 50.00 | −43.65 (1.122) | −16.27 (16.275) | −52.92 (10.017) |
| Median | −33.33 | −33.73 | 50.00 | −43.65 | −16.27 | −52.92 |
| Q1, Q3 | −33.33, −33.33 | −38.89, −28.57 | 50.00, 50.00 | −44.44, −42.86 | −27.78, −4.76 | −60.00, −45.83 |
| Min, Max | −33.3, −33.3 | −38.9, −28.6 | 50.0, 50.0 | −44.4, −42.9 | −27.8, −4.8 | −60.0, −45.8 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean (SD) | 15.33 (7.371) | 11.13 (4.802) | 12.33 (7.572) | 13.38 (5.528) | 13.82 (6.013) |
| Median | 18.00 | 9.75 | 9.00 | 14.25 | 11.83 |
| Q1, Q3 | 7.00, 21.00 | 8.00, 14.25 | 7.00, 21.00 | 8.75, 18.00 | 9.00, 18.00 |
| Min, Max | 7.0, 21.0 | 7.0, 18.0 | 7.0, 21.0 | 7.0, 18.0 | 7.0, 24.0 |
| Weekly Symptomatic | | | | | |
| RP Attacks | | | | | |
| n | 3 | 4 | 3 | 4 | 10 |
| Mean (SD) | 10.00 (4.583) | 8.00 (4.243) | 7.67 (5.508) | 9.75 (3.403) | 9.77 (4.864) |
| Median | 11.00 | 7.50 | 5.00 | 10.50 | 10.50 |
| Q1, Q3 | 5.00, 14.00 | 4.50, 11.50 | 4.00, 14.00 | 7.50, 12.00 | 5.00, 13.00 |
| Min, Max | 5.0, 14.0 | 4.0, 13.0 | 4.0, 14.0 | 5.0, 13.0 | 4.0, 18.0 |
| Change from | | | | | |
| Baseline | | | | | |
| n | 3 | 4 | 3 | 4 | 10 |
| Mean (SD) | −5.33 (2.887) | −3.13 (1.931) | −4.67 (2.082) | −3.63 (2.926) | −4.05 (4.645) |
| Median | −7.00 | −3.50 | −4.00 | −3.50 | −4.50 |
| Q1, Q3 | −7.00, −2.00 | −4.50, −1.75 | −7.00, −3.00 | −6.00, −1.25 | −7.00, −2.00 |
| Min, Max | −7.0, −2.0 | −5.0, −0.5 | −7.0, −3.0 | −7.0, −0.5 | −11.0, 6.0 |
| Percent Change | | | | | |
| from Baseline | | | | | |
| n | 3 | 4 | 3 | 4 | 10 |
| Mean (SD) | −33.60 (5.164) | −29.96 (18.401) | −40.21 (6.009) | −25.00 (14.410) | −27.65 (30.918) |
| Median | −33.33 | −35.32 | −42.86 | −28.17 | −36.11 |
| Q1, Q3 | −38.89, −28.57 | −43.65, −16.27 | −44.44, −33.33 | −33.73, −16.27 | −44.44, −27.78 |
| Min, Max | −38.9, −28.6 | −44.4, −4.8 | −44.4, −33.3 | −38.9, −4.8 | −60.0, 50.0 |

TABLE A-3
Weekly Symptomatic RP Attacks Change and Percent Change from Baseline by Baseline RCS. ITT Population - Part A.
Frequency of weekly symptomatic RP attacks is defined as the total number of symptomatic RP attacks divided by the
number of days with available diary data within the last 7 days of the dosing period (excluding the last dosing day)
then multiplied by 7. Baseline is calculated as total number of symptomatic RP attacks during the last 7 days of
screening period (excluding the first dosing day) divided by the number of days with available data then multiplied
by 7. Only symptomatic RP attacks, defined as RP attacks with patient reported finger color changes associated with
at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean (SD) | 21.00 | 12.50 (7.778) | | 9.00 | 18.00 | 17.83 (8.721) |
| Median | 21.00 | 12.50 | | 9.00 | 18.00 | 17.83 |
| Q1, Q3 | 21.00, 21.00 | 7.00, 18.00 | | 9.00, 9.00 | 18.00, 18.00 | 11.67, 24.00 |
| Min, Max | 21.0, 21.0 | 7.0, 18.0 | | 9.0, 9.0 | 18.0, 18.0 | 11.7, 24.0 |
| Weekly Symptomatic | | | | | | |
| RP Attacks | | | | | | |
| n | 1 | 2 | | 1 | 1 | 2 |
| Mean (SD) | 14.00 | 8.00 (4.243) | | 5.00 | 13.00 | 8.83 (5.893) |
| Median | 14.00 | 8.00 | | 5.00 | 13.00 | 8.83 |
| Q1, Q3 | 14.00, 14.00 | 5.00, 11.00 | | 5.00, 5.00 | 13.00, 13.00 | 4.67, 13.00 |
| Min, Max | 14.0, 14.0 | 5.0, 11.0 | | 5.0, 5.0 | 13.0, 13.0 | 4.7, 13.0 |
| Change from | | | | | | |
| Baseline | | | | | | |
| n | 1 | 2 | | 1 | 1 | 2 |
| Mean (SD) | −7.00 | −4.50 (3.536) | | −4.00 | −5.00 | −9.00 (2.828) |
| Median | −7.00 | −4.50 | | −4.00 | −5.00 | −9.00 |
| Q1, Q3 | −7.00, −7.00 | −7.00, −2.00 | | −4.00, −4.00 | −5.00, −5.00 | −11.00, −7.00 |
| Min, Max | −7.0, −7.0 | −7.0, −2.0 | | −4.0, −4.0 | −5.0, −5.0 | −11.0, −7.0 |
| Percent Change | | | | | | |
| from Baseline | | | | | | |
| n | 1 | 2 | | 1 | 1 | 2 |
| Mean (SD) | −33.33 | −33.73 (7.296) | | −44.44 | −27.78 | −52.92 (10.017) |
| Median | −33.33 | −33.73 | | −44.44 | −27.78 | −52.92 |
| Q1, Q3 | −33.33, −33.33 | −38.89, −28.57 | | −44.44, −44.44 | −27.78, −27.78 | −60.00, −45.83 |
| Min, Max | −33.3, −33.3 | −38.9, −28.6 | | −44.4, −44.4 | −27.8, −27.8 | −60.0, −45.8 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean (SD) | 15.33 (7.371) | 13.50 (6.364) | 15.00 (8.485) | 14.33 (6.351) | 15.52 (6.380) |
| Median | 18.00 | 13.50 | 15.00 | 18.00 | 18.00 |
| Q1, Q3 | 7.00, 21.00 | 9.00, 18.00 | 9.00, 21.00 | 7.00, 18.00 | 9.00, 21.00 |
| Min, Max | 7.0, 21.0 | 9.0, 18.0 | 9.0, 21.0 | 7.0, 18.0 | 7.0, 24.0 |
| Weekly Symptomatic | | | | | |
| RP Attacks | | | | | |
| n | 3 | 2 | 2 | 3 | 7 |
| Mean (SD) | 10.00 (4.583) | 9.00 (5.657) | 9.50 (6.364) | 9.67 (4.163) | 9.38 (4.297) |
| Median | 11.00 | 9.00 | 9.50 | 11.00 | 11.00 |
| Q1, Q3 | 5.00, 14.00 | 5.00, 13.00 | 5.00, 14.00 | 5.00, 13.00 | 5.00, 13.00 |
| Min, Max | 5.0, 14.0 | 5.0, 13.0 | 5.0, 14.0 | 5.0, 13.0 | 4.7, 14.0 |
| Change from | | | | | |
| Baseline | | | | | |
| n | 3 | 2 | 2 | 3 | 7 |
| Mean (SD) | −5.33 (2.887) | −4.50 (0.707) | −5.50 (2.121) | −4.67 (2.517) | −6.14 (2.854) |
| Median | −7.00 | −4.50 | −5.50 | −5.00 | −7.00 |
| Q1, Q3 | −7.00, −2.00 | −5.00, −4.00 | −7.00, −4.00 | −7.00, −2.00 | −7.00, −4.00 |
| Min, Max | −7.0, −2.0 | −5.0, −4.0 | −7.0, −4.0 | −7.0, −2.0 | −11.0, −2.0 |
| Percent Change | | | | | |
| from Baseline | | | | | |
| n | 3 | 2 | 2 | 3 | 7 |
| Mean (SD) | −33.60 (5.164) | −36.11 (11.785) | −38.89 (7.857) | −31.75 (6.199) | −39.84 (11.396) |
| Median | −33.33 | −36.11 | −38.89 | −28.57 | −38.89 |
| Q1, Q3 | −38.89, −28.57 | −44.44, −27.78 | −44.44, −33.33 | −38.89, −27.78 | −45.83, −28.57 |
| Min, Max | −38.9, −28.6 | −44.4, −27.8 | −44.4, −33.3 | −38.9, −27.8 | −60.0, −27.8 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean (SD) | | | 8.00 (5.657) | 7.00 | 10.50 | |
| Median | | | 8.00 | 7.00 | 10.50 | |
| Q1, Q3 | | | 4.00, 12.00 | 7.00, 7.00 | 10.50, 10.50 | |
| Min, Max | | | 4.0, 12.0 | 7.0, 7.0 | 10.5, 10.5 | |
| Weekly Symptomatic | | | | | | |
| RP Attacks | | | | | | |
| n | | | 2 | 1 | 1 | |
| Mean (SD) | | | 12.50 (7.778) | 4.00 | 10.00 | |
| Median | | | 12.50 | 4.00 | 10.00 | |
| Q1, Q3 | | | 7.00, 18.00 | 4.00, 4.00 | 10.00, 10.00 | |
| Min, Max | | | 7.0, 18.0 | 4.0, 4.0 | 10.0, 10.0 | |
| Change from | | | | | | |
| Baseline | | | | | | |
| n | | | 2 | 1 | 1 | |
| Mean (SD) | | | 4.50 (2.121) | −3.00 | −0.50 | |
| Median | | | 4.50 | −3.00 | −0.50 | |
| Q1, Q3 | | | 3.00, 6.00 | −3.00, −3.00 | −0.50, −0.50 | |
| Min, Max | | | 3.0, 6.0 | −3.0, −3.0 | −0.5, −0.5 | |
| Percent Change | | | | | | |
| From Baseline | | | | | | |
| n | | | 2 | 1 | 1 | |
| Mean (SD) | | | 62.50 (17.678) | −42.86 | −4.76 | |
| Median | | | 62.50 | −42.86 | −4.76 | |
| Q1, Q3 | | | 50.00, 75.00 | −42.86, −42.86 | −4.76, −4.76 | |
| Min, Max | | | 50.0, 75.0 | −42.9, −42.9 | −4.8, −4.8 | |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean (SD) | | 8.75 (2.475) | 7.00 | 10.50 | 8.38 (3.591) |
| Median | | 8.75 | 7.00 | 10.50 | 8.75 |
| Q1, Q3 | | 7.00, 10.50 | 7.00, 7.00 | 10.50, 10.50 | 5.50, 11.25 |
| Min, Max | | 7.0, 10.5 | 7.0, 7.0 | 10.5, 10.5 | 4.0, 12.0 |
| Weekly Symptomatic | | | | | |
| RP Attacks | | | | | |
| n | | 2 | 1 | 1 | 4 |
| Mean (SD) | | 7.00 (4.243) | 4.00 | 10.00 | 9.75 (6.021) |
| Median | | 7.00 | 4.00 | 10.00 | 8.50 |
| Q1, Q3 | | 4.00, 10.00 | 4.00, 4.00 | 10.00, 10.00 | 5.50, 14.00 |
| Min, Max | | 4.0, 10.0 | 4.0, 4.0 | 10.0, 10.0 | 4.0, 18.0 |
| Change from | | | | | |
| Baseline | | | | | |
| n | | 2 | 1 | 1 | 4 |
| Mean (SD) | | −1.75 (1.768) | −3.00 | −0.50 | 1.38 (3.945) |
| Median | | −1.75 | −3.00 | −0.50 | 1.25 |
| Q1, Q3 | | −3.00, −0.50 | −3.00, −3.00 | −0.50, −0.50 | −1.75, 4.50 |
| Min, Max | | −3.0, −0.5 | −3.0, −3.0 | −0.5, −0.5 | −3.0, 6.0 |
| Percent Change | | | | | |
| from Baseline | | | | | |
| n | | 2 | 1 | 1 | 4 |
| Mean (SD) | | −23.81 (26.937) | −42.86 | −4.76 | 19.35 (53.190) |
| Median | | −23.81 | −42.86 | −4.76 | 22.62 |
| Q1, Q3 | | −42.86, −4.76 | −42.86, −42.86 | −4.76, −4.76 | −23.81, 62.50 |
| Min, Max | | −42.9, −4.8 | −42.9, −42.9 | −4.8, −4.8 | −42.9, 75.0 |

TABLE A-4
Weekly Symptomatic RP Attacks Change and Percent Change from Baseline
(Excluding Outliers) by Baseline RCS. ITT Population - Part A.
Frequency of weekly symptomatic RP attacks is defined as the total number of symptomatic RP attacks divided by the
number of days with available diary data within the last 7 days of the dosing period (excluding the last dosing day)
then multiplied by 7. Baseline is calculated as total number of symptomatic RP attacks during the last 7 days of
screening period (excluding the first dosing day) divided by the number of days with available data then multiplied
by 7. Only symptomatic RP attacks, defined as RP attacks with patient reported finger color changes associated with
at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean (SD) | 21.00 | 12.50 (7.778) | | 9.00 | 18.00 | 17.83 (8.721) |
| Median | 21.00 | 12.50 | | 9.00 | 18.00 | 17.83 |
| Q1, Q3 | 21.00, 21.00 | 7.00, 18.00 | | 9.00, 9.00 | 18.00, 18.00 | 11.67, 24.00 |
| Min, Max | 21.0, 21.0 | 7.0, 18.0 | | 9.0, 9.0 | 18.0, 18.0 | 11.7, 24.0 |
| Weekly Symptomatic | | | | | | |
| RP Attacks | | | | | | |
| n | 1 | 2 | | 1 | 1 | 2 |
| Mean (SD) | 14.00 | 8.00 (4.243) | | 5.00 | 13.00 | 8.83 (5.893) |
| Median | 14.00 | 8.00 | | 5.00 | 13.00 | 8.83 |
| Q1, Q3 | 14.00, 14.00 | 5.00, 11.00 | | 5.00, 5.00 | 13.00, 13.00 | 4.67, 13.00 |
| Min, Max | 14.0, 14.0 | 5.0, 11.0 | | 5.0, 5.0 | 13.0, 13.0 | 4.7, 13.0 |
| Change from | | | | | | |
| Baseline | | | | | | |
| n | 1 | 2 | | 1 | 1 | 2 |
| Mean (SD) | −7.00 | −4.50 (3.536) | | −4.00 | −5.00 | −9.00 (2.828) |
| Median | −7.00 | −4.50 | | −4.00 | −5.00 | −9.00 |
| Q1, Q3 | −7.00, −7.00 | −7.00, −2.00 | | −4.00, −4.00 | −5.00, −5.00 | −11.00, −7.00 |
| Min, Max | −7.0, −7.0 | −7.0, −2.0 | | −4.0, −4.0 | −5.0, −5.0 | −11.0, −7.0 |
| Percent Change | | | | | | |
| From Baseline | | | | | | |
| n | 1 | 2 | | 1 | 1 | 2 |
| Mean (SD) | −33.33 | −33.73 (7.296) | | −44.44 | −27.78 | −52.92 (10.017) |
| Median | −33.33 | −33.73 | | −44.44 | −27.78 | −52.92 |
| Q1, Q3 | −33.33, −33.33 | −38.89, −28.57 | | −44.44, −44.44 | −27.78, −27.78 | −60.00, −45.83 |
| Min, Max | −33.3, −33.3 | −38.9, −28.6 | | −44.4, −44.4 | −27.8, −27.8 | −60.0, −45.8 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean (SD) | 15.33 (7.371) | 13.50 (6.364) | 15.00 (8.485) | 14.33 (6.351) | 15.52 (6.380) |
| Median | 18.00 | 13.50 | 15.00 | 18.00 | 18.00 |
| Q1, Q3 | 7.00, 21.00 | 9.00, 18.00 | 9.00, 21.00 | 7.00, 18.00 | 9.00, 21.00 |
| Min, Max | 7.0, 21.0 | 9.0, 18.0 | 9.0, 21.0 | 7.0, 18.0 | 7.0, 24.0 |
| Weekly Symptomatic | | | | | |
| RP Attacks | | | | | |
| n | 3 | 2 | 2 | 3 | 7 |
| Mean (SD) | 10.00 (4.583) | 9.00 (5.657) | 9.50 (6.364) | 9.67 (4.163) | 9.38 (4.297) |
| Median | 11.00 | 9.00 | 9.50 | 11.00 | 11.00 |
| Q1, Q3 | 5.00, 14.00 | 5.00, 13.00 | 5.00, 14.00 | 5.00, 13.00 | 5.00, 13.00 |
| Min, Max | 5.0, 14.0 | 5.0, 13.0 | 5.0, 14.0 | 5.0, 13.0 | 4.7, 14.0 |
| Change from | | | | | |
| Baseline | | | | | |
| n | 3 | 2 | 2 | 3 | 7 |
| Mean (SD) | −5.33 (2.887) | −4.50 (0.707) | −5.50 (2.121) | −4.67 (2.517) | −6.14 (2.854) |
| Median | −7.00 | −4.50 | −5.50 | −5.00 | −7.00 |
| Q1, Q3 | −7.00, −2.00 | −5.00, −4.00 | −7.00, −4.00 | −7.00, −2.00 | −7.00, −4.00 |
| Min, Max | −7.0, −2.0 | −5.0, −4.0 | −7.0, −4.0 | −7.0, −2.0 | −11.0, −2.0 |
| Percent Change | | | | | |
| from Baseline | | | | | |
| n | 3 | 2 | 2 | 3 | 7 |
| Mean (SD) | −33.60 (5.164) | −36.11 (11.785) | −38.89 (7.857) | −31.75 (6.199) | −39.84 (11.396) |
| Median | −33.33 | −36.11 | −38.89 | −28.57 | −38.89 |
| Q1, Q3 | −38.89, −28.57 | −44.44, −27.78 | −44.44, −33.33 | −38.89, −27.78 | −45.83, −28.57 |
| Min, Max | −38.9, −28.6 | −44.4, −27.8 | −44.4, −33.3 | −38.9, −27.8 | −60.0, −27.8 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean (SD) | | | 12.00 | 7.00 | 10.50 | |
| Median | | | 12.00 | 7.00 | 10.50 | |
| Q1, Q3 | | | 12.00, 12.00 | 7.00, 7.00 | 10.50, 10.50 | |
| Min, Max | | | 12.0, 12.0 | 7.0, 7.0 | 10.5, 10.5 | |
| Weekly Symptomatic | | | | | | |
| RP Attacks | | | | | | |
| n | | | 1 | 1 | 1 | |
| Mean (SD) | | | 18.00 | 4.00 | 10.00 | |
| Median | | | 18.00 | 4.00 | 10.00 | |
| Q1, Q3 | | | 18.00, 18.00 | 4.00, 4.00 | 10.00, 10.00 | |
| Min, Max | | | 18.0, 18.0 | 4.0, 4.0 | 10.0, 10.0 | |
| Change from | | | | | | |
| Baseline | | | | | | |
| n | | | 1 | 1 | 1 | |
| Mean (SD) | | | 6.00 | −3.00 | −0.50 | |
| Median | | | 6.00 | −3.00 | −0.50 | |
| Q1, Q3 | | | 6.00, 6.00 | −3.00, −3.00 | −0.50, −0.50 | |
| Min, Max | | | 6.0, 6.0 | −3.0, −3.0 | −0.5, −0.5 | |
| Percent Change | | | | | | |
| from Baseline | | | | | | |
| n | | | 1 | 1 | 1 | |
| Mean (SD) | | | 50.00 | −42.86 | −4.76 | |
| Median | | | 50.00 | −42.86 | −4.76 | |
| Q1, Q3 | | | 50.00, 50.00 | −42.86, −42.86 | −4.76, −4.76 | |
| Min, Max | | | 50.0, 50.0 | −42.9, −42.9 | −4.8, −4.8 | |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean (SD) | | 8.75 (2.475) | 7.00 | 10.50 | 9.83 (2.566) |
| Median | | 8.75 | 7.00 | 10.50 | 10.50 |
| Q1, Q3 | | 7.00, 10.50 | 7.00, 7.00 | 10.50, 10.50 | 7.00, 12.00 |
| Min, Max | | 7.0, 10.5 | 7.0, 7.0 | 10.5, 10.5 | 7.0, 12.0 |
| Weekly Symptomatic | | | | | |
| RP Attacks | | | | | |
| n | | 2 | 1 | 1 | 3 |
| Mean (SD) | | 7.00 (4.243) | 4.00 | 10.00 | 10.67 (7.024) |
| Median | | 7.00 | 4.00 | 10.00 | 10.00 |
| Q1, Q3 | | 4.00, 10.00 | 4.00, 4.00 | 10.00, 10.00 | 4.00, 18.00 |
| Min, Max | | 4.0, 10.0 | 4.0, 4.0 | 10.0, 10.0 | 4.0, 18.0 |
| Change from | | | | | |
| Baseline | | | | | |
| n | | 2 | 1 | 1 | 3 |
| Mean (SD) | | −1.75 (1.768) | −3.00 | −0.50 | 0.83 (4.646) |
| Median | | −1.75 | −3.00 | −0.50 | −0.50 |
| Q1, Q3 | | −3.00, −0.50 | −3.00, −3.00 | −0.50, −0.50 | −3.00, 6.00 |
| Min, Max | | −3.0, −0.5 | −3.0, −3.0 | −0.5, −0.5 | −3.0, 6.0 |
| Percent Change | | | | | |
| from Baseline | | | | | |
| n | | 2 | 1 | 1 | 3 |
| Mean (SD) | | −23.81 (26.937) | −42.86 | −4.76 | 0.79 (46.677) |
| Median | | −23.81 | −42.86 | −4.76 | −4.76 |
| Q1, Q3 | | −42.86, −4.76 | −42.86, −42.86 | −4.76, −4.76 | −42.86, 50.00 |
| Min, Max | | −42.9, −4.8 | −42.9, −42.9 | −4.8, −4.8 | −42.9, 50.0 |

TABLE A-5
Weekly RP Attacks (All Attacks) Change and Percent Change from Baseline.
ITT Population - Part A.
Frequency of weekly RP attacks is defined as the total number of RP attacks
divided by the number of days with available diary data within the last 7
days of the dosing period (excluding the last dosing day) then multiplied by 7.
Baseline is calculated as total number of RP attacks during the last 7 days of
screening period (excluding the first dosing day) divided by the number of days
with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 32.00 | 14.67 | 10.00 | 10.00 | 15.92 | 17.83 |
| (SD) | | (7.542) | (4.243) | (1.414) | (4.360) | (8.721) |
| Median | 32.00 | 14.67 | 10.00 | 10.00 | 15.92 | 17.83 |
| Q1, Q3 | 32.00, | 9.33, | 7.00, | 9.00, | 12.83, | 11.67, |
| | 32.00 | 20.00 | 13.00 | 11.00 | 19.00 | 24.00 |
| Min, Max | 32.0, | 9.3, | 7.0, | 9.0, | 12.8, | 11.7, |
| | 32.0 | 20.0 | 13.0 | 11.0 | 19.0 | 24.0 |
| Weekly RP | | | | | | |
| Attacks | | | | | | |
| n | 1 | 2 | 2 | 2 | 2 | 2 |
| Mean | 23.00 | 8.00 | 13.00 | 5.50 | 13.00 | 9.83 |
| (SD) | | (4.243) | (8.485) | (0.707) | (2.828) | (7.307) |
| Median | 23.00 | 8.00 | 13.00 | 5.50 | 13.00 | 9.83 |
| Q1, Q3 | 23.00, | 5.00, | 7.00, | 5.00, | 11.00, | 4.67, |
| | 23.00 | 11.00 | 19.00 | 6.00 | 15.00 | 15.00 |
| Min, Max | 23.0, | 5.0, | 7.0, | 5.0, | 11.0, | 4.7, |
| | 23.0 | 11.0 | 19.0 | 6.0 | 15.0 | 15.0 |
| Change | | | | | | |
| from | | | | | | |
| Baseline | | | | | | |
| n | 1 | 2 | 2 | 2 | 2 | 2 |
| Mean | −9.00 | −6.67 | 3.00 | −4.50 | −2.92 | −8.00 |
| (SD) | | (3.300) | (4.243) | (0.707) | (1.532) | (1.414) |
| Median | −9.00 | −6.67 | 3.00 | −4.50 | −2.92 | −8.00 |
| Q1, Q3 | −9.00, | −9.00, | 0.00, | −5.00, | −4.00, | −9.00, |
| | −9.00 | −4.33 | 6.00 | −4.00 | −1.83 | −7.00 |
| Min, Max | −9.0, | −9.0, | 0.0, | −5.0, | −4.0, | −9.0, |
| | −9.0 | −4.3 | 6.0 | −4.0 | −1.8 | −7.0 |
| Percent | | | | | | |
| Change | | | | | | |
| from | | | | | | |
| Baseline | | | | | | |
| n | 1 | 2 | 2 | 2 | 2 | 2 |
| Mean | −28.13 | −45.71 | 23.08 | −44.95 | −17.67 | −48.75 |
| (SD) | | (1.010) | (32.636) | (0.714) | (4.785) | (15.910) |
| Median | −28.13 | −45.71 | 23.08 | −44.95 | −17.67 | −48.75 |
| Q1, Q3 | −28.13, | −46.43, | 0.00, | −45.45, | −21.05, | −60.00, |
| | −28.13 | −45.00 | 46.15 | −44.44 | −14.29 | −37.50 |
| Min, Max | −28.1, | −46.4, | 0.0, | −45.5, | −21.1, | −60.0, |
| | −28.1 | −45.0 | 46.2 | −44.4 | −14.3 | −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 20.44 | 12.96 | 17.33 | 15.29 | 15.35 |
| (SD) | (11.340) | (4.321) | (12.741) | (5.082) | (7.600) |
| Median | 20.00 | 11.92 | 11.00 | 15.92 | 12.83 |
| Q1, Q3 | 9.33, | 10.00, | 9.00, | 11.08, | 9.33, |
| | 32.00 | 15.92 | 32.00 | 19.50 | 20.00 |
| Min, Max | 9.3, | 9.0, | 9.0, | 9.3, | 7.0, |
| | 32.0 | 19.0 | 32.0 | 20.0 | 32.0 |
| Weekly RP | | | | | |
| Attacks | | | | | |
| n | 3 | 4 | 3 | 4 | 11 |
| Mean | 13.00 | 9.25 | 11.33 | 10.50 | 11.06 |
| (SD) | (9.165) | (4.646) | (10.116) | (4.123) | (6.270) |
| Median | 11.00 | 8.50 | 6.00 | 11.00 | 11.00 |
| Q1, Q3 | 5.00, | 5.50, | 5.00, | 8.00, | 5.00, |
| | 23.00 | 13.00 | 23.00 | 13.00 | 15.00 |
| Min, Max | 5.0, | 5.0, | 5.0, | 5.0, | 4.7, |
| | 23.0 | 15.0 | 23.0 | 15.0 | 23.0 |
| Change | | | | | |
| from | | | | | |
| Baseline | | | | | |
| n | 3 | 4 | 3 | 4 | 11 |
| Mean | −7.44 | −3.71 | −6.00 | −4.79 | −4.29 |
| (SD) | (2.694) | (1.336) | (2.646) | (3.017) | (4.527) |
| Median | −9.00 | −4.00 | −5.00 | −4.17 | −4.33 |
| Q1, Q3 | −9.00, | −4.50, | −9.00, | −6.67, | −9.00, |
| | −4.33 | −2.92 | −4.00 | −2.92 | −1.83 |
| Min, Max | −9.0, | −5.0, | −9.0, | −9.0, | −9.0, |
| | −4.3 | −1.8 | −4.0 | −1.8 | 6.0 |
| Percent | | | | | |
| Change | | | | | |
| from | | | | | |
| Baseline | | | | | |
| n | 3 | 4 | 3 | 4 | 11 |
| Mean | −39.85 | −31.31 | −39.34 | −31.69 | −26.92 |
| (SD) | (10.180) | (15.996) | (9.727) | (16.436) | (29.706) |
| Median | −45.00 | −32.75 | −44.44 | −33.03 | −37.50 |
| Q1, Q3 | −46.43, | −44.95, | −45.45, | −45.71, | −45.45, |
| | −28.13 | −17.67 | −28.13 | −17.67 | −14.29 |
| Min, Max | −46.4, | −45.5, | −45.5, | −46.4, | −60.0, |
| | −28.1 | −14.3 | −28.1 | −14.3 | 46.2 |

TABLE A-6
Weekly RP Attacks (All Attacks) Change and Percent Change
from Baseline (Excluding Outliers). ITT Population - Part A.
Frequency of weekly RP attacks is defined as the total number
of RP attacks divided by the number of days with available diary
data within the last 7 days of the dosing period (excluding the
last dosing day) then multiplied by 7. Baseline is calculated as
total number of RP attacks during the last 7 days of screening
period (excluding the first dosing day) divided by the number
of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 32.00 | 14.67 | 13.00 | 10.00 | 15.92 | 17.83 |
| (SD) | | (7.542) | | (1.414) | (4.360) | (8.721) |
| Median | 32.00 | 14.67 | 13.00 | 10.00 | 15.92 | 17.83 |
| Q1, Q3 | 32.00, | 9.33, | 13.00, | 9.00, | 12.83, | 11.67, |
| | 32.00 | 20.00 | 13.00 | 11.00 | 19.00 | 24.00 |
| Min, Max | 32.0, | 9.3, | 13.0, | 9.0, | 12.8, | 11.7, |
| | 32.0 | 20.0 | 13.0 | 11.0 | 19.0 | 24.0 |
| Weekly RP | | | | | | |
| Attacks | | | | | | |
| n | 1 | 2 | 1 | 2 | 2 | 2 |
| Mean | 23.00 | 8.00 | 19.00 | 5.50 | 13.00 | 9.83 |
| (SD) | | (4.243) | | (0.707) | (2.828) | (7.307) |
| Median | 23.00 | 8.00 | 19.00 | 5.50 | 13.00 | 9.83 |
| Q1, Q3 | 23.00, | 5.00, | 19.00, | 5.00, | 11.00, | 4.67, |
| | 23.00 | 11.00 | 19.00 | 6.00 | 15.00 | 15.00 |
| Min, Max | 23.0, | 5.0, | 19.0, | 5.0, | 11.0, | 4.7, |
| | 23.0 | 11.0 | 19.0 | 6.0 | 15.0 | 15.0 |
| Change | | | | | | |
| from | | | | | | |
| Baseline | | | | | | |
| n | 1 | 2 | 1 | 2 | 2 | 2 |
| Mean | −9.00 | −6.67 | 6.00 | −4.50 | −2.92 | −8.00 |
| (SD) | | (3.300) | | (0.707) | (1.532) | (1.414) |
| Median | −9.00 | −6.67 | 6.00 | −4.50 | −2.92 | −8.00 |
| Q1, Q3 | −9.00, | −9.00, | 6.00, | −5.00, | −4.00, | −9.00, |
| | −9.00 | −4.33 | 6.00 | −4.00 | −1.83 | −7.00 |
| Min, Max | −9.0, | −9.0, | 6.0, | −5.0, | −4.0, | −9.0, |
| | −9.0 | −4.3 | 6.0 | −4.0 | −1.8 | −7.0 |
| Percent | | | | | | |
| Change | | | | | | |
| from | | | | | | |
| Baseline | | | | | | |
| n | 1 | 2 | 1 | 2 | 2 | 2 |
| Mean | −28.13 | −45.71 | 46.15 | −44.95 | −17.67 | −48.75 |
| (SD) | | (1.010) | | (0.714) | (4.785) | (15.910) |
| Median | −28.13 | −45.71 | 46.15 | −44.95 | −17.67 | −48.75 |
| Q1, Q3 | −28.13, | −46.43, | 46.15, | −45.45, | −21.05, | −60.00, |
| | −28.13 | −45.00 | 46.15 | −44.44 | −14.29 | −37.50 |
| Min, Max | −28.1, | −46.4, | 46.2, | −45.5, | −21.1, | −60.0, |
| | −28.1 | −45.0 | 46.2 | −44.4 | −14.3 | −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 20.44 | 12.96 | 17.33 | 15.29 | 16.18 |
| (SD) | (11.340) | (4.321) | (12.741) | (5.082) | (7.460) |
| Median | 20.00 | 11.92 | 11.00 | 15.92 | 12.92 |
| Q1, Q3 | 9.33, | 10.00, | 9.00, | 11.08, | 11.00, |
| | 32.00 | 15.92 | 32.00 | 19.50 | 20.00 |
| Min, Max | 9.3, | 9.0, | 9.0, | 9.3, | 9.0, |
| | 32.0 | 19.0 | 32.0 | 20.0 | 32.0 |
| Weekly RP | | | | | |
| Attacks | | | | | |
| n | 3 | 4 | 3 | 4 | 10 |
| Mean | 13.00 | 9.25 | 11.33 | 10.50 | 11.47 |
| (SD) | (9.165) | (4.646) | (10.116) | (4.123) | (6.454) |
| Median | 11.00 | 8.50 | 6.00 | 11.00 | 11.00 |
| Q1, Q3 | 5.00, | 5.50, | 5.00, | 8.00, | 5.00, |
| | 23.00 | 13.00 | 23.00 | 13.00 | 15.00 |
| Min, Max | 5.0, | 5.0, | 5.0, | 5.0, | 4.7, |
| | 23.0 | 15.0 | 23.0 | 15.0 | 23.0 |
| Change | | | | | |
| from | | | | | |
| Baseline | | | | | |
| n | 3 | 4 | 3 | 4 | 10 |
| Mean | −7.44 | −3.71 | −6.00 | −4.79 | −4.72 |
| (SD) | (2.694) | (1.336) | (2.646) | (3.017) | (4.530) |
| Median | −9.00 | −4.00 | −5.00 | −4.17 | −4.67 |
| Q1, Q3 | −9.00, | −4.50, | −9.00, | −6.67, | −9.00, |
| | −4.33 | −2.92 | −4.00 | −2.92 | −4.00 |
| Min, Max | −9.0, | −5.0, | −9.0, | −9.0, | −9.0, |
| | −4.3 | −1.8 | −4.0 | −1.8 | 6.0 |
| Percent | | | | | |
| Change | | | | | |
| from | | | | | |
| Baseline | | | | | |
| n | 3 | 4 | 3 | 4 | 10 |
| Mean | −39.85 | −31.31 | −39.34 | −31.69 | −29.61 |
| (SD) | (10.180) | (15.996) | (9.727) | (16.436) | (29.865) |
| Median | −45.00 | −32.75 | −44.44 | −33.03 | −40.97 |
| Q1, Q3 | −46.43, | −44.95, | −45.45, | −45.71, | −45.45, |
| | −28.13 | −17.67 | −28.13 | −17.67 | −21.05 |
| Min, Max | −46.4, | −45.5, | −45.5, | −46.4, | −60.0, |
| | −28.1 | −14.3 | −28.1 | −14.3 | 46.2 |

TABLE A-7
Weekly RP Attacks (All Attacks) Change and Percent
Change from Baseline by Baseline RCS. ITT Population - Part A.
Frequency of weekly RP attacks is defined as the total number
of RP attacks divided by the number of days with available diary
data within the last 7 days of the dosing period (excluding the
last dosing day) then multiplied by 7. Baseline is calculated as
total number of RP attacks during the last 7 days of screening
period (excluding the first dosing day) divided by the number
of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 32.00 | 14.67 | | 9.00 | 19.00 | 17.83 |
| (SD) | | (7.542) | | | | (8.721) |
| Median | 32.00 | 14.67 | | 9.00 | 19.00 | 17.83 |
| Q1, Q3 | 32.00, | 9.33, | | 9.00, | 19.00, | 11.67, |
| | 32.00 | 20.00 | | 9.00 | 19.00 | 24.00 |
| Min, Max | 32.0, | 9.3, | | 9.0, | 19.0, | 11.7, |
| | 32.0 | 20.0 | | 9.0 | 19.0 | 24.0 |
| Weekly RP | | | | | | |
| Attacks | | | | | | |
| n | 1 | 2 | | 1 | 1 | 2 |
| Mean | 23.00 | 8.00 | | 5.00 | 15.00 | 9.83 |
| (SD) | | (4.243) | | | | (7.307) |
| Median | 23.00 | 8.00 | | 5.00 | 15.00 | 9.83 |
| Q1, Q3 | 23.00, | 5.00, | | 5.00, | 15.00, | 4.67, |
| | 23.00 | 11.00 | | 5.00 | 15.00 | 15.00 |
| Min, Max | 23.0, | 5.0, | | 5.0, | 15.0, | 4.7, |
| | 23.0 | 11.0 | | 5.0 | 15.0 | 15.0 |
| Change | | | | | | |
| from | | | | | | |
| Baseline | | | | | | |
| n | 1 | 2 | | 1 | 1 | 2 |
| Mean | −9.00 | −6.67 | | −4.00 | −4.00 | −8.00 |
| (SD) | | (3.300) | | | | (1.414) |
| Median | −9.00 | −6.67 | | −4.00 | −4.00 | −8.00 |
| Q1, Q3 | −9.00, | −9.00, | | −4.00, | −4.00, | −9.00, |
| | −9.00 | −4.33 | | −4.00 | −4.00 | −7.00 |
| Min, Max | −9.0, | −9.0, | | −4.0, | −4.0, | −9.0, |
| | −9.0 | −4.3 | | −4.0 | −4.0 | −7.0 |
| Percent | | | | | | |
| Change | | | | | | |
| from | | | | | | |
| Baseline | | | | | | |
| n | 1 | 2 | | 1 | 1 | 2 |
| Mean | −28.13 | −45.71 | | −44.44 | −21.05 | −48.75 |
| (SD) | | (1.010) | | | | (15.910) |
| Median | −28.13 | −45.71 | | −44.44 | −21.05 | −48.75 |
| Q1, Q3 | −28.13, | −46.43, | | −44.44, | −21.05, | −60.00, |
| | −28.13 | −45.00 | | −44.44 | −21.05 | −37.50 |
| Min, Max | −28.1, | −46.4, | | −44.4, | −21.1, | −60.0, |
| | −28.1 | −45.0 | | −44.4 | −21.1 | −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil + | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 20.44 | 14.00 | 20.50 | 16.11 | 17.86 |
| (SD) | (11.340) | (7.071) | (16.263) | (5.891) | (8.496) |
| Median | 20.00 | 14.00 | 20.50 | 19.00 | 19.00 |
| Q1, Q3 | 9.33, | 9.00, | 9.00, | 9.33, | 9.33, |
| | 32.00 | 19.00 | 32.00 | 20.00 | 24.00 |
| Min, Max | 9.3, | 9.0, | 9.0, | 9.3, | 9.0, |
| | 32.0 | 19.0 | 32.0 | 20.0 | 32.0 |
| Weekly RP | | | | | |
| Attacks | | | | | |
| n | 3 | 2 | 2 | 3 | 7 |
| Mean | 13.00 | 10.00 | 14.00 | 10.33 | 11.24 |
| (SD) | (9.165) | (7.071) | (12.728) | (5.033) | (6.925) |
| Median | 11.00 | 10.00 | 14.00 | 11.00 | 11.00 |
| Q1, Q3 | 5.00, | 5.00, | 5.00, | 5.00, | 5.00, |
| | 23.00 | 15.00 | 23.00 | 15.00 | 15.00 |
| Min, Max | 5.0, | 5.0, | 5.0, | 5.0, | 4.7, |
| | 23.0 | 15.0 | 23.0 | 15.0 | 23.0 |
| Change | | | | | |
| from | | | | | |
| Baseline | | | | | |
| n | 3 | 2 | 2 | 3 | 7 |
| Mean | −7.44 | −4.00 | −6.50 | −5.78 | −6.62 |
| (SD) | (2.694) | (0.000) | (3.536) | (2.795) | (2.453) |
| Median | −9.00 | −4.00 | −6.50 | −4.33 | −7.00 |
| Q1, Q3 | −9.00, | −4.00, | −9.00, | −9.00, | −9.00, |
| | −4.33 | −4.00 | −4.00 | −4.00 | −4.00 |
| Min, Max | −9.0, | −4.0, | −9.0, | −9.0, | −9.0, |
| | −4.3 | −4.0 | −4.0 | −4.0 | −4.0 |
| Percent | | | | | |
| Change | | | | | |
| from | | | | | |
| Baseline | | | | | |
| n | 3 | 2 | 2 | 3 | 7 |
| Mean | −39.85 | −32.75 | −36.28 | −37.49 | −40.36 |
| (SD) | (10.180) | (16.541) | (11.540) | (14.256) | (12.854) |
| Median | −45.00 | −32.75 | −36.28 | −45.00 | −44.44 |
| Q1, Q3 | −46.43, | −44.44, | −44.44, | −46.43, | −46.43, |
| | −28.13 | −21.05 | −28.13 | −21.05 | −28.13 |
| Min, Max | −46.4, | −44.4, | −44.4, | −46.4, | −60.0, |
| | −28.1 | −21.1 | −28.1 | −21.1 | −21.1 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 10.00 | 11.00 | 12.83 | |
| (SD) | | | (4.243) | | | |
| Median | | | 10.00 | 11.00 | 12.83 | |
| Q1, Q3 | | | 7.00, | 11.00, | 12.83, | |
| | | | 13.00 | 11.00 | 12.83 | |
| Min, Max | | | 7.0, | 11.0, | 12.8, | |
| | | | 13.0 | 11.0 | 12.8 | |
| Weekly RP | | | | | | |
| Attacks | | | | | | |
| n | | | 2 | 1 | 1 | |
| Mean | | | 13.00 | 6.00 | 11.00 | |
| (SD) | | | (8.485) | | | |
| Median | | | 13.00 | 6.00 | 11.00 | |
| Q1, Q3 | | | 7.00, | 6.00, | 11.00, | |
| | | | 19.00 | 6.00 | 11.00 | |
| Min, Max | | | 7.0, | 6.0, | 11.0, | |
| | | | 19.0 | 6.0 | 11.0 | |
| Change | | | | | | |
| from | | | | | | |
| Baseline | | | | | | |
| n | | | 2 | 1 | 1 | |
| Mean | | | 3.00 | −5.00 | −1.83 | |
| (SD) | | | (4.243) | | | |
| Median | | | 3.00 | −5.00 | −1.83 | |
| Q1, Q3 | | | 0.00, | −5.00, | −1.83, | |
| | | | 6.00 | −5.00 | −1.83 | |
| Min, Max | | | 0.0, | −5.0, | −1.8, | |
| | | | 6.0 | −5.0 | −1.8 | |
| Percent | | | | | | |
| Change | | | | | | |
| from | | | | | | |
| Baseline | | | | | | |
| n | | | 2 | 1 | 1 | |
| Mean | | | 23.08 | −45.45 | −14.29 | |
| (SD) | | | (32.636) | | | |
| Median | | | 23.08 | −45.45 | −14.29 | |
| Q1, Q3 | | | 0.00, | −45.45, | −14.29, | |
| | | | 46.15 | −45.45 | −14.29 | |
| Min, Max | | | 0.0, | −45.5, | −14.3, | |
| | | | 46.2 | −45.5 | −14.3 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil + | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 11.92 | 11.00 | 12.83 | 10.96 |
| (SD) | | (1.296) | | | (2.790) |
| Median | | 11.92 | 11.00 | 12.83 | 11.92 |
| Q1, Q3 | | 11.00, | 11.00, | 12.83, | 9.00, |
| | | 12.83 | 11.00 | 12.83 | 12.92 |
| Min, Max | | 11.0, | 11.0, | 12.8, | 7.0, |
| | | 12.8 | 11.0 | 12.8 | 13.0 |
| Weekly RP | | | | | |
| Attacks | | | | | |
| n | | 2 | 1 | 1 | 4 |
| Mean | | 8.50 | 6.00 | 11.00 | 10.75 |
| (SD) | | (3.536) | | | (5.909) |
| Median | | 8.50 | 6.00 | 11.00 | 9.00 |
| Q1, Q3 | | 6.00, | 6.00, | 11.00, | 6.50, |
| | | 11.00 | 6.00 | 11.00 | 15.00 |
| Min, Max | | 6.0, | 6.0, | 11.0, | 6.0, |
| | | 11.0 | 6.0 | 11.0 | 19.0 |
| Change | | | | | |
| from | | | | | |
| Baseline | | | | | |
| n | | 2 | 1 | 1 | 4 |
| Mean | | −3.42 | −5.00 | −1.83 | −0.21 |
| (SD) | | (2.239) | | | (4.626) |
| Median | | −3.42 | −5.00 | −1.83 | −0.92 |
| Q1, Q3 | | −5.00, | −5.00, | −1.83, | −3.42, |
| | | −1.83 | −5.00 | −1.83 | 3.00 |
| Min, Max | | −5.0, | −5.0, | −1.8, | −5.0, |
| | | −1.8 | −5.0 | −1.8 | 6.0 |
| Percent | | | | | |
| Change | | | | | |
| from | | | | | |
| Baseline | | | | | |
| n | | 2 | 1 | 1 | 4 |
| Mean | | −29.87 | −45.45 | −14.29 | −3.40 |
| (SD) | | (22.040) | | | (38.097) |
| Median | | −29.87 | −45.45 | −14.29 | −7.14 |
| Q1, Q3 | | −45.45, | −45.45, | −14.29, | −29.87, |
| | | −14.29 | −45.45 | −14.29 | 23.08 |
| Min, Max | | −45.5, | −45.5, | −14.3, | −45.5, |
| | | −14.3 | −45.5 | −14.3 | 46.2 |

TABLE A-8
Weekly RP Attacks (All Attacks) Change and Percent Change from
Baseline (Excluding Outliers) by Baseline RCS. ITT Population - Part A.
Frequency of weekly RP attacks is defined as the total number of RP
attacks divided by the number of days with available diary data within
the last 7 days of the dosing period (excluding the last dosing day) then
multiplied by 7. Baseline is calculated as total number of RP attacks
during the last 7 days of screening period (excluding the first dosing day)
divided by the number of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 32.00 | 14.67 | | 9.00 | 19.00 | 17.83 |
| (SD) | | (7.542) | | | | (8.721) |
| Median | 32.00 | 14.67 | | 9.00 | 19.00 | 17.83 |
| Q1, Q3 | 32.00, | 9.33, | | 9.00, | 19.00, | 11.67, |
| | 32.00 | 20.00 | | 9.00 | 19.00 | 24.00 |
| Min, Max | 32.0, | 9.3, | | 9.0, | 19.0, | 11.7, |
| | 32.0 | 20.0 | | 9.0 | 19.0 | 24.0 |
| Weekly RP | | | | | | |
| Attacks | | | | | | |
| n | 1 | 2 | | 1 | 1 | 2 |
| Mean | 23.00 | 8.00 | | 5.00 | 15.00 | 9.83 |
| (SD) | | (4.243) | | | | (7.307) |
| Median | 23.00 | 8.00 | | 5.00 | 15.00 | 9.83 |
| Q1, Q3 | 23.00, | 5.00, | | 5.00, | 15.00, | 4.67, |
| | 23.00 | 11.00 | | 5.00 | 15.00 | 15.00 |
| Min, Max | 23.0, | 5.0, | | 5.0, | 15.0, | 4.7, |
| | 23.0 | 11.0 | | 5.0 | 15.0 | 15.0 |
| Change | | | | | | |
| from | | | | | | |
| Baseline | | | | | | |
| n | 1 | 2 | | 1 | 1 | 2 |
| Mean | −9.00 | −6.67 | | −4.00 | −4.00 | −8.00 |
| (SD) | | (3.300) | | | | (1.414) |
| Median | −9.00 | −6.67 | | −4.00 | −4.00 | −8.00 |
| Q1, Q3 | −9.00, | −9.00, | | −4.00, | −4.00, | −9.00, |
| | −9.00 | −4.33 | | −4.00 | −4.00 | −7.00 |
| Min, Max | −9.0, | −9.0, | | −4.0, | −4.0, | −9.0, |
| | −9.0 | −4.3 | | −4.0 | −4.0 | −7.0 |
| Percent | | | | | | |
| Change | | | | | | |
| from | | | | | | |
| Baseline | | | | | | |
| n | 1 | 2 | | 1 | 1 | 2 |
| Mean | −28.13 | −45.71 | | −44.44 | −21.05 | −48.75 |
| (SD) | | (1.010) | | | | (15.910) |
| Median | −28.13 | −45.71 | | −44.44 | −21.05 | −48.75 |
| Q1, Q3 | −28.13, | −46.43, | | −44.44, | −21.05, | −60.00, |
| | −28.13 | −45.00 | | −44.44 | −21.05 | −37.50 |
| Min, Max | −28.1, | −46.4, | | −44.4, | −21.1, | −60.0, |
| | −28.1 | −45.0 | | −44.4 | −21.1 | −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 20.44 | 14.00 | 20.50 | 16.11 | 17.86 |
| (SD) | (11.340) | (7.071) | (16.263) | (5.891) | (8.496) |
| Median | 20.00 | 14.00 | 20.50 | 19.00 | 19.00 |
| Q1, Q3 | 9.33, | 9.00, | 9.00, | 9.33, | 9.33, |
| | 32.00 | 19.00 | 32.00 | 20.00 | 24.00 |
| Min, Max | 9.3, | 9.0, | 9.0, | 9.3, | 9.0, |
| | 32.0 | 19.0 | 32.0 | 20.0 | 32.0 |
| Weekly RP | | | | | |
| Attacks | | | | | |
| n | 3 | 2 | 2 | 3 | 7 |
| Mean | 13.00 | 10.00 | 14.00 | 10.33 | 11.24 |
| (SD) | (9.165) | (7.071) | (12.728) | (5.033) | (6.925) |
| Median | 11.00 | 10.00 | 14.00 | 11.00 | 11.00 |
| Q1, Q3 | 5.00, | 5.00, | 5.00, | 5.00, | 5.00, |
| | 23.00 | 15.00 | 23.00 | 15.00 | 15.00 |
| Min, Max | 5.0, | 5.0, | 5.0, | 5.0, | 4.7, |
| | 23.0 | 15.0 | 23.0 | 15.0 | 23.0 |
| Change | | | | | |
| from | | | | | |
| Baseline | | | | | |
| n | 3 | 2 | 2 | 3 | 7 |
| Mean | −7.44 | −4.00 | −6.50 | −5.78 | −6.62 |
| (SD) | (2.694) | (0.000) | (3.536) | (2.795) | (2.453) |
| Median | −9.00 | −4.00 | −6.50 | −4.33 | −7.00 |
| Q1, Q3 | −9.00, | −4.00, | −9.00, | −9.00, | −9.00, |
| | −4.33 | −4.00 | −4.00 | −4.00 | −4.00 |
| Min, Max | −9.0, | −4.0, | −9.0, | −9.0, | −9.0, |
| | −4.3 | −4.0 | −4.0 | −4.0 | −4.0 |
| Percent | | | | | |
| Change | | | | | |
| from | | | | | |
| Baseline | | | | | |
| n | 3 | 2 | 2 | 3 | 7 |
| Mean | −39.85 | −32.75 | −36.28 | −37.49 | −40.36 |
| (SD) | (10.180) | (16.541) | (11.540) | (14.256) | (12.854) |
| Median | −45.00 | −32.75 | −36.28 | −45.00 | −44.44 |
| Q1, Q3 | −46.43, | −44.44, | −44.44, | −46.43, | −46.43, |
| | −28.13 | −21.05 | −28.13 | −21.05 | −28.13 |
| Min, Max | −46.4, | −44.4, | −44.4, | −46.4, | −60.0, |
| | −28.1 | −21.1 | −28.1 | −21.1 | −21.1 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 13.00 | 11.00 | 12.83 | |
| (SD) | | | | | | |
| Median | | | 13.00 | 11.00 | 12.83 | |
| Q1, Q3 | | | 13.00, | 11.00, | 12.83, | |
| | | | 13.00 | 11.00 | 12.83 | |
| Min, Max | | | 13.0, | 11.0, | 12.8, | |
| | | | 13.0 | 11.0 | 12.8 | |
| Weekly RP | | | | | | |
| Attacks | | | | | | |
| n | | | 1 | 1 | 1 | |
| Mean | | | 19.00 | 6.00 | 11.00 | |
| (SD) | | | | | | |
| Median | | | 19.00 | 6.00 | 11.00 | |
| Q1, Q3 | | | 19.00, | 6.00, 6.00 | 11.00, | |
| | | | 19.00 | | 11.00 | |
| Min, Max | | | 19.0, | 6.0, | 11.0, | |
| | | | 19.0 | 6.0 | 11.0 | |
| Change | | | | | | |
| from | | | | | | |
| Baseline | | | | | | |
| n | | | 1 | 1 | 1 | |
| Mean | | | 6.00 | −5.00 | −1.83 | |
| (SD) | | | | | | |
| Median | | | 6.00 | −5.00 | −1.83 | |
| Q1, Q3 | | | 6.00, | −5.00, | −1.83, | |
| | | | 6.00 | −5.00 | −1.83 | |
| Min, Max | | | 6.0, | −5.0, | −1.8, | |
| | | | 6.0 | −5.0 | −1.8 | |
| Percent | | | | | | |
| Change | | | | | | |
| from | | | | | | |
| Baseline | | | | | | |
| n | | | 1 | 1 | 1 | |
| Mean | | | 46.15 | −45.45 | −14.29 | |
| (SD) | | | | | | |
| Median | | | 46.15 | −45.45 | −14.29 | |
| Q1, Q3 | | | 46.15, | −45.45, | −14.29, | |
| | | | 46.15 | −45.45 | −14.29 | |
| Min, Max | | | 46.2, | −45.5, | −14.3, | |
| | | | 46.2 | −45.5 | −14.3 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 11.92 | 11.00 | 12.83 | 12.28 |
| (SD) | | (1.296) | | | (1.110) |
| Median | | 11.92 | 11.00 | 12.83 | 12.83 |
| Q1, Q3 | | 11.00, | 11.00, | 12.83, | 11.00, |
| | | 12.83 | 11.00 | 12.83 | 13.00 |
| Min, Max | | 11.0, | 11.0, | 12.8, | 11.0, |
| | | 12.8 | 11.0 | 12.8 | 13.0 |
| Weekly RP | | | | | |
| Attacks | | | | | |
| n | | 2 | 1 | 1 | 3 |
| Mean | | 8.50 | 6.00 | 11.00 | 12.00 |
| (SD) | | (3.536) | | | (6.557) |
| Median | | 8.50 | 6.00 | 11.00 | 11.00 |
| Q1, Q3 | | 6.00, | 6.00, | 11.00, | 6.00, |
| | | 11.00 | 6.00 | 11.00 | 19.00 |
| Min, Max | | 6.0, | 6.0, | 11.0, | 6.0, |
| | | 11.0 | 6.0 | 11.0 | 19.0 |
| Change | | | | | |
| from | | | | | |
| Baseline | | | | | |
| n | | 2 | 1 | 1 | 3 |
| Mean | | −3.42 | −5.00 | −1.83 | −0.28 |
| (SD) | | (2.239) | | | (5.663) |
| Median | | −3.42 | −5.00 | −1.83 | −1.83 |
| Q1, Q3 | | −5.00, | −5.00, | −1.83, | −5.00, |
| | | −1.83 | −5.00 | −1.83 | 6.00 |
| Min, Max | | −5.0, | −5.0, | −1.8, | −5.0, |
| | | −1.8 | −5.0 | −1.8 | 6.0 |
| Percent | | | | | |
| Change | | | | | |
| from | | | | | |
| Baseline | | | | | |
| n | | 2 | 1 | 1 | 3 |
| Mean | | −29.87 | −45.45 | −14.29 | −4.53 |
| (SD) | | (22.040) | | | (46.577) |
| Median | | −29.87 | −45.45 | −14.29 | −14.29 |
| Q1, Q3 | | −45.45, | −45.45, | −14.29, | −45.45, |
| | | −14.29 | −45.45 | −14.29 | 46.15 |
| Min, Max | | −45.5, | −45.5, | −14.3, | −45.5, |
| | | −14.3 | −45.5 | −14.3 | 46.2 |

Tables B-1 to B-8 include data that relates to the duration of symptomatic Raynaud's attacks.

TABLE B-1
Average Duration of Weekly Symptomatic RP Attacks Change
and Percent Change from Baseline. ITT Population - Part A.
Average duration (minutes) of weekly symptomatic RP attacks
is defined as the total duration of symptomatic RP attacks
divided by total number of symptomatic RP attacks within the
last 7 days of the dosing period (excluding the last dosing day).
Baseline is calculated as total duration of symptomatic RP attacks
during the last 7 days of screening period (excluding the first
dosing day) divided by the total number of symptomatic RP attacks.
Only symptomatic RP attacks, defined as RP attacks with patient
reported finger color changes associated with at least one symptom
(pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 48.38 | 19.86 | 271.54 | 38.17 | 51.11 | 56.42 |
| (SD) | | (9.232) | (369.051) | (28.845) | (54.997) | (66.350) |
| Median | 48.38 | 19.86 | 271.54 | 38.17 | 51.11 | 56.42 |
| Q1, Q3 | 48.38, | 13.33, | 10.58, | 17.78, | 12.22, | 9.50, |
| | 48.38 | 26.39 | 532.50 | 58.57 | 90.00 | 103.33 |
| Min, Max | 48.4, | 13.3, | 10.6, | 17.8, | 12.2, | 9.5, |
| | 48.4 | 26.4 | 532.5 | 58.6 | 90.0 | 103.3 |
| Average | | | | | | |
| Duration | | | | | | |
| n | 1 | 2 | 2 | 2 | 2 | 1 |
| Mean | 31.64 | 16.73 | 412.29 | 28.38 | 39.23 | 98.08 |
| (SD) | | (19.413) | (574.575) | (14.672) | (33.615) | |
| Median | 31.64 | 16.73 | 412.29 | 28.38 | 39.23 | 98.08 |
| Q1, Q3 | 31.64, | 3.00, | 6.00, | 18.00, | 15.46, | 98.08, |
| | 31.64 | 30.45 | 818.57 | 38.75 | 63.00 | 98.08 |
| Min, Max | 31.6, | 3.0, | 6.0, | 18.0, | 15.5, | 98.1, |
| | 31.6 | 30.5 | 818.6 | 38.8 | 63.0 | 98.1 |
| Change | | | | | | |
| from | | | | | | |
| Baseline | | | | | | |
| n | 1 | 2 | 2 | 2 | 2 | 1 |
| Mean | −16.74 | −3.13 | 140.74 | −9.80 | −11.88 | −5.26 |
| (SD) | | (10.182) | (205.524) | (14.173) | (21.382) | |
| Median | −16.74 | −3.13 | 140.74 | −9.80 | −11.88 | −5.26 |
| Q1, Q3 | −16.74, | −10.33, | −4.58, | −19.82, | −27.00, | −5.26, |
| | −16.74 | 4.07 | 286.07 | 0.22 | 3.24 | −5.26 |
| Min, Max | −16.7, | −10.3, | −4.6, | −19.8, | −27.0, | −5.3, |
| | −16.7 | 4.1 | 286.1 | 0.2 | 3.2 | −5.3 |
| Percent | | | | | | |
| Change | | | | | | |
| from | | | | | | |
| Baseline | | | | | | |
| n | 1 | 2 | 2 | 2 | 2 | 1 |
| Mean | −34.60 | −31.05 | 5.21 | −16.30 | −1.75 | −5.09 |
| (SD) | | (65.695) | (68.610) | (24.813) | (39.954) | |
| Median | −34.60 | −31.05 | 5.21 | −16.30 | −1.75 | −5.09 |
| Q1, Q3 | −34.60, | −77.50, | −43.31, | −33.84, | −30.00, | −5.09, |
| | −34.60 | 15.41 | 53.72 | 1.25 | 26.50 | −5.09 |
| Min, Max | −34.6, | −77.5, | −43.3, | −33.8, | −30.0, | −5.1, |
| | −34.6 | 15.4 | 53.7 | 1.3 | 26.5 | −5.1 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 29.37 | 44.64 | 41.58 | 35.49 | 83.87 |
| (SD) | (17.713) | (36.625) | (21.231) | (36.907) | (152.403) |
| Median | 26.39 | 38.17 | 48.38 | 19.86 | 26.39 |
| Q1, Q3 | 13.33, | 15.00, | 17.78, | 12.78, | 12.22, |
| | 48.38 | 74.29 | 58.57 | 58.19 | 90.00 |
| Min, Max | 13.3, | 12.2, | 17.8, | 12.2, | 9.5, |
| | 48.4 | 90.0 | 58.6 | 90.0 | 532.5 |
| Average | | | | | |
| Duration | | | | | |
| n | 3 | 4 | 3 | 4 | 10 |
| Mean | 21.70 | 33.80 | 29.46 | 27.98 | 112.30 |
| (SD) | (16.205) | (22.084) | (10.545) | (25.905) | (249.796) |
| Median | 30.45 | 28.38 | 31.64 | 22.96 | 31.05 |
| Q1, Q3 | 3.00, | 16.73, | 18.00, | 9.23, | 15.46, |
| | 31.64 | 50.88 | 38.75 | 46.73 | 63.00 |
| Min, Max | 3.0, | 15.5, | 18.0, | 3.0, | 3.0, |
| | 31.6 | 63.0 | 38.8 | 63.0 | 818.6 |
| Change | | | | | |
| from | | | | | |
| Baseline | | | | | |
| n | 3 | 4 | 3 | 4 | 10 |
| Mean | −7.67 | −10.84 | −12.11 | −7.51 | 20.99 |
| (SD) | (10.655) | (14.859) | (10.793) | (14.576) | (93.700) |
| Median | −10.33 | −9.80 | −16.74 | −3.55 | −4.92 |
| Q1, Q3 | −16.74, | −23.41, | −19.82, | −18.67, | −16.74, |
| | 4.07 | 1.73 | 0.22 | 3.65 | 3.24 |
| Min, Max | −16.7, | −27.0, | −19.8, | −27.0, | −27.0, |
| | 4.1 | 3.2 | 0.2 | 4.1 | 286.1 |
| Percent | | | | | |
| Change | | | | | |
| from | | | | | |
| Baseline | | | | | |
| n | 3 | 4 | 3 | 4 | 10 |
| Mean | −32.23 | −9.02 | −22.40 | −16.40 | −12.74 |
| (SD) | (46.499) | (28.423) | (20.481) | (47.506) | (38.518) |
| Median | −34.60 | −14.38 | −33.84 | −7.30 | −17.54 |
| Q1, Q3 | −77.50, | −31.92, | −34.60, | −53.75, | −34.60, |
| | 15.41 | 13.88 | 1.25 | 20.96 | 15.41 |
| Min, Max | −77.5, | −33.8, | −34.6, | −77.5, | −77.5, |
| | 15.4 | 26.5 | 1.3 | 26.5 | 53.7 |

TABLE B-2
Average Duration of Weekly Symptomatic RP Attacks Change and Percent
Change from Baseline (Excluding Outliers). ITT Population - Part A.
Average duration (minutes) of weekly symptomatic RP attacks is defined as the total duration of symptomatic
RP attacks divided by total number of symptomatic RP attacks within the last 7 days of the dosing period
(excluding the last dosing day). Baseline is calculated as total duration of symptomatic RP attacks during
the last 7 days of screening period (excluding the first dosing day) divided by the total number of symptomatic
RP attacks. Only symptomatic RP attacks, defined as RP attacks with patient reported finger color changes
associated with at least one symptom (pain, numbness, tingling), were included in the derivations.

| | | | | | Cilnidipine | | Cilnidipine | | |
|---|---|---|---|---|---|---|---|---|---|
| | | | | | 10 mg + | | 20 mg + | | |
| | Cilnidipine | Cilnidipine | | Tadalafil | Tadalafil | | Tadalafil | | |
| | 10 mg | 20 mg | | 5 mg | 5 mg | | 5 mg | | Placebo |
| Statistics | (N = 1) | (N = 2) | | (N = 2) | (N = 2) | | (N = 2) | | (N = 2) |
| Baseline | | | | | | | | | |
| n | 1 | 2 | | 1 | 2 | | 2 | | 2 |
| Mean (SD) | 48.38 | 19.86 | (9.232) | 10.58 | 38.17 | (28.845) | 51.11 | (54.997) | 56.42 (66.350) |
| Median | 48.38 | 19.86 | | 10.58 | 38.17 | | 51.11 | | 56.42 |
| Q1, Q3 | 48.38, 48.38 | 13.33, 26.39 | | 10.58, 10.58 | 17.78, 58.57 | | 12.22, 90.00 | | 9.50, 103.33 |
| Min, Max | 48.4, 48.4 | 13.3, 26.4 | | 10.6, 10.6 | 17.8, 58.6 | | 12.2, 90.0 | | 9.5, 103.3 |
| Average | | | | | | | | | |
| Duration | | | | | | | | | |
| n | 1 | 2 | | 1 | 2 | | 2 | | 1 |
| Mean (SD) | 31.64 | 16.73 | (19.413) | 6.00 | 28.38 | (14.672) | 39.23 | (33.615) | 98.08 |
| Median | 31.64 | 16.73 | | 6.00 | 28.38 | | 39.23 | | 98.08 |
| Q1, Q3 | 31.64, 31.64 | 3.00, 30.45 | | 6.00, 6.00 | 18.00, 38.75 | | 15.46, 63.00 | | 98.08, 98.08 |
| Min, Max | 31.6, 31.6 | 3.0, 30.5 | | 6.0, 6.0 | 18.0, 38.8 | | 15.5, 63.0 | | 98.1, 98.1 |
| Change from | | | | | | | | | |
| Baseline | | | | | | | | | |
| n | 1 | 2 | | 1 | 2 | | 2 | | 1 |
| Mean (SD) | −16.74 | −3.13 | (10.182) | −4.58 | −9.80 | (14.173) | −11.88 | (21.382) | −5.26 |
| Median | −16.74 | −3.13 | | −4.58 | −9.80 | | −11.88 | | −5.26 |
| Q1, Q3 | −16.74, −16.74 | −10.33, 4.07 | | −4.58, −4.58 | −19.82, 0.22 | | −27.00, 3.24 | | −5.26, −5.26 |
| Min, Max | −16.7, −16.7 | −10.3, 4.1 | | −4.6, −4.6 | −19.8, 0.2 | | −27.0, 3.2 | | −5.3, −5.3 |
| Percent | | | | | | | | | |
| Change from | | | | | | | | | |
| Baseline | | | | | | | | | |
| n | 1 | 2 | | 1 | 2 | | 2 | | 1 |
| Mean (SD) | −34.60 | −31.05 | (65.695) | −43.31 | −16.30 | (24.813) | −1.75 | (39.954) | −5.09 |

| | | | | | | |
|---|---|---|---|---|---|---|
| Median | −34.60 | −31.05 | −43.31 | −16.30 | −1.75 | −5.09 |
| Q1, Q3 | −34.60, −34.60 | −77.50, 15.41 | −43.31, −43.31 | −33.84, 1.25 | −30.00, 26.50 | −5.09, −5.09 |
| Min, Max | −34.6, −34.6 | −77.5, 15.4 | −43.3, −43.3 | −33.8, 1.3 | −30.0, 26.5 | −5.1, −5.1 |

| | | | | | Pooled | | Pooled | | | |
|---|---|---|---|---|---|---|---|---|---|---|
| | | | | | Cilnidipine | | Cilnidipine | | | |
| | | | | | 10 mg and | | 20 mg and | | | |
| | | | | | Cilnidipine | | Cilnidipine | | | |
| | | | Pooled | | 10 mg + | | 20 mg + | | | |
| | Pooled | | Cilnidipine + | | Tadalafil | | Tadalafil | | All | |
| | Cilnidipine | | Tadalafil | | 5 mg | | 5 mg | | Participants | |
| Statistics | (N = 3) | | (N = 4) | | (N = 3) | | (N = 4) | | (N = 11) | |
| Baseline | | | | | | | | | | |
| n | 3 | | 4 | | 3 | | 4 | | 10 | |
| Mean (SD) | 29.37 | (17.713) | 44.64 | (36.625) | 41.58 | (21.231) | 35.49 | (36.907) | 39.01 | (34.759) |
| Median | 26.39 | | 38.17 | | 48.38 | | 19.86 | | 22.08 | |
| Q1, Q3 | 13.33, 48.38 | | 15.00, 74.29 | | 17.78, 58.57 | | 12.78, 58.19 | | 12.22, 58.57 | |
| Min, Max | 13.3, 48.4 | | 12.2, 90.0 | | 17.8, 58.6 | | 12.2, 90.0 | | 9.5, 103.3 | |
| Average | | | | | | | | | | |
| Duration | | | | | | | | | | |
| n | 3 | | 4 | | 3 | | 4 | | 9 | |
| Mean (SD) | 21.70 | (16.205) | 33.80 | (22.084) | 29.46 | (10.545) | 27.98 | (25.905) | 33.82 | (30.274) |
| Median | 30.45 | | 28.38 | | 31.64 | | 22.96 | | 30.45 | |
| Q1, Q3 | 3.00, 31.64 | | 16.73, 50.88 | | 18.00, 38.75 | | 9.23, 46.73 | | 15.46, 38.75 | |
| Min, Max | 3.0, 31.6 | | 15.5, 63.0 | | 18.0, 38.8 | | 3.0, 63.0 | | 3.0, 98.1 | |
| Change from | | | | | | | | | | |
| Baseline | | | | | | | | | | |
| n | 3 | | 4 | | 3 | | 4 | | 9 | |
| Mean (SD) | −7.67 | (10.655) | −10.84 | (14.859) | −12.11 | (10.793) | −7.51 | (14.576) | −8.47 | (10.836) |
| Median | −10.33 | | −9.80 | | −16.74 | | −3.55 | | −5.26 | |
| Q1, Q3 | −16.74, 4.07 | | −23.41, 1.73 | | −19.82, 0.22 | | −18.67, 3.65 | | −16.74, 0.22 | |
| Min, Max | −16.7, 4.1 | | −27.0, 3.2 | | −19.8, 0.2 | | −27.0, 4.1 | | −27.0, 4.1 | |
| Percent | | | | | | | | | | |
| Change from | | | | | | | | | | |
| Baseline | | | | | | | | | | |
| n | 3 | | 4 | | 3 | | 4 | | 9 | |
| Mean (SD) | −32.23 | (46.499) | −9.02 | (28.423) | −22.40 | (20.481) | −16.40 | (47.506) | −20.13 | (32.489) |

| | | | | | |
|---|---|---|---|---|---|
| Median | −34.60 | −14.38 | −33.84 | −7.30 | −30.00 |
| Q1, Q3 | −77.50, 15.41 | −31.92, 13.88 | −34.60, 1.25 | −53.75, 20.96 | −34.60, 1.25 |
| Min, Max | −77.5, 15.4 | −33.8, 26.5 | −34.6, 1.3 | −77.5, 26.5 | −77.5, 26.5 |

TABLE B-3
Average Duration of Weekly Symptomatic RP Attacks Change and Percent
Change from Baseline by Baseline RCS. ITT Population - Part A.
Average duration (minutes) of weekly symptomatic RP attacks is defined as the total duration of symptomatic
RP attacks divided by total number of symptomatic RP attacks within the last 7 days of the dosing period
(excluding the last dosing day). Baseline is calculated as total duration of symptomatic RP attacks during
the last 7 days of screening period (excluding the first dosing day) divided by the total number of symptomatic
RP attacks. Only symptomatic RP attacks, defined as RP attacks with patient reported finger color changes
associated with at least one symptom (pain, numbness, tingling), were included in the derivations.

| | | | | | Cilnidipine | Cilnidipine | |
|---|---|---|---|---|---|---|---|
| | | | | | 10 mg + | 20 mg + | |
| | Cilnidipine | Cilnidipine | | Tadalafil | Tadalafil | Tadalafil | |
| | 10 mg | 20 mg | | 5 mg | 5 mg | 5 mg | Placebo |
| Statistics | (N = 1) | (N = 2) | | (N = 2) | (N = 2) | (N = 2) | (N = 2) |
| Baseline | | | | | | | |
| n | 1 | 2 | | | 1 | 1 | 2 |
| Mean (SD) | 48.38 | 19.86 | (9.232) | | 17.78 | 12.22 | 56.42 (66.350) |
| Median | 48.38 | 19.86 | | | 17.78 | 12.22 | 56.42 |
| Q1, Q3 | 48.38, 48.38 | 13.33, 26.39 | | | 17.78, 17.78 | 12.22, 12.22 | 9.50, 103.33 |
| Min, Max | 48.4, 48.4 | 13.3, 26.4 | | | 17.8, 17.8 | 12.2, 12.2 | 9.5, 103.3 |
| Average | | | | | | | |
| Duration | | | | | | | |
| n | 1 | 2 | | | 1 | 1 | 1 |
| Mean (SD) | 31.64 | 16.73 | (19.413) | | 18.00 | 15.46 | 98.08 |
| Median | 31.64 | 16.73 | | | 18.00 | 15.46 | 98.08 |
| Q1, Q3 | 31.64, 31.64 | 3.00, 30.45 | | | 18.00, 18.00 | 15.46, 15.46 | 98.08, 98.08 |
| Min, Max | 31.6, 31.6 | 3.0, 30.5 | | | 18.0, 18.0 | 15.5, 15.5 | 98.1, 98.1 |
| Change from | | | | | | | |
| Baseline | | | | | | | |
| n | 1 | 2 | | | 1 | 1 | 1 |
| Mean (SD) | −16.74 | −3.13 | (10.182) | | 0.22 | 3.24 | −5.26 |
| Median | −16.74 | −3.13 | | | 0.22 | 3.24 | −5.26 |
| Q1, Q3 | −16.74, −16.74 | −10.33, 4.07 | | | 0.22, 0.22 | 3.24, 3.24 | −5.26, −5.26 |
| Min, Max | −16.7, −16.7 | −10.3, 4.1 | | | 0.2, 0.2 | 3.2, 3.2 | −5.3, −5.3 |
| Percent | | | | | | | |
| Change from | | | | | | | |
| Baseline | | | | | | | |
| n | 1 | 2 | | | 1 | 1 | 1 |
| Mean (SD) | −34.60 | −31.05 | (65.695) | | 1.25 | 26.50 | −5.09 |

| | | | | | |
|---|---|---|---|---|---|
| Median | −34.60 | −31.05 | 1.25 | 26.50 | −5.09 |
| Q1, Q3 | −34.60, −34.60 | −77.50, 15.41 | 1.25, 1.25 | 26.50, 26.50 | −5.09, −5.09 |
| Min, Max | −34.6, −34.6 | −77.5, 15.4 | 1.3, 1.3 | 26.5, 26.5 | −5.1, −5.1 |

| | | | | | Pooled | | Pooled | | | |
|---|---|---|---|---|---|---|---|---|---|---|
| | | | | | Cilnidipine | | Cilnidipine | | | |
| | | | | | 10 mg and | | 20 mg and | | | |
| | | | | | Cilnidipine | | Cilnidipine | | | |
| | | | Pooled | | 10 mg + | | 20 mg + | | | |
| | Pooled | | Cilnidipine + | | Tadalafil | | Tadalafil | | All | |
| | Cilnidipine | | Participant | | 5 mg | | 5 mg | | Participants | |
| | (N = 3) | | (N = 4) | | (N = 3) | | (N = 4) | | (N = 11) | |
| Baseline | | | | | | | | | | |
| n | 3 | | 2 | | 2 | | 3 | | 7 | |
| Mean (SD) | 29.37 | (17.713) | 15.00 | (3.928) | 33.08 | (21.640) | 17.31 | (7.878) | 32.99 | (33.738) |
| Median | 26.39 | | 15.00 | | 33.08 | | 13.33 | | 17.78 | |
| Q1, Q3 | 13.33, 48.38 | | 12.22, 17.78 | | 17.78, 48.38 | | 12.22, 26.39 | | 12.22, 48.38 | |
| Min, Max | 13.3, 48.4 | | 12.2, 17.8 | | 17.8, 48.4 | | 12.2, 26.4 | | 9.5, 103.3 | |
| Average | | | | | | | | | | |
| Duration | | | | | | | | | | |
| n | 3 | | 2 | | 2 | | 3 | | 6 | |
| Mean (SD) | 21.70 | (16.205) | 16.73 | (1.795) | 24.82 | (9.647) | 16.31 | (13.747) | 32.77 | (33.692) |
| Median | 30.45 | | 16.73 | | 24.82 | | 15.46 | | 24.23 | |
| Q1, Q3 | 3.00, 31.64 | | 15.46, 18.00 | | 18.00, 31.64 | | 3.00, 30.45 | | 15.46, 31.64 | |
| Min, Max | 3.0, 31.6 | | 15.5, 18.0 | | 18.0, 31.6 | | 3.0, 30.5 | | 3.0, 98.1 | |
| Change from | | | | | | | | | | |
| Baseline | | | | | | | | | | |
| n | 3 | | 2 | | 2 | | 3 | | 6 | |
| Mean (SD) | −7.67 | (10.655) | 1.73 | (2.133) | −8.26 | (11.993) | −1.01 | (8.085) | −4.13 | (8.236) |
| Median | −10.33 | | 1.73 | | −8.26 | | 3.24 | | −2.52 | |
| Q1, Q3 | −16.74, 4.07 | | 0.22, 3.24 | | −16.74, 0.22 | | −10.33, 4.07 | | −10.33, 3.24 | |
| Min, Max | −16.7, 4.1 | | 0.2, 3.2 | | −16.7, 0.2 | | −10.3, 4.1 | | −16.7, 4.1 | |
| Percent | | | | | | | | | | |
| Change from | | | | | | | | | | |
| Baseline | | | | | | | | | | |
| n | 3 | | 2 | | 2 | | 3 | | 6 | |
| Mean (SD) | −32.23 | (46.499) | 13.88 | (17.857) | −16.67 | (25.347) | −11.86 | (57.113) | −12.34 | (38.098) |

| | | | | | |
|---|---|---|---|---|---|
| Median | −34.60 | 13.88 | −16.67 | 15.41 | −1.92 |
| Q1, Q3 | −77.50, 15.41 | 1.25, 26.50 | −34.60, 1.25 | −77.50, 26.50 | −34.60, 15.41 |
| Min, Max | −77.5, 15.4 | 1.3, 26.5 | −34.6, 1.3 | −77.5, 26.5 | −77.5, 26.5 |

| | | | | | Cilnidipine | Cilnidipine | |
|---|---|---|---|---|---|---|---|
| | | | | | 10 mg + | 20 mg + | |
| | Cilnidipine | Cilnidipine | Tadalafil | | Tadalafil | Tadalafil | |
| | 10 mg | 20 mg | 5 mg | | 5 mg | 5 mg | Placebo |
| Statistics | (N = 1) | (N = 2) | (N=2) | | (N = 2) | (N = 2) | (N = 2) |
| Baseline | | | | | | | |
| n | | | 2 | | 1 | 1 | |
| Mean (SD) | | | 271.54 | (369.051) | 58.57 | 90.00 | |
| Median | | | 271.54 | | 58.57 | 90.00 | |
| Q1, Q3 | | | 10.58, 532.50 | | 58.57, 58.57 | 90.00, 90.00 | |
| Min, Max | | | 10.6, 532.5 | | 58.6, 58.6 | 90.0, 90.0 | |
| Average | | | | | | | |
| Duration | | | | | | | |
| n | | | 2 | | 1 | 1 | |
| Mean (SD) | | | 412.29 | (574.575) | 38.75 | 63.00 | |
| Median | | | 412.29 | | 38.75 | 63.00 | |
| Q1, Q3 | | | 6.00, 818.57 | | 38.75, 38.75 | 63.00, 63.00 | |
| Min, Max | | | 6.0, 818.6 | | 38.8, 38.8 | 63.0, 63.0 | |
| Change from | | | | | | | |
| Baseline | | | | | | | |
| n | | | 2 | | 1 | 1 | |
| Mean (SD) | | | 140.74 | (205.524) | −19.82 | −27.00 | |
| Median | | | 140.74 | | −19.82 | −27.00 | |
| Q1, Q3 | | | −4.58, 286.07 | | −19.82, −19.82 | −27.00, −27.00 | |
| Min, Max | | | −4.6, 286.1 | | −19.8, −19.8 | −27.0, −27.0 | |
| Percent | | | | | | | |
| Change from | | | | | | | |
| Baseline | | | | | | | |
| n | | | 2 | | 1 | 1 | |
| Mean (SD) | | | 5.21 | (68.610) | −33.84 | −30.00 | |

| | | | |
|---|---|---|---|
| Median | 5.21 | −33.84 | −30.00 |
| Q1, Q3 | −43.31, 53.72 | −33.84, −33.84 | −30.00, −30.00 |
| Min, Max | −43.3, 53.7 | −33.8, −33.8 | −30.0, −30.0 |

| | | | | Pooled | Cilnidipine | | |
|---|---|---|---|---|---|---|---|
| | | | | Cilnidipine | Pooled | | |
| | | | | 10 mg and | 20 mg and | | |
| | | | | Cilnidipine | Cilnidipine | | |
| | | Pooled | | 10 mg + | 20 mg + | | |
| | Pooled | Cilnidipine + | | Tadalafil | Tadalafil | All | |
| | Cilnidipine | Tadalafil | | 5 mg | 5 mg | Participants | |
| Statistics | (N = 3) | (N = 4) | | (N = 3) | (N = 4) | (N = 11) | |
| Baseline | | | | | | | |
| n | | 2 | | 1 | 1 | 4 | |
| Mean (SD) | | 74.29 | (22.223) | 58.57 | 90.00 | 172.91 | (241.938) |
| Median | | 74.29 | | 58.57 | 90.00 | 74.29 | |
| Q1, Q3 | | 58.57, 90.00 | | 58.57, 58.57 | 90.00, 90.00 | 34.58, 311.25 | |
| Min, Max | | 58.6, 90.0 | | 58.6, 58.6 | 90.0, 90.0 | 10.6, 532.5 | |
| Average | | | | | | | |
| Duration | | | | | | | |
| n | | 2 | | 1 | 1 | 4 | |
| Mean (SD) | | 50.88 | (17.147) | 38.75 | 63.00 | 231.58 | (392.024) |
| Median | | 50.88 | | 38.75 | 63.00 | 50.88 | |
| Q1, Q3 | | 38.75, 63.00 | | 38.75, 38.75 | 63.00, 63.00 | 22.38, 440.79 | |
| Min, Max | | 38.8, 63.0 | | 38.8, 38.8 | 63.0, 63.0 | 6.0, 818.6 | |
| Change from | | | | | | | |
| Baseline | | | | | | | |
| n | | 2 | | 1 | 1 | 4 | |
| Mean (SD) | | −23.41 | (5.076) | −19.82 | −27.00 | 58.67 | (151.891) |
| Median | | −23.41 | | −19.82 | −27.00 | −12.20 | |
| Q1, Q3 | | −27.00, −19.82 | | −19.82, −19.82 | −27.00, −27.00 | −23.41, 140.74 | |
| Min, Max | | −27.0, | | −19.8−19.8, −19.8 | −27.0, −27.0 | −27.0, 286.1 | |
| Percent | | | | | | | |
| Change from | | | | | | | |
| Baseline | | | | | | | |
| n | | 2 | | 1 | 1 | 4 | |
| Mean (SD) | | −31.92 | (2.716) | −33.84 | −30.00 | −13.36 | (45.068) |

| | | | | |
|---|---|---|---|---|
| Median | −31.92 | −33.84 | −30.00 | −31.92 |
| Q1, Q3 | −33.84, −30.00 | −33.84, −33.84 | −30.00, −30.00 | −38.57, 11.86 |
| Min, Max | −33.8, | −30.0−33.8, −33.8 | −30.0, −30.0 | −43.3, 53.7 |

TABLE B-4
Average Duration of Weekly Symptomatic RP Attacks Change and Percent
Change from Baseline (Excluding Outliers) by Baseline RCS. ITT Population - Part A.
Average duration (minutes) of weekly symptomatic RP attacks is defined as the total duration of symptomatic
RP attacks divided by total number of symptomatic RP attacks within the last 7 days of the dosing period
(excluding the last dosing day). Baseline is calculated as total duration of symptomatic RP attacks during
the last 7 days of screening period (excluding the first dosing day) divided by the total number of symptomatic
RP attacks. Only symptomatic RP attacks, defined as RP attacks with patient reported finger color changes
associated with at least one symptom (pain, numbness, tingling), were included in the derivations.

| | | | | | Cilnidipine | Cilnidipine | |
|---|---|---|---|---|---|---|---|
| | | | | | 10 mg + | 20 mg + | |
| | Cilnidipine | Cilnidipine | | Tadalafil | Tadalafil | Tadalafil | |
| | 10 mg | 20 mg | | 5 mg | 5 mg | 5 mg | Placebo |
| Statistics | (N = 1) | (N = 2) | | (N = 2) | (N = 2) | (N = 2) | (N = 2) |
| Baseline | | | | | | | |
| n | 1 | 2 | | | 1 | 1 | 2 |
| Mean (SD) | 48.38 | 19.86 | (9.232) | | 17.78 | 12.22 | 56.42 (66.350) |
| Median | 48.38 | 19.86 | | | 17.78 | 12.22 | 56.42 |
| Q1, Q3 | 48.38, 48.38 | 13.33, 26.39 | | | 17.78, 17.78 | 12.22, 12.22 | 9.50, 103.33 |
| Min, Max | 48.4, 48.4 | 13.3, 26.4 | | | 17.8, 17.8 | 12.2, 12.2 | 9.5, 103.3 |
| Average | | | | | | | |
| Duration | | | | | | | |
| n | 1 | 2 | | | 1 | 1 | 1 |
| Mean (SD) | 31.64 | 16.73 | (19.413) | | 18.00 | 15.46 | 98.08 |
| Median | 31.64 | 16.73 | | | 18.00 | 15.46 | 98.08 |
| Q1, Q3 | 31.64, 31.64 | 3.00, 30.45 | | | 18.00, 18.00 | 15.46, 15.46 | 98.08, 98.08 |
| Min, Max | 31.6, 31.6 | 3.0, 30.5 | | | 18.0, 18.0 | 15.5, 15.5 | 98.1, 98.1 |
| Change from | | | | | | | |
| Baseline | | | | | | | |
| n | 1 | 2 | | | 1 | 1 | 1 |
| Mean (SD) | −16.74 | −3.13 | (10.182) | | 0.22 | 3.24 | −5.26 |
| Median | −16.74 | −3.13 | | | 0.22 | 3.24 | −5.26 |
| Q1, Q3 | −16.74, −16.74 | −10.33, 4.07 | | | 0.22, 0.22 | 3.24, 3.24 | −5.26, −5.26 |
| Min, Max | −16.7, −16.7 | −10.3, 4.1 | | | 0.2, 0.2 | 3.2, 3.2 | −5.3, −5.3 |
| Percent | | | | | | | |
| Change from | | | | | | | |
| Baseline | | | | | | | |
| n | 1 | 2 | | | 1 | 1 | 1 |
| Mean (SD) | −34.60 | −31.05 | (65.695) | | 1.25 | 26.50 | −5.09 |

| | | | | | |
|---|---|---|---|---|---|
| Median | −34.60 | −31.05 | 1.25 | 26.50 | −5.09 |
| Q1, Q3 | −34.60, −34.60 | −77.50, 15.41 | 1.25, 1.25 | 26.50, 26.50 | −5.09, −5.09 |
| Min, Max | −34.6, −34.6 | −77.5, 15.4 | 1.3, 1.3 | 26.5, 26.5 | −5.1, −5.1 |

| | | | | | Pooled | | Pooled | | | |
|---|---|---|---|---|---|---|---|---|---|---|
| | | | | | Cilnidipine | | Cilnidipine | | | |
| | | | | | 10 mg and | | 20 mg and | | | |
| | | | | | Cilnidipine | | Cilnidipine | | | |
| | | | Pooled | | 10 mg + | | 20 mg + | | | |
| | Pooled | | Cilnidipine + | | Tadalafil | | Tadalafil | | All | |
| | Cilnidipine | | Tadalafil | | 5 mg | | 5 mg | | Participants | |
| Statistics | (N = 3) | | (N = 4) | | (N = 3) | | (N = 4) | | (N = 11) | |
| Baseline | | | | | | | | | | |
| n | 3 | | 2 | | 2 | | 3 | | 7 | |
| Mean (SD) | 29.37 | (17.713) | 15.00 | (3.928) | 33.08 | (21.640) | 17.31 | (7.878) | 32.99 | (33.738) |
| Median | 26.39 | | 15.00 | | 33.08 | | 13.33 | | 17.78 | |
| Q1, Q3 | 13.33, 48.38 | | 12.22, 17.78 | | 17.78, 48.38 | | 12.22, 26.39 | | 12.22, 48.38 | |
| Min, Max | 13.3, 48.4 | | 12.2, 17.8 | | 17.8, 48.4 | | 12.2, 26.4 | | 9.5, 103.3 | |
| Average | | | | | | | | | | |
| Duration | | | | | | | | | | |
| n | 3 | | 2 | | 2 | | 3 | | 6 | |
| Mean (SD) | 21.70 | (16.205) | 16.73 | (1.795) | 24.82 | (9.647) | 16.31 | (13.747) | 32.77 | (33.692) |
| Median | 30.45 | | 16.73 | | 24.82 | | 15.46 | | 24.23 | |
| Q1, Q3 | 3.00, 31.64 | | 15.46, 18.00 | | 18.00, 31.64 | | 3.00, 30.45 | | 15.46, 31.64 | |
| Min, Max | 3.0, 31.6 | | 15.5, 18.0 | | 18.0, 31.6 | | 3.0, 30.5 | | 3.0, 98.1 | |
| Change from | | | | | | | | | | |
| Baseline | | | | | | | | | | |
| n | 3 | | 2 | | 2 | | 3 | | 6 | |
| Mean (SD) | −7.67 | (10.655) | 1.73 | (2.133) | −8.26 | (11.993) | −1.01 | (8.085) | −4.13 | (8.236) |
| Median | −10.33 | | 1.73 | | −8.26 | | 3.24 | | −2.52 | |
| Q1, Q3 | −16.74, 4.07 | | 0.22, 3.24 | | −16.74, 0.22 | | −10.33, 4.07 | | −10.33, 3.24 | |
| Min, Max | −16.7, 4.1 | | 0.2, 3.2 | | −16.7, 0.2 | | −10.3, 4.1 | | −16.7, 4.1 | |
| Percent | | | | | | | | | | |
| Change from | | | | | | | | | | |
| Baseline | | | | | | | | | | |
| n | 3 | | 2 | | 2 | | 3 | | 6 | |
| Mean (SD) | −32.23 | (46.499) | 13.88 | (17.857) | −16.67 | (25.347) | −11.86 | (57.113) | −12.34 | (38.098) |

| | | | | | |
|---|---|---|---|---|---|
| Median | −34.60 | 13.88 | −16.67 | 15.41 | −1.92 |
| Q1, Q3 | −77.50, 15.41 | 1.25, 26.50 | −34.60, 1.25 | −77.50, 26.50 | −34.60, 15.41 |
| Min, Max | −77.5, 15.4 | 1.3, 26.5 | −34.6, 1.3 | −77.5, 26.5 | −77.5, 26.5 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean (SD) | | | 10.58 | 58.57 | 90.00 | |
| Median | | | 10.58 | 58.57 | 90.00 | |
| Q1, Q3 | | | 10.58, 10.58 | 58.57, 58.57 | 90.00, 90.00 | |
| Min, Max | | | 10.6, 10.6 | 58.6, 58.6 | 90.0, 90.0 | |
| Average | | | | | | |
| Duration | | | | | | |
| n | | | 1 | 1 | 1 | |
| Mean (SD) | | | 6.00 | 38.75 | 63.00 | |
| Median | | | 6.00 | 38.75 | 63.00 | |
| Q1, Q3 | | | 6.00, 6.00 | 38.75, 38.75 | 63.00, 63.00 | |
| Min, Max | | | 6.0, 6.0 | 38.8, 38.8 | 63.0, 63.0 | |
| Change from | | | | | | |
| Baseline | | | | | | |
| n | | | 1 | 1 | 1 | |
| Mean (SD) | | | −4.58 | −19.82 | −27.00 | |
| Median | | | −4.58 | −19.82 | −27.00 | |
| Q1, Q3 | | | −4.58, −4.58 | −19.82, −19.82 | −27.00, −27.00 | |
| Min, Max | | | −4.6, −4.6 | −19.8, −19.8 | −27.0, −27.0 | |
| Percent | | | | | | |
| Change from | | | | | | |
| Baseline | | | | | | |
| n | | | 1 | 1 | 1 | |
| Mean (SD) | | | −43.31 | −33.84 | −30.00 | |
| Median | | | −43.31 | −33.84 | −30.00 | |
| Q1, Q3 | | | −43.31, −43.31 | −33.84, −33.84 | −30.00, −30.00 | |
| Min, Max | | | −43.3, −43.3 | −33.8, −33.8 | −30.0, −30.0 | |

| | | | | Pooled | Pooled | | |
|---|---|---|---|---|---|---|---|
| | | | | Cilnidipine | Cilnidipine | | |
| | | | | 10 mg and | 20 mg and | | |
| | | | | Cilnidipine | Cilnidipine | | |
| | | Pooled | | 10 mg + | 20 mg + | | |
| | Pooled | Cilnidipine + | | Tadalafil | Tadalafil | All | |
| | Cilnidipine | Tadalafil | | 5 mg | 5 mg | Participants | |
| Statistics | (N = 3) | (N = 4) | | (N = 3) | (N = 4) | (N = 11) | |
| Baseline | | | | | | | |
| n | | 2 | | 1 | 1 | 3 | |
| Mean (SD) | | 74.29 | (22.223) | 58.57 | 90.00 | 53.05 | (39.995) |
| Median | | 74.29 | | 58.57 | 90.00 | 58.57 | |
| Q1, Q3 | | 58.57, 90.00 | | 58.57, 58.57 | 90.00, 90.00 | 10.58, 90.00 | |
| Min, Max | | 58.6, 90.0 | | 58.6, 58.6 | 90.0, 90.0 | 10.6, 90.0 | |
| Average | | | | | | | |
| Duration | | | | | | | |
| n | | 2 | | 1 | 1 | 3 | |
| Mean (SD) | | 50.88 | (17.147) | 38.75 | 63.00 | 35.92 | (28.605) |
| Median | | 50.88 | | 38.75 | 63.00 | 38.75 | |
| Q1, Q3 | | 38.75, 63.00 | | 38.75, 38.75 | 63.00, 63.00 | 6.00, 63.00 | |
| Min, Max | | 38.8, 63.0 | | 38.8, 38.8 | 63.0, 63.0 | 6.0, 63.0 | |
| Change from | | | | | | | |
| Baseline | | | | | | | |
| n | | 2 | | 1 | | 3 | |
| Mean (SD) | | −23.41 | (5.076) | −19.82 | −27.00 | −17.13 | (11.447) |
| Median | | −23.41 | | −19.82 | −27.00 | −19.82 | |
| Q1, Q3 | | −27.00, −19.82 | | −19.82, −19.82 | −27.00, −27.00 | −27.00, −4.58 | |
| Min, Max | | −27.0, −19.8 | | −19.8, −19.8 | −27.0, −27.0 | −27.0, −4.6 | |
| Percent | | | | | | | |
| Change from | | | | | | | |
| Baseline | | | | | | | |
| n | | 2 | | 1 | 1 | 3 | |
| Mean (SD) | | −31.92 | (2.716) | −33.84 | −30.00 | −35.72 | (6.849) |

| | | | | |
|---|---|---|---|---|
| Median | −31.92 | −33.84 | −30.00 | −33.84 |
| Q1, Q3 | −33.84, −30.00 | −33.84, −33.84 | −30.00, −30.00 | −43.31, −30.00 |
| Min, Max | −33.8, −30.0 | −33.8, −33.8 | −30.0, −30.0 | −43.3, −30.0 |

TABLE B-5
Average Duration of Weekly RP Attacks (All Attacks) Change and Percent Change from Baseline. ITT Population - Part A.
Average duration (minutes) of weekly RP attacks is defined as the total duration of RP attacks divided
by total number of symptomatic RP attacks within the last 7 days of the dosing period (excluding
the last dosing day). Baseline is calculated as total duration of RP attacks during the last 7 days
of screening period (excluding the first dosing day) divided by the total number of RP attacks.

| | | | | | | Cilnidipine | | Cilnidipine | |
|---|---|---|---|---|---|---|---|---|---|
| | | | | | | 10 mg + | | 20 mg + | |
| | Cilnidipine | Cilnidipine | | Tadalafil | | Tadalafil | | Tadalafil | |
| | 10 mg | 20 mg | | 5 mg | | 5 mg | | 5 mg | Placebo |
| Statistics | (N = 1) | (N = 2) | | (N = 2) | | (N = 2) | | (N = 2) | (N = 2) |
| Baseline | | | | | | | | | |
| n | 1 | 2 | | 2 | | 2 | | 2 | 2 |
| Mean (SD) | 40.00 | 17.81 | (10.165) | 320.46 | (437.753) | 36.16 | (25.999) | 46.96 (49.294) | 56.42 (66.350) |
| Median | 40.00 | 17.81 | | 320.46 | | 36.16 | | 46.96 | 56.42 |
| Q1, Q3 | 40.00, 40.00 | 10.63, 25.00 | | 10.92, 630.00 | | 17.78, 54.55 | | 12.11, 81.82 | 9.50, 103.33 |
| Min, Max | 40.0, 40.0 | 10.6, 25.0 | | 10.9, 630.0 | | 17.8, 54.5 | | 12.1, 81.8 | 9.5, 103.3 |
| Average | | | | | | | | | |
| Duration | | | | | | | | | |
| n | 1 | 2 | | 2 | | 2 | | 2 | 1 |
| Mean (SD) | 31.52 | 16.73 | (19.413) | 412.26 | (574.612) | 26.08 | (11.432) | 38.50 (34.267) | 92.33 |
| Median | 31.52 | 16.73 | | 412.26 | | 26.08 | | 38.50 | 92.33 |
| Q1, Q3 | 31.52, 31.52 | 3.00, 30.45 | | 5.95, 818.57 | | 18.00, 34.17 | | 14.27, 62.73 | 92.33, 92.33 |
| Min, Max | 31.5, 31.5 | 3.0, 30.5 | | 5.9, 818.6 | | 18.0, 34.2 | | 14.3, 62.7 | 92.3, 92.3 |
| Change from | | | | | | | | | |
| Baseline | | | | | | | | | |
| n | 1 | 2 | | 2 | | 2 | | 2 | 1 |
| Mean (SD) | −8.48 | −1.09 | (9.249) | 91.80 | (136.858) | −10.08 | (14.567) | −8.46 (15.028) | −11.00 |
| Median | −8.48 | −1.09 | | 91.80 | | −10.08 | | −8.46 | −11.00 |
| Q1, Q3 | −8.48, −8.48 | −7.63, 5.45 | | −4.98, 188.57 | | −20.38, 0.22 | | −19.09, 2.16 | −11.00, −11.00 |
| Min, Max | −8.5, −8.5 | −7.6, 5.5 | | −5.0, 188.6 | | −20.4, 0.2 | | −19.1, 2.2 | −11.0, −11.0 |
| Percent | | | | | | | | | |
| Change from | | | | | | | | | |
| Baseline | | | | | | | | | |
| n | 1 | 2 | | 2 | | 2 | | 2 | 1 |
| Mean (SD) | −21.20 | −24.97 | (66.173) | −7.81 | (53.375) | −18.06 | (27.302) | −2.74 (29.125) | −10.65 |

| | | | | | | |
|---|---|---|---|---|---|---|
| Median | −21.20 | −24.97 | −7.81 | −18.06 | −2.74 | −10.65 |
| Q1, Q3 | −21.20, −21.20 | −71.76, 21.82 | −45.55, 29.93 | −37.36, 1.25 | −23.33, 17.86 | −10.65, −10.65 |
| Min, Max | −21.2, −21.2 | −71.8, 21.8 | −45.6, 29.9 | −37.4, 1.3 | −23.3, 17.9 | −10.6, −10.6 |

| | | | | | Pooled | | Cilnidipine | | | |
|---|---|---|---|---|---|---|---|---|---|---|
| | | | | | Cilnidipine | | Pooled | | | |
| | | | | | 10 mg and | | 20 mg and | | | |
| | | | | | Cilnidipine | | Cilnidipine | | | |
| | | | Pooled | | 10 mg + | | 20 mg + | | | |
| | Cilnidipine | | Cilnidipine + | | Tadalafil | | Tadalafil | | All | |
| | Pooled | | Tadalafil | | 5 mg | | 5 mg | | Participants | |
| Statistics | (N = 3) | | (N = 4) | | (N = 3) | | (N = 4) | | (N = 11) | |
| Baseline | | | | | | | | | | |
| n | 3 | | 4 | | 3 | | 4 | | 11 | |
| Mean (SD) | 25.21 | (14.689) | 41.56 | (32.775) | 37.44 | (18.517) | 32.39 | (33.580) | 90.51 | (181.698) |
| Median | 25.00 | | 36.16 | | 40.00 | | 18.55 | | 25.00 | |
| Q1, Q3 | 10.63, 40.00 | | 14.94, 68.18 | | 17.78, 54.55 | | 11.37, 53.41 | | 10.92, 81.82 | |
| Min, Max | 10.6, 40.0 | | 12.1, 81.8 | | 17.8, 54.5 | | 10.6, 81.8 | | 9.5, 630.0 | |
| Average | | | | | | | | | | |
| Duration | | | | | | | | | | |
| n | 3 | | 4 | | 3 | | 4 | | 10 | |
| Mean (SD) | 21.66 | (16.168) | 32.29 | (22.053) | 27.90 | (8.672) | 27.61 | (25.981) | 111.10 | (250.056) |
| Median | 30.45 | | 26.08 | | 31.52 | | 22.36 | | 30.99 | |
| Q1, Q3 | 3.00, 31.52 | | 16.13, 48.45 | | 18.00, 34.17 | | 8.63, 46.59 | | 14.27, 62.73 | |
| Min, Max | 3.0, 31.5 | | 14.3, 62.7 | | 18.0, 34.2 | | 3.0, 62.7 | | 3.0, 818.6 | |
| Change from | | | | | | | | | | |
| Baseline | | | | | | | | | | |
| n | 3 | | 4 | | 3 | | 4 | | 10 | |
| Mean (SD) | −3.55 | (7.809) | −9.27 | (12.119) | −9.54 | (10.342) | −4.77 | (11.043) | 12.49 | (62.441) |
| Median | −7.63 | | −9.43 | | −8.48 | | −2.73 | | −6.30 | |
| Q1, Q3 | −8.48, 5.45 | | −19.73, 1.19 | | −20.38, 0.22 | | −13.36, 3.81 | | −11.00, 2.16 | |
| Min, Max | −8.5, 5.5 | | −20.4, 2.2 | | −20.4, 0.2 | | −19.1, 5.5 | | −20.4, 188.6 | |
| Percent | | | | | | | | | | |
| Change from | | | | | | | | | | |
| Baseline | | | | | | | | | | |
| n | 3 | | 4 | | 3 | | 4 | | 10 | |
| Mean (SD) | −23.71 | (46.842) | −10.40 | (24.686) | −19.10 | (19.390) | −13.86 | (43.671) | −13.90 | (32.469) |

| | | | | | |
|---|---|---|---|---|---|
| Median | −21.20 | −11.04 | −21.20 | −2.74 | −15.92 |
| Q1, Q3 | −71.76, 21.82 | −30.35, 9.55 | −37.36, 1.25 | −47.55, 19.84 | −37.36, 17.86 |
| Min, Max | −71.8, 21.8 | −37.4, 17.9 | −37.4, 1.3 | −71.8, 21.8 | −71.8, 29.9 |

TABLE B-6
Average Duration of Weekly RP Attacks (All Attacks) Change and
Percent Change from Baseline (Excluding Outliers). ITT Population - Part A.
Average duration (minutes) of weekly RP attacks is defined as the total duration of RP attacks divided
by total number of symptomatic RP attacks within the last 7 days of the dosing period (excluding
the last dosing day). Baseline is calculated as total duration of RP attacks during the last 7 days
of screening period (excluding the first dosing day) divided by the total number of RP attacks.

| | | | | | Cilnidipine | | Cilnidipine | |
|---|---|---|---|---|---|---|---|---|
| | | | | | 10 mg + | | 20 mg + | |
| | Cilnidipine | Cilnidipine | | Tadalafil | Tadalafil | | Tadalafil | |
| | 10 mg | 20 mg | | 5 mg | 5 mg | | 5 mg | Placebo |
| Statistics | (N = 1) | (N = 2) | | (N = 2) | (N = 2) | | (N = 2) | (N = 2) |
| Baseline | | | | | | | | |
| n | 1 | 2 | | 1 | 2 | | 2 | 2 |
| Mean (SD) | 40.00 | 17.81 | (10.165) | 10.92 | 36.16 | (25.999) | 46.96 (49.294) | 56.42 (66.350) |
| Median | 40.00 | 17.81 | | 10.92 | 36.16 | | 46.96 | 56.42 |
| Q1, Q3 | 40.00, 40.00 | 10.63, 25.00 | | 10.92, 10.92 | 17.78, 54.55 | | 12.11, 81.82 | 9.50, 103.33 |
| Min, Max | 40.0, 40.0 | 10.6, 25.0 | | 10.9, 10.9 | 17.8, 54.5 | | 12.1, 81.8 | 9.5, 103.3 |
| Average | | | | | | | | |
| Duration | | | | | | | | |
| n | 1 | 2 | | 1 | 2 | | 2 | 1 |
| Mean (SD) | 31.52 | 16.73 | (19.413) | 5.95 | 26.08 | (11.432) | 38.50 (34.267) | 92.33 |
| Median | 31.52 | 16.73 | | 5.95 | 26.08 | | 38.50 | 92.33 |
| Q1, Q3 | 31.52, 31.52 | 3.00, 30.45 | | 5.95, 5.95 | 18.00, 34.17 | | 14.27, 62.73 | 92.33, 92.33 |
| Min, Max | 31.5, 31.5 | 3.0, 30.5 | | 5.9, 5.9 | 18.0, 34.2 | | 14.3, 62.7 | 92.3, 92.3 |
| Change from | | | | | | | | |
| Baseline | | | | | | | | |
| n | 1 | 2 | | 1 | 2 | | 2 | 1 |
| Mean (SD) | −8.48 | −1.09 | (9.249) | −4.98 | −10.08 | (14.567) | −8.46 (15.028) | −11.00 |
| Median | −8.48 | −1.09 | | −4.98 | −10.08 | | −8.46 | −11.00 |
| Q1, Q3 | −8.48, −8.48 | −7.63, 5.45 | | −4.98, −4.98 | −20.38, 0.22 | | −19.09, 2.16 | −11.00, −11.00 |
| Min, Max | −8.5, −8.5 | −7.6, 5.5 | | −5.0, −5.0 | −20.4, 0.2 | | −19.1, 2.2 | −11.0, −11.0 |
| Percent | | | | | | | | |
| Change from | | | | | | | | |
| Baseline | | | | | | | | |
| n | 1 | 2 | | 1 | 2 | | 2 | 1 |
| Mean (SD) | −21.20 | −24.97 | (66.173) | −45.55 | −18.06 | (27.302) | −2.74 (29.125) | −10.65 |

| | | | | | | |
|---|---|---|---|---|---|---|
| Median | −21.20 | −24.97 | −45.55 | −18.06 | −2.74 | −10.65 |
| Q1, Q3 | −21.20, −21.20 | −71.76, 21.82 | −45.55, −45.55 | −37.36, 1.25 | −23.33, 17.86 | −10.65, −10.65 |
| Min, Max | −21.2, −21.2 | −71.8, 21.8 | −45.6, −45.6 | −37.4, 1.3 | −23.3, 17.9 | −10.6, −10.6 |

| Pooled | Pooled |
|---|---|
| Cilnidipine | Cilnidipine |
| 10 mg and | 20 mg and |
| Cilnidipine | Cilnidipine |

| | | |
|---|---|---|
| Pooled | 10 mg + | 20 mg + |

| | Pooled | Cilnidipine + | Tadalafil | Tadalafil | All |
|---|---|---|---|---|---|
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |

| | | | | | | | | | | |
|---|---|---|---|---|---|---|---|---|---|---|
| Mean (SD) | 25.21 | (14.689) | 41.56 | (32.775) | 37.44 | (18.517) | 32.39 | (33.580) | 36.56 | (33.315) |

| | | | | | |
|---|---|---|---|---|---|
| Median | 25.00 | 36.16 | 40.00 | 18.55 | 21.39 |
| Q1, Q3 | 10.63, 40.00 | 14.94, 68.18 | 17.78, 54.55 | 11.37, 53.41 | 10.92, 54.55 |
| Min, Max | 10.6, 40.0 | 12.1, 81.8 | 17.8, 54.5 | 10.6, 81.8 | 9.5, 103.3 |
| Average | | | | | |
| Duration | | | | | |
| n | 3 | 4 | 3 | 4 | 9 |

| | | | | | | | | | | |
|---|---|---|---|---|---|---|---|---|---|---|
| Mean (SD) | 21.66 | (16.168) | 32.29 | (22.053) | 27.90 | (8.672) | 27.61 | (25.981) | 32.49 | (28.765) |

| | | | | | |
|---|---|---|---|---|---|
| Median | 30.45 | 26.08 | 31.52 | 22.36 | 30.45 |
| Q1, Q3 | 3.00, 31.52 | 16.13, 48.45 | 18.00, 34.17 | 8.63, 46.59 | 14.27, 34.17 |
| Min, Max | 3.0, 31.5 | 14.3, 62.7 | 18.0, 34.2 | 3.0, 62.7 | 3.0, 92.3 |
| Change from | | | | | |
| Baseline | | | | | |
| n | 3 | 4 | 3 | 4 | 9 |

| | | | | | | | | | | |
|---|---|---|---|---|---|---|---|---|---|---|
| Mean (SD) | −3.55 | (7.809) | −9.27 | (12.119) | −9.54 | (10.342) | −4.77 | (11.043) | −7.08 | (8.937) |

| | | | | | |
|---|---|---|---|---|---|
| Median | −7.63 | −9.43 | −8.48 | −2.73 | −7.63 |
| Q1, Q3 | −8.48, 5.45 | −19.73, 1.19 | −20.38, 0.22 | −13.36, 3.81 | −11.00, 0.22 |
| Min, Max | −8.5, 5.5 | −20.4, 2.2 | −20.4, 0.2 | −19.1, 5.5 | −20.4, 5.5 |
| Percent | | | | | |
| Change from | | | | | |
| Baseline | | | | | |
| n | 3 | 4 | 3 | 4 | 9 |

| | | | | | | | | | | |
|---|---|---|---|---|---|---|---|---|---|---|
| Mean (SD) | −23.71 | (46.842) | −10.40 | (24.686) | −19.10 | (19.390) | −13.86 | (43.671) | −18.77 | (30.318) |

| | | | | | |
|---|---|---|---|---|---|
| Median | −21.20 | −11.04 | −21.20 | −2.74 | −21.20 |
| Q1, Q3 | −71.76, 21.82 | −30.35, 9.55 | −37.36, 1.25 | −47.55, 19.84 | −37.36, 1.25 |
| Min, Max | −71.8, 21.8 | −37.4, 17.9 | −37.4, 1.3 | −71.8, 21.8 | −71.8, 21.8 |

TABLE B-7
Average Duration of Weekly RP Attacks (All Attacks) Change and Percent
Change from Baseline by Baseline RCS. ITT Population - Part A.
Average duration (minutes) of weekly RP attacks is defined as the total duration
of RP attacks divided by total number of symptomatic RP attacks within the last 7
days of the dosing period (excluding the last dosing day). Baseline is calculated
as total duration of RP attacks during the last 7 days of screening period
(excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 40.00 | 17.81 | | 17.78 | 12.11 | 56.42 |
| (SD) | | (10.165) | | | | (66.350) |
| Median | 40.00 | 17.81 | | 17.78 | 12.11 | 56.42 |
| Q1, Q3 | 40.00, 40.00 | 10.63, 25.00 | | 17.78, 17.78 | 12.11, 12.11 | 9.50, 103.33 |
| Min, Max | 40.0, 40.0 | 10.6, 25.0 | | 17.8, 17.8 | 12.1, 12.1 | 9.5, 103.3 |
| Average Duration | | | | | | |
| n | 1 | 2 | | 1 | 1 | 1 |
| Mean | 31.52 | 16.73 | | 18.00 | 14.27 | 92.33 |
| (SD) | | (19.413) | | | | |
| Median | 31.52 | 16.73 | | 18.00 | 14.27 | 92.33 |
| Q1, Q3 | 31.52, 31.52 | 3.00, 30.45 | | 18.00, 18.00 | 14.27, 14.27 | 92.33, 92.33 |
| Min, Max | 31.5, 31.5 | 3.0, 30.5 | | 18.0, 18.0 | 14.3, 14.3 | 92.3, 92.3 |
| Change from Baseline | | | | | | |
| n | 1 | 2 | | 1 | 1 | 1 |
| Mean | −8.48 | −1.09 | | 0.22 | 2.16 | −11.00 |
| (SD) | | (9.249) | | | | |
| Median | −8.48 | −1.09 | | 0.22 | 2.16 | −11.00 |
| Q1, Q3 | −8.48, −8.48 | −7.63, 5.45 | | 0.22, 0.22 | 2.16, 2.16 | −11.00, −11.00 |
| Min, Max | −8.5, −8.5 | −7.6, 5.5 | | 0.2, 0.2 | 2.2, 2.2 | −11.0, −11.0 |
| Percent Change from Baseline | | | | | | |
| n | 1 | 2 | | 1 | 1 | 1 |
| Mean | −21.20 | −24.97 | | 1.25 | 17.86 | −10.65 |
| (SD) | | (66.173) | | | | |
| Median | −21.20 | −24.97 | | 1.25 | 17.86 | −10.65 |
| Q1, Q3 | −21.20, −21.20 | −71.76, 21.82 | | 1.25, 1.25 | 17.86, 17.86 | −10.65, −10.65 |
| Min, Max | −21.2, −21.2 | −71.8, 21.8 | | 1.3, 1.3 | 17.9, 17.9 | −10.6, −10.6 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 25.21 | 14.94 | 28.89 | 15.91 | 31.19 |
| (SD) | (14.689) | (4.011) | (15.713) | (7.907) | (33.561) |
| Median | 25.00 | 14.94 | 28.89 | 12.11 | 17.78 |
| Q1, Q3 | 10.63, 40.00 | 12.11, 17.78 | 17.78, 40.00 | 10.63, 25.00 | 10.63, 40.00 |
| Min, Max | 10.6, 40.0 | 12.1, 17.8 | 17.8, 40.0 | 10.6, 25.0 | 9.5, 103.3 |
| Average Duration | | | | | |
| n | 3 | 2 | 2 | 3 | 6 |
| Mean | 21.66 | 16.13 | 24.76 | 15.91 | 31.60 |
| (SD) | (16.168) | (2.640) | (9.561) | (13.801) | (31.601) |
| Median | 30.45 | 16.13 | 24.76 | 14.27 | 24.23 |
| Q1, Q3 | 3.00, 31.52 | 14.27, 18.00 | 18.00, 31.52 | 3.00, 30.45 | 14.27, 31.52 |
| Min, Max | 3.0, 31.5 | 14.3, 18.0 | 18.0, 31.5 | 3.0, 30.5 | 3.0, 92.3 |
| Change from Baseline | | | | | |
| n | 3 | 2 | 2 | 3 | 6 |
| Mean | −3.55 | 1.19 | −4.13 | 0.00 | −3.21 |
| (SD) | (7.809) | (1.371) | (6.152) | (6.803) | (6.688) |
| Median | −7.63 | 1.19 | −4.13 | 2.16 | −3.70 |
| Q1, Q3 | −8.48, 5.45 | 0.22, 2.16 | −8.48, 0.22 | −7.63, 5.45 | −8.48, 2.16 |
| Min, Max | −8.5, 5.5 | 0.2, 2.2 | −8.5, 0.2 | −7.6, 5.5 | −11.0, 5.5 |
| Percent Change from Baseline | | | | | |
| n | 3 | 2 | 2 | 3 | 6 |
| Mean | −23.71 | 9.55 | −9.97 | −10.70 | −10.45 |
| (SD) | (46.842) | (11.742) | (15.871) | (52.923) | (34.218) |
| Median | −21.20 | 9.55 | −9.97 | 17.86 | −4.70 |
| Q1, Q3 | −71.76, 21.82 | 1.25, 17.86 | −21.20, 1.25 | −71.76, 21.82 | −21.20, 17.86 |
| Min, Max | −71.8, 21.8 | 1.3, 17.9 | −21.2, 1.3 | −71.8, 21.8 | −71.8, 21.8 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 320.46 | 54.55 | 81.82 | |
| (SD) | | | (437.753) | | | |
| Median | | | 320.46 | 54.55 | 81.82 | |
| Q1, Q3 | | | 10.92, 630.00 | 54.55, 54.55 | 81.82, 81.82 | |
| Min, Max | | | 10.9, 630.0 | 54.5, 54.5 | 81.8, 81.8 | |
| Average Duration | | | | | | |
| n | | | 2 | 1 | 1 | |
| Mean | | | 412.26 | 34.17 | 62.73 | |
| (SD) | | | (574.612) | | | |
| Median | | | 412.26 | 34.17 | 62.73 | |
| Q1, Q3 | | | 5.95, 818.57 | 34.17, 34.17 | 62.73, 62.73 | |
| Min, Max | | | 5.9, 818.6 | 34.2, 34.2 | 62.7, 62.7 | |
| Change from Baseline | | | | | | |
| n | | | 2 | 1 | 1 | |
| Mean | | | 91.80 | −20.38 | −19.09 | |
| (SD) | | | (136.858) | | | |
| Median | | | 91.80 | −20.38 | −19.09 | |
| Q1, Q3 | | | −4.98, 188.57 | −20.38, −20.38 | −19.09, −19.09 | |
| Min, Max | | | −5.0, 188.6 | −20.4, −20.4 | −19.1, −19.1 | |
| Percent Change from Baseline | | | | | | |
| n | | | 2 | 1 | 1 | |
| Mean | | | −7.81 | −37.36 | −23.33 | |
| (SD) | | | (53.375) | | | |
| Median | | | −7.81 | −37.36 | −23.33 | |
| Q1, Q3 | | | −45.55, 29.93 | −37.36, −37.36 | −23.33, −23.33 | |
| Min, Max | | | −45.6, 29.9 | −37.4, −37.4 | −23.3, −23.3 | |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 68.18 | 54.55 | 81.82 | 194.32 |
| (SD) | | (19.285) | | | (291.916) |
| Median | | 68.18 | 54.55 | 81.82 | 68.18 |
| Q1, Q3 | | 54.55, 81.82 | 54.55, 54.55 | 81.82, 81.82 | 32.73, 355.91 |
| Min, Max | | 54.5, 81.8 | 54.5, 54.5 | 81.8, 81.8 | 10.9, 630.0 |
| Average Duration | | | | | |
| n | | 2 | 1 | 1 | 4 |
| Mean | | 48.45 | 34.17 | 62.73 | 230.35 |
| (SD) | | (20.195) | | | (392.830) |
| Median | | 48.45 | 34.17 | 62.73 | 48.45 |
| Q1, Q3 | | 34.17, 62.73 | 34.17, 34.17 | 62.73, 62.73 | 20.06, 440.65 |
| Min, Max | | 34.2, 62.7 | 34.2, 34.2 | 62.7, 62.7 | 5.9, 818.6 |
| Change from Baseline | | | | | |
| n | | 2 | 1 | 1 | 4 |
| Mean | | −19.73 | −20.38 | −19.09 | 36.03 |
| (SD) | | (0.911) | | | (101.932) |
| Median | | −19.73 | −20.38 | −19.09 | −12.03 |
| Q1, Q3 | | −20.38, −19.09 | −20.38, −20.38 | −19.09, −19.09 | −19.73, 91.80 |
| Min, Max | | −20.4, −19.1 | −20.4, −20.4 | −19.1, −19.1 | −20.4, 188.6 |
| Percent Change from Baseline | | | | | |
| n | | 2 | 1 | 1 | 4 |
| Mean | | −30.35 | −37.36 | −23.33 | −19.08 |
| (SD) | | (9.919) | | | (33.937) |
| Median | | −30.35 | −37.36 | −23.33 | −30.35 |
| Q1, Q3 | | −37.36, −23.33 | −37.36, −37.36 | −23.33, −23.33 | −41.46, 3.30 |
| Min, Max | | −37.4, −23.3 | −37.4, −37.4 | −23.3, −23.3 | −45.6, 29.9 |

TABLE B-8
Average Duration of Weekly RP Attacks (All Attacks) Change and Percent
Change from Baseline (Excluding Outliers) by Baseline RCS. ITT Population - Part A.
Average duration (minutes) of weekly RP attacks is defined as the total duration of RP
attacks divided by total number of symptomatic RP attacks within the last 7 days of the
dosing period (excluding the last dosing day). Baseline is calculated as total duration
of RP attacks during the last 7 days of screening period (excluding the first dosing day)
divided by the total number of RP attacks.

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | 1 | 2 | | 1 | 1 | 2 |
| Mean | 40.00 | 17.81 | | 17.78 | 12.11 | 56.42 |
| (SD) | | (10.165) | | | | (66.350) |
| Median | 40.00 | 17.81 | | 17.78 | 12.11 | 56.42 |
| Q1, Q3 | 40.00, 40.00 | 10.63, 25.00 | | 17.78, 17.78 | 12.11, 12.11 | 9.50, 103.33 |
| Min, Max | 40.0, 40.0 | 10.6, 25.0 | | 17.8, 17.8 | 12.1, 12.1 | 9.5, 103.3 |
| Average Duration | | | | | | |
| n | 1 | 2 | | 1 | 1 | 1 |
| Mean | 31.52 | 16.73 | | 18.00 | 14.27 | 92.33 |
| (SD) | | (19.413) | | | | |
| Median | 31.52 | 16.73 | | 18.00 | 14.27 | 92.33 |
| Q1, Q3 | 31.52, 31.52 | 3.00, 30.45 | | 18.00, 18.00 | 14.27, 14.27 | 92.33, 92.33 |
| Min, Max | 31.5, 31.5 | 3.0, 30.5 | | 18.0, 18.0 | 14.3, 14.3 | 92.3, 92.3 |
| Change from Baseline | | | | | | |
| n | 1 | 2 | | 1 | 1 | 1 |
| Mean | −8.48 | −1.09 | | 0.22 | 2.16 | −11.00 |
| (SD) | | (9.249) | | | | |
| Median | −8.48 | −1.09 | | 0.22 | 2.16 | −11.00 |
| Q1, Q3 | −8.48, −8.48 | −7.63, 5.45 | | 0.22, 0.22 | 2.16, 2.16 | −11.00, −11.00 |
| Min, Max | −8.5, −8.5 | −7.6, 5.5 | | 0.2, 0.2 | 2.2, 2.2 | −11.0, −11.0 |
| Percent Change from Baseline | | | | | | |
| n | 1 | 2 | | 1 | 1 | 1 |
| Mean | −21.20 | −24.97 | | 1.25 | 17.86 | −10.65 |
| (SD) | | (66.173) | | | | |
| Median | −21.20 | −24.97 | | 1.25 | 17.86 | −10.65 |
| Q1, Q3 | −21.20, −21.20 | −71.76, 21.82 | | 1.25, 1.25 | 17.86, 17.86 | −10.65, −10.65 |
| Min, Max | −21.2, −21.2 | −71.8, 21.8 | | 1.3, 1.3 | 17.9, 17.9 | −10.6, −10.6 |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | 3 | 2 | 2 | 3 | 7 |
| Mean | 25.21 | 14.94 | 28.89 | 15.91 | 31.19 |
| (SD) | (14.689) | (4.011) | (15.713) | (7.907) | (33.561) |
| Median | 25.00 | 14.94 | 28.89 | 12.11 | 17.78 |
| Q1, Q3 | 10.63, 40.00 | 12.11, 17.78 | 17.78, 40.00 | 10.63, 25.00 | 10.63, 40.00 |
| Min, Max | 10.6, 40.0 | 12.1, 17.8 | 17.8, 40.0 | 10.6, 25.0 | 9.5, 103.3 |
| Average Duration | | | | | |
| n | 3 | 2 | 2 | 3 | 6 |
| Mean | 21.66 | 16.13 | 24.76 | 15.91 | 31.60 |
| (SD) | (16.168) | (2.640) | (9.561) | (13.801) | (31.601) |
| Median | 30.45 | 16.13 | 24.76 | 14.27 | 24.23 |
| Q1, Q3 | 3.00, 31.52 | 14.27, 18.00 | 18.00, 31.52 | 3.00, 30.45 | 14.27, 31.52 |
| Min, Max | 3.0, 31.5 | 14.3, 18.0 | 18.0, 31.5 | 3.0, 30.5 | 3.0, 92.3 |
| Change from Baseline | | | | | |
| n | 3 | 2 | 2 | 3 | 6 |
| Mean | −3.55 | 1.19 | −4.13 | 0.00 | −3.21 |
| (SD) | (7.809) | (1.371) | (6.152) | (6.803) | (6.688) |
| Median | −7.63 | 1.19 | −4.13 | 2.16 | −3.70 |
| Q1, Q3 | −8.48, 5.45 | 0.22, 2.16 | −8.48, 0.22 | −7.63, 5.45 | −8.48, 2.16 |
| Min, Max | −8.5, 5.5 | 0.2, 2.2 | −8.5, 0.2 | −7.6, 5.5 | −11.0, 5.5 |
| Percent Change from Baseline | | | | | |
| n | 3 | 2 | 2 | 3 | 6 |
| Mean | −23.71 | 9.55 | −9.97 | −10.70 | −10.45 |
| (SD) | (46.842) | (11.742) | (15.871) | (52.923) | (34.218) |
| Median | −21.20 | 9.55 | −9.97 | 17.86 | −4.70 |
| Q1, Q3 | −71.76, 21.82 | 1.25, 17.86 | −21.20, 1.25 | −71.76, 21.82 | −21.20, 17.86 |
| Min, Max | −71.8, 21.8 | 1.3, 17.9 | −21.2, 1.3 | −71.8, 21.8 | −71.8, 21.8 |

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | | | 1 | 1 | 1 | |
| Mean | | | 10.92 | 54.55 | 81.82 | |
| (SD) | | | | | | |
| Median | | | 10.92 | 54.55 | 81.82 | |
| Q1, Q3 | | | 10.92, 10.92 | 54.55, 54.55 | 81.82, 81.82 | |
| Min, Max | | | 10.9, 10.9 | 54.5, 54.5 | 81.8, 81.8 | |
| Average Duration | | | | | | |
| n | | | 1 | 1 | 1 | |
| Mean | | | 5.95 | 34.17 | 62.73 | |
| (SD) | | | | | | |
| Median | | | 5.95 | 34.17 | 62.73 | |
| Q1, Q3 | | | 5.95, 5.95 | 34.17, 34.17 | 62.73, 62.73 | |
| Min, Max | | | 5.9, 5.9 | 34.2, 34.2 | 62.7, 62.7 | |
| Change from Baseline | | | | | | |
| n | | | 1 | 1 | 1 | |
| Mean | | | −4.98 | −20.38 | −19.09 | |
| (SD) | | | | | | |
| Median | | | −4.98 | −20.38 | −19.09 | |
| Q1, Q3 | | | −4.98, −4.98 | −20.38, −20.38 | −19.09, −19.09 | |
| Min, Max | | | −5.0, −5.0 | −20.4, −20.4 | −19.1, −19.1 | |
| Percent Change from Baseline | | | | | | |
| n | | | 1 | 1 | 1 | |
| Mean | | | −45.55 | −37.36 | −23.33 | |
| (SD) | | | | | | |
| Median | | | −45.55 | −37.36 | −23.33 | |
| Q1, Q3 | | | −45.55, −45.55 | −37.36, −37.36 | −23.33, −23.33 | |
| Min, Max | | | −45.6, −45.6 | −37.4, −37.4 | −23.3, −23.3 | |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | | 2 | 1 | 1 | 3 |
| Mean | | 68.18 | 54.55 | 81.82 | 49.10 |
| (SD) | | (19.285) | | | (35.760) |
| Median | | 68.18 | 54.55 | 81.82 | 54.55 |
| Q1, Q3 | | 54.55, 81.82 | 54.55, 54.55 | 81.82, 81.82 | 10.92, 81.82 |
| Min, Max | | 54.5, 81.8 | 54.5, 54.5 | 81.8, 81.8 | 10.9, 81.8 |
| Average Duration | | | | | |
| n | | 2 | 1 | 1 | 3 |
| Mean | | 48.45 | 34.17 | 62.73 | 34.28 |
| (SD) | | (20.195) | | | (28.390) |
| Median | | 48.45 | 34.17 | 62.73 | 34.17 |
| Q1, Q3 | | 34.17, 62.73 | 34.17, 34.17 | 62.73, 62.73 | 5.95, 62.73 |
| Min, Max | | 34.2, 62.7 | 34.2, 34.2 | 62.7, 62.7 | 5.9, 62.7 |
| Change from Baseline | | | | | |
| n | | 2 | 1 | 1 | 3 |
| Mean | | −19.73 | −20.38 | −19.09 | −14.82 |
| (SD) | | (0.911) | | | (8.545) |
| Median | | −19.73 | −20.38 | −19.09 | −19.09 |
| Q1, Q3 | | −20.38, −19.09 | −20.38, −20.38 | −19.09, −19.09 | −20.38, −4.98 |
| Min, Max | | −20.4, −19.1 | −20.4, −20.4 | −19.1, −19.1 | −20.4, −5.0 |
| Percent Change from Baseline | | | | | |
| n | | 2 | 1 | 1 | 3 |
| Mean | | −30.35 | −37.36 | −23.33 | −35.42 |
| (SD) | | (9.919) | | | (11.237) |
| Median | | −30.35 | −37.36 | −23.33 | −37.36 |
| Q1, Q3 | | −37.36, −23.33 | −37.36, −37.36 | −23.33, −23.33 | −45.55, −23.33 |
| Min, Max | | −37.4, −23.3 | −37.4, −37.4 | −23.3, −23.3 | −45.6, −23.3 |

Tables C-1 to C-8 include data that relates to the severity of symptomatic Raynaud's attacks.

TABLE C-1
Average Severity of Weekly Symptomatic RP Attacks Changeand Percent
Change from Baseline. ITT Population - Part A.
Average severity (0-10) of weekly symptomatic RP attacks is defined as the
total severity scores of symptomatic RP attacks divided by total number of
symptomatic RP attacks within the last 7 days of the dosing period (excluding
the last dosing day). Baseline is calculated as total severity scores of symptomatic
RP attacks during the last 7 days of screening period (excluding the first dosing day)
divided by the total number of symptomatic RP attacks. Only symptomatic RP attacks, defined
as RP attacks with patient reported finger color changes associated with at least one
symptom (pain, numbness, tingling), were included in the derivations.

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | 1 | 2 | 2 | 2 | 2 | 2 |
| Mean | 3.49 | 3.74 | 7.68 | 5.99 | 5.56 | 3.82 |
| (SD) | | (1.430) | (2.398) | (0.520) | (1.823) | (0.055) |
| Median | 3.49 | 3.74 | 7.68 | 5.99 | 5.56 | 3.82 |
| Q1, Q3 | 3.49, 3.49 | 2.73, 4.75 | 5.98, 9.38 | 5.62, 6.36 | 4.27, 6.84 | 3.78, 3.86 |
| Min, Max | 3.5, 3.5 | 2.7, 4.8 | 6.0, 9.4 | 5.6, 6.4 | 4.3, 6.8 | 3.8, 3.9 |
| Average Severity | | | | | | |
| n | 1 | 2 | 2 | 2 | 2 | 2 |
| Mean | 3.77 | 2.30 | 7.08 | 4.06 | 4.51 | 3.43 |
| (SD) | | (0.141) | (3.406) | (1.644) | (0.774) | (0.601) |
| Median | 3.77 | 2.30 | 7.08 | 4.06 | 4.51 | 3.43 |
| Q1, Q3 | 3.77, 3.77 | 2.20, 2.40 | 4.67, 9.48 | 2.90, 5.23 | 3.96, 5.06 | 3.00, 3.85 |
| Min, Max | 3.8, 3.8 | 2.2, 2.4 | 4.7, 9.5 | 2.9, 5.2 | 4.0, 5.1 | 3.0, 3.9 |
| Change from Baseline | | | | | | |
| n | 1 | 2 | 2 | 2 | 2 | 2 |
| Mean | 0.28 | −1.44 | −0.60 | −1.93 | −1.05 | −0.39 |
| (SD) | | (1.571) | (1.008) | (1.124) | (1.049) | (0.656) |
| Median | 0.28 | −1.44 | −0.60 | −1.93 | −1.05 | −0.39 |
| Q1, Q3 | 0.28, 0.28 | −2.55, −0.33 | −1.32, 0.11 | −2.72, −1.13 | −1.79, −0.31 | −0.86, 0.07 |
| Min, Max | 0.3, 0.3 | −2.6, −0.3 | −1.3, 0.1 | −2.7, −1.1 | −1.8, −0.3 | −0.9, 0.1 |
| Percent Change from Baseline | | | | | | |
| n | 1 | 2 | 2 | 2 | 2 | 2 |
| Mean | 8.05 | −32.85 | −10.43 | −33.11 | −16.64 | −10.20 |
| (SD) | | (29.464) | (16.377) | (21.645) | (13.424) | (17.040) |
| Median | 8.05 | −32.85 | −10.43 | −33.11 | −16.64 | −10.20 |
| Q1, Q3 | 8.05, 8.05 | −53.68, −12.02 | −22.01, 1.16 | −48.42, −17.81 | −26.14, −7.15 | −22.25, 1.85 |
| Min, Max | 8.0, 8.0 | −53.7, −12.0 | −22.0, 1.2 | −48.4, −17.8 | −26.1, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | 3 | 4 | 3 | 4 | 11 |
| Mean | 3.66 | 5.77 | 5.16 | 4.65 | 5.19 |
| (SD) | (1.021) | (1.123) | (1.489) | (1.700) | (1.902) |
| Median | 3.49 | 5.99 | 5.62 | 4.51 | 4.75 |
| Q1, Q3 | 2.73, 4.75 | 4.94, 6.60 | 3.49, 6.36 | 3.50, 5.80 | 3.78, 6.36 |
| Min, Max | 2.7, 4.8 | 4.3, 6.8 | 3.5, 6.4 | 2.7, 6.8 | 2.7, 9.4 |
| Average Severity | | | | | |
| n | 3 | 4 | 3 | 4 | 11 |
| Mean | 2.79 | 4.29 | 3.97 | 3.40 | 4.23 |
| (SD) | (0.855) | (1.080) | (1.175) | (1.354) | (2.016) |
| Median | 2.40 | 4.51 | 3.77 | 3.18 | 3.85 |
| Q1, Q3 | 2.20, 3.77 | 3.43, 5.14 | 2.90, 5.23 | 2.30, 4.51 | 2.90, 5.06 |
| Min, Max | 2.2, 3.8 | 2.9, 5.2 | 2.9, 5.2 | 2.2, 5.1 | 2.2, 9.5 |
| Change from Baseline | | | | | |
| n | 3 | 4 | 3 | 4 | 11 |
| Mean | −0.87 | −1.49 | −1.19 | −1.24 | −0.96 |
| (SD) | (1.490) | (1.023) | (1.502) | (1.114) | (1.052) |
| Median | −0.33 | −1.46 | −1.13 | −1.06 | −0.86 |
| Q1, Q3 | −2.55, 0.28 | −2.26, −0.72 | −2.72, 0.28 | −2.17, −0.32 | −1.79, 0.07 |
| Min, Max | −2.6, 0.3 | −2.7, −0.3 | −2.7, 0.3 | −2.6, −0.3 | −2.7, 0.3 |
| Percent Change from Baseline | | | | | |
| n | 3 | 4 | 3 | 4 | 11 |
| Mean | −19.22 | −24.88 | −19.39 | −24.75 | −18.04 |
| (SD) | (31.490) | (17.512) | (28.267) | (20.904) | (19.728) |
| Median | −12.02 | −21.97 | −17.81 | −19.08 | −17.81 |
| Q1, Q3 | −53.68, 8.05 | −37.28, −12.48 | −48.42, 8.05 | −39.91, −9.58 | −26.14, 1.16 |
| Min, Max | −53.7, 8.0 | −48.4, −7.2 | −48.4, 8.0 | −53.7, −7.2 | −53.7, 8.0 |

TABLE C-2
Average Severity of Weekly Symptomatic RP Attacks Change and Percent Change
from Baseline (Excluding Outliers). ITT Population - Part A.
Average severity (0-10) of weekly symptomatic RP attacks is defined as the total
severity scores of symptomatic RP attacks divided by total number of symptomatic
RP attacks within the last 7 days of the dosing period (excluding the last dosing day).
Baseline is calculated as total severity scores of symptomatic RP attacks during the last 7 days
of screening period (excluding the first dosing day) divided by the total number of symptomatic RP attacks.
Only symptomatic RP attacks, defined as RP attacks with patient reported finger color changes associated
with at least one symptom (pain, numbness, tingling), were included in the derivations.

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | 1 | 2 | 1 | 2 | 2 | 2 |
| Mean | 3.49 | 3.74 | 5.98 | 5.99 | 5.56 | 3.82 |
| (SD) | | (1.430) | | (0.520) | (1.823) | (0.055) |
| Median | 3.49 | 3.74 | 5.98 | 5.99 | 5.56 | 3.82 |
| Q1, Q3 | 3.49, 3.49 | 2.73, 4.75 | 5.98, 5.98 | 5.62, 6.36 | 4.27, 6.84 | 3.78, 3.86 |
| Min, Max | 3.5, 3.5 | 2.7, 4.8 | 6.0, 6.0 | 5.6, 6.4 | 4.3, 6.8 | 3.8, 3.9 |
| Average Severity | | | | | | |
| n | 1 | 2 | 1 | 2 | 2 | 2 |
| Mean | 3.77 | 2.30 | 4.67 | 4.06 | 4.51 | 3.43 |
| (SD) | | (0.141) | | (1.644) | (0.774) | (0.601) |
| Median | 3.77 | 2.30 | 4.67 | 4.06 | 4.51 | 3.43 |
| Q1, Q3 | 3.77, 3.77 | 2.20, 2.40 | 4.67, 4.67 | 2.90, 5.23 | 3.96, 5.06 | 3.00, 3.85 |
| Min, Max | 3.8, 3.8 | 2.2, 2.4 | 4.7, 4.7 | 2.9, 5.2 | 4.0, 5.1 | 3.0, 3.9 |
| Change from Baseline | | | | | | |
| n | 1 | 2 | 1 | | | 2 |
| Mean | 0.28 | −1.44 | −1.32 | −1.93 | −1.05 | −0.39 |
| (SD) | | (1.571) | | (1.124) | (1.049) | (0.656) |
| Median | 0.28 | −1.44 | −1.32 | −1.93 | −1.05 | −0.39 |
| Q1, Q3 | 0.28, 0.28 | −2.55, −0.33 | −1.32, −1.32 | −2.72, −1.13 | −1.79, −0.31 | −0.86, 0.07 |
| Min, Max | 0.3, 0.3 | −2.6, −0.3 | −1.3, −1.3 | −2.7, −1.1 | −1.8, −0.3 | −0.9, 0.1 |
| Percent Change from Baseline | | | | | | |
| n | 1 | 2 | 1 | 2 | 2 | 2 |
| Mean | 8.05 | −32.85 | −22.01 | −33.11 | −16.64 | −10.20 |
| (SD) | | (29.464) | | (21.645) | (13.424) | (17.040) |
| Median | 8.05 | −32.85 | −22.01 | −33.11 | −16.64 | −10.20 |
| Q1, Q3 | 8.05, 8.05 | −53.68, −12.02 | −22.01, −22.01 | −48.42, −17.81 | −26.14, −7.15 | −22.25, 1.85 |
| Min, Max | 8.0, 8.0 | −53.7, −12.0 | −22.0, −22.0 | −48.4, −17.8 | −26.1, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | 3 | 4 | 3 | 4 | 10 |
| Mean | 3.66 | 5.77 | 5.16 | 4.65 | 4.77 |
| (SD) | (1.021) | (1.123) | (1.489) | (1.700) | (1.370) |
| Median | 3.49 | 5.99 | 5.62 | 4.51 | 4.51 |
| Q1, Q3 | 2.73, 4.75 | 4.94, 6.60 | 3.49, 6.36 | 3.50, 5.80 | 3.78, 5.98 |
| Min, Max | 2.7, 4.8 | 4.3, 6.8 | 3.5, 6.4 | 2.7, 6.8 | 2.7, 6.8 |
| Average Severity | | | | | |
| n | 3 | 4 | 3 | 4 | 10 |
| Mean | 2.79 | 4.29 | 3.97 | 3.40 | 3.70 |
| (SD) | (0.855) | (1.080) | (1.175) | (1.354) | (1.068) |
| Median | 2.40 | 4.51 | 3.77 | 3.18 | 3.81 |
| Q1, Q3 | 2.20, 3.77 | 3.43, 5.14 | 2.90, 5.23 | 2.30, 4.51 | 2.90, 4.67 |
| Min, Max | 2.2, 3.8 | 2.9, 5.2 | 2.9, 5.2 | 2.2, 5.1 | 2.2, 5.2 |
| Change from Baseline | | | | | |
| n | 3 | 4 | 3 | 4 | 10 |
| Mean | −0.87 | −1.49 | −1.19 | −1.24 | −1.07 |
| (SD) | (1.490) | (1.023) | (1.502) | (1.114) | (1.044) |
| Median | −0.33 | −1.46 | −1.13 | −1.06 | −1.00 |
| Q1, Q3 | −2.55, 0.28 | −2.26, −0.72 | −2.72, 0.28 | −2.17, −0.32 | −1.79, −0.31 |
| Min, Max | −2.6, 0.3 | −2.7, −0.3 | −2.7, 0.3 | −2.6, −0.3 | −2.7, 0.3 |
| Percent Change from Baseline | | | | | |
| n | 3 | 4 | 3 | 4 | 10 |
| Mean | −19.22 | −24.88 | −19.39 | −24.75 | −19.96 |
| (SD) | (31.490) | (17.512) | (28.267) | (20.904) | (19.683) |
| Median | −12.02 | −21.97 | −17.81 | −19.08 | −19.91 |
| Q1, Q3 | −53.68, 8.05 | −37.28, −12.48 | −48.42, 8.05 | −39.91, −9.58 | −26.14, −7.15 |
| Min, Max | −53.7, 8.0 | −48.4, −7.2 | −48.4, 8.0 | −53.7, −7.2 | −53.7, 8.0 |

TABLE C-3
Average Severity of Weekly Symptomatic RP Attacks Change and
Percent Change from Baseline by Baseline RCS. ITT Population - Part A.
Average severity (0-10) of weekly symptomatic RP attacks is defined as the
total severity scores of symptomatic RP attacks divided by total number of symptomatic
RP attacks within the last 7 days of the dosing period (excluding the last dosing day).
Baseline is calculated as total severity scores of symptomatic RP attacks during the last 7 days
of screening period (excluding the first dosing day) divided by the total number of symptomatic RP attacks.
Only symptomatic RP attacks, defined as RP attacks with patient reported finger color changes associated
with at least one symptom (pain, numbness, tingling), were included in the derivations.

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | 1 | 2 | | 1 | 1 | 2 |
| Mean | 3.49 | 3.74 | | 5.62 | 4.27 | 3.82 |
| (SD) | | (1.430) | | | | (0.055) |
| Median | 3.49 | 3.74 | | 5.62 | 4.27 | 3.82 |
| Q1, Q3 | 3.49, 3.49 | 2.73, 4.75 | | 5.62, 5.62 | 4.27, 4.27 | 3.78, 3.86 |
| Min, Max | 3.5, 3.5 | 2.7, 4.8 | | 5.6, 5.6 | 4.3, 4.3 | 3.8, 3.9 |
| Average Severity | | | | | | |
| n | 1 | 2 | | 1 | 1 | 2 |
| Mean | 3.77 | 2.30 | | 2.90 | 3.96 | 3.43 |
| (SD) | | (0.141) | | | | (0.601) |
| Median | 3.77 | 2.30 | | 2.90 | 3.96 | 3.43 |
| Q1, Q3 | 3.77, 3.77 | 2.20, 2.40 | | 2.90, 2.90 | 3.96, 3.96 | 3.00, 3.85 |
| Min, Max | 3.8, 3.8 | 2.2, 2.4 | | 2.9, 2.9 | 4.0, 4.0 | 3.0, 3.9 |
| Change from Baseline | | | | | | |
| n | 1 | 2 | | 1 | 1 | 2 |
| Mean | 0.28 | −1.44 | | −2.72 | −0.31 | −0.39 |
| (SD) | | (1.571) | | | | (0.656) |
| Median | 0.28 | −1.44 | | −2.72 | −0.31 | −0.39 |
| Q1, Q3 | 0.28, 0.28 | −2.55, −0.33 | | −2.72, −2.72 | −0.31, −0.31 | −0.86, 0.07 |
| Min, Max | 0.3, 0.3 | −2.6, −0.3 | | −2.7, −2.7 | −0.3, −0.3 | −0.9, 0.1 |
| Percent Change from Baseline | | | | | | |
| n | 1 | 2 | | 1 | 1 | 2 |
| Mean | 8.05 | −32.85 | | −48.42 | −7.15 | −10.20 |
| (SD) | | (29.464) | | | | (17.040) |
| Median | 8.05 | −32.85 | | −48.42 | −7.15 | −10.20 |
| Q1, Q3 | 8.05, 8.05 | −53.68, −12.02 | | −48.42, −48.42 | −7.15, −7.15 | −22.25, 1.85 |
| Min, Max | 8.0, 8.0 | −53.7, −12.0 | | −48.4, −48.4 | −7.2, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | 3 | 2 | 2 | 3 | 7 |
| Mean | 3.66 | 4.94 | 4.56 | 3.91 | 4.07 |
| (SD) | (1.021) | (0.959) | (1.507) | (1.056) | (0.929) |
| Median | 3.49 | 4.94 | 4.56 | 4.27 | 3.86 |
| Q1, Q3 | 2.73, 4.75 | 4.27, 5.62 | 3.49, 5.62 | 2.73, 4.75 | 3.49, 4.75 |
| Min, Max | 2.7, 4.8 | 4.3, 5.6 | 3.5, 5.6 | 2.7, 4.8 | 2.7, 5.6 |
| Average Severity | | | | | |
| n | 3 | 2 | 2 | 3 | 7 |
| Mean | 2.79 | 3.43 | 3.34 | 2.85 | 3.15 |
| (SD) | (0.855) | (0.751) | (0.616) | (0.964) | (0.717) |
| Median | 2.40 | 3.43 | 3.34 | 2.40 | 3.00 |
| Q1, Q3 | 2.20, 3.77 | 2.90, 3.96 | 2.90, 3.77 | 2.20, 3.96 | 2.40, 3.85 |
| Min, Max | 2.2, 3.8 | 2.9, 4.0 | 2.9, 3.8 | 2.2, 4.0 | 2.2, 4.0 |
| Change from Baseline | | | | | |
| n | 3 | 2 | 2 | 3 | 7 |
| Mean | −0.87 | −1.51 | −1.22 | −1.06 | −0.92 |
| (SD) | (1.490) | (1.709) | (2.124) | (1.290) | (1.229) |
| Median | −0.33 | −1.51 | −1.22 | −0.33 | −0.33 |
| Q1, Q3 | −2.55, 0.28 | −2.72, −0.31 | −2.72, 0.28 | −2.55, −0.31 | −2.55, 0.07 |
| Min, Max | −2.6, 0.3 | −2.7, −0.3 | −2.7, 0.3 | −2.6, −0.3 | −2.7, 0.3 |
| Percent Change from Baseline | | | | | |
| n | 3 | 2 | 2 | 3 | 7 |
| Mean | −19.22 | −27.79 | −20.18 | −24.28 | −19.09 |
| (SD) | (31.490) | (29.181) | (39.929) | (25.577) | (23.925) |
| Median | −12.02 | −27.79 | −20.18 | −12.02 | −12.02 |
| Q1, Q3 | −53.68, 8.05 | −48.42, −7.15 | −48.42, 8.05 | −53.68, −7.15 | −48.42, 1.85 |
| Min, Max | −53.7, 8.0 | −48.4, −7.2 | −48.4, 8.0 | −53.7, −7.2 | −53.7, 8.0 |

| | | | | Cilnidipine | Cilnidipine | |
|---|---|---|---|---|---|---|
| | Cilnidipine | Cilnidipine | Tadalafil | 10 mg + | 20 mg + | |
| | 10 mg | 20 mg | 5 mg | Tadalafil | Tadalafil | Placebo |
| Statistics | (N = 1) | (N = 2) | (N = 2) | 5 mg (N = 2) | 5 mg (N = 2) | (N = 2) |
| Baseline | | | | | | |
| n | | | 2 | 1 | 1 | |
| Mean | | | 7.68 | 6.36 | 6.84 | |
| (SD) | | | (2.398) | | | |
| Median | | | 7.68 | 6.36 | 6.84 | |
| Q1, Q3 | | | 5.98, 9.38 | 6.36, 6.36 | 6.84, 6.84 | |
| Min, Max | | | 6.0, 9.4 | 6.4, 6.4 | 6.8, 6.8 | |
| Average Severity | | | | | | |
| n | | | 2 | 1 | 1 | |
| Mean | | | 7.08 | 5.23 | 5.06 | |
| (SD) | | | (3.406) | | | |
| Median | | | 7.08 | 5.23 | 5.06 | |
| Q1, Q3 | | | 4.67, 9.48 | 5.23, 5.23 | 5.06, 5.06 | |
| Min, Max | | | 4.7, 9.5 | 5.2, 5.2 | 5.1, 5.1 | |
| Change from Baseline | | | | | | |
| n | | | 2 | 1 | 1 | |
| Mean | | | −0.60 | −1.13 | −1.79 | |
| (SD) | | | (1.008) | | | |
| Median | | | −0.60 | −1.13 | −1.79 | |
| Q1, Q3 | | | −1.32, 0.11 | −1.13, −1.13 | −1.79, −1.79 | |
| Min, Max | | | −1.3, 0.1 | −1.1, −1.1 | −1.8, −1.8 | |
| Percent Change from Baseline | | | | | | |
| n | | | 2 | 1 | 1 | |
| Mean | | | −10.43 | −17.81 | −26.14 | |
| (SD) | | | (16.377) | | | |
| Median | | | −10.43 | −17.81 | −26.14 | |
| Q1, Q3 | | | −22.01, 1.16 | −17.81, −17.81 | −26.14, −26.14 | |
| Min, Max | | | −22.0, 1.2 | −17.8, −17.8 | −26.1, −26.1 | |

| | | | Pooled | Pooled | |
|---|---|---|---|---|---|
| | | | Cilnidipine | Cilnidipine | |
| | | | 10 mg and | 20 mg and | |
| | | Pooled | Cilnidipine | Cilnidipine | |
| | Pooled | Cilnidipine + | 10 mg + | 20 mg + | All |
| | Cilnidipine | Tadalafil | Tadalafil | Tadalafil | Participants |
| Statistics | (N = 3) | (N = 4) | 5 mg (N = 3) | 5 mg (N = 4) | (N = 11) |
| Baseline | | | | | |
| n | | 2 | 1 | 1 | 4 |
| Mean | | 6.60 | 6.36 | 6.84 | 7.14 |
| (SD) | | (0.345) | | | (1.531) |
| Median | | 6.60 | 6.36 | 6.84 | 6.60 |
| Q1, Q3 | | 6.36, 6.84 | 6.36, 6.36 | 6.84, 6.84 | 6.17, 8.11 |
| Min, Max | | 6.4, 6.8 | 6.4, 6.4 | 6.8, 6.8 | 6.0, 9.4 |
| Average Severity | | | | | |
| n | | 2 | 1 | 1 | 4 |
| Mean | | 5.14 | 5.23 | 5.06 | 6.11 |
| (SD) | | (0.120) | | | (2.263) |
| Median | | 5.14 | 5.23 | 5.06 | 5.14 |
| Q1, Q3 | | 5.06, 5.23 | 5.23, 5.23 | 5.06, 5.06 | 4.86, 7.35 |
| Min, Max | | 5.1, 5.2 | 5.2, 5.2 | 5.1, 5.1 | 4.7, 9.5 |
| Change from Baseline | | | | | |
| n | | 2 | 1 | 1 | 4 |
| Mean | | −1.46 | −1.13 | −1.79 | −1.03 |
| (SD) | | (0.464) | | | (0.809) |
| Median | | −1.46 | −1.13 | −1.79 | −1.22 |
| Q1, Q3 | | −1.79, −1.13 | −1.13, −1.13 | −1.79, −1.79 | −1.55, −0.51 |
| Min, Max | | −1.8, −1.1 | −1.1, −1.1 | −1.8, −1.8 | −1.8, 0.1 |
| Percent Change from Baseline | | | | | |
| n | | 2 | 1 | 1 | 4 |
| Mean | | −21.97 | −17.81 | −26.14 | −16.20 |
| (SD) | | (5.888) | | | (12.059) |
| Median | | −21.97 | −17.81 | −26.14 | −19.91 |
| Q1, Q3 | | −26.14, −17.81 | −17.81, −17.81 | −26.14, −26.14 | −24.07, −8.33 |
| Min, Max | | −26.1, −17.8 | −17.8, −17.8 | −26.1, −26.1 | −26.1, 1.2 |

TABLE C-4
Average Severity of Weekly Symptomatic RP Attacks Change and Percent Change from Baseline
(Excluding Outliers) by Baseline RCS. ITT Population - Part A. Average severity (0-10) of
weekly symptomatic RP attacks is defined as the total severity scores of symptomatic RP
attacks divided by total number of symptomatic RP attacks within the last 7 days of the
dosing period (excluding the last dosing day). Baseline is calculated as total severity
scores of symptomatic RP attacks during the last 7 days of screening period (excluding the
first dosing day) divided by the total number of symptomatic RP attacks. Only symptomatic
RP attacks, defined as RP attacks with patient reported finger color changes associated
with at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.49 | 3.74 | | 5.62 | 4.27 | 3.82 |
| (SD) | | (1.430) | | | | (0.055) |
| Median | 3.49 | 3.74 | | 5.62 | 4.27 | 3.82 |
| Q1, Q3 | 3.49, 3.49 | 2.73, 4.75 | | 5.62, 5.62 | 4.27, 4.27 | 3.78, 3.86 |
| Min, Max | 3.5, 3.5 | 2.7, 4.8 | | 5.6, 5.6 | 4.3, 4.3 | 3.8, 3.9 |
| Average | | | | | | |
| Severity | | | | | | |
| n | 1 | 2 | | 1 | 1 | 2 |
| Mean | 3.77 | 2.30 | | 2.90 | 3.96 | 3.43 |
| (SD) | | (0.141) | | | | (0.601) |
| Median | 3.77 | 2.30 | | 2.90 | 3.96 | 3.43 |
| Q1, Q3 | 3.77, 3.77 | 2.20, 2.40 | | 2.90, 2.90 | 3.96, 3.96 | 3.00, 3.85 |
| Min, Max | 3.8, 3.8 | 2.2, 2.4 | | 2.9, 2.9 | 4.0, 4.0 | 3.0, 3.9 |
| Change | | | | | | |
| from | | | | | | |
| Baseline | | | | | | |
| n | 1 | 2 | | 1 | 1 | 2 |
| Mean | 0.28 | −1.44 | | −2.72 | −0.31 | −0.39 |
| (SD) | | (1.571) | | | | (0.656) |
| Median | 0.28 | −1.44 | | −2.72 | −0.31 | −0.39 |
| Q1, Q3 | 0.28, 0.28 | −2.55, −0.33 | | −2.72, −2.72 | −0.31, −0.31 | −0.86, 0.07 |
| Min, Max | 0.3, 0.3 | −2.6, −0.3 | | −2.7, −2.7 | −0.3, −0.3 | −0.9, 0.1 |
| Percent | | | | | | |
| Change | | | | | | |
| from | | | | | | |
| Baseline | | | | | | |
| n | 1 | 2 | | 1 | 1 | 2 |
| Mean | 8.05 | −32.85 | | −48.42 | −7.15 | −10.20 |
| (SD) | | (29.464) | | | | (17.040) |
| Median | 8.05 | −32.85 | | −48.42 | −7.15 | −10.20 |
| Q1, Q3 | 8.05, 8.05 | −53.68, −12.02 | | −48.42, −48.42 | −7.15, −7.15 | −22.25, 1.85 |
| Min, Max | 8.0, 8.0 | −53.7, −12.0 | | −48.4, −48.4 | −7.2, −7.2 | −22.2, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 3.66 | 4.94 | 4.56 | 3.91 | 4.07 |
| (SD) | (1.021) | (0.959) | (1.507) | (1.056) | (0.929) |
| Median | 3.49 | 4.94 | 4.56 | 4.27 | 3.86 |
| Q1, Q3 | 2.73, 4.75 | 4.27, 5.62 | 3.49, 5.62 | 2.73, 4.75 | 3.49, 4.75 |
| Min, Max | 2.7, 4.8 | 4.3, 5.6 | 3.5, 5.6 | 2.7, 4.8 | 2.7, 5.6 |
| Average | | | | | |
| Severity | | | | | |
| n | 3 | 2 | 2 | 3 | 7 |
| Mean | 2.79 | 3.43 | 3.34 | 2.85 | 3.15 |
| (SD) | (0.855) | (0.751) | (0.616) | (0.964) | (0.717) |
| Median | 2.40 | 3.43 | 3.34 | 2.40 | 3.00 |
| Q1, Q3 | 2.20, 3.77 | 2.90, 3.96 | 2.90, 3.77 | 2.20, 3.96 | 2.40, 3.85 |
| Min, Max | 2.2, 3.8 | 2.9, 4.0 | 2.9, 3.8 | 2.2, 4.0 | 2.2, 4.0 |
| Change | | | | | |
| from | | | | | |
| Baseline | | | | | |
| n | 3 | 2 | 2 | 3 | 7 |
| Mean | −0.87 | −1.51 | −1.22 | −1.06 | −0.92 |
| (SD) | (1.490) | (1.709) | (2.124) | (1.290) | (1.229) |
| Median | −0.33 | −1.51 | −1.22 | −0.33 | −0.33 |
| Q1, Q3 | −2.55, 0.28 | −2.72, −0.31 | −2.72, 0.28 | −2.55, −0.31 | −2.55, 0.07 |
| Min, Max | −2.6, 0.3 | −2.7, −0.3 | −2.7, 0.3 | −2.6, −0.3 | −2.7, 0.3 |
| Percent | | | | | |
| Change | | | | | |
| from | | | | | |
| Baseline | | | | | |
| n | 3 | 2 | 2 | 3 | 7 |
| Mean | −19.22 | −27.79 | −20.18 | −24.28 | −19.09 |
| (SD) | (31.490) | (29.181) | (39.929) | (25.577) | (23.925) |
| Median | −12.02 | −27.79 | −20.18 | −12.02 | −12.02 |
| Q1, Q3 | −53.68, 8.05 | −48.42, −7.15 | −48.42, 8.05 | −53.68, −7.15 | −48.42, 1.85 |
| Min, Max | −53.7, 8.0 | −48.4, −7.2 | −48.4, 8.0 | −53.7, −7.2 | −53.7, 8.0 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 5.98 | 6.36 | 6.84 | |
| (SD) | | | | | | |
| Median | | | 5.98 | 6.36 | 6.84 | |
| Q1, Q3 | | | 5.98, 5.98 | 6.36, 6.36 | 6.84, 6.84 | |
| Min, Max | | | 6.0, 6.0 | 6.4, 6.4 | 6.8, 6.8 | |
| Average | | | | | | |
| Severity | | | | | | |
| n | | | 1 | 1 | 1 | |
| Mean | | | 4.67 | 5.23 | 5.06 | |
| (SD) | | | | | | |
| Median | | | 4.67 | 5.23 | 5.06 | |
| Q1, Q3 | | | 4.67, 4.67 | 5.23, 5.23 | 5.06, 5.06 | |
| Min, Max | | | 4.7, 4.7 | 5.2, 5.2 | 5.1, 5.1 | |
| Change | | | | | | |
| from | | | | | | |
| Baseline | | | | | | |
| n | | | 1 | 1 | 1 | |
| Mean | | | −1.32 | −1.13 | −1.79 | |
| (SD) | | | | | | |
| Median | | | −1.32 | −1.13 | −1.79 | |
| Q1, Q3 | | | −1.32, −1.32 | −1.13, −1.13 | −1.79, −1.79 | |
| Min, Max | | | −1.3, −1.3 | −1.1, −1.1 | −1.8, −1.8 | |
| Percent | | | | | | |
| Change | | | | | | |
| from | | | | | | |
| Baseline | | | | | | |
| n | | | 1 | 1 | 1 | |
| Mean | | | −22.01 | −17.81 | −26.14 | |
| (SD) | | | | | | |
| Median | | | −22.01 | −17.81 | −26.14 | |
| Q1, Q3 | | | −22.01, −22.01 | −17.81, −17.81 | −26.14, −26.14 | |
| Min, Max | | | −22.0, −22.0 | −17.8, −17.8 | −26.1, −26.1 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 6.60 | 6.36 | 6.84 | 6.39 |
| (SD) | | (0.345) | | | (0.432) |
| Median | | 6.60 | 6.36 | 6.84 | 6.36 |
| Q1, Q3 | | 6.36, 6.84 | 6.36, 6.36 | 6.84, 6.84 | 5.98, 6.84 |
| Min, Max | | 6.4, 6.8 | 6.4, 6.4 | 6.8, 6.8 | 6.0, 6.8 |
| Average | | | | | |
| Severity | | | | | |
| n | | 2 | 1 | 1 | 3 |
| Mean | | 5.14 | 5.23 | 5.06 | 4.98 |
| (SD) | | (0.120) | | | (0.286) |
| Median | | 5.14 | 5.23 | 5.06 | 5.06 |
| Q1, Q3 | | 5.06, 5.23 | 5.23, 5.23 | 5.06, 5.06 | 4.67, 5.23 |
| Min, Max | | 5.1, 5.2 | 5.2, 5.2 | 5.1, 5.1 | 4.7, 5.2 |
| Change | | | | | |
| from | | | | | |
| Baseline | | | | | |
| n | | 2 | 1 | 1 | 3 |
| Mean | | −1.46 | −1.13 | −1.79 | −1.41 |
| (SD) | | (0.464) | | | (0.339) |
| Median | | −1.46 | −1.13 | −1.79 | −1.32 |
| Q1, Q3 | | −1.79, −1.13 | −1.13, −1.13 | −1.79, −1.79 | −1.79, −1.13 |
| Min, Max | | −1.8, −1.1 | −1.1, −1.1 | −1.8, −1.8 | −1.8, −1.1 |
| Percent | | | | | |
| Change | | | | | |
| from | | | | | |
| Baseline | | | | | |
| n | | 2 | 1 | 1 | 3 |
| Mean | | −21.97 | −17.81 | −26.14 | −21.98 |
| (SD) | | (5.888) | | | (4.164) |
| Median | | −21.97 | −17.81 | −26.14 | −22.01 |
| Q1, Q3 | | −26.14, −17.81 | −17.81, −17.81 | −26.14, −26.14 | −26.14, −17.81 |
| Min, Max | | −26.1, −17.8 | −17.8, −17.8 | −26.1, −26.1 | −26.1, −17.8 |

TABLE C-5
Average Severity of Weekly RP Attacks (All Attacks) Change and Percent Change from Baseline. ITT Population
- Part A. Average severity (0-10) of weekly RP attacks is defined as the total severity scores of RP
attacks divided by total number of RP attacks within the last 7 days of the dosing period (excluding
the last dosing day). Baseline is calculated as total severity scores of RP attacks during the last 7
days of screening period (excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 3.03 | 3.37 | 7.49 | 5.92 | 5.36 | 3.82 |
| (SD) | | (1.087) | (2.457) | (0.421) | (1.746) | (0.055) |
| Median | 3.03 | 3.37 | 7.49 | 5.92 | 5.36 | 3.82 |
| Q1, Q3 | 3.03, 3.03 | 2.60, 4.14 | 5.75, 9.23 | 5.62, 6.22 | 4.12, 6.59 | 3.78, 3.86 |
| Min, Max | 3.0, 3.0 | 2.6, 4.1 | 5.8, 9.2 | 5.6, 6.2 | 4.1, 6.6 | 3.8, 3.9 |
| Average | | | | | | |
| Severity | | | | | | |
| n | 1 | 2 | 2 | 2 | 2 | 2 |
| Mean | 3.05 | 2.30 | 7.02 | 3.90 | 4.39 | 3.37 |
| (SD) | | (0.141) | (3.479) | (1.414) | (0.943) | (0.676) |
| Median | 3.05 | 2.30 | 7.02 | 3.90 | 4.39 | 3.37 |
| Q1, Q3 | 3.05, 3.05 | 2.20, 2.40 | 4.56, 9.48 | 2.90, 4.90 | 3.73, 5.06 | 2.89, 3.85 |
| Min, Max | 3.0, 3.0 | 2.2, 2.4 | 4.6, 9.5 | 2.9, 4.9 | 3.7, 5.1 | 2.9, 3.9 |
| Change | | | | | | |
| from | | | | | | |
| Baseline | | | | | | |
| n | 1 | 2 | 2 | 2 | 2 | 2 |
| Mean | 0.02 | −1.07 | −0.47 | −2.02 | −0.96 | −0.45 |
| (SD) | | (1.229) | (1.022) | (0.993) | (0.804) | (0.732) |
| Median | 0.02 | −1.07 | −0.47 | −2.02 | −0.96 | −0.45 |
| Q1, Q3 | 0.02, 0.02 | −1.94, −0.20 | −1.19, 0.25 | −2.72, −1.32 | −1.53, −0.39 | −0.97, 0.07 |
| Min, Max | 0.0, 0.0 | −1.9, −0.2 | −1.2, 0.3 | −2.7, −1.3 | −1.5, −0.4 | −1.0, 0.1 |
| Percent | | | | | | |
| Change | | | | | | |
| from | | | | | | |
| Baseline | | | | | | |
| n | 1 | 2 | 2 | 2 | 2 | 2 |
| Mean | 0.55 | −27.26 | −8.97 | −34.81 | −16.40 | −11.58 |
| (SD) | | (27.673) | (16.585) | (19.248) | (9.657) | (18.995) |
| Median | 0.55 | −27.26 | −8.97 | −34.81 | −16.40 | −11.58 |
| Q1, Q3 | 0.55, 0.55 | −46.83, −7.69 | −20.69, 2.76 | −48.42, −21.20 | −23.23, −9.57 | −25.01, 1.85 |
| Min, Max | 0.5, 0.5 | −46.8, −7.7 | −20.7, 2.8 | −48.4, −21.2 | −23.2, −9.6 | −25.0, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 3.26 | 5.64 | 4.96 | 4.36 | 4.99 |
| (SD) | (0.793) | (1.087) | (1.694) | (1.651) | (1.918) |
| Median | 3.03 | 5.92 | 5.62 | 4.13 | 4.14 |
| Q1, Q3 | 2.60, 4.14 | 4.87, 6.40 | 3.03, 6.22 | 3.36, 5.36 | 3.78, 6.22 |
| Min, Max | 2.6, 4.1 | 4.1, 6.6 | 3.0, 6.2 | 2.6, 6.6 | 2.6, 9.2 |
| Average | | | | | |
| Severity | | | | | |
| n | 3 | 4 | 3 | 4 | 11 |
| Mean | 2.55 | 4.15 | 3.62 | 3.35 | 4.09 |
| (SD) | (0.443) | (1.022) | (1.114) | (1.328) | (2.036) |
| Median | 2.40 | 4.31 | 3.05 | 3.06 | 3.73 |
| Q1, Q3 | 2.20, 3.05 | 3.31, 4.98 | 2.90, 4.90 | 2.30, 4.39 | 2.89, 4.90 |
| Min, Max | 2.2, 3.0 | 2.9, 5.1 | 2.9, 4.9 | 2.2, 5.1 | 2.2, 9.5 |
| Change | | | | | |
| from | | | | | |
| Baseline | | | | | |
| n | 3 | 4 | 3 | 4 | 11 |
| Mean | −0.71 | −1.49 | −1.34 | −1.02 | −0.90 |
| (SD) | (1.071) | (0.957) | (1.370) | (0.850) | (0.944) |
| Median | −0.20 | −1.42 | −1.32 | −0.96 | −0.97 |
| Q1, Q3 | −1.94, 0.02 | −2.13, −0.86 | −2.72, 0.02 | −1.73, −0.30 | −1.53, 0.02 |
| Min, Max | −1.9, 0.0 | −2.7, −0.4 | −2.7, 0.0 | −1.9, −0.2 | −2.7, 0.3 |
| Percent | | | | | |
| Change | | | | | |
| from | | | | | |
| Baseline | | | | | |
| n | 3 | 4 | 3 | 4 | 11 |
| Mean | −17.99 | −25.60 | −23.02 | −21.83 | −17.95 |
| (SD) | (25.311) | (16.357) | (24.534) | (18.046) | (17.879) |
| Median | −7.69 | −22.21 | −21.20 | −16.40 | −20.69 |
| Q1, Q3 | −46.83, 0.55 | −35.82, −15.38 | −48.42, 0.55 | −35.03, −8.63 | −25.01, 0.55 |
| Min, Max | −46.8, 0.5 | −48.4, −9.6 | −48.4, 0.5 | −46.8, −7.7 | −48.4, 2.8 |

TABLE C-6
Average Severity of Weekly RP Attacks (All Attacks) Change and Percent Change from Baseline
(Excluding Outliers). ITT Population - Part A. Average severity (0-10) of weekly RP attacks
is defined as the total severity scores of RP attacks divided by total number of RP attacks
within the last 7 days of the dosing period (excluding the last dosing day). Baseline is
calculated as total severity scores of RP attacks during the last 7 days of screening period
(excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 3.03 | 3.37 | 5.75 | 5.92 | 5.36 | 3.82 |
| (SD) | | (1.087) | | (0.421) | (1.746) | (0.055) |
| Median | 3.03 | 3.37 | 5.75 | 5.92 | 5.36 | 3.82 |
| Q1, Q3 | 3.03, 3.03 | 2.60, 4.14 | 5.75, 5.75 | 5.62, 6.22 | 4.12, 6.59 | 3.78, 3.86 |
| Min, Max | 3.0, 3.0 | 2.6, 4.1 | 5.8, 5.8 | 5.6, 6.2 | 4.1, 6.6 | 3.8, 3.9 |
| Average | | | | | | |
| Severity | | | | | | |
| n | 1 | 2 | 1 | 2 | 2 | 2 |
| Mean | 3.05 | 2.30 | 4.56 | 3.90 | 4.39 | 3.37 |
| (SD) | | (0.141) | | (1.414) | (0.943) | (0.676) |
| Median | 3.05 | 2.30 | 4.56 | 3.90 | 4.39 | 3.37 |
| Q1, Q3 | 3.05, 3.05 | 2.20, 2.40 | 4.56, 4.56 | 2.90, 4.90 | 3.73, 5.06 | 2.89, 3.85 |
| Min, Max | 3.0, 3.0 | 2.2, 2.4 | 4.6, 4.6 | 2.9, 4.9 | 3.7, 5.1 | 2.9, 3.9 |
| Change | | | | | | |
| from | | | | | | |
| Baseline | | | | | | |
| n | 1 | 2 | 1 | 2 | 2 | 2 |
| Mean | 0.02 | −1.07 | −1.19 | −2.02 | −0.96 | −0.45 |
| (SD) | | (1.229) | | (0.993) | (0.804) | (0.732) |
| Median | 0.02 | −1.07 | −1.19 | −2.02 | −0.96 | −0.45 |
| Q1, Q3 | 0.02, 0.02 | −1.94, −0.20 | −1.19, −1.19 | −2.72, −1.32 | −1.53, −0.39 | −0.97, 0.07 |
| Min, Max | 0.0, 0.0 | −1.9, −0.2 | −1.2, −1.2 | −2.7, −1.3 | −1.5, −0.4 | −1.0, 0.1 |
| Percent | | | | | | |
| Change | | | | | | |
| from | | | | | | |
| Baseline | | | | | | |
| n | 1 | 2 | 1 | 2 | 2 | 2 |
| Mean | 0.55 | −27.26 | −20.69 | −34.81 | −16.40 | −11.58 |
| (SD) | | (27.673) | | (19.248) | (9.657) | (18.995) |
| Median | 0.55 | −27.26 | −20.69 | −34.81 | −16.40 | −11.58 |
| Q1, Q3 | 0.55, 0.55 | −46.83, −7.69 | −20.69, −20.69 | −48.42, −21.20 | −23.23, −9.57 | −25.01, 1.85 |
| Min, Max | 0.5, 0.5 | −46.8, −7.7 | −20.7, −20.7 | −48.4, −21.2 | −23.2, −9.6 | −25.0, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 3.26 | 5.64 | 4.96 | 4.36 | 4.57 |
| (SD) | (0.793) | (1.087) | (1.694) | (1.651) | (1.377) |
| Median | 3.03 | 5.92 | 5.62 | 4.13 | 4.13 |
| Q1, Q3 | 2.60, 4.14 | 4.87, 6.40 | 3.03, 6.22 | 3.36, 5.36 | 3.78, 5.75 |
| Min, Max | 2.6, 4.1 | 4.1, 6.6 | 3.0, 6.2 | 2.6, 6.6 | 2.6, 6.6 |
| Average | | | | | |
| Severity | | | | | |
| n | 3 | 4 | 3 | 4 | 10 |
| Mean | 2.55 | 4.15 | 3.62 | 3.35 | 3.55 |
| (SD) | (0.443) | (1.022) | (1.114) | (1.328) | (1.027) |
| Median | 2.40 | 4.31 | 3.05 | 3.06 | 3.39 |
| Q1, Q3 | 2.20, 3.05 | 3.31, 4.98 | 2.90, 4.90 | 2.30, 4.39 | 2.89, 4.56 |
| Min, Max | 2.2, 3.0 | 2.9, 5.1 | 2.9, 4.9 | 2.2, 5.1 | 2.2, 5.1 |
| Change | | | | | |
| from | | | | | |
| Baseline | | | | | |
| n | 3 | 4 | 3 | 4 | 10 |
| Mean | −0.71 | −1.49 | −1.34 | −1.02 | −1.02 |
| (SD) | (1.071) | (0.957) | (1.370) | (0.850) | (0.910) |
| Median | −0.20 | −1.42 | −1.32 | −0.96 | −1.08 |
| Q1, Q3 | −1.94, 0.02 | −2.13, −0.86 | −2.72, 0.02 | −1.73, −0.30 | −1.53, −0.20 |
| Min, Max | −1.9, 0.0 | −2.7, −0.4 | −2.7, 0.0 | −1.9, −0.2 | −2.7, 0.1 |
| Percent | | | | | |
| Change | | | | | |
| from | | | | | |
| Baseline | | | | | |
| n | 3 | 4 | 3 | 4 | 10 |
| Mean | −17.99 | −25.60 | −23.02 | −21.83 | −20.02 |
| (SD) | (25.311) | (16.357) | (24.534) | (18.046) | (17.400) |
| Median | −7.69 | −22.21 | −21.20 | −16.40 | −20.95 |
| Q1, Q3 | −46.83, 0.55 | −35.82, −15.38 | −48.42, 0.55 | −35.03, −8.63 | −25.01, −7.69 |
| Min, Max | −46.8, 0.5 | −48.4, −9.6 | −48.4, 0.5 | −46.8, −7.7 | −48.4, 1.9 |

TABLE C-7
Average Severity of Weekly RP Attacks (All Attacks) Change and Percent Change from Baseline
by Baseline RCS. ITT Population - Part A. Average severity (0-10) of weekly RP attacks
is defined as the total severity scores of RP attacks divided by total number of RP attacks
within the last 7 days of the dosing period (excluding the last dosing day). Baseline is
calculated as total severity scores of RP attacks during the last 7 days of screening period
(excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.03 | 3.37 | | 5.62 | 4.12 | 3.82 |
| (SD) | | (1.087) | | | | (0.055) |
| Median | 3.03 | 3.37 | | 5.62 | 4.12 | 3.82 |
| Q1, Q3 | 3.03, 3.03 | 2.60, 4.14 | | 5.62, 5.62 | 4.12, 4.12 | 3.78, 3.86 |
| Min, Max | 3.0, 3.0 | 2.6, 4.1 | | 5.6, 5.6 | 4.1, 4.1 | 3.8, 3.9 |
| Average | | | | | | |
| Severity | | | | | | |
| n | 1 | 2 | | 1 | 1 | 2 |
| Mean | 3.05 | 2.30 | | 2.90 | 3.73 | 3.37 |
| (SD) | | (0.141) | | | | (0.676) |
| Median | 3.05 | 2.30 | | 2.90 | 3.73 | 3.37 |
| Q1, Q3 | 3.05, 3.05 | 2.20, 2.40 | | 2.90, 2.90 | 3.73, 3.73 | 2.89, 3.85 |
| Min, Max | 3.0, 3.0 | 2.2, 2.4 | | 2.9, 2.9 | 3.7, 3.7 | 2.9, 3.9 |
| Change | | | | | | |
| from | | | | | | |
| Baseline | | | | | | |
| n | 1 | 2 | | 1 | 1 | 2 |
| Mean | 0.02 | −1.07 | | −2.72 | −0.39 | −0.45 |
| (SD) | | (1.229) | | | | (0.732) |
| Median | 0.02 | −1.07 | | −2.72 | −0.39 | −0.45 |
| Q1, Q3 | 0.02, 0.02 | −1.94, −0.20 | | −2.72, −2.72 | −0.39, −0.39 | −0.97, 0.07 |
| Min, Max | 0.0, 0.0 | −1.9, −0.2 | | −2.7, −2.7 | −0.4, −0.4 | −1.0, 0.1 |
| Percent | | | | | | |
| Change | | | | | | |
| from | | | | | | |
| Baseline | | | | | | |
| n | 1 | 2 | | 1 | 1 | 2 |
| Mean | 0.55 | −27.26 | | −48.42 | −9.57 | −11.58 |
| (SD) | | (27.673) | | | | (18.995) |
| Median | 0.55 | −27.26 | | −48.42 | −9.57 | −11.58 |
| Q1, Q3 | 0.55, 0.55 | −46.83, −7.69 | | −48.42, −48.42 | −9.57, −9.57 | −25.01, 1.85 |
| Min, Max | 0.5, 0.5 | −46.8, −7.7 | | −48.4, −48.4 | −9.6, −9.6 | −25.0, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 3.26 | 4.87 | 4.33 | 3.62 | 3.88 |
| (SD) | (0.793) | (1.061) | (1.832) | (0.883) | (0.960) |
| Median | 3.03 | 4.87 | 4.33 | 4.12 | 3.86 |
| Q1, Q3 | 2.60, 4.14 | 4.12, 5.62 | 3.03, 5.62 | 2.60, 4.14 | 3.03, 4.14 |
| Min, Max | 2.6, 4.1 | 4.1, 5.6 | 3.0, 5.6 | 2.6, 4.1 | 2.6, 5.6 |
| Average | | | | | |
| Severity | | | | | |
| n | 3 | 2 | 2 | 3 | 7 |
| Mean | 2.55 | 3.31 | 2.97 | 2.78 | 3.00 |
| (SD) | (0.443) | (0.585) | (0.105) | (0.830) | (0.616) |
| Median | 2.40 | 3.31 | 2.97 | 2.40 | 2.90 |
| Q1, Q3 | 2.20, 3.05 | 2.90, 3.73 | 2.90, 3.05 | 2.20, 3.73 | 2.40, 3.73 |
| Min, Max | 2.2, 3.0 | 2.9, 3.7 | 2.9, 3.0 | 2.2, 3.7 | 2.2, 3.9 |
| Change | | | | | |
| from | | | | | |
| Baseline | | | | | |
| n | 3 | 2 | 2 | 3 | 7 |
| Mean | −0.71 | −1.56 | −1.35 | −0.84 | −0.88 |
| (SD) | (1.071) | (1.646) | (1.937) | (0.952) | (1.074) |
| Median | −0.20 | −1.56 | −1.35 | −0.39 | −0.39 |
| Q1, Q3 | −1.94, 0.02 | −2.72, −0.39 | −2.72, 0.02 | −1.94, −0.20 | −1.94, 0.02 |
| Min, Max | −1.9, 0.0 | −2.7, −0.4 | −2.7, 0.0 | −1.9, −0.2 | −2.7, 0.1 |
| Percent | | | | | |
| Change | | | | | |
| from | | | | | |
| Baseline | | | | | |
| n | 3 | 2 | 2 | 3 | 7 |
| Mean | −17.99 | −28.99 | −23.94 | −21.36 | −19.30 |
| (SD) | (25.311) | (27.470) | (34.624) | (22.073) | (21.249) |
| Median | −7.69 | −28.99 | −23.94 | −9.57 | −9.57 |
| Q1, Q3 | −46.83, 0.55 | −48.42, −9.57 | −48.42, 0.55 | −46.83, −7.69 | −46.83, 0.55 |
| Min, Max | −46.8, 0.5 | −48.4, −9.6 | −48.4, 0.5 | −46.8, −7.7 | −48.4, 1.9 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 7.49 | 6.22 | 6.59 | |
| (SD) | | | (2.457) | | | |
| Median | | | 7.49 | 6.22 | 6.59 | |
| Q1, Q3 | | | 5.75, 9.23 | 6.22, 6.22 | 6.59, 6.59 | |
| Min, Max | | | 5.8, 9.2 | 6.2, 6.2 | 6.6, 6.6 | |
| Average | | | | | | |
| Severity | | | | | | |
| n | | | 2 | 1 | 1 | |
| Mean | | | 7.02 | 4.90 | 5.06 | |
| (SD) | | | (3.479) | | | |
| Median | | | 7.02 | 4.90 | 5.06 | |
| Q1, Q3 | | | 4.56, 9.48 | 4.90, 4.90 | 5.06, 5.06 | |
| Min, Max | | | 4.6, 9.5 | 4.9, 4.9 | 5.1, 5.1 | |
| Change | | | | | | |
| from | | | | | | |
| Baseline | | | | | | |
| n | | | 2 | 1 | 1 | |
| Mean | | | −0.47 | −1.32 | −1.53 | |
| (SD) | | | (1.022) | | | |
| Median | | | −0.47 | −1.32 | −1.53 | |
| Q1, Q3 | | | −1.19, 0.25 | −1.32, −1.32 | −1.53, −1.53 | |
| Min, Max | | | −1.2, 0.3 | −1.3, −1.3 | −1.5, −1.5 | |
| Percent | | | | | | |
| Change | | | | | | |
| from | | | | | | |
| Baseline | | | | | | |
| n | | | 2 | 1 | 1 | |
| Mean | | | −8.97 | −21.20 | −23.23 | |
| (SD) | | | (16.585) | | | |
| Median | | | −8.97 | −21.20 | −23.23 | |
| Q1, Q3 | | | −20.69, 2.76 | −21.20, −21.20 | −23.23, −23.23 | |
| Min, Max | | | −20.7, 2.8 | −21.2, −21.2 | −23.2, −23.2 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 6.40 | 6.22 | 6.59 | 6.95 |
| (SD) | | (0.264) | | | (1.559) |
| Median | | 6.40 | 6.22 | 6.59 | 6.40 |
| Q1, Q3 | | 6.22, 6.59 | 6.22, 6.22 | 6.59, 6.59 | 5.99, 7.91 |
| Min, Max | | 6.2, 6.6 | 6.2, 6.2 | 6.6, 6.6 | 5.8, 9.2 |
| Average | | | | | |
| Severity | | | | | |
| n | | 2 | 1 | 1 | 4 |
| Mean | | 4.98 | 4.90 | 5.06 | 6.00 |
| (SD) | | (0.113) | | | (2.330) |
| Median | | 4.98 | 4.90 | 5.06 | 4.98 |
| Q1, Q3 | | 4.90, 5.06 | 4.90, 4.90 | 5.06, 5.06 | 4.73, 7.27 |
| Min, Max | | 4.9, 5.1 | 4.9, 4.9 | 5.1, 5.1 | 4.6, 9.5 |
| Change | | | | | |
| from | | | | | |
| Baseline | | | | | |
| n | | 2 | 1 | 1 | 4 |
| Mean | | −1.42 | −1.32 | −1.53 | −0.95 |
| (SD) | | (0.150) | | | (0.813) |
| Median | | −1.42 | −1.32 | −1.53 | −1.25 |
| Q1, Q3 | | −1.53, −1.32 | −1.32, −1.32 | −1.53, −1.53 | −1.42, −0.47 |
| Min, Max | | −1.5, −1.3 | −1.3, −1.3 | −1.5, −1.5 | −1.5, 0.3 |
| Percent | | | | | |
| Change | | | | | |
| from | | | | | |
| Baseline | | | | | |
| n | | 2 | 1 | 1 | 4 |
| Mean | | −22.21 | −21.20 | −23.23 | −15.59 |
| (SD) | | (1.435) | | | (12.283) |
| Median | | −22.21 | −21.20 | −23.23 | −20.95 |
| Q1, Q3 | | −23.23, −21.20 | −21.20, −21.20 | −23.23, −23.23 | −22.21, −8.97 |
| Min, Max | | −23.2, −21.2 | −21.2, −21.2 | −23.2, −23.2 | −23.2, 2.8 |

TABLE C-8
Average Severity of Weekly RP Attacks (All Attacks) Change and Percent Change from Baseline
(Excluding Outliers) by Baseline RCS. ITT Population - Part A. Average severity (0-10) of weekly
RP attacks is defined as the total severity scores of RP attacks divided by total number of
RP attacks within the last 7 days of the dosing period (excluding the last dosing day). Baseline
is calculated as total severity scores of RP attacks during the last 7 days of screening period
(excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.03 | 3.37 | | 5.62 | 4.12 | 3.82 |
| (SD) | | (1.087) | | | | (0.055) |
| Median | 3.03 | 3.37 | | 5.62 | 4.12 | 3.82 |
| Q1, Q3 | 3.03, 3.03 | 2.60, 4.14 | | 5.62, 5.62 | 4.12, 4.12 | 3.78, 3.86 |
| Min, Max | 3.0, 3.0 | 2.6, 4.1 | | 5.6, 5.6 | 4.1, 4.1 | 3.8, 3.9 |
| Average | | | | | | |
| Severity | | | | | | |
| n | 1 | 2 | | 1 | 1 | 2 |
| Mean | 3.05 | 2.30 | | 2.90 | 3.73 | 3.37 |
| (SD) | | (0.141) | | | | (0.676) |
| Median | 3.05 | 2.30 | | 2.90 | 3.73 | 3.37 |
| Q1, Q3 | 3.05, 3.05 | 2.20, 2.40 | | 2.90, 2.90 | 3.73, 3.73 | 2.89, 3.85 |
| Min, Max | 3.0, 3.0 | 2.2, 2.4 | | 2.9, 2.9 | 3.7, 3.7 | 2.9, 3.9 |
| Change | | | | | | |
| from | | | | | | |
| Baseline | | | | | | |
| n | 1 | 2 | | 1 | 1 | 2 |
| Mean | 0.02 | −1.07 | | −2.72 | −0.39 | −0.45 |
| (SD) | | (1.229) | | | | (0.732) |
| Median | 0.02 | −1.07 | | −2.72 | −0.39 | −0.45 |
| Q1, Q3 | 0.02, 0.02 | −1.94, −0.20 | | −2.72, −2.72 | −0.39, −0.39 | −0.97, 0.07 |
| Min, Max | 0.0, 0.0 | −1.9, −0.2 | | −2.7, −2.7 | −0.4, −0.4 | −1.0, 0.1 |
| Percent | | | | | | |
| Change | | | | | | |
| from | | | | | | |
| Baseline | | | | | | |
| n | 1 | 2 | | 1 | 1 | 2 |
| Mean | 0.55 | −27.26 | | −48.42 | −9.57 | −11.58 |
| (SD) | | (27.673) | | | | (18.995) |
| Median | 0.55 | −27.26 | | −48.42 | −9.57 | −11.58 |
| Q1, Q3 | 0.55, 0.55 | −46.83, −7.69 | | −48.42, −48.42 | −9.57, −9.57 | −25.01, 1.85 |
| Min, Max | 0.5, 0.5 | −46.8, −7.7 | | −48.4, −48.4 | −9.6, −9.6 | −25.0, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 3.26 | 4.87 | 4.33 | 3.62 | 3.88 |
| (SD) | (0.793) | (1.061) | (1.832) | (0.883) | (0.960) |
| Median | 3.03 | 4.87 | 4.33 | 4.12 | 3.86 |
| Q1, Q3 | 2.60, 4.14 | 4.12, 5.62 | 3.03, 5.62 | 2.60, 4.14 | 3.03, 4.14 |
| Min, Max | 2.6, 4.1 | 4.1, 5.6 | 3.0, 5.6 | 2.6, 4.1 | 2.6, 5.6 |
| Average | | | | | |
| Severity | | | | | |
| n | 3 | 2 | 2 | 3 | 7 |
| Mean | 2.55 | 3.31 | 2.97 | 2.78 | 3.00 |
| (SD) | (0.443) | (0.585) | (0.105) | (0.830) | (0.616) |
| Median | 2.40 | 3.31 | 2.97 | 2.40 | 2.90 |
| Q1, Q3 | 2.20, 3.05 | 2.90, 3.73 | 2.90, 3.05 | 2.20, 3.73 | 2.40, 3.73 |
| Min, Max | 2.2, 3.0 | 2.9, 3.7 | 2.9, 3.0 | 2.2, 3.7 | 2.2, 3.9 |
| Change | | | | | |
| from | | | | | |
| Baseline | | | | | |
| n | 3 | 2 | 2 | 3 | 7 |
| Mean | −0.71 | −1.56 | −1.35 | −0.84 | −0.88 |
| (SD) | (1.071) | (1.646) | (1.937) | (0.952) | (1.074) |
| Median | −0.20 | −1.56 | −1.35 | −0.39 | −0.39 |
| Q1, Q3 | −1.94, 0.02 | −2.72, −0.39 | −2.72, 0.02 | −1.94, −0.20 | −1.94, 0.02 |
| Min, Max | −1.9, 0.0 | −2.7, −0.4 | −2.7, 0.0 | −1.9, −0.2 | −2.7, 0.1 |
| Percent | | | | | |
| Change | | | | | |
| from | | | | | |
| Baseline | | | | | |
| n | 3 | 2 | 2 | 3 | 7 |
| Mean | −17.99 | −28.99 | −23.94 | −21.36 | −19.30 |
| (SD) | (25.311) | (27.470) | (34.624) | (22.073) | (21.249) |
| Median | −7.69 | −28.99 | −23.94 | −9.57 | −9.57 |
| Q1, Q3 | −46.83, 0.55 | −48.42, −9.57 | −48.42, 0.55 | −46.83, −7.69 | −46.83, 0.55 |
| Min, Max | −46.8, 0.5 | −48.4, −9.6 | −48.4, 0.5 | −46.8, −7.7 | −48.4, 1.9 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 5.75 | 6.22 | 6.59 | |
| (SD) | | | | | | |
| Median | | | 5.75 | 6.22 | 6.59 | |
| Q1, Q3 | | | 5.75, 5.75 | 6.22, 6.22 | 6.59, 6.59 | |
| Min, Max | | | 5.8, 5.8 | 6.2, 6.2 | 6.6, 6.6 | |
| Average | | | | | | |
| Severity | | | | | | |
| n | | | 1 | 1 | 1 | |
| Mean | | | 4.56 | 4.90 | 5.06 | |
| (SD) | | | | | | |
| Median | | | 4.56 | 4.90 | 5.06 | |
| Q1, Q3 | | | 4.56, 4.56 | 4.90, 4.90 | 5.06, 5.06 | |
| Min, Max | | | 4.6, 4.6 | 4.9, 4.9 | 5.1, 5.1 | |
| Change | | | | | | |
| from | | | | | | |
| Baseline | | | | | | |
| n | | | 1 | 1 | 1 | |
| Mean | | | −1.19 | −1.32 | −1.53 | |
| (SD) | | | | | | |
| Median | | | −1.19 | −1.32 | −1.53 | |
| Q1, Q3 | | | −1.19, −1.19 | −1.32, −1.32 | −1.53, −1.53 | |
| Min, Max | | | −1.2, −1.2 | −1.3, −1.3 | −1.5, −1.5 | |
| Percent | | | | | | |
| Change | | | | | | |
| from | | | | | | |
| Baseline | | | | | | |
| n | | | 1 | 1 | 1 | |
| Mean | | | −20.69 | −21.20 | −23.23 | |
| (SD) | | | | | | |
| Median | | | −20.69 | −21.20 | −23.23 | |
| Q1, Q3 | | | −20.69, −20.69 | −21.20, −21.20 | −23.23, −23.23 | |
| Min, Max | | | −20.7, −20.7 | −21.2, −21.2 | −23.2, −23.2 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 6.40 | 6.22 | 6.59 | 6.19 |
| (SD) | | (0.264) | | | (0.419) |
| Median | | 6.40 | 6.22 | 6.59 | 6.22 |
| Q1, Q3 | | 6.22, 6.59 | 6.22, 6.22 | 6.59, 6.59 | 5.75, 6.59 |
| Min, Max | | 6.2, 6.6 | 6.2, 6.2 | 6.6, 6.6 | 5.8, 6.6 |
| Average | | | | | |
| Severity | | | | | |
| n | | 2 | 1 | 1 | 3 |
| Mean | | 4.98 | 4.90 | 5.06 | 4.84 |
| (SD) | | (0.113) | | | (0.254) |
| Median | | 4.98 | 4.90 | 5.06 | 4.90 |
| Q1, Q3 | | 4.90, 5.06 | 4.90, 4.90 | 5.06, 5.06 | 4.56, 5.06 |
| Min, Max | | 4.9, 5.1 | 4.9, 4.9 | 5.1, 5.1 | 4.6, 5.1 |
| Change | | | | | |
| from | | | | | |
| Baseline | | | | | |
| n | | 2 | 1 | 1 | 3 |
| Mean | | −1.42 | −1.32 | −1.53 | −1.35 |
| (SD) | | (0.150) | | | (0.172) |
| Median | | −1.42 | −1.32 | −1.53 | −1.32 |
| Q1, Q3 | | −1.53, −1.32 | −1.32, −1.32 | −1.53, −1.53 | −1.53, −1.19 |
| Min, Max | | −1.5, −1.3 | −1.3, −1.3 | −1.5, −1.5 | −1.5, −1.2 |
| Percent | | | | | |
| Change | | | | | |
| from | | | | | |
| Baseline | | | | | |
| n | | 2 | 1 | 1 | 3 |
| Mean | | −22.21 | −21.20 | −23.23 | −21.71 |
| (SD) | | (1.435) | | | (1.341) |
| Median | | −22.21 | −21.20 | −23.23 | −21.20 |
| Q1, Q3 | | −23.23, −21.20 | −21.20, −21.20 | −23.23, −23.23 | −23.23, −20.69 |
| Min, Max | | −23.2, −21.2 | −21.2, −21.2 | −23.2, −23.2 | −23.2, −20.7 |

TABLE D-1
Average Daily RCS Change and Percent Change from Baseline. ITT Population - Part A.
Average daily RCS is defined as the total RCS divided by the number of days with
available diary data during the dosing period. Baseline is calculated as the total RCS
divided by the number of days with available diary data during the screening period.
If there were multiple daily RCS scores, the latest daily RCS was used.

| | | | | | | Cilnidipine | | Cilnidipine | | | |
|---|---|---|---|---|---|---|---|---|---|---|---|
| | | | | | | 10 mg + | | 20 mg + | | | |
| | Cilnidipine | Cilnidipine | | Tadalafil | | Tadalafil | | Tadalafil | | | |
| | 10 mg | 20 mg | | 5 mg | | 5 mg | | 5 mg | | Placebo | |
| Statistics | (N = 1) | (N = 2) | | (N = 2) | | (N = 2) | | (N = 2) | | (N = 2) | |
| Baseline | | | | | | | | | | | |
| n | 1 | 2 | | 2 | | 2 | | 2 | | 2 | |
| Mean (SD) | 2.74 | 3.35 | (0.778) | 7.72 | (2.378) | 4.73 | (2.660) | 6.01 | (3.445) | 3.18 | (0.089) |
| Median | 2.74 | 3.35 | | 7.72 | | 4.73 | | 6.01 | | 3.18 | |
| Q1, Q3 | 2.74, 2.74 | 2.80, 3.90 | | 6.04, 9.40 | | 2.85, 6.61 | | 3.58, 8.45 | | 3.12, 3.24 | |
| Min, Max | 2.7, 2.7 | 2.8, 3.9 | | 6.0, 9.4 | | 2.9, 6.6 | | 3.6, 8.5 | | 3.1, 3.2 | |
| Average | | | | | | | | | | | |
| Daily RCS | | | | | | | | | | | |
| n | 1 | 2 | | 2 | | 2 | | 2 | | 2 | |
| Mean (SD) | 1.76 | 2.90 | (1.730) | 7.05 | (3.672) | 2.77 | (2.025) | 4.30 | (1.112) | 2.71 | (0.507) |
| Median | 1.76 | 2.90 | | 7.05 | | 2.77 | | 4.30 | | 2.71 | |
| Q1, Q3 | 1.76, 1.76 | 1.68, 4.13 | | 4.46, 9.65 | | 1.34, 4.20 | | 3.52, 5.09 | | 2.35, 3.07 | |
| Min, Max | 1.8, 1.8 | 1.7, 4.1 | | 4.5, 9.7 | | 1.3, 4.2 | | 3.5, 5.1 | | 2.4, 3.1 | |
| Change from | | | | | | | | | | | |
| Baseline | | | | | | | | | | | |
| n | 1 | 2 | | 2 | | 2 | | 2 | | 2 | |
| Mean (SD) | −0.98 | −0.45 | (0.953) | −0.67 | (1.294) | −1.96 | (0.635) | −1.71 | (2.333) | −0.47 | (0.418) |
| Median | −0.98 | −0.45 | | −0.67 | | −1.96 | | −1.71 | | −0.47 | |
| Q1, Q3 | −0.98, −0.98 | −1.12, 0.22 | | −1.58, 0.25 | | −2.41, −1.51 | | −3.36, −0.06 | | −0.77, −0.18 | |
| Min, Max | −1.0, −1.0 | −1.1, 0.2 | | −1.6, 0.3 | | −2.4, −1.5 | | −3.4, −0.1 | | −0.8, −0.2 | |
| Percent | | | | | | | | | | | |
| Change from | | | | | | | | | | | |
| Baseline | | | | | | | | | | | |
| n | 1 | 2 | | 2 | | 2 | | 2 | | 2 | |
| Mean (SD) | −35.74 | −17.16 | (32.420) | −11.76 | (20.390) | −44.79 | (11.766) | −20.71 | (26.931) | −15.03 | (13.569) |

| Median | −35.74 | −17.16 | −11.76 | −44.79 | −20.71 | −15.03 |
|---|---|---|---|---|---|---|
| Q1, Q3 | −35.74, −35.74 | −40.08, 5.77 | −26.18, 2.66 | −53.11, −36.47 | −39.75, −1.67 | −24.62, −5.43 |
| Min, Max | −35.7, −35.7 | −40.1, 5.8 | −26.2, 2.7 | −53.1, −36.5 | −39.8, −1.7 | −24.6, −5.4 |

| | | | | | Pooled | | Pooled | | | |
|---|---|---|---|---|---|---|---|---|---|---|
| | | | | | Cilnidipine | | Cilnidipine | | | |
| | | | | | 10 mg and | | 20 mg and | | | |
| | | | | | Cilnidipine | | Cilnidipine | | | |
| | | | Pooled | | 10 mg + | | 20 mg + | | | |
| | Pooled | | Cilnidipine + | | Tadalafil | | Tadalafil | | All | |
| | Cilnidipine | | Tadalafil | | 5 mg | | 5 mg | | Participants | |
| Statistics | (N = 3) | | (N = 4) | | (N = 3) | | (N = 4) | | (N = 11) | |
| Baseline | | | | | | | | | | |
| n | 3 | | 4 | | 3 | | 4 | | 11 | |
| Mean (SD) | 3.15 | (0.652) | 5.37 | (2.620) | 4.07 | (2.203) | 4.68 | (2.554) | 4.79 | (2.427) |

| | | | | | |
|---|---|---|---|---|---|
| Median | 2.80 | 5.09 | 2.85 | 3.74 | 3.58 |

| | | | | | |
|---|---|---|---|---|---|
| Q1, Q3 | 2.74, 3.90 | 3.21, 7.53 | 2.74, 6.61 | 3.19, 6.18 | 2.85, 6.61 |
| Min, Max | 2.7, 3.9 | 2.9, 8.5 | 2.7, 6.6 | 2.8, 8.5 | 2.7, 9.4 |

| Average | | | | | | | | | | |
|---|---|---|---|---|---|---|---|---|---|---|
| Daily RCS | | | | | | | | | | |
| n | 3 | | 4 | | 3 | | 4 | | 11 | |
| Mean (SD) | 2.52 | (1.389) | 3.54 | (1.602) | 2.43 | (1.545) | 3.60 | (1.438) | 3.75 | (2.324) |

| | | | | | |
|---|---|---|---|---|---|
| Median | 1.76 | 3.86 | 1.76 | 3.82 | 3.52 |

| | | | | | |
|---|---|---|---|---|---|
| Q1, Q3 | 1.68, 4.13 | 2.43, 4.65 | 1.34, 4.20 | 2.60, 4.61 | 1.76, 4.46 |
| Min, Max | 1.7, 4.1 | 1.3, 5.1 | 1.3, 4.2 | 1.7, 5.1 | 1.3, 9.7 |

| Change from | | | | | | | | | | |
|---|---|---|---|---|---|---|---|---|---|---|
| Baseline | | | | | | | | | | |
| n | 3 | | 4 | | 3 | | 4 | | 11 | |
| Mean (SD) | −0.63 | (0.740) | −1.84 | (1.404) | −1.64 | (0.723) | −1.08 | (1.627) | −1.05 | (1.133) |

| | | | | | |
|---|---|---|---|---|---|
| Median | −0.98 | −1.96 | −1.51 | −0.59 | −0.98 |

| | | | | | |
|---|---|---|---|---|---|
| Q1, Q3 | −1.12, 0.22 | −2.89, −0.79 | −2.41, −0.98 | −2.24, 0.08 | −1.58, −0.06 |
| Min, Max | −1.1, 0.2 | −3.4, −0.1 | −2.4, −1.0 | −3.4, 0.2 | −3.4, 0.3 |

| Percent | | | | | | | | | | |
|---|---|---|---|---|---|---|---|---|---|---|
| Change from | | | | | | | | | | |
| Baseline | | | | | | | | | | |
| n | 3 | | 4 | | 3 | | 4 | | 11 | |
| Mean (SD) | −23.35 | (25.311) | −32.75 | (21.937) | −41.77 | (9.825) | −18.93 | (24.420) | −23.15 | (20.211) |

| | | | | | |
|---|---|---|---|---|---|
| Median | −35.74 | −38.11 | −36.47 | −20.71 | −26.18 |

| | | | | | |
|---|---|---|---|---|---|
| Q1, Q3 | −40.08, 5.77 | −46.43, −19.07 | −53.11, −35.74 | −39.92, 2.05 | −39.75, −1.67 |
| Min, Max | −40.1, 5.8 | −53.1, −1.7 | −53.1, −35.7 | −40.1, 5.8 | −53.1, 5.8 |

Tables D-1 and D-2 include Raynaud's Condition Score (RCS) data.

TABLE D-2
Average Daily RCS Change and Percent Change from Baseline (Excluding Outliers). ITT Population - Part A.
Average daily RCS is defined as the total RCS divided by the number of days with
available diary data during the dosing period. Baseline is calculated as the total RCS
divided by the number of days with available diary data during the screening period.
If there were multiple daily RCS scores, the latest daily RCS was used.

| | | | | | Cilnidipine | | Cilnidipine | | | |
|---|---|---|---|---|---|---|---|---|---|---|
| | | | | | 10 mg + | | 20 mg + | | | |
| | Cilnidipine | Cilnidipine | | Tadalafil | Tadalafil | | Tadalafil | | | |
| | 10 mg | 20 mg | | 5 mg | 5 mg | | 5 mg | | Placebo | |
| Statistics | (N = 1) | (N = 2) | | (N = 2) | (N = 2) | | (N = 2) | | (N = 2) | |
| Baseline | | | | | | | | | | |
| n | 1 | 2 | | 1 | 2 | | 2 | | 2 | |
| Mean (SD) | 2.74 | 3.35 | (0.778) | 6.04 | 4.73 | (2.660) | 6.01 | (3.445) | 3.18 | (0.089) |
| Median | 2.74 | 3.35 | | 6.04 | 4.73 | | 6.01 | | 3.18 | |
| Q1, Q3 | 2.74, 2.74 | 2.80, 3.90 | | 6.04, 6.04 | 2.85, 6.61 | | 3.58, 8.45 | | 3.12, 3.24 | |
| Min, Max | 2.7, 2.7 | 2.8, 3.9 | | 6.0, 6.0 | 2.9, 6.6 | | 3.6, 8.5 | | 3.1, 3.2 | |
| Average | | | | | | | | | | |
| Daily RCS | | | | | | | | | | |
| n | 1 | 2 | | 1 | 2 | | 2 | | 2 | |
| Mean (SD) | 1.76 | 2.90 | (1.730) | 4.46 | 2.77 | (2.025) | 4.30 | (1.112) | 2.71 | (0.507) |
| Median | 1.76 | 2.90 | | 4.46 | 2.77 | | 4.30 | | 2.71 | |
| Q1, Q3 | 1.76, 1.76 | 1.68, 4.13 | | 4.46, 4.46 | 1.34, 4.20 | | 3.52, 5.09 | | 2.35, 3.07 | |
| Min, Max | 1.8, 1.8 | 1.7, 4.1 | | 4.5, 4.5 | 1.3, 4.2 | | 3.5, 5.1 | | 2.4, 3.1 | |
| Change from | | | | | | | | | | |
| Baseline | | | | | | | | | | |
| n | 1 | 2 | | 1 | 2 | | 2 | | 2 | |
| Mean (SD) | −0.98 | −0.45 | (0.953) | −1.58 | −1.96 | (0.635) | −1.71 | (2.333) | −0.47 | (0.418) |
| Median | −0.98 | −0.45 | | −1.58 | −1.96 | | −1.71 | | −0.47 | |
| Q1, Q3 | −0.98, −0.98 | −1.12, 0.22 | | −1.58, −1.58 | −2.41, −1.51 | | −3.36, −0.06 | | −0.77, −0.18 | |
| Min, Max | −1.0, −1.0 | −1.1, 0.2 | | −1.6, −1.6 | −2.4, −1.5 | | −3.4, −0.1 | | −0.8, −0.2 | |
| Percent | | | | | | | | | | |
| Change from | | | | | | | | | | |
| Baseline | | | | | | | | | | |
| n | 1 | 2 | | 1 | 2 | | 2 | | 2 | |
| Mean (SD) | −35.74 | −17.16 | (32.420) | −26.18 | −44.79 | (11.766) | −20.71 | (26.931) | −15.03 | (13.569) |

| Median | −35.74 | −17.16 | −26.18 | −44.79 | −20.71 | −15.03 |
|---|---|---|---|---|---|---|
| Q1, Q3 | −35.74, −35.74 | −40.08, 5.77 | −26.18, −26.18 | −53.11, −36.47 | −39.75, −1.67 | −24.62, −5.43 |
| Min, Max | −35.7, −35.7 | −40.1, 5.8 | −26.2, −26.2 | −53.1, −36.5 | −39.8, −1.7 | −24.6, −5.4 |

| | | | | | Pooled | | Pooled | | | |
|---|---|---|---|---|---|---|---|---|---|---|
| | | | | | Cilnidipine | | Cilnidipine | | | |
| | | | | | 10 mg and | | 20 mg and | | | |
| | | | | | Cilnidipine | | Cilnidipine | | | |
| | | | Pooled | | 10 mg + | | 20 mg + | | | |
| | Pooled | | Cilnidipine + | | Tadalafil | | Tadalafil | | All | |
| | Cilnidipine | | Tadalafil | | 5 mg | | 5 mg | | Participants | |
| Statistics | (N = 3) | | (N = 4) | | (N = 3) | | (N = 4) | | (N = 11) | |
| Baseline | | | | | | | | | | |
| n | 3 | | 4 | | 3 | | 4 | | 10 | |
| Mean (SD) | 3.15 | (0.652) | 5.37 | (2.620) | 4.07 | (2.203) | 4.68 | (2.554) | 4.33 | (1.987) |

| | | | | | |
|---|---|---|---|---|---|
| Median | 2.80 | 5.09 | 2.85 | 3.74 | 3.41 |

| | | | | | |
|---|---|---|---|---|---|
| Q1, Q3 | 2.74, 3.90 | 3.21, 7.53 | 2.74, 6.61 | 3.19, 6.18 | 2.85, 6.04 |
| Min, Max | 2.7, 3.9 | 2.9, 8.5 | 2.7, 6.6 | 2.8, 8.5 | 2.7, 8.5 |

| Average | | | | | | | | | | |
|---|---|---|---|---|---|---|---|---|---|---|
| Daily RCS | | | | | | | | | | |
| n | 3 | | 4 | | 3 | | 4 | | 10 | |
| Mean (SD) | 2.52 | (1.389) | 3.54 | (1.602) | 2.43 | (1.545) | 3.60 | (1.438) | 3.16 | (1.320) |

| | | | | | |
|---|---|---|---|---|---|
| Median | 1.76 | 3.86 | 1.76 | 3.82 | 3.29 |

| | | | | | |
|---|---|---|---|---|---|
| Q1, Q3 | 1.68, 4.13 | 2.43, 4.65 | 1.34, 4.20 | 2.60, 4.61 | 1.76, 4.20 |
| Min, Max | 1.7, 4.1 | 1.3, 5.1 | 1.3, 4.2 | 1.7, 5.1 | 1.3, 5.1 |

| Change from | | | | | | | | | | |
|---|---|---|---|---|---|---|---|---|---|---|
| Baseline | | | | | | | | | | |
| n | 3 | | 4 | | 3 | | 4 | | 10 | |
| Mean (SD) | −0.63 | (0.740) | −1.84 | (1.404) | −1.64 | (0.723) | −1.08 | (1.627) | −1.17 | (1.105) |

| | | | | | |
|---|---|---|---|---|---|
| Median | −0.98 | −1.96 | −1.51 | −0.59 | −1.05 |

| | | | | | |
|---|---|---|---|---|---|
| Q1, Q3 | −1.12, 0.22 | −2.89, −0.79 | −2.41, −0.98 | −2.24, 0.08 | −1.58, −0.18 |
| Min, Max | −1.1, 0.2 | −3.4, −0.1 | −2.4, −1.0 | −3.4, 0.2 | −3.4, 0.2 |

| Percent | | | | | | | | | | |
|---|---|---|---|---|---|---|---|---|---|---|
| Change from | | | | | | | | | | |
| Baseline | | | | | | | | | | |
| n | 3 | | 4 | | 3 | | 4 | | 10 | |
| Mean (SD) | −23.35 | (25.311) | −32.75 | (21.937) | −41.77 | (9.825) | −18.93 | (24.420) | −25.73 | (19.300) |

| | | | | | |
|---|---|---|---|---|---|
| Median | −35.74 | −38.11 | −36.47 | −20.71 | −30.96 |

| | | | | | |
|---|---|---|---|---|---|
| Q1, Q3 | −40.08, 5.77 | −46.43, −19.07 | −53.11, −35.74 | −39.92, 2.05 | −39.75, −5.43 |
| Min, Max | −40.1, 5.8 | −53.1, −1.7 | −53.1, −35.7 | −40.1, 5.8 | −53.1, 5.8 |

### Results and Discussion

The study treatment is well-tolerated and no adverse events or serious adverse events have been reported in any treated patients.

All cilnidipine and cilnidipine plus tadalafil treated patients (n=7) showed a decrease in the weekly frequency of Raynaud attacks in a population of both milder and more severe disease based on Raynaud Condition Scores at baseline. In 6 of 7 of these patients the average frequency of Raynaud's attacks decreased more than 25%, a clinically meaningful improvement. This improvement was not seen in patients treated with tadalafil alone, who demonstrated an increase in attack frequency.

When considering attacks reported by patients as their typical attacks, all pooled cilnidipine monotherapy or cilnidipine plus tadalafil treated patients (n=7) achieved the primary endpoint of the study producing a 25% or greater reduction in weekly attack frequency (−31 to −40%).

In patients with less severe disease at baseline, as delineated by a baseline Raynaud Condition Score of <5.0, all cilnidipine and cilnidipine plus tadalafil treated patients (n=5) demonstrated a reduction in the weekly frequency of attacks by at least 25% (range 28%-44% reduction).

In patients with more severe disease at baseline, as delineated by a Raynaud Condition Score of >5.0, (n=2) two of two cilnidipine plus tadalafil treated patients demonstrated a reduction in the weekly frequency of attacks but in only one of these patients was the reduction greater than the 25% threshold (43% reduction). Tadalafil monotherapy also increased the frequency of attacks in the one treated patient who had a baseline RCS>5.0.

When considering attacks reported by the patient as their typical Raynaud attacks, a dose response was seen (n=3) with the reduction in weekly frequency of attacks increasing to 46% from 28%, with an increase in dose from 10 mg to 20 mg of cilnidipine.

When considering attacks reported by the patient as their typical Raynaud attacks, in patients with more severe disease at baseline as determined by a baseline RCS>5.0, on average, cilnidipine plus tadalafil treated patients (cilnidipine at either 10 mg or 20 mg) had a significant reduction in the weekly frequency of attacks (−30%)

While tadalafil monotherapy at a 5 mg dose in this small sample of patients (n=2) seemed ineffective (frequency of attacks increased) adding this same dose of tadalafil to cilnidipine seemed to increase benefit with reductions in frequency, severity of attacks, duration of attacks and on RCS scores.

When considering attacks reported by the patient as their typical Raynaud attacks, pooled datasets of all cilnidipine monotherapy patients (n=3) (at either 10 or 20 mg daily) or all pooled cilnidipine plus tadalafil treated patients (n=4), or pooled cilnidipine 10 mg with or without tadalafil (n=3) or pooled cilnidipine 20 mg with or without tadalafil, all met the study primary endpoint of a reduction of at least 25% or greater in the weekly frequency of attacks (−33 to −40%)

Assessing the effect of treatment on the duration of Raynaud attacks, all cilnidipine or cilnidipine plus tadalafil treated patients (n=7) demonstrated a decrease greater than that seen in placebo treated patients. Tadalafil monotherapy patients (n=2) saw on average an increase in the duration of their reported attacks during treatment. Of the 7 cilnidipine or cilnidipine plus tadalafil treated patients, this reduction exceeded a 25% threshold in 4 of 7 patients treated. In the single placebo patient who complied with the study protocol, duration decreased only 5%. Pooled cilnidipine plus tadalafil treated patients (n=4) had a 24.3% average reduction in duration of attacks during treatment. The ability of cilnidipine to decrease duration of attacks occurred regardless of whether disease was mild or severe at baseline as determined by RCS score at baseline.

When considering the severity of attacks as reported by patients, a dose response appears to be present with 20 mg reducing severity more than 10 mg of cilnidipine as monotherapy (n=3). Cilnidipine plus tadalafil (n=4) decreased severity more than tadalafil monotherapy (n=2). Cilnidipine plus tadalafil treated patients (n=4) had a 26% reduction in severity as reported with attacks. The reduction in severity appeared to be slightly greater in patients with milder disease (RCS<5.0, (n=2), −31%) than patients with more severe disease (RCS>5.0, n=2, −22%). In patients with more severe disease at baseline (RCS>5.0, adding cilnidipine to tadalafil produced a greater reduction in severity than treating with tadalafil alone (n=2 for cilnidipine plus tadalafil, 22.2% reduction in severity, n=2 for tadalafil monotherapy, 6.3% reduction in severity.

The benefit of adding tadalafil to cilnidipine in combination appears to be seen in its effect on RCS during the study. At both 10 mg and 20 mg doses of cilnidipine, the reduction in RCS was greater in combination with tadalafil 5 mg. (n=3 cilnidipine monotherapy versus n=4 cilnidipine plus tadalafil dual therapy). 3 of 4 cilnidipine plus tadalafil treated patients achieved a clinically meaningful >25% reduction in their baseline RCS while on treatment. A result this positive has not been seen in previous drug trials in this population and the FDA considers improvements in RCS scores to be an approvable endpoint for a treatment for these patients.

### Example 4. Two-Phase, Phase 2 Study, Assessing the Safety of Cilnidipine in SSc-RP and Dose Effect of a Combination of Cilnidipine and Tadalafil

Introduction: A two-phase, phase 2 study, evaluation the safety of cilnidipine in SSc-RP and determination of whether a dose effect existed for cilnidipine at two doses, and whether addition of tadalafil to these doses complemented efficacy and its effect on safety.

### Summary of Methods and Protocol:

### Part A

Part A of the trial employed a dose-finding, parallel arm design. The intention was to include six patients in each of the six arms. In this part, the objective was to determine the appropriate dosage of cilnidipine and tadalafil, as well as their combination, for further evaluation.

### Part B

Part B, is designed as a prospective, double-blind, randomized, placebo-controlled, two-way crossover trial. It aims to enroll a total of 72 patients, with a statistical power of over 80%. The primary goal of Part B is to assess whether cilnidipine has a significant effect (>25% reduction) on the weekly frequency of Raynaud's episodes in patients experiencing more than one attack per day during a two-week screening period.

### Interventions

The study evaluated the following interventions: cilnidipine at doses of 10 mg and 20 mg, tadalafil at a dose of 5 mg, administered once daily, as well as placebo.

### Medication Dispensation and Diary Maintenance

Study medication and matching placebo were provided to patients in kits at the time of randomization, following the screening process. Participants were instructed to maintain a daily electronic diary using a cell-phone based case report form (CRF). If necessary, patients were allowed to supplement the electronic diary with a paper record.

### Treatment and Assessment

Patients received the assigned intervention for a duration of two weeks. Subsequently, they returned to the clinic for assessments. After treatment discontinuation, patients were followed up for safety purposes.

### Concomitant Medications

Throughout the study, patients were permitted to continue their current stable doses of medications prescribed for the management of Raynaud's and other concurrent conditions.

### Study Endpoints

The study evaluated multiple endpoints, including the weekly frequency of Raynaud's attacks (primary endpoint), Raynaud Condition Score (RCS), pain severity, attack duration, Scleroderma Health Assessment Questionnaire (SHAQ), Patient-Reported Outcome (PRO), UCLA Gastrointestinal Tract (GIT) 2.0 assessment for gastrointestinal dysfunction, endothelial function assessed by Endo-PAT, thermography, and pharmacokinetics (PK).

### Detailed Protocol:

Detailed summary: A randomized, double-blind, placebo-controlled phase 2a study to assess the safety and efficacy of cilnidipine (10 mg and 20 mg) alone and in combination with 5 mg tadalafil in participants with diagnosis of secondary Raynaud's disease, also referred to as reconnoiter-1: randomized evaluation of the benefit of cilnidipine dose on the nature, observational indices, temperature changes, and overall effect in secondary Raynaud's disease.

### Overall Study Design

A schematic of the study design is provided in FIG. 1. This is a randomized, double-blind, placebo-controlled Phase 2a study to assess the safety and efficacy of cilnidipine alone and in combination with tadalafil, in participants who have frequent attacks of secondary RP mostly resulting from SSc. Oversight for the study will be provided by a DSMB.

Participants will undergo a screening period beginning up to 10 days prior to randomization. The initial screening and capacity will be conducted via phone at the start of the screening period with eligibility finalized prior to randomization on Day 0. Participants will be required to provide informed consent in a 2-step process at screening (commencement of diary use will be considered implied consent for the screening period) and at randomization (Day 0) before undertaking any study-specific procedures or assessments. Only participants who meet all of the inclusion and none of the exclusion criteria will be randomized.

The study consists of two parts.

Part A—double-blind, placebo-controlled, parallel-group, dose selection, will assess the safety and efficacy of two doses of cilnidipine (10 mg and 20 mg), alone and in combination with tadalafil. A total of 36 participants will be randomized to one of six prespecified treatment arms. Refer to FIG. 1 for treatment arms in Part A of the study. Dosing last for 12 (±2) days in which participants will self-administer daily doses of assigned treatment in the morning. Each participant will take one capsule and one tablet to blind the active therapy being received. Study visits and assessments will occur as delineated in the SoA presented in FIG. 2.

The data from Part A of the study will be reviewed by an unblinded DSMB prior to selecting the cilnidipine dose and confirming the sample size estimates for the randomized double-blind phase (Part B). The first review will occur after approximately 50% of participants have completed the study.

The data obtained and reviewed from 27 participants enrolled in Part A was sufficient to 1) confirm safety, tolerability, and potential efficacy of cilnidipine in patients with SSc-RP and 2) select the cilnidipine dose (20 mg) for Part B for continued evaluation of efficacy, safety, and tolerability. The Committee also agreed that the co-administration of tadalafil with cilnidipine did not provide significant additional benefit to the higher dose of cilnidipine chosen and may add some minor adverse events; therefore, the Committee recommended modifying Part B to a 2-way crossover design (CIL 20>placebo, placebo>CIL 20).

Part B—Double-blind, placebo-controlled, 2-way crossover will assess the safety and efficacy of cilnidipine 20 mg (the dose selected in Part A). A total of 38 participants (19 in each sequence) with a diagnosis of SSc-RP will be randomized into one of two prespecified treatment sequences in a 2-way crossover design.

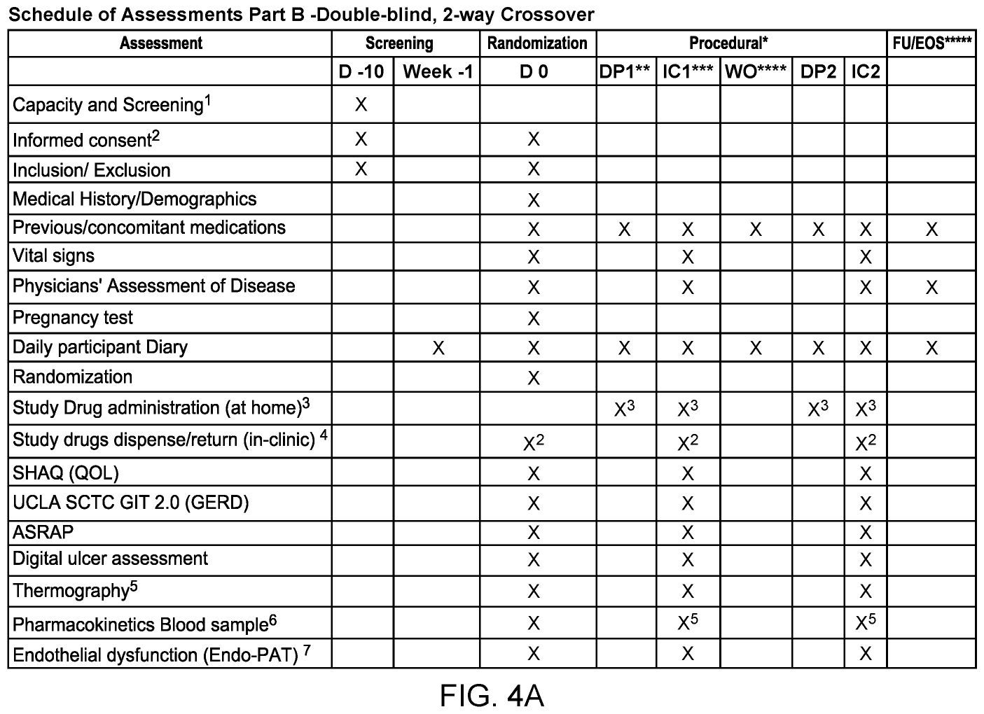

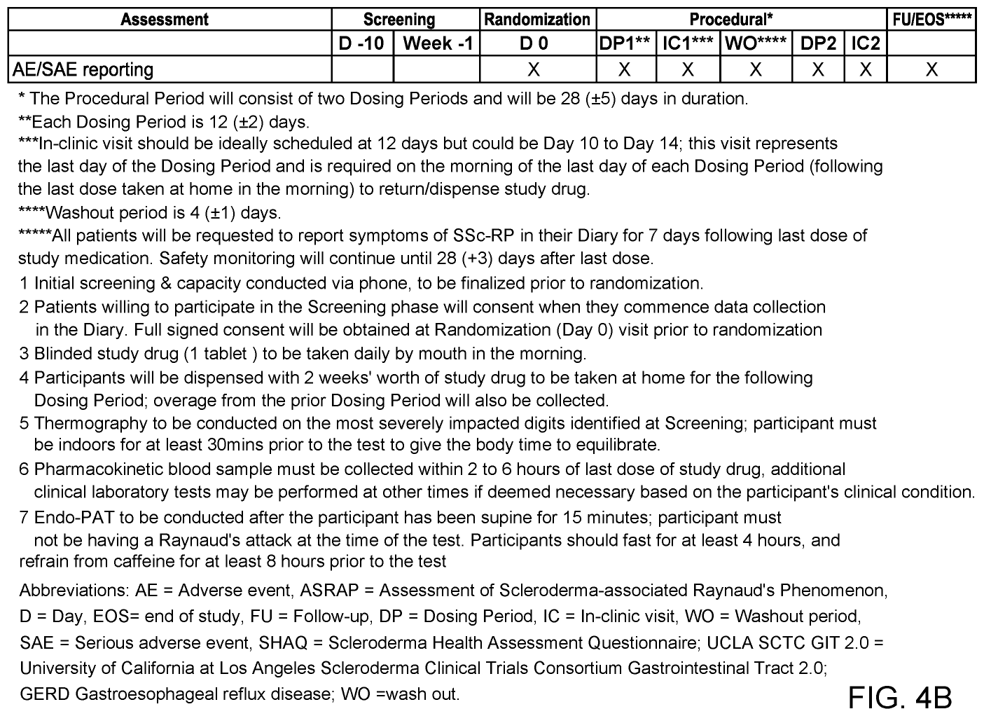

Refer to FIG. 1 for treatment sequences in Part B of the study. Each participant will undergo two dosing periods in which they will receive a different treatment each dosing period followed by a 4 (±1) day washout period. Each dosing period will last for 12 (±2) days in which participants will self-administer daily doses of assigned treatment in the morning. At all dosing periods, each participant will take one tablet to blind the active therapy being received. Study visits and assessments will occur as delineated in the SoA presented in FIGS. 4A and 4B.

For both Part A and B of the study, participants are required to visit the clinic on last day of each dosing period (i.e., day 10 to 14) to return/dispense study drug and conduct in person study assessments. Participants will be dispensed with 2 weeks' worth of study drug to be taken at home for the following dosing period; overage from the prior dosing period will also be collected.

Patients will be assessed for the occurrence of efficacy endpoints for each dosing period via the patient reported diary and the in-clinic visit. Safety information will be collected from randomization until patient follow-up is complete (28+3 days after the last dosing period) and assessed for each dosing period.

### Dose Selection, Sample Size Confirmation and Safety Oversight

The role of the DSMB, will be set out in a DSMB Charter. The DSMB will conduct a review of the efficacy and safety data from Part A of the study. The first review will occur after approximately 50% of the participants have completed the study. Subsequent reviews will occur as needed prior to commencement of Part B of the study to assess the risk: benefit of cilnidipine (10 mg and 20 mg), alone and in combination with tadalafil.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.
   Serious adverse events will be monitored (by the DSMB in Part A and Drug Safety Officer in Part B) on an ongoing basis throughout the study.

Efficacy and safety data from the 27 participants who completed Part A was included in the analyses. The benefit/risk profile demonstrated and recommended that the study proceed directly to Part B (powered crossover phase) without full completion of Part A. That the data from Part A is sufficient to 1) confirm safety, tolerability, and potential efficacy of cilnidipine in patients with SSc-RP and 2) select the cilnidipine dose (20 mg) for Part B for continued evaluation of efficacy, safety, and tolerability. The Committee also agreed that the co-administration of tadalafil with cilnidipine did not provide significant additional benefit to the higher dose of cilnidipine chosen and may add some minor adverse events; therefore, the Committee recommended modifying Part B to a 2-way crossover design (CIL 20>placebo, placebo>CIL 20).

Participants will receive study treatment (cilnidipine 20 mg or placebo tablet) in a blinded fashion. Each dosing period will last for 12 days (±2 days) in which participants will take daily doses of assigned treatment in the morning. The schematic for dosing is presented for Part A (Table S1) and Part B (Table S2) below and FIG. 1.

TABLE S1
Part A, Double-blind, Parallel-group, Dose Selection

| Arm | N | Treatment |
|---|---|---|
| A | 6 | P |
| B | 6 | C10 |
| C | 6 | C20 |
| D | 6 | T05 |
| E | 6 | C10 + T05 |
| F | 6 | C20 + T05 |

Abbreviations: C = cilnidipine, P = placebo, T = tadalafil, N = number of participants, C10 = C 10 mg, C20 = C 20 mg; T05 = T 5 mg.

TABLE S2
Part B, Double-blind, Placebo-controlled, 2-way crossover

| Sequence | | Dosing | Dosing |
|---|---|---|---|
| Number | N | Period 1 | Period 2 |
| 1 | 19 | P | C20 |
| 2 | 19 | C20 | P |

Abbreviations: C20 = cilnidipine 20 mg, P = placebo, N = number of participants

Number of Participants (Planned): Up to 65 participants will be enrolled in this study: 27 (revised from 36 based on DSMB recommendation) in the parallel-group dose selection phase (Part A) and 38 in the 2-way crossover phase (Part B). Participants who complete Part A without any major protocol deviations or compliance issues will be invited to participate in Part B. Participants would need to consent and meet all eligibility criteria again in order to be randomized into Part B. Dropouts will not be replaced.

### Diagnosis and Main Criteria for Inclusion:

Participants aged 18-90 years and diagnosed with severe secondary Raynaud's disease (Raynaud's Condition Score [RCS]≥40 and at least a 2-phase color change in fingers of pallor, cyanosis, and/or reactive hyperemia in response to cold exposure or emotion) mostly resulting from SSc and exhibiting regular and frequent RP attacks (averaging at least one attack per day) during the screening period.

### Investigational Product, Dosage and Mode of Administration:

### Cilnidipine

For Part A, Cilnidipine 10 mg and cilnidipine 20 mg for oral administration, will be provided to the site in cartons containing 16 tablets sealed in blister packs.
For Part B, Cilnidipine 20 mg (the dose selected in Part A) will be provided to the site in cartons containing 16 tablets sealed in blister packs.

### Tadalafil

For Part A, Tadalafil 5 mg, for oral administration will be over encapsulated (and back filled with inert capsule filler consisting of only maize starch and pre-gelatinized maize starch, so that the internal tablet cannot be detected) and provided in bottles containing 16 capsules to the site. Tadalafil will not be provided for Part B.

Medications will be dispensed during the preceding in-clinic study visit for self-administration by the participant once daily in the morning for a 12 (±2) day period. The duration of the Dosing period will be confirmed by the study staff when the in-clinic study visit is scheduled.

### Investigational Plan

### Study Rationale

No cure exists for patients suffering with SSc. The exact cause is unknown, however SSc is thought to result from a combination of factors, including autoimmune, genetic, and environmental triggers. Symptoms can include painful episodes in the extremities with color changes of the fingers or toes (e.g., RP), digital ulcers, skin thickening or hardening, capillary changes (assessed using nailfold capillaroscopy), swelling of the hands or legs, and general pain. Available therapies only provide symptomatic treatment with limited efficacy and safety. New treatments are needed to better manage the symptoms and directly address the underlying disease processes of SSc-RP. This study will evaluate the efficacy and safety of cilnidipine (Profervia®) monotherapy compared to placebo for the treatment of patients with secondary RP primarily due to SSc; the study will also include evaluation of combination therapy of cilnidipine and tadalafil to determine if the combination of cilnidipine with a low dose of the PDE5 inhibitor tadalafil will provide additional or synergistic benefits for the study patients. The pharmacology and safety profile of cilnidipine make it a potentially more efficacious and safe treatment for SSc than the currently available medications, with the added potential benefits of diminishing the major symptoms of the disease including improvement of Raynaud's attacks (reduction of frequency and severity), as well as possibly addressing and improving the underlying pathologic processes contributing to disease progression, including fibrosis and endothelial/vascular dysfunction. It is further anticipated that the pharmacokinetic profile of cilnidipine make it better suited for the treatment of SSc-RP patients than other currently approved CCBs, since peak blood levels of the drug after oral dosing are reached within 2 hours, which is more rapidly than other CCBs.

### Dosage Rationale

### Cilnidipine

Cilnidipine is approved for the treatment of hypertension in Japan, India, China, and South Korea in doses of 5 mg up to 20 mg. It is taken orally, once a day, usually in the morning. First approved in Japan in 1995, there is greater than 25 years of safety experience at these doses.

Cilnidipine's pharmacokinetics, with a more rapid time to achieve maximal plasma concentrations (2 hour Tₘₐₓ) than the CCBs amlodipine (10 hours) or extended release nifedipine (3 hours), may increase cilnidipine's suitability for treating Raynaud's symptoms in SSc patients compared to other CCBs, in that these symptoms commonly occur early in the day. Cilnidipine is also dosed once daily compared to nifedipine's three times a day dosing schedule.

### Duration of Treatment:

Part A, Double-blind, Parallel-group, Dose Selection: Participants enrolled will participate for up to 29 (±5) days:
Screening: up to 10 days (−3 days)
Treatment: 12 (±2) days
Follow-up: 7 days (+3 days)
   Part B, Double-blind, Placebo-controlled, 2-way crossover: Participants enrolled will participate for up to 63 days (±8 days):
Screening: up to 10 days (−3 days)
Treatment: 28 days (two s each lasting for 12 (±2) days, separated by a 4 (±1) day washout period)
Follow-up: 28 days (+3 days)

### Reference Therapy, Dosage, and Mode of Administration:

Placebo: Placebo tablets (matching cilnidipine) and placebo capsules (matching tadalafil, Part A only), for oral administration, will be provided to the site.

### Study Objectives And Endpoints

### Objectives

### Primary Efficacy Objectives:

The primary efficacy objective of the study is to evaluate the effect of cilnidipine on the frequency of weekly RP attacks compared with placebo in participants with SSc-RP.

### Secondary Efficacy Objectives:

The secondary efficacy objective of the study is to evaluate the effect of cilnidipine on all the clinical features of SSc-RP, including symptoms and disability associated with SSc in addition to RP.

### Safety Objectives:

The safety objective of the study is to evaluate the safety of cilnidipine compared to placebo in participants with SSc-RP.

### Exploratory Objectives:

To assess the impact of treatment on 1) endothelial function and 2) severity and impact of Raynaud's phenomenon, in participants with SSc-RP

To evaluate the efficacy and safety of combination therapy (cilnidipine 10 mg and 20 mg, in combination with tadalafil) on all efficacy and safety endpoints (Part A only)

### Primary Efficacy Endpoint:

Percentage change from baseline in frequency of weekly RP attacks.

### Secondary Efficacy Endpoints:

Change from baseline in frequency of weekly RP attacks.
Change from Baseline in average duration of weekly RP attacks.
Change from Baseline in average severity of weekly RP attacks.
Change from Baseline in average daily RCS.
Change from baseline in highest (most severe) pain score recorded during weekly RP attacks.
Change from baseline in average pain score recorded during weekly RP attacks.
Change from baseline in net digital ulcer burden.
Change from baseline in distal-dorsal difference (DDD) of the affected index finger sites.
Change from baseline in participant quality of life and other aspects of scleroderma disease measured using the Scleroderma Health Assessment Questionnaire (SHAQ).
Change from baseline in participant gastrointestinal symptoms (of sclerosis) as assessed with the UCLA SCTC GIT 2.0 questionnaire.
Change from baseline in Raynaud-visual analog scale (VAS).
Change from baseline in physician assessment of disease.
Measurement of cilnidipine drug levels taken 2 to 6 hours following the last dose.
The time to reach maximum degree of efficacy (in days) compared to baseline.
The time to return to baseline symptom severity after termination of dosing.
Impact of daily ambient temperature on symptomatic RP attacks.
Use of rescue medications for breakthrough symptoms.

### Exploratory Efficacy Endpoints:

Change from baseline in endothelial function as measured by reactive hyperemia index (RHI) using Endo-PAT
Change from baseline in novel patient-reported outcomes (ASRAP score) which assesses the severity and impact of RP in SSc (Part B).
Change from baseline in combination endpoint (frequency of weekly RP attacks and ASRAP score) (Part B).
Change from baseline in reported ocular symptoms (Part B)
Effect of combination therapy (Cilnidipine 10 mg+Tadalafil 5 mg, and Cilnidipine 20 mg+Tadalafil 5 mg) on all efficacy endpoints (Part A only).

### Safety Endpoints:

Incidence of treatment emergent adverse events (AEs) and serious adverse events (SAEs), including clinically significant vital signs. For Part A safety will be assessed from the time of randomization until 7 days following the last protocol dose. For Part B, participants will be followed for 28 days from the last study dose.
Serious adverse events will be monitored by a DSMB (Part A) and Drug Safety Officer (Part B) on an ongoing basis throughout the study.
Adverse events will be coded using the MedDRA and summarized by SOC, PT, and treatment group.

### Statistical Methods:

Part A data will be analyzed in exploratory fashion to support cilnidipine dose selection and treatment effect check for sample size confirmation for Part B. For Part B data, mixed effects model will be used for analysis of the continuous efficacy endpoints in the crossover design. For nominal data, Chi-square tests will be applied. Generalized Estimating Equations method will be used, as appropriate, for adjusting for potential confounding factors. Safety endpoints will be summarized by treatment group. No multiple comparison adjustment will be used to control alpha for the multiple comparisons.

### Statistical Analyses:

Data from Part A and Part B will be analyzed separately. The primary and secondary efficacy analyses will be based on the Modified Intent-To-Treat (mITT) population. Analyses based on the Intent-To-Treat (ITT) and PP population for Part B will be considered secondary and confirmatory. All safety analyses will be performed on the safety population. Subgroup analyses will also be performed.

Additional exploratory analyses may be performed and will be documented in the statistical analysis plan. Any deviation from planned analyses described in this protocol will also be documented in the statistical analysis plan. Data will be summarized by treatment group for treatment effect comparison according to crossover design; participants receiving each treatment will be pooled from all periods. Baseline will be defined as screening assessments for change from baseline analyses for all periods.

### Sample Size Calculation:

The sample size for Part B was calculated based on the available data from Part A at the time of protocol specified data review Assuming a 2-sided 0.05 alpha for the comparison of cilnidipine 20 mg versus placebo, between-participants standard deviation (SD) of 35, a correlation between the two measurements on the same participant of 0.2, and a 25% dropout rate, it is estimated that a total sample size of 38 participants (19 in each treatment sequence) is needed to obtain complete data from 28 participants (14 in each treatment sequence), for

≥80% power to detect a decrease of 25 in percentage change from baseline in weekly RP attacks in a 2×2 crossover design.

### Pharmacokinetics and Pharmacodynamics

### Pharmacokinetics

Cilnidipine achieves maximal plasma concentration in about 2 hours, which is more rapid than other CCBs (e.g., amlodipine at 10 hours or extended use nifedipine at 3 hours), which may increase cilnidipine's suitability for treating SSc patients. See S3 for a summary of pharmacokinetic (PK) parameters for cilnidipine.

TABLE S3
PK Parameters for 10 mg Cilnidipine,
Healthy Volunteers, single oral dose

| Pharmacokinetic Parameters | Mean | |
|---|---|---|
| Cₘₐₓ (ng/mL) | 8.9 | (±4.2) |
| Tₘₐₓ (hr) | 2 | (0.5-5) |
| AUCₗₐₛₜ (ng*hr/mL) | 37.0 | (±17.9) |
| AUC_{inf} (ng*h/mL) | 41.2 | (±20.4) |
| CL/F (L/hr) | 304.7 | (±147.5) |
| T_{1/2} (hr) | 7.5 | (±1.0) |

Source: Lee, et al., Drug Des Devel Ther. 2014; 8: 1781-1788. 2014
*Mean (±standard deviation), except for Tₘₐₓ for which median (minimum − maximum) is presented.
Abbreviations: AUC_{inf} = area under the plasma concentration time curve from time zero extrapolated to infinity. AUCₗₐₛₜ = area under the plasma concentration time curve to the last observation. Cₘₐₓ = maximum plasma concentration. CL/F = apparent clearance. Tₘₐₓ = time to reach Cₘₐₓ.

### Pharmacodynamics

The mechanism of action of cilnidipine offers unique potential benefits for SSc participants that differentiate it from other dihydropyridine CCBs. In hypertensive patients, cilnidipine has been shown to have similar equipotent efficacy when compared to other calcium channel antagonist hypertensive treatments, while exhibiting a better safety profile. This is due to cilnidipine's N-type Ca channel selectivity, in addition to its L-type Ca channel activity.

Currently approved CCBs have primarily L-channel Ca activity and little or no N-type activity. Because of its improved safety profile, cilnidipine can be dosed at higher dose levels than other non-N-selective CCBs, engendering greater efficacy in reducing blood pressure.

Unlike other CCBs, cilnidipine with its primarily N-type Ca channel activity also inhibits sympathetic nervous system activity, dilates venules in addition to arterioles, improves endothelial structure and function, and may provide analgesic effects. Cilnidipine has also demonstrated anti-fibrotic effects in nonclinical studies as well as additional renal and cardiovascular effects in clinical studies. Cilnidipine also is a potent inhibitor of the purinergic P2X7R pathway, and studies have shown that fibroblasts from patients with SSc show upregulation of this receptor and that it promotes a fibrogenic phenotype in their fibroblasts. These additional pharmacodynamic properties of cilnidipine address several key factors of SSc and may provide superior treatment for SSc participants than currently available treatments. This is a first in human (FIH) study of cilnidipine and tadalafil combination. No clinical studies of cilnidipine and tadalafil combination have been conducted to date. However, based on the clinical use of CCBs in combination with PDE5 inhibitors, no drug-drug interaction is expected.

### Safety Criteria for Stopping or Unblinding Treatment

Administration of study drug may be paused, and emergency unblinding of treatment conducted following consultation between the Investigator, the Medical Monitor, and the Sponsor representative under the following circumstances:
Symptoms of possible allergic phenomena: rash, hives, urticaria, changes in breathing or wheezing.
Systolic BP (SBP)<90 mmHg
Reduction in BP (relative to baseline values) considered to be significant in the opinion of the Investigator on an absolute basis or consistent with symptoms (dizziness, light-headedness) suggestive of being related to reduction in BP.
Two separate SBP measurements of <90 mmHg, taken at rest (where baseline SBP exceeded 110 mmHg), even in the absence of symptoms, shall be considered sufficient reason to unblind the study for that patient.
Dizziness, when going from a recumbent to standing position and/or if accompanied by a reduction in BP.
Dizziness in the absence of a reduction in BP should be considered on an individual participant basis, as to its relative degree of severity, as to whether the participant continues in the study.
Unexpected adverse event or reaction.

### Study Termination

The study will be completed as planned unless:
New information or other evaluation regarding the safety of the study medication indicates a change in the known risk/benefit profile for the compound, such that the risk/benefit is no longer acceptable for participants participating in the study. This may be determined by the Sponsor, the Investigator, the HREC or regulatory authorities.
The study is terminated by the Sponsor for administrative reasons.

### 4. Participant Population

The study will be conducted in participants aged 18-90 years, diagnosed with severe secondary Raynaud's disease (RCS≥40 and at least a 2-phase color change in fingers of pallor, cyanosis, and/or reactive hyperemia in response to cold exposure or emotion) mostly resulting from SSc (defined by consensus criteria 2013 American College of Rheumatology [ACR]) and exhibiting a frequency of attacks (at least one per day) during the screening period.
Women of childbearing potential will be included and are subject to contraceptive requirements during the study from screening until study completion, including the follow-up period, and for at least 30 days after the last dose of study drug (see Section 4.2). Women of childbearing potential must demonstrate negative pregnancy testing at screening. This is in line with regulatory Guidance on Nonclinical Safety Studies for the Conduct of Human Clinical Trials and Marketing Authorization for Pharmaceuticals (US FDA Guidance document, January 2010).

### Number of Participants

Up to, 76 participants will be enrolled in this study. Thirty-six participants were planned for the parallel-group dose selection phase (Part A), however following the DSMB recommendation to proceed directly to Part B (the powered cross-over phase), Part A was stopped early with 27 participants randomized. In Part B, 38 participants will be randomized in a 2-way crossover design. Participants who complete Part A without any major protocol deviations or compliance issues will be invited to participate in Part B. Following completion of Part A, participants would need to provide written informed consent and meet all eligibility criteria again in order to be randomized into Part B. Sample size assumptions account for a dropout rate of 25%, therefore dropouts will not be replaced.

### Participant Inclusion Criteria

To be eligible for this study, a participant has to meet all of the following inclusion criteria:
1. Male or female participants, aged 18 to 90 years (inclusive at the time of informed consent).
2. Severe secondary Raynaud's disease (defined as RCS at baseline of ≥40) based on ACR criteria mostly resulting from SSc.
3. Regular and frequent Raynaud's attacks (averaging at least one attack per day) during the screening week (in participants with at least a 2-phase color change in fingers of pallor, cyanosis, and/or reactive hyperemia in response to cold exposure or emotion).
4. Willingness to complete the daily diary entry's during the screening period.
5. Participants must be willing and able to provide written informed consent after the nature of the study has been explained and prior to the commencement of any study procedures.
6. Willingness to forego therapies for Raynaud's during study participation is preferred (except for selective serotonin reuptake inhibitors such as fluoxetine which should not be discontinued) Ongoing treatment with CCBs and other routine therapies for SSc-RP is permitted however, if it is not clinically feasible to stop therapy, the participant has been on a stable dose for the last 2 months and meets all eligibility criteria. Rescue therapy (with acetaminophen, nonsteroidal anti-inflammatory drug [NSAIDs], other codeine analgesics, fluoxetine, angiotensin II receptor blockers (ARBs) such as losartan) or CCBs will be allowed to manage breakthrough symptoms of SSc-RP but must be documented).
7. Participants who are on a stable dose (no change in the last 2 months) of a CCB for hypertension or PDE5 inhibitors for pulmonary hypertension may continue these agents at this stable dose as long as they meet other inclusion criteria during screening.
8. Have agreed not to use (or initiate treatment with) other investigational therapies or unapproved therapies during the course of the study outside of protocol allowances for rescue medication (such medications include but are not limited to: nitroglycerin, topical creams, fenoldopam, nimodipine, amlodipine, fluoxetine, pregabalin, gabapentin, verapamil, sildenafil, tadalafil, vardenafil).
9. Women of childbearing potential (WOCBP) who have agreed to use an effective method of contraception during the study and for 30 days after the last study dose. Acceptable methods of birth control in this study include oral contraceptive, barrier control, or implanted devices. A woman must agree not to donate eggs (ova; oocytes) for purposes of reproduction for at least 30 days after last dose of study drug.
10. Post-menopausal females, aged over 45 years who have not had a period for at least 12 months, and are not using hormonal contraception, or females documented as permanently sterile (e.g., bilateral tubal ligation; hysterectomy, bilateral salpingectomy).
11. Negative urine pregnancy test on randomization day (WOCBP only).
12. All sexually active men whose partner is a WOCBP (due to potential risk of drug exposure through the ejaculate) who agree to a barrier method of birth control during the study and for 30 days after the last dose of the study drug. All men must agree not to donate sperm for at least 30 days after receiving last dose of study drug.
13. Participants must have clinical laboratory values within normal range as specified by the testing laboratory at their most recent pre-screening sample, unless deemed not clinically significant by the Investigator or delegate. Lab sample may be within the last 2 months as long as there have been no significant clinical changes since the last labs and no new medications (e.g. Diuretics) have been introduced that are known to be associated with changes in clinical chemistry or hematology values (e.g. potassium with diuretics or cyclosporine associated with aplastic anemia).

### Participant Exclusion Criteria

A participant who meets any of the following exclusion criteria must be excluded from the study:
1. Primary Raynaud's disease.
2. History of Raynaud's attacks of sufficient severity as to require in-patient hospitalization (within the last 6 months).
3. The SBP of <95 mm Hg during randomization visit (Day 0).
4. Participants with an allergy to dihydropyridine CCBs that results in clinical findings such as profound hypotension, hives, rash, urticaria, wheezing and changes in breathing (Common treatment limiting adverse events [AEs] that occur with CCBs nifedipine and amlodipine such as edema, headache, heart rate changes, tachycardia, fatigue, constipation, flushing, drowsiness, dizziness should not limit enrollment into this study).
5. History of other chronic pain condition that could confound recording of pain scores during the study period.
6. Any prior or ongoing medical conditions, medical history, physical findings, or laboratory abnormality that in the opinion of the Investigator(s), would contraindicate administration of the study medication, or interfere with the study evaluations, or interfere with the participant's ability to comply with the study protocol.
7. Cognitive or language difficulties that would impair completion of the study assessments.
8. Use of any investigational product (IP) or investigational medical device or participation in investigational drug studies within 30 days prior to enrollment in the study.
9. Those receiving nitrates (nitro-dur, nitroglycerin) or similar agents that have vasodilatory effects (e.g., nicorandil) prescribed to treat angina, alpha blockers, PDE inhibitors, prostacyclins or endothelin antagonists for whom dose is not stable for >2 months
10. History of orthostatic hypertension, dizziness or fainting spells, acute coronary or cerebrovascular event within 3 months of study enrollment.
11. History of major thoracic, abdominal, or vascular surgery within 6 months of study enrollment; History of sympathectomy in the hand which is symptomatic for RP.
12. Severe cardiomyopathy, severe valvular heart disease, chronic kidney disease (CKD) stage 3 or greater, evidence of malignancy, end stage lung disease.
13. Pregnant or lactating women.
14. Women of childbearing potential (WOCBP) unable to comply with contraceptive requirements during the study period.
15. Males with partners who are WOCBP and are unable to comply with the contraceptive requirements during the study.
16. History of drug (including recreational use of inhaled amyl nitrates or party poppers) or excess alcohol use that in the opinion of the Investigator(s) would affect the participant's ability to reliably participate in the study. NHMRC guidelines for regular alcohol consumption in healthy adults are no more than 10 standard drinks per week and no more than four standard drinks on any one day.
17. Heavy smokers of tobacco products of any type (defined as >10 cigarettes per day). Light smokers who agree to keep their smoking stable for the duration of their trial participation are eligible.

### Screen Failures

Screen failures are defined as participants who consent to participate in the clinical study but are not subsequently randomized in the study. A minimal set of screen failure information is required to ensure transparent reporting of screen failure participants to meet the Consolidated Standards of Reporting Trials publishing requirements and to respond to queries from regulatory authorities. Minimal information includes screen failure details and eligibility criteria. Participants who withdraw from the study, for any reason, prior to randomization will be considered screen failures. Individuals who do not meet the criteria for participation in this study (screen failure) may be re-screened following a one month waiting period. Re-screened participants should be assigned a new participant number.

### Participant Replacement

All participants who are randomized will be followed and included in the primary ITT analysis. Dropouts will not be replaced.

### Participant Withdrawal Criteria

Participants may withdraw their consent to participate in the study at any time. If a participant withdraws consent, the date and reason for consent withdrawal should be documented. Participants will be encouraged to remain in the clinic to complete all necessary assessments and until the Investigator deems that it is safe to be discharged. Participant data will be included in the analysis up to the date of the withdrawal of consent.

Apart from withdrawal of consent, reasons for early termination of individual participants can include:
Protocol deviations or participant non-compliance (must be specified on the appropriate eCRF)
Serious or severe AEs
Administrative decision by the Investigator or the Sponsor
Death
Other (must be specified).

The primary reason for withdrawal will be identified and recorded on the appropriate eCRF, along with the date of withdrawal.

In accordance with applicable regulations, a participant has the right to withdraw from the study, at any time and for any reason, without prejudice to future medical care. If a participant is withdrawn because of an AE, the Investigator must arrange for the participant to have appropriate follow-up care until the AE is resolved or has stabilized. Unresolved AEs will be followed until the last scheduled Follow-up/End of Study (EOS) visit or until the Investigator(s) determine that further follow-up is no longer indicated. In addition to AEs, other reasons for removal of participants from the study might include, but are not limited to, withdrawal of consent, administrative decision by the Investigator or the Sponsor, protocol deviation, or participant noncompliance.

If a participant asks or decides to withdraw from the study, all efforts will be made to complete and report the observations, especially those related to the listed primary and secondary objectives, as thoroughly as possible up to the date of withdrawal. Wherever possible, the tests and evaluations, including those listed for the EOS/follow-up visit, should be performed for all participants who discontinue prior to the completion of the study.

### Treatments

### Cilnidipine

Cilnidipine is an orally administered dihydropyridine CCB that dilates blood vessels, increases blood flow, inhibits sympathetic nervous system activity, and improves endothelial structure and function. Please refer to IB for more information on composition of cilnidipine tablet.

Profervia® tablets are white film-coated tablets. Each tablet contains cilnidipine (10 or 20 mg) with microcrystalline cellulose, lactose, magnesium stearate, sodium starch glycollate, Opadry white, polyvinyl alcohol, titanium dioxide, macrogol, talc, and purified water.

Cilnidipine is commercially available and should be used only in accordance with this study protocol and IB. Cilnidipine 10 mg and 20 mg oral tablets will be provided to the site in cartons containing 16 tablets sealed in blister packs.

### Tadalafil

Tadalafil for oral administration belong to a class of medications called PDE5 inhibitors.

Tadalafil is commercially available and should be used only in accordance with this study protocol. Please refer to the pharmacy manual and product information sheet for more information on composition and storage information for tadalafil. Tadalafil will be over encapsulated (and back filled with inert capsule filler consisting only of maize starch and pre-gelatinized maize starch, so that the internal tablet cannot be detected) and provided in bottles containing 16 capsules to the site.

### Reference Products

Placebo tablets (matching cilnidipine) and placebo capsules (matching tadalafil) for oral administration will be provided for this study.

### Dosage and Treatment Periods

In Part A, each participant will take daily one capsule and one tablet to blind the active therapy being received. In Part B, each participant will only take one tablet daily. All medications for each dosing period (each dosing period will last for 12 days [±2 days]) will be dispensed during the preceding in-clinic visit and then self-administered by the participant once daily, orally, in the morning.

If a participant accidentally misses a dose, they should be advised to take the dose on the same day as soon as they realize. Only one dose should be taken each day. If more than one dose is lost, the participant should notify the study staff so that their in-clinic visit can be adjusted if needed.

Part A, Double-blind, Parallel-group, Dose Selection Study drug will be self-administered daily for 12 (±2) days. Each participant will receive only one treatment. Please refer to FIG. 2 for more details.

Part B, double-blind, placebo-controlled, 2-way crossover study drug will be self-administered daily in two dosing periods separated by a four-day (±1) washout period. Each participant will receive a different treatment during each dosing period, with a total of two treatments received. Please refer to FIGS. 4A and 4B for more detail.

### Method of Assigning Participants to Treatment

### Randomization

A randomization list will be prepared using a statistical software package by a Biostatistician. Each participant will be provided with a unique screening number post-documentation of informed consent. Once deemed eligible, the participant will be assigned a sequential randomization number prior to first dosing. Participants who consent to screening but then withdraw from the study or fail eligibility, for any reason, prior to randomization will be considered screen failures.

Participants who complete Part A without any major protocol deviations or compliance issues will be invited to participate in Part B. Those who consent to be rescreened for Part B will be provided with a second unique screening number. If deemed eligible, the participant will be assigned a sequential randomization number specific to Part B.

### Concomitant Medications

All medications, including over the counter medications, vitamins, and herbal supplements, taken during the screening period will be reviewed by the Investigator to determine whether these medications render the participant as suitable for inclusion in the study.

Concomitant medications of interest will be captured electronically from the start of the screening period until study completion.

Treatment prior to enrollment with therapies for SSc-RP including but not limited to CCBs, nitroglycerin, topical creams fenoldopam, nimodipine, fluoxetine, pregabalin, gabapentin, sildenafil, tadalafil, vardenafil are permitted. In order to be eligible, participants must be willing to forego these therapies for SSc-RP at the start of the screening period and for the duration of the study. Participants who are on a stable dose (no change in dose in prior 2 months) of a CCB for hypertension or sildenafil for pulmonary hypertension may continue these agents at this stable dose as long as they meet other inclusion criteria during screening. Similarly, participants who are receiving CCBs to manage their symptoms of SSc-RP and are unwilling to stop treatment for the duration of the study would still be eligible if the participant's dose has been stable for the past 2 months. During the study participants will be able to decrease the dosage of their prior CCB for safety reasons only; no increase in dosage will be allowed during the study period. The use of any other IP or investigational medical device within 30 days prior to screening is prohibited.

Prior therapy or concomitant therapy (after study drug administration) with any medications, including both prescription and non-prescription drugs should be discussed with the Investigator and Sponsor's MM before study drug administration, except in the case of necessary treatment of AEs or where appropriate medical care necessitates that therapy should begin before the Investigator can consult with the Sponsor's MM.

### Rescue Medicine

Medications required as rescue therapy can be taken to manage breakthrough symptoms of SSc-RP but must be recorded in the participant Diary. First-line therapy may include acetaminophen, NSAIDs, or other codeine-based analgesics. These rescue medications may be taken for the duration of symptoms of a Raynaud's attack. For participants in whom first-line rescue therapy is not effective, additional rescue medication therapy may be started per Investigator discretion and could include fluoxetine, ARBs such as losartan or CCBs. Rescue therapy should continue as long as clinically needed during an acute attack, but then patients should return to the pre-rescue study medication regimen. All participants receiving rescue therapy should continue in the study undergoing subsequent dosing periods through study completion.

### Treatment Compliance

All doses will be self-administered by participants remote from study sites (at home). For each dosing period, participants will be dispensed with two weeks' worth of study medication and will be asked to return the unused study medication on the last day of each dosing period at the time of in-clinic visit. The treatment compliance will be noted by the Investigator(s) during the in-clinic visit.

### Blinding

This study is double-blind. To maintain the blind, all study medication will be provided to the site in a blinded fashion. Cilnidipine tablets and matching placebo will be supplied in cartons containing 16 tablets sealed in blister packs, identical in appearance. Tadalafil will be provided in an over encapsulated form. The capsules, tadalafil, and placebo will be identical in appearance and weight and will be supplied in bottles containing 16 capsules. Each study drug will be labeled with a unique ID number. The interactive voice response system (IVRS) will have access to the treatment arm assignment for each individual ID number.

### In the Event of an Emergency: Unblinding a Code

It is recognized that, in the course of clinical practice, it may be necessary for the treating physician to have knowledge of the treatment assignment to ensure the safety of a study participant. This circumstance is extraordinary and will likely impact a minor fraction of the enrolled participants. Unblinding will be done via the IVRS. The treating physician is encouraged to contact the Sponsor MM in this circumstance. The Sponsor and DSMB will monitor all episodes of unblinding very carefully.

### Study Schedule

The SoAs for Part A and Part B of the study are provided in FIG. 2 and FIGS. 4A and 4B. Where possible, assessments should be conducted in order of least invasive to most invasive. This study consists of four periods:
Screening period (begins with initial participant contact through participant completion of the screening Diary)
Randomization period (from the time participant eligibility is confirmed and the participant randomized until immediately before the 1^{st} dose of study drug)
Procedural period (from the first dose of study drug in the first dosing period until the last day of dosing)
Follow-up period (from the end of the procedural period through completion of follow-up for safety i.e., 7 days for Part A and 28 days for Part B).

### Part A—Double-Blind, Placebo-Controlled, Parallel-Group, Dose Selection

In Part A, the procedural period will require only one dosing period i.e. participants will receive only one treatment during the procedural period. Within the procedural period for Part A there will be two sub-periods:
Daily at home dosing (first ten to fourteen days of the dosing period)
In-clinic visit (the last day of the dosing period that occurs on Day 12 [±2 days]).

### Part B Double-Blind, Placebo-Controlled, 2-Way Crossover

In Part B, the procedural period will require two dosing periods i.e. participants will receive two different treatments in a 2-way crossover design. Within the procedural period for Part B there will be two sub-periods associated with each dosing period/treatment received:
Daily at home dosing (first ten to fourteen days of the dosing period)
In-clinic visit (the last day of the dosing period that occurs on Day 12 [±2 days]) One washout period (four days of no dosing) is required between each dosing period).

### Screening (Day −10 to Day −1)

Prior to enrolling in the study, and before performance of any procedures, potential participants will be contacted via phone to discuss the details of the study and assess their eligibility and willingness to comply with all study procedures and duration. A copy of the Informed Consent Form (ICF) will be emailed to the patient in conjunction with this discussion. If the participant seems eligible and is interested in participating in the screening period, then they will be asked to start using a diary to record the daily clinical features and symptoms of their SSc-RP for the next 7 to 10 days. Commencement of diary use will be considered implied consent for the screening period, the data from which will be used to confirm eligibility and future baseline analyses assuming the participant is randomized.

During screening, the Diary will collect the following data to confirm eligibility and serve as the baseline measure for efficacy endpoints should the participant be randomized:
Number of daily Raynaud's attacks
Duration of each attack
Symptoms of each attack, including numbness, pain, tingling, color changes
Severity of each attack (all symptoms of the attack to be considered including tingling, numbness, pain, color changes)
Location of participant during each attack (inside/outside)
Pain score of each attack—using 11-point Likert scale, a validated pain scale which can be used to record intensity of pain.
Daily RCS— a validated outcome measure used to assess the level of difficulty experienced due to RP each day.
   Rescue medications taken to manage breakthrough symptoms of SSc-RP (including acetaminophen, NSAIDs, other codeine-based medicines, fluoxetine, ARBs such as losartan, CCBs) as well as other concomitant medications will also be captured and assessed during screening.

### Randomization Day (Day 0)

Participants will be scheduled to visit the clinic for randomization (Day 0) assessments between days 7 to 10 of the screening period. Only participants who seem eligible based on Diary compliance and frequency of RP attacks will be requested to visit clinic for randomization. During the visit the participant will be provided with another copy of the ICF. Prior to being asked to sign the consent form, participants will be given time to review study information and ask any questions.

After the consent form is signed and the following assessments will be carried out:
Medical history/demographics
Previous/concomitant medications
Vital signs
Physician's Assessment of Disease (including RP, scleroderma, ulcers, ocular symptoms, overall health)
Pregnancy test
Review of daily participant dairy
SHAQ which includes Raynaud's VAS to be completed by the participant with review by physician designee
Assessment of gastrointestinal symptoms (of sclerosis) using the UCLA SCTC GIT 2.0 questionnaire, to be completed by the participant with review by physician designee
Assessment of Systemic sclerosis-associated Raynaud's Phenomenon (ASRAP) questionnaire to be completed by the participant with review by physician designee (Part B)
Digital ulcer assessment
Thermography
Endo-PAT
Blood sample for PK
Inclusion/Exclusion
Randomization
AE/SAE reporting
Dispensing study medication
   Note: The data collected for assessments that are performed first time on randomization (Day 0) visit (vital signs, digital ulcer assessment, and Endo-PAT) will serve as baseline measure for efficacy endpoints for those assessments.

Routine hospital tests including hematology, biochemistry, inflammatory markers (C-reactive protein [CRP] and erythrocyte sedimentation rate [ESR]), antibody status (serum anti-topoisomerase [anti-Scl 70]) and nailfold capillaroscopy should be conducted as clinically indicated per standard of care but are not required per protocol. If conducted, results will be collected in the eCRF and used to describe the severity of disease in the baseline demographic and disease data.

### Dosing Periods

### Daily at Home Dosing (12 Days (+2 Days))

During the dosing period, participants will be required to self-administer the assigned study medication once daily in the morning at home. Participants will also be required to complete their Diary daily to capture the clinical features and symptoms of their SSc-RP, and report concomitant medications including rescue therapy (if any).

### In-Clinic Visit (Last Day of the Dosing Period)

Participants will be required to visit the clinic following each dosing period—dosing day 10 to Day 14. The day of the in-clinic visit is considered the last day of dosing in each dosing period. After taking their last dose of study medication in the morning at home, the following assessments/procedures will take place during the in-clinic visit by the physician or designee, with results recorded in the eCRF:
Vital signs
Blood sample for PK
Daily participant E-dairy review for the most recent dosing sequence
Scleroderma Health Assessment Questionnaire (SHAQ) which includes Raynaud's VAS to be completed by the participant with review by physician designee
Assessment of gastrointestinal symptoms (of sclerosis) using the UCLA SCTC GIT 2.0 questionnaire, to be completed by the participant with review by physician designee
Assessment of Systemic sclerosis-associated Raynaud's Phenomenon (ASRAP) questionnaire, to be completed by the participant with review by physician designee (Part B)
Physician's Assessment of Disease (including RP, scleroderma, ulcers, ocular symptoms, overall health)
Concomitant medications
Thermography (for two most symptomatic areas in terms of Raynaud's)
Endothelial dysfunction (Endo-PAT)
AE/SAE reporting
Dispensing/returning study medication
   Note: On the last clinic visit at the end of last dosing period (dosing period 1 for Part A and dosing period 2 for Part B), pregnancy test will be performed for WOCBP.

### Washout Period

In Part B, each dosing period will be separated by a 4-day (±1 day) washout period. The washout period will commence the day after the in-clinic visit during which participants will not take any study medication. During the washout period, participants will be required to complete the daily participant Diary, reporting their symptoms of SSc-RP, and use of any concomitant medications. Once the 4-day washout is completed the participant will commence the daily at home dosing for the study medication dispensed at the previous in-clinic visit. No washout period is required after the second and final in-clinic visit. After this visit participants will proceed directly to follow-up.

### Follow-Up

All Participants will be followed for 7 days following completion of the final dosing period for their symptoms of SSc-RP, during which time they will be requested to continue to complete their patient Diary. Participants will also be requested to report use of any concomitant medications and any AEs/SAEs during the Follow-up period. Participants in Part B will be followed for AEs/SAEs until 28 days following the last dose of study medication. Follow-up of AEs/SAEs will cease at 7 days for participants in Part A. This visit marks the end of participation in this study.

### Early Termination (If Applicable)

Participants who withdraw early from the study will be encouraged to return to the clinic for an EOS assessment. The following procedures will be conducted:
Participants will be requested to complete the Diary for 7 days following termination
Participants will be requested to report use of concomitant medications of interest for 7 days following termination
Participants will be requested to report any AEs/SAEs for 7 (Part A) to 28 (Part B) days following termination. This visit marks the end of participation for participants that withdraw early from the study.

### Pharmacokinetic Assessments

### Blood Sample Collection

One 4 mL blood sample will be obtained during each in-clinic visit within 2 to 6 hours of last dose of study drug in that dosing period as delineated in the SoA (FIG. 2 and FIGS. 4A and 4B). The level of cilnidipine in blood will be measured following last dose of the dosing period. Sample handling details will be provided in the PK manual. The actual collection time of each sample must be recorded in the source data documentation, on the collection tube and in the eCRF.

### Efficacy Assessments

### Efficacy Parameters

Study procedures should be completed as delineated in the SoAs (FIG. 2 and FIGS. 4A and 4B).

### Diary

The Sponsor-developed participant-informed Diary will be used in this study to record data. Participant will be required to keep and fill the Diary daily as delineated in the SoAs (FIG. 2 and FIGS. 4A and 4B).
The relevant metrics measured by this tool daily are:
Study medication
SSc-RP symptoms (Reporting an attack including duration of attack)
Severity of the attack considering all symptoms of the attack e.g. tingling, numbness, pain, color changes (VAS 0-10 cm scale)
Participant's location at the time of the attack (inside home/outside home)
Selecting symptoms experienced during the attack (tingling, numbness, pain, color changes, other)
Pain rating during the attack (11-point Likert scale)
The RCS based on how much difficulty participants had with Raynaud's today, how many attacks the participant had, and how long they lasted. Participants will also be asked to consider how much pain, numbness, or other symptoms the Raynaud's caused in fingers (including painful sores), and how much the Raynaud's alone affected the use of hands today (VAS 0-10 cm scale).

### Metrics Measured at the In-Clinic Visit (Once in a Dosing Period) are:

Digital ulcer severity (VAS 0-10 cm scale)—Applicable only if participant has digital ulcers.
Scleroderma Health Assessment Questionnaire which includes Raynaud's VAS measuring participant quality of life over the past 7 days
UCLA SCTC GIT 2.0 questionnaire, assessing gastrointestinal symptoms (of sclerosis) over the past 7 days.
Assessment of Systemic sclerosis-associated Raynaud's Phenomenon (ASRAP) questionnaire, measuring participant experience with Raynaud's symptoms over the past 7 days.
   Note: Digital ulcer severity (VAS 0-10 cm scale), SHAQ, UCLA SCTC GIT 2.0 and ASRAP questionnaire will be done on paper in-clinic. eExternal temperature will be a feed into the Diary database based on participant's location.

### Physician Assessment of Disease

The Physician will assess the below at each in-clinic visit, details of which will be recorded in the eCRF.
Rating severity of participant's Raynaud's disease
Rating severity of participant's scleroderma symptoms
Rating severity of participant's ulcers how many ulcers, how many considered new, for each ulcer: location and diameter of the ulcer
Rating overall health of participant for past week
Any ocular symptoms, and the nature of these symptoms as ocular manifestations are common in patients with SSc (Kozikowska 2020), and range in incidence and prevalence from over 80% having symptoms of dry eye disease, and >75% having eyelid symptoms and conjunctival symptoms in >70% of patients.
Patient symptoms will be recorded in severity with the patient asked to rate any ocular symptoms or discomfort or pain on a 0-10 scale and the physician will record these answers in the eCRF. AE/SAE reporting.
   Drug accountability, including dispensing and returning of the study medication will also be recorded at each visit.

### Scleroderma Health Assessment Questionnaire (SHAQ)

The standard, validated, patient reported outcome measures tool for SSc patients, the SHAQ, will be collected at the time points specified in the study schedules (FIG. 2 and FIGS. 4A and 4B) to assess the participant's quality of life. The SHAQ includes a Raynaud's VAS that will also be reported separately.

### UCLA Scleroderma Clinical Trials Consortium Gastrointestinal Tract 2.0 (UCLA SCTC GIT 2.0)

The standard, validated, patient reported outcome measures tool for SSc patients, the UCLA SCTC GIT 2.0 will be collected at the time points specified in the study schedules (FIG. 2 and FIGS. 4A and 4B) to assess gastrointestinal symptoms (of sclerosis).

Assessment of Systemic Sclerosis-associated Raynaud's Phenomenon (ASRAP) A novel patient-reported outcome (PRO) questionnaire will be completed by the participant at the time points specified in the Part B study schedule FIGS. 4A and 4B), to assess the severity and impact on daily life of RP in SSc.

### Thermography

Thermography assessments will be performed at the time points specified in the study schedules (FIG. 2 and FIGS. 4A and 4B). Thermography will be conducted on the most severely impacted digits identified at screening; the participant must be indoors for at least 30 minutes prior to the test to give the body time to equilibrate. Photos will be taken with the help of Fluor thermographic camera of these two areas at times specified in SoAs.

### Endothelial Dysfunction (Endo-PAT)

Assessments for endothelial dysfunction will be performed using Endo-PAT at timepoints specified in the study schedules (FIG. 2 and FIGS. 4A and 4B). The Endo-PAT is a diagnostic device used to assess endothelial vasodilator function in a rapid and non-invasive fashion. The below points should be considered before assessment is started:
1. Prior to the study, the participant should fast for at least 4 hours, and should refrain from caffeine, vitamins or medications that might affect vascular tone for at least 8 hours. The participant must reconfirm their abstinence from tobacco and may use the restroom prior to the assessment.
2. The Endo-PAT assessment should be conducted in a quiet, dimly lit, temperature-controlled (25° C. for at least 30 minutes) exam room to reduce fluctuations in vascular tone.
3. Cell phones or paging devices should be silenced, and restrictive clothing that could interfere with blood flow to the arms should be removed. The participant should also remove watches, rings, or other jewelry on the hands or fingers.
4. Participant's fingers should be inspected for any deformities or injuries that could affect the study. The probes should not be placed on a finger that is cut or injured. Fingernails should not extend more than 5 mm or ⅕ of an inch beyond the tip of the finger tissue.
5. The index finger is recommended for the study; however, if this finger is unsuitable, a different digit (except the thumb) may be used, as long as the same finger is used on both hands.
6. The participant should be supine and comfortable for 15 minutes so as to attain a cardiovascular steady state.

### Safety Assessments

### Safety Parameters

Study procedures should be completed as delineated in the SoA (FIG. 2 and FIGS. 4A and 4B). Any unscheduled procedures required for urgent evaluation of safety concerns must take precedence over all routine scheduled procedures.

### Demographic/Medical History

Medical history (including alcohol and smoking status), date of birth, age (calculated), weight, sex, ethnicity, and race will be recorded at randomization (Day 0) visit.

### Vital Signs

Vital signs (SBP, DBP, pulse rate, temperature) will be measured at the time points specified in the SoA (FIG. 2 and FIGS. 4A and 4B) with participants resting for at least 5 minutes in a supine position. When the time of vital signs measurement coincides with a blood draw, the vital signs will be taken before the scheduled blood draw where possible, ensuring the blood draw is within the window specified in the protocol.

Additional vital signs may be performed at other times if deemed necessary.

### Laboratory Assessments

Routine hospital laboratory tests including hematology, biochemistry, inflammatory markers (CRP and ESR), and antibody status (Scl-70) should be conducted as clinically indicated per standard of care but are not required per protocol. If conducted, results will be collected in the eCRF and used to describe the severity of disease in the baseline demographic and disease data. Additional clinical laboratory tests may be performed at other times if deemed necessary, based on the participant's clinical condition.

### Pregnancy Testing

A urine pregnancy test will be performed at the randomization (Day 0) visit and on the last clinic visit at the end of last dosing period for WOCBP only.

### Adverse and Serious Adverse Events

In this study, AEs and SAEs will be reported for all participants from the time of randomization until the completion of the Follow-up/EOS visit. Adverse events that are ongoing at the EOS visit will be marked as Not Recovered/Not resolved on the AE eCRF page (see Section 9.4.4). The Investigator will do full AE review during in-clinic visit. All spontaneously volunteered and enquired for, as well as observed AEs, will be recorded in the participant's medical records and the eCRF.

### Complications of the Disease Under Study

Clinical features and symptoms of SSc-RP must be recorded as endpoints in the electronic data collection tools provided by the Sponsor, as well as in the source documents and should not be reported as AEs.

### Definition of Adverse Events

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.

### Events Meeting the Definition of an AE Include:

Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE).
   Events that do not Meet the Definition of an AE Include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen.
   If there is evidence of an AE through report or observation, the Investigator or designee will evaluate further and record the following information:
Time of onset and resolution
Severity
Seriousness
Causality/relation to study treatment
Action taken regarding study drug
Action taken regarding AE
Outcome.

### Severity of an Adverse Event

Severity of AEs will be graded by the Investigator as one of:
Mild (Grade 1): A type of AE that is usually transient and may require only minimal treatment or therapeutic intervention. The event does not generally interfere with usual activities of daily living.
Moderate (Grade 2): A type of AE that is usually alleviated with additional specific therapeutic intervention. The event interferes with usual activities of daily living, causing discomfort but poses no significant or permanent risk of harm to the research participant.
Severe (Grade 3): A type of AE that interrupts usual activities of daily living, or significantly affects clinical status, or may require intensive therapeutic intervention.
Life-threatening (Grade 4): A type of AE that places the participant at immediate risk of death.
Death (Grade 5): Events that result in death.

### Causal Relationship of an Adverse Event

The Investigator will assess the relationship between study drug and the occurrence of each AE. The Investigator's assessment of the relationship of each AE to study drug will be recorded in the source documents and the eCRF. Alternative causes, such as medical history, concomitant therapy, other risk factors, and the temporal relationship of the event to the study drug should be considered and investigated, if appropriate. The following definitions are general guidelines to help assign grade of attribution:
Not related: The event is clearly related to other factors such as the participant's environment or clinical state, therapeutic interventions or concomitant drugs administered to the participant. This is especially so when an event occurs prior to the commencement of treatment with the study drug.
Unlikely: The temporal association, participant history, and/or circumstances are such that the study drug is not likely to have had an association with the observed event. Other conditions, including concurrent illness, progression, or expression of the disease state, or reaction to a concomitant drug administered appear to explain the event.
Possible: The event follows a reasonable temporal sequence from the time of study drug administration or follows a known response to the study drug but could have been produced by other factors such as the participant's clinical state, other therapeutic interventions, or concomitant drugs administered to the participant.
Probable: The event follows a reasonable temporal sequence from the time of study drug administration and follows a known response to the study drug and cannot be reasonably explained by other factors such as the participant's clinical state, other therapeutic interventions, or concomitant drugs administered to the participant.
Definite: The event follows a reasonable temporal sequence from the time of study drug administration or control abates upon discontinuation or cannot be explained by known characteristics of the participant's clinical state.
   Action Taken with Investigational Products

Should the Investigator need to alter the administration of the study drug from the procedure described in the protocol due to the wellbeing and safety of the participant then the action taken will be recorded on the AE eCRF page, as one of the following options:
Dose Reduced
Drug Interrupted
Drug Withdrawn
Not Applicable
Other.

### Outcome

Outcome of an AE will be recorded on the AE eCRF as follows:
Recovered/Resolved
Recovering/Resolving
Recovered/Resolved with Sequelae
Not Recovered/Not Resolving
Fatal
Unknown.

### Definition of Serious Adverse Event

An SAE is an AE occurring during any study phase (i.e. baseline, treatment, washout, or follow-up), and at any dose of the study drug (active or placebo), that fulfills one or more of the following:
Results in death
It is immediately life-threatening
It requires in participant hospitalization or prolongation of existing hospitalization
It results in persistent or significant disability or incapacity
Results in a congenital abnormality or birth defect.
It is an important medical event that may jeopardize the participant or may require medical intervention to prevent one of the outcomes listed above.
   Important medical events that may not be one of the above may be considered an SAE by the Investigator when, based upon appropriate medical judgment, they are considered clinically significant and may jeopardize the participant, or may require medical or surgical intervention to prevent one of the outcomes listed above.

An AE is considered “life-threatening” if, in the opinion of either the Investigator or the Sponsor, its occurrence places the participant at immediate risk of death. It does not include an AE that, had it occurred in a more severe form, might have caused death.

### Notification of a Serious Adverse Event

All SAEs, regardless of relationship to the study drug, during the period starting from the time of randomization through to the EOS will be recorded in the eCRF. The Investigator will do full SAE review during in-clinic visit. All SAEs will be recorded in the participant's medical records and the eCRF.

Once the Investigator becomes aware of an SAE, they must report the SAE to Sponsor within 24 hours of knowledge of the event.

If requested, supporting de-identified source documents (e.g., hospital discharge summary, autopsy report when available, results of relevant diagnostic tests completed to evaluate the event) will also be sent to the Sponsor.

A written SAE report must include a full description of the event including the below parameters:
Diagnosis or description of event
Onset date
Severity assessment
Causal relationship to the IP
Assessment of seriousness of the event
Corrective treatment administered for the SAE
Action taken related to study drug include the following: dose interruption, dose delay, dose reduction or study drug discontinuation
Outcome of event and end date.
   The Sponsor is responsible for notifying the relevant regulatory authorities of certain events. It is the Investigator's responsibility to notify the HREC of all SAEs in accordance with the HREC SAE reporting policy. The Investigator will also be notified of all unexpected, serious, drug-related events that occur during the clinical study. The investigational site is responsible for notifying its HREC of these additional SAEs.
   Note: Disease progression will not be considered a reportable event.

### Clinical Laboratory Abnormalities and Other Abnormal Assessments as Adverse Events and Serious Adverse Events

Abnormal laboratory findings or other abnormal assessments (e.g. vital signs) per se are not reported as AEs. However, those abnormal findings that are deemed clinically significant by the Investigator(s) and/or delegate or are associated with signs and/or symptoms must be recorded as AEs if they meet the definition of an AE (and recorded as an SAE if they meet the criteria of being serious) as previously described. Clinically significant abnormal laboratory or other abnormal findings that are detected after randomization or that are present at baseline and worsen after randomization are included as AEs (and SAEs if serious).

The Investigator(s) should exercise medical and scientific judgment in deciding whether an abnormal laboratory finding, or other abnormal assessment is clinically significant. To be considered clinically significant, the abnormality should be associated with a clinically evident sign or symptom or be likely to result in an evident sign or symptom in the near term. A clinically significant laboratory abnormality in the absence of clinical symptoms may jeopardize the participant and may require intervention to prevent immediate consequences. For example, a markedly low serum glucose concentration may not be accompanied by coma or convulsions yet be of a magnitude to require glucose administration to prevent such sequelae.

### Recording Adverse Events

Adverse events spontaneously reported by the participant and/or in response to an open question from the study personnel or revealed by observation will be recorded in accordance with the Investigator's normal clinical practice and on the AE page of the eCRF during the study at the investigational site.

However, abnormal values that constitute an SAE or lead to discontinuation of administration of study drug must be reported and recorded as an AE. The AE term should be reported in standard medical terminology when possible. For each AE, the Investigator will evaluate and report the onset (date and time), resolution (date and time), intensity, causality, action taken, serious outcome (if applicable), and whether or not it caused the participant to discontinue the study. AEs that occur during the study must be documented in the participant's medical record, on the AE eCRF and on the SAE report form. If an SAE report is completed, pertinent laboratory data should be recorded on the SAE form, preferably with baseline values and copies of laboratory reports.

In addition, if the abnormal assessment meets the criteria for being serious, the SAE form must also be completed. A diagnosis, if known, or clinical signs or symptoms if the diagnosis is unknown, rather than the clinically significant laboratory finding or abnormal assessment, should be used to complete the AE/SAE page. If no diagnosis is known and clinical signs or symptoms are not present, then the abnormal finding should be recorded.

### Follow-Up of Adverse Events and Serious Adverse Events

All AEs and SAEs will be followed for the duration of the study. The Investigator is responsible for ensuring that follow-up includes any supplemental investigations as may be indicated to elucidate as completely as practical the nature and/or causality of the AE/SAE. This may include additional laboratory tests or investigations or consultation with other health care professionals.

The Sponsor may request that the Investigator perform or arrange for the conduct of supplemental measurements and/or evaluations. If a participant dies during participation in the study or during a recognized Follow-up period, the Sponsor should be provided with a copy of any post-mortem findings, including histopathology.

### Pregnancy

Pregnancy testing should be performed in all WOCBP as per the SoA and the pregnancy results should be captured in the eCRF. All WOCBP will be instructed to contact the Investigator immediately if they suspect they might be pregnant (e.g., missed or late menstrual period) at any time during the study. Male participants will contact the Investigator immediately if they suspect they may have fathered a child during the study treatment period. When possible, the partner's pregnancies should be followed (to term) to determine the outcome.

If a participant becomes pregnant during the clinical study, the Investigator will report the details on a pregnancy form to the Sponsor/assigned designee within 24 hours of knowledge of the pregnancy. Even though participants agree to withdraw or terminate the clinical trial, the Investigator should follow-up and document the process and results of all the pregnancies.

If a male participant's female partner becomes pregnant while enrolled in the study, a pregnancy form should be completed. Abortions (spontaneous, accidental, or therapeutic) must also be reported to the Sponsor. Congenital anomalies/birth defects always meet SAE criteria, and should therefore, be expeditiously reported as an SAE, using the previously described process for SAE reporting. A pregnancy form should also have been previously completed and will need to be updated to reflect the outcome of the pregnancy. The Investigator must report any pregnancy (including pregnancy of a male participant's partner), even if no AE has occurred, on a Pregnancy Report Form within 24 hours of the Investigator becoming aware of the pregnancy.

### Study Drug Materials and Management

### Study Drug Packaging and Labeling

The Sponsor is responsible for the preparation and labeling and providing details of batch numbers, safety, and stability data. The study drug will be labeled in accordance with local regulatory requirements and will be shipped at a temperature below 25° C. within a dry place.

### Study Drug Storage

Upon receipt, the study drug must be stored at controlled room temperature (15° C. to 25° C.) in a tightly closed container. The drug should be protected from excess heat and light and should be kept out of reach of children. The Investigator or designee will be fully responsible for the security, accessibility, and storage of the study drug while it is at the investigational facility.

### Administration

The Investigator or designee is responsible for the education of study staff and participants as to the correct administration of the study drug.

### Study Drug Accountability

A record will be maintained by the investigational site that will account for all dispensing and return of any used and unused study drug. At the end of the study, the study drug will be reconciled, and a copy of the record given to the study monitor.

### Study Drug Handling and Disposal

On completion of the study, any surplus study drug supplies will be destroyed upon receipt of written approval from the Sponsor. Evidence of the destruction of any surplus study drug will be supplied to the study monitor. If no supplies remain, this will be documented in the dispensing record.

### Statistics

Statistical methods will be further outlined in the statistical analysis plan (SAP) and approved by the Sponsor. Procedures outlined in the SAP will supersede protocol specified statistical methods in the event of divergence.

Part A and Part B data will be analyzed separately. Analysis of Part A data will be mainly exploratory to support cilnidipine dose selection and treatment effect check for sample size confirmation for Part B. For Part B, the primary and secondary efficacy analyses will be based on the mITT population. Analyses based on the ITT and PP population will be considered secondary and confirmatory. All safety analyses will be performed on the safety population.

In general, descriptive statistics (e.g. arithmetic mean, SD, median, minimum and maximum) will be calculated for continuous safety data by treatment and protocol specified time point, while frequency summary (e.g. number of observed and percentage of each categories) will be applied for categorical safety data by treatment and protocol specified time point.

### Sample Size

Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned. After reviewing the results from Part A: Run-in phase, the sample size for Part B may be adjusted.

Following from the decision of the committee to exclude tadalafil from Part B of the study, the design was changed to a two-treatments, two-periods (2×2) crossover. The sample size for Part B was calculated based on the available data from Part A at the time of the protocol specified data review. Assuming a 2-sided 0.05 alpha for the comparison of cilnidipine 20 mg versus placebo, a between-participants standard deviation (SD) of 35, a correlation between the two measurements on the same participant of 0.2, and a 25% dropout rate, it is estimated that a total sample size of 38 participants (19 in each treatment sequence) is needed, in order to obtain complete data from 28 participants (14 in each treatment sequence), for ≥80% power to detect a decrease of 25 in percentage change from baseline in weekly RP attacks in a 2×2 crossover design.

### Analysis Populations

Participant inclusion into each population will be determined prior to the final analysis.

### Intent-To-Treat (ITT) Population

All participants who are entered into the study and complete screening, sign an informed consent for the study and randomized will be included in the ITT population.

Modified Intent-To-Treat (mITT) Population

All participants who are entered into the study and complete screening, sign an informed consent for the study and randomized and have post-baseline attack data (primary endpoint data) will be included in the mITT population.

### Per Protocol Population

All participants who complete the study with all dosing periods (for Part B— at least 5 days of dosing within the last 7 days treatment) and meet all eligibility criteria, and without any major/important protocol deviations, will be included in the PP Analysis Population.

### Pharmacokinetic Population

All participants who receive any amount of active study drug and have sufficiently evaluable concentration time profile to allow determination of at least one PK parameter will be included in the PK population. An evaluable PK profile will be determined at the discretion of the pharmacokineticist following examination of participants with dosing or protocol deviations that could potentially affect the PK profile. The PK population will be used for the summaries of all PK data.

### Safety Population

All randomized participants who received study drug will be included in Safety population and will be classified according to the actual treatment received.

### Statistical Methods

### Participant Disposition

Participant disposition will be analyzed using counts and percentages. The number and percentage of screened participants, enrolled participants, treated participants, participants discontinued from the study and study treatment, as well as the primary reason for discontinuation will be analyzed and listed.

### Demographics, Medical History, and Baseline Characteristics

Demography and baseline characteristics data will be summarized using descriptive statistics. The following demographic variables will be summarized by dose level: race, gender, age, height and weight, concomitant diseases (Hypertension, peripheral vascular disease, diabetes, CKD and stage, osteoporosis, history and type of heart arrhythmia).

In addition, the following baseline characteristics of Raynauds Disease will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

### Prior and Concomitant Medication

Prior and concomitant medications will be coded using the most current version of the WHO drug dictionary available at the start of the study. Prior and concomitant medications will be listed by participant and summarized by treatment using anatomical therapeutic chemical (ATC) and preferred name.

### Treatment Compliance and Exposure

Treatment compliance and exposure will be summarized and listed by treatment for all participants in the Safety population.

### Analysis of Primary Endpoint

Percent change from baseline evaluation for frequency of weekly RP attacks will be the primary efficacy endpoint. Data collected in the last 7 days of each dosing period will be used for this analysis. Screening assessments will be used as baseline for the analysis of all periods. No multiple comparison adjustment will be used to control alpha for the multiple comparisons. Mixed effects model will be used for analysis of the primary endpoint according to the crossover design. Other efficacy endpoints of continuous variables will be analyzed using similar methodology. For nominal data, chi-square tests will be applied. Generalized Estimating Equations method will be used, as appropriate, for adjusting for potential confounding factors. In addition, the final analysis will assess whether in this study of severe Raynaud's disease participants, the minimally important difference, previously concluded of 14-15 points on the 100 point RCS scale has been achieved in the cilnidipine dose group. It also will record the percentage of participants achieving a PASS (34 point difference from baseline on a 0-100 VAS) in each treatment group.

### Analysis of Secondary Endpoints

The secondary endpoints of change from baseline evaluation will be compared among treatment groups using mixed effects model. Kaplan-Meier method will be used to evaluate time to event endpoints. To evaluate the impact of daily ambient temperature on symptomatic Raynaud's attack, logistic regression will be used for temperature versus the occurrence of Raynaud's attack (Yes/No).

The effect of temperature on the severity score of Raynaud's attacks and difference of using rescue medication between treatment groups will be evaluated by Chi-square test. The impact of therapy in sympathetic activity will be assessed by mixed model for repeated measures.

### Safety Analyses

All safety assessments, including AEs, laboratory evaluations, vital signs, and other safety assessments will be analyzed using the Safety population.

### Adverse Event

Adverse events will be coded using the most current version of the MedDRA® Version 22.0 or higher. The analysis of AEs will be based on the concept of treatment emergent AEs. Treatment emergent AEs will be tabulated by treatment group and will include the number of participants for whom the event occurred, the severity, and relationship to study drug. Treatment emergent AEs (TEAEs) leading to discontinuation and SAEs with onset after the start of study drug will also be summarized. All AEs and SAEs (including those with onset or worsening before the start of study drug) through the end of the study will be listed.

### Laboratory Evaluations

Baseline laboratory evaluations will be listed and summarized by treatment.

### Vital Signs

Vital signs (BP [systolic and diastolic], pulse rate, and oral temperature) will be listed and summarized by treatment and protocol specified collection time point. Observed and change from baseline will be summarized at each protocol specified collection time point.

### Other Safety Assessments

The following assessments will be listed by participant:
Pregnancy Test
Raynaud's function assessment by physician.

### Pharmacokinetics

Plasma concentrations and actual blood sampling times will be listed by treatment and protocol specified time point and summarized using descriptive statistics number of measurements, arithmetic mean, SD, and % CV, geometric mean, minimum, median, and maximum—at each scheduled time point. Individual and mean plasma concentration-time profiles will also be presented graphically for each treatment. Pharmacokinetic parameters will be computed from the individual plasma concentrations using a non-compartmental approach.

Value for elimination rate constant (kel), elimination half-life (t½), Area under the plasma concentration-time curve from time zero to infinity (AUC0-inf), apparent total clearance of the drug from plasma after oral administration (CL/F) or apparent volume of distribution during terminal phase after non-intravenous administration (Vz/F) will not be reported. Additional analyses will be performed as deemed necessary upon review of the data.

### Ethics

### Ethics Review

The final study protocol, including the final version of the ICF, must be approved or given a favorable opinion in writing by an HREC as appropriate. The Investigator must submit written approval to the Sponsor before they can enroll any participant into the study.

The Principal Investigator (PI) is responsible for informing the HREC of any amendment to the protocol in accordance with local requirements. In addition, the HREC must approve all advertising used to recruit participants for the study. The protocol must be re-approved by the HREC upon receipt of amendments and annually, as local regulations require.
The PI is also responsible for providing the HREC with reports of any reportable serious adverse drug reactions from any other study conducted with the study drug (active). The Sponsor will provide this information to the PI. Progress reports and notifications of serious adverse drug reactions will be provided to the HREC according to local regulations and guidelines.

### Ethical Conduct of the Study

The study will be performed in accordance with ethical principles that have their origin in the Declaration of Helsinki (Ethical Principles for Medical Research Involving Human Subjects) and are consistent with ICH GCP applicable regulatory requirements.

Results: Patients in the study were administered the Scleroderma Health Assessment Questionnaire (SHAQ) at the time of study entry and during clinic visits following the treatment period. The study is a two-part, parallel arm design, prospective, double-blind, randomized study conducted in Australia (Flinders Medical Center). 27 patients were randomized into the first phase of the study, following which a DSMB meeting was held, which reviewed data on the primary study endpoint and key Raynaud's secondary endpoints. Data on the SHAQ was not available for the DSMB to review. The mITT population, in which any data was available post treatment was 24 patients was the analysis population for this first part of the study. The second part (i.e., part B) of the study will randomize 36 patients in a double blind, randomized, prospective, placebo controlled, crossover study. In the first part (i.e., Part A) of the study for which data is presented, patients were randomized into 6 groups, cilnidipine at two doses (10 and 20 mg) alone and in combination with tadalafil 5 mg, tadalafil 5 mg alone, and placebo. The DSMB found both doses of cilnidipine to be effective at reducing Raynaud's endpoints and increased effect with 20 mg compared to 10 mg of cilnidipine. The DSMB recommended that the study proceed into its double blind prospective cross overpowered second phase and compare cilnidipine 20 mg alone to placebo. Data from the SHAQ analysis suggests the following and is first presented in summary format and then data tables are provided.
a. Cilnidipine treatment may reduce patient-reported SSc disease disability compared to placebo
b. Cilnidipine treatment may reduce patient-reported SSc disease alternative disability compared to placebo
c. Cilnidipine treatment may reduce patient-reported SSc disease pain* compared to placebo
d. Cilnidipine treatment may reduce patient-reported SSc disease severity compared to placebo
e. Cilnidipine treatment may reduce patient-reported SSc disease skin ulcer severity rating compared to placebo
f Cilnidipine treatment may reduce patient-reported SSc disease breathing difficulty compared to placebo
g. Cilnidipine treatment, in combination with tadalafil (a known −P glycoprotein P inhibitor that increases brain passage and central concentrations of cilnidipine (cilnidipine when given orally is 9800 protein bound, largely by PGP)) may further reduce patient-reported SSc disease pain compared to placebo. SSc disease pain comprises not only pain from Raynaud's but joint pain, headache, odynophagia, back pain, pain from skin ulceration, and neuropathic pain).
   Table E includes results of the Scleroderma Health Assessment Questionnaire.

TABLE E

| | Pooled Cilnidipine | Placebo |
|---|---|---|
| Statistics | (N = 7) | (N = 4) |
| Baseline | | |
| n | 7 | 4 |
| Mean (SD) | 2.700 (2.3424) | 2.000 (1.7963) |
| Median | 2.900 | 1.300 |
| Q1, Q3 | 0.400, 3.400 | 0.800, 3.200 |
| Min, Max | 0.00, 7.10 | 0.80, 4.60 |
| In-Clinic Visit (Day 12) | | |
| n | 7 | 4 |
| Mean (SD) | 1.486 (1.7743) | 2.700 (1.9511) |
| Median | 1.200 | 2.950 |
| Q1, Q3 | 0.200, 1.900 | 1.400, 4.000 |
| Min, Max | 0.00, 5.20 | 0.10, 4.80 |
| Change from Baseline | | |
| n | 7 | 4 |
| Mean (SD) | −1.214 (0.7946) | 0.700 (1.3089) |
| Median | −1.500 | 0.550 |
| Q1, Q3 | −1.800, −0.400 | −0.250, 1.650 |
| Min, Max | −1.90, 0.20 | −0.70, 2.40 |
| Percent Change | | |
| from Baseline | | |
| n | 6 | 4 |
| Mean (SD) | −59.80 (25.642) | 66.71 (165.704) |
| Median | −55.86 | 27.17 |
| Q1, Q3 | −76.19, −44.12 | −41.58, 175.00 |
| Min, Max | −100.0, −26.8 | −87.5, 300.0 |

Table E shows that in the pooled cilnidipine patients who received either 10 mg or 20 mg of cilnidipine (n=7), there was a mean 60% decrease in patient reported disease severity on the SHAQ, versus an increase in severity in 4 placebo treated patients.
Table F shows a summary table comparing 20 mg of cilnidipine to placebo treated patients on the SHAQ parameters in the mITT population. Table F shows substantial benefits in the SHAQ for 20 mg treated and pooled cilnidipine patients versus placebo patients on most of the parameters measured.

TABLE F

| | Pooled | Cilnidipine | |
|---|---|---|---|
| | Cilnidipine | 20 mg | Placebo |
| PARAMETER | (n = 7) | (n = 3) | (n = 4) |
| % change in | −31% | −53% | +7% |
| Pain | | | |
| % change in | −25% | −50% | −16% |
| Standard | | | |
| Disability Index | | | |
| % change in | −13% | −42% | −13% |
| Alternative | | | |
| Disability Index | | | |
| % improvement | 41% (median) | 67% (median) | −7% (worsening) |
| in Breathing | | | |
| % improvement | 19% | 35% | −4% (worsening) |
| in VAS Digital | | | |
| Ulcers | | | |
| % change VAS | −17% | −49% | −42% |
| Raynauds | | | |
| % change | −60% (i.e., | −63% (i.e., | 67% (i.e., |
| overall SSc | improvement) | improvement) | worsening) |
| Disease Severity | | | |

Pain on the SHAQ outcome measure on the first line in table F is a composite of all pain experienced in patients with SSc, comprising joint pain, back pain, headache, odynophagia, skin ulcer related discomfort, Raynaud's, and neuropathic pain. Aisa Pharma believes, based on the biologic attributes and actions of cilnidipine that differ from most dihydropyridine calcium channel antagonists that cilnidipine might have direct analgesic effects in certain pain conditions and in fact has been designated to receive evaluation through the NIH-NINDS Preclinical Pain Screening Platform Program to investigate the drug in multiple in vitro and in vivo preclinical models of pain that are validated and predictive of effects in man.

Analysis of data that may support an increased analgesic effect of cilnidipine on Raynaud's pain, when given in combination with tadalafil, which increases brain concentrations of cilnidipine by competitively inhibiting the protein (p-glycoprotein p) which is largely responsible for 98% protein binding of cilnidipine in plasma when the drug is given orally, inhibiting transmission across the blood-brain barrier. Given the small numbers of patients in the first part of the study, baseline pain differences vary amongst the groups, but a trend towards an increased reduction of Raynaud's VAS pain is seen in both the 10 mg cilnidipine (*) and 20 mg cilnidipine (†) groups.

Table G shows data that supports an increased analgesic effect of cilnidipine on Raynaud's pain.

| | | | | Percent |
|---|---|---|---|---|
| | | | | Change from |
| | Baseline | | | Baseline |
| | Average | Average | Change From | (red = reduction, |
| Group | Pain Score | Pain Score | Baseline | black = increase) |
| (N = 24) | [Mean (SD)] | [Mean (SD)] | [Mean (SD)] | [Mean (SD)] |
| Cilnidipine | 1.76 (0.885) | 1.28 (1.993) | −0.47 (2.025) | −26.89 (89.725)* |
| 20 mg, mean | | | | |
| (SD) (n = 3) | | | | |
| Cilnidipine | 2.35 (1.758) | 2.82 (2.268) | 0.47 (0.558) | 11.76 (27.478)† |
| 10 mg, mean | | | | |
| (SD) (n = 4) | | | | |
| Tadalafil | 5.47 (3.586) | 5.84 (4.768) | 0.37 (2.219) | 17.20 (64.838) |
| 5 mg, (n = 4) | | | | |
| Cilnidipine | 5.23 (1.445) | 2.26 (2.208) | −2.97 (1.399) | −61.84 (34.512)* |
| 10 mg + | | | | |
| Tadalafil | | | | |
| 5 mg, (n = 5) | | | | |
| Cilnidipine | 4.16 (2.427) | 3.39 (3.692) | −0.77 (2.677) | −24.00 (54.180)† |
| 20 mg + | | | | |
| Tadalafil | | | | |
| 5 mg, (n = 4) | | | | |
| Placebo, mean | 2.31 (1.623) | 1.68 (0.828) | −0.63 (1.346) | −3.92 (67.665) |
| (SD) (n = 4) | | | | |

Tables H-O shows listings for each parameter on the SHAQ assessment in the MITT population.
Table H is a SHAQ-Change from baseline and Percent change from baseline-Modified ITT Population-Part A. Standard disability index.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 4 | |
| Mean (SD) | 0.656 | (0.7731) | 0.250 | (0.2165) | 0.594 | (0.4719) | 0.550 | (0.4108) | 0.781 | (0.8377) | 0.813 | (0.6654) |
| Median | 0.563 | | 0.375 | | 0.625 | | 0.500 | | 0.750 | | 0.813 | |
| Q1, Q3 | 0.000, 1.313 | | 0.000, 0.375 | | 0.250, 0.938 | | 0.250, 0.500 | | 0.063, 1.500 | | 0.375, 1.250 | |
| Min, Max | 0.00, 1.50 | | 0.00, 0.38 | | 0.00, 1.13 | | 0.25, 1.25 | | 0.00, 1.63 | | 0.00, 1.63 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 0.656 | (0.7731) | 0.125 | (0.1250) | 0.531 | (0.4130) | 0.391 | (0.3370) | 0.714 | (0.7786) | 0.719 | (0.7526) |
| Median | 0.563 | | 0.125 | | 0.563 | | 0.250 | | 0.554 | | 0.563 | |
| Q1, Q3 | 0.000, 1.313 | | 0.000, 0.250 | | 0.250, 0.813 | | 0.188, 0.500 | | 0.125, 1.304 | | 0.188, 1.250 | |
| Min, Max | 0.00, 1.50 | | 0.00, 0.25 | | 0.00, 1.00 | | 0.13, 1.13 | | 0.00, 1.75 | | 0.00, 1.75 | |
| Change from | | | | | | | | | | | | |
| Baseline | | | | | | | | | | | | |
| n | 4 | | 3 | | 4 | | 8 | | 4 | | 4 | |
| Mean (SD) | 0.0 | (0.0000) | −0.125 | (0.1250) | −0.063 | (0.0722) | −0.141 | (0.2449) | −0.067 | (0.3063) | −0.094 | (0.2772) |
| Median | 0.000 | | −0.125 | | −0.063 | | −0.188 | | 0.063 | | 0.000 | |
| Q1, Q3 | 0.000, 0.000 | | −0.250, 0.000 | | −0.125, 0.000 | | −0.313, −0.063 | | −0.259, 0.125 | | −0.250, 0.063 | |
| Min, Max | 0.00, 0.00 | | −0.25, 0.00 | | −0.13, 0.00 | | −0.38, 0.38 | | −0.52, 0.13 | | −0.50, 0.13 | |
| Percent | | | | | | | | | | | | |
| Change from | | | | | | | | | | | | |
| Baseline | | | | | | | | | | | | |
| n | 2 | | 2 | | 3 | | 8 | | 3 | | 3 | |
| Mean (SD) | 0.0 | (0.000) | −50.00 | (23.570) | −9.26 | (8.486) | −16.88 | (72.896) | 23.34 | (70.153) | −16.48 | (35.421) |

| Median | 0.00 | −50.00 | −11.11 | −37.50 | 7.69 | 0.00 |
|---|---|---|---|---|---|---|
| Q1, Q3 | 0.00, 0.00 | −66.67, −33.33 | −16.67, 0.00 | −62.50, −5.00 | −37.66, 100.00 | −57.14, 7.69 |
| Min, Max | 0.0, 0.0 | −66.7, −33.3 | −16.7, 0.0 | −75.0, 150.0 | −37.7, 100.0 | −57.1, 7.7 |

TABLE I
Change from baseline and Percent change from baseline-Modified ITT Population-Part A. Parameter is the alternative disability index.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 4 | |
| Mean (SD) | 0.500 | (0.5863) | 0.208 | (0.1909) | 0.469 | (0.5141) | 0.425 | (0.4644) | 0.688 | (0.7672) | 0.594 | (0.4719) |
| Median | 0.438 | | 0.250 | | 0.375 | | 0.250 | | 0.563 | | 0.625 | |
| Q1, Q3 | 0.000, 1.000 | | 0.000, 0.375 | | 0.063, 0.875 | | 0.250, 0.250 | | 0.063, 1.313 | | 0.250, 0.938 | |
| Min, Max | 0.00, 1.13 | | 0.00, 0.38 | | 0.00, 1.13 | | 0.13, 1.25 | | 0.00, 1.63 | | 0.00, 1.13 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 0.563 | (0.6575) | 0.125 | (0.1250) | 0.375 | (0.4787) | 0.328 | (0.3532) | 0.616 | (0.6594) | 0.531 | (0.75242) |
| Median | 0.500 | | 0.125 | | 0.250 | | 0.250 | | 0.482 | | 0.438 | |
| Q1, Q3 | 0.000, 1.125 | | 0.000, 0.250 | | 0.000, 0.750 | | 0.125, 0.375 | | 0.125, 1.107 | | 0.188, 0.875 | |
| Min, Max | 0.00, 1.25 | | 0.00, 0.25 | | 0.00, 1.00 | | 0.00, 1.13 | | 0.00, 1.50 | | 0.00, 1.25 | |
| Change from | | | | | | | | | | | | |
| Baseline | | | | | | | | | | | | |
| n | 4 | | 3 | | 4 | | 8 | | 4 | | 4 | |
| Mean (SD) | 0.063 | (0.2165) | −0.083 | (0.0722) | −0.094 | (0.0625) | −0.031 | (0.1860) | −0.071 | (0.1756) | −0.063 | (0.2165) |
| Median | 0.000 | | −0.125 | | −0.125 | | −0.063 | | −0.063 | | 0.000 | |
| Q1, Q3 | −0.063, 0.188 | | −0.125, 0.000 | | −0.125, −0.063 | | −0.125, −0.000 | | −0.205, 0.063 | | −0.188, 0.063 | |
| Min, Max | −0.13, 0.38 | | −0.13, 0.00 | | −0.13, 0.00 | | −0.25, 0.38 | | −0.29, 0.13 | | −0.38, 0.13 | |
| Percent | | | | | | | | | | | | |
| Change from | | | | | | | | | | | | |
| Baseline | | | | | | | | | | | | |
| n | 2 | | 2 | | 3 | | 8 | | 3 | | 3 | |
| Mean (SD) | 15.87 | (38.161) | −41.67 | (11.785) | −43.70 | (48.956) | 11.25 | (121.941) | 21.25 | (68.998) | −12.96 | (32.553) |

| Median | 15.87 | −41.67 | −20.00 | −5.00 | −7.69 | 0.00 |
|---|---|---|---|---|---|---|
| Q1, Q3 | −11.11, 42.86 | −50.00, −33.33 | −100.00, −11.11 | −50.00, 0.00 | −28.57, 100.00 | −50.00, 11.11 |
| Min, Max | −11.1, 42.9 | −50.0, −33.3 | −100.0, −11.1 | −100.0, 300.0 | −28.6, 100.0 | −50.0, 11.1 |

TABLE J
Change from baseline and Percent change from baseline-Modified ITT Population-Part A. Visual Analog Scale-Pain.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 3 | | 5 | | 3 | | 4 | |
| Mean (SD) | 1.975 | (1.5283) | 2.167 | (1.7010) | 5.967 | (3.4790) | 4.320 | (2.5352) | 3.167 | (1.1240) | 3.325 | (2.2366) |
| Median | 2.150 | | 1.500 | | 7.400 | | 3.200 | | 2.900 | | 3.500 | |
| Q1, Q3 | 0.850, 3.100 | | 0.900, 4.100 | | 2.000, 8.500 | | 3.100, 5.200 | | 2.200, 4.400 | | 1.500, 5.150 | |
| Min, Max | 0.00, 3.60 | | 0.90, 4.10 | | 2.00, 8.50 | | 1.80, 8.30 | | 2.20, 4.40 | | 0.70, 5.60 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 1.875 | (1.6641) | 1.067 | (0.9074) | 5.300 | (4.1577) | 3.463 | (1.5250) | 2.700 | (1.7907) | 4.100 | (3.1145) |
| Median | 1.950 | | 0.700 | | 5.850 | | 4.050 | | 2.150 | | 4.450 | |
| Q1, Q3 | 0.500, 3.250 | | 0.400, 2.100 | | 1.900, 8.700 | | 2.600, 4.200 | | 1.650, 3.750 | | 1.600, 6.600 | |
| Min, Max | 0.00, 3.60 | | 0.40, 2.10 | | 0.30, 9.20 | | 0.50, 5.50 | | 1.20, 5.30 | | 0.30, 7.20 | |
| Change from | | | | | | | | | | | | |
| Baseline | | | | | | | | | | | | |
| n | 4 | | 3 | | 3 | | 8 | | 3 | | 4 | |
| Mean (SD) | −0.100 | (0.4243) | −1.100 | (0.7937) | 1.000 | (0.4359) | −0.438 | (1.3362) | −0.300 | (1.3115) | 0.775 | (1.2285) |
| Median | 0.000 | | −0.800 | | 0.800 | | −0.550 | | −0.100 | | 0.500 | |
| Q1, Q3 | −0.350, 0.150 | | −2.000, −0.500 | | 0.700, 1.500 | | −1.200, 0.900 | | −1.700, 0.900 | | −0.000, 1.550 | |
| Min, Max | −0.70, 0.30 | | −2.00, −0.50 | | 0.70, 1.50 | | −2.80, 1.00 | | −1.70, 0.90 | | −0.40, 2.50 | |
| Percent | | | | | | | | | | | | |
| Change from | | | | | | | | | | | | |
| Baseline | | | | | | | | | | | | |
| n | 3 | | 3 | | 3 | | 8 | | 3 | | 4 | |
| Mean (SD) | −9.88 | (27.711) | −52.56 | (3.454) | 31.35 | (37.825) | −9.34 | (36.544) | −14.24 | (40.419) | 7.32 | (46.946) |

| Median | 0.00 | −53.33 | 10.81 | −16.83 | −4.55 | 16.61 |
|---|---|---|---|---|---|---|
| Q1, Q3 | −41.18, 11.54 | −55.56, −48.78 | 8.24, 75.00 | −28.16, 28.13 | −58.62, 20.45 | −25.00, 39.64 |
| Min, Max | −41.2, 11.5 | −55.6, −48.8 | 8.2, 75.0 | −72.2, 31.3 | −58.6, 20.5 | −57.1, 53.2 |

TABLE K
Change from baseline and Percent change from baseline-Modified ITT Population-Part A. Visual Analog Scale-Intestinal Problems.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 3 | |
| Mean (SD) | 2.975 | (3.4568) | 0.833 | (0.7638) | 3.150 | (3.7009) | 2.020 | (1.9930) | 0.925 | (0.9069) | 1.100 | (1.5620) |
| Median | 2.550 | | 1.000 | | 2.600 | | 1.100 | | 0.950 | | 0.300 | |
| Q1, Q3 | 0.050, 5.900 | | 0.000, 1.500 | | 0.050, 6.250 | | 0.700, 2.500 | | 0.150, 1.700 | | 0.100, 2.900 | |
| Min, Max | 0.00, 6.80 | | 0.00, 1.50 | | 0.00, 7.40 | | 0.50, 5.30 | | 0.00, 1.80 | | 0.10, 2.90 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 1.100 | (1.8221) | 1.133 | (1.0599) | 3.025 | (4.3162) | 1.663 | (1.9227) | 1.175 | (2.2187) | 0.450 | (0.4509) |
| Median | 0.300 | | 1.300 | | 1.450 | | 1.350 | | 0.100 | | 0.300 | |
| Q1, Q3 | 0.000, 2.200 | | 0.000, 2.100 | | 0.050, 6.000 | | 0.350, 1.950 | | 0.000, 2.350 | | 0.150, 0.750 | |
| Min, Max | 0.00, 3.80 | | 0.00, 2.10 | | 0.00, 9.20 | | 0.00, 6.00 | | 0.00, 4.50 | | 0.10, 1.10 | |
| Change from | | | | | | | | | | | | |
| Baseline | | | | | | | | | | | | |
| n | 4 | | 3 | | 4 | | 8 | | 4 | | 3 | |
| Mean (SD) | −1.875 | (2.1838) | 0.300 | (0.3000) | −0.125 | (1.6800) | −0.538 | (2.1547) | −0.250 | (1.8982) | −0.567 | (1.0970) |
| Median | −1.550 | | 0.300 | | 0.000 | | −0.800 | | −0.150 | | −0.200 | |
| Q1, Q3 | −3.700, −0.050 | | 0.000, 0.600 | | −1.150, 0.900 | | −2.000, 0.600 | | −0.950, 1.450 | | −1.800, 0.300 | |
| Min, Max | −4.40, 0.00 | | 0.00, 0.60 | | −2.30, 1.80 | | −3.40, 3.50 | | −1.60, 2.90 | | −1.80, 0.30 | |
| Percent | | | | | | | | | | | | |
| Change from | | | | | | | | | | | | |
| Baseline | | | | | | | | | | | | |
| n | 3 | | 2 | | 3 | | 8 | | 3 | | 3 | |
| Mean (SD) | −77.37 | (29.418) | 35.00 | (7.071) | −6.92 | (35.225) | −2.66 | (99.762) | −2.55 | (159.269) | 57.09 | (210.380) |

| Median | −88.00 | 35.00 | 0.00 | −40.00 | −88.89 | −62.07 |
|---|---|---|---|---|---|---|
| Q1, Q3 | −100.00, −44.12 | 30.00, 40.00 | −45.10, 24.32 | −82.08, 90.00 | −100.00, 181.25 | −66.67, 300.00 |
| Min, Max | −100.0, −44.1 | 30.0, 40.0 | −45.1, 24.3 | −100.0, 142.9 | −100.0, 181.3 | −66.7, 300.0 |

TABLE L
Change from baseline and Percent change from baseline-Modified ITT Population-Part A. Visual Analog Scale-Breathing Problems

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 4 | |
| Mean (SD) | 1.875 | (3.7500) | 1.133 | (0.9018) | 3.800 | (3.0144) | 1.220 | (0.9550) | 1.025 | (1.4930) | 0.525 | (0.5852) |
| Median | 0.000 | | 1.200 | | 4.050 | | 1.200 | | 0.450 | | 0.250 | |
| Q1, Q3 | 0.000, 3.750 | | 0.200, 2.000 | | 1.550, 6.050 | | 0.800, 1.500 | | 0.050, 2.000 | | 0.200, 0.850 | |
| Min, Max | 0.00, 7.50 | | 0.20, 2.00 | | 0.00, 7.10 | | 0.00, 2.60 | | 0.00, 3.20 | | 0.20, 1.40 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 1.575 | (3.1500) | 0.300 | (0.2646) | 4.300 | (4.8840) | 3.188 | (4.2680) | 1.950 | (3.3131) | 0.450 | (0.3416) |
| Median | 0.000 | | 0.400 | | 4.000 | | 1.300 | | 0.450 | | 0.400 | |
| Q1, Q3 | 0.000, 3.150 | | 0.000, 0.500 | | 0.100, 8.500 | | 0.350, 6.100 | | 0.100, 3.800 | | 0.200, 0.700 | |
| Min, Max | 0.00, 6.30 | | 0.00, 0.50 | | 0.00, 9.20 | | 0.00, 10.00 | | 0.00, 6.90 | | 0.10, 0.90 | |
| Change from | | | | | | | | | | | | |
| Baseline | | | | | | | | | | | | |
| n | 4 | | 3 | | 4 | | 8 | | 4 | | 4 | |
| Mean (SD) | −0.300 | (0.6000) | −0.833 | (1.1504) | 0.500 | (2.5599) | 1.450 | (4.1604) | 0.925 | (1.9138) | −0.075 | (0.3500) |
| Median | 0.000 | | −0.800 | | 1.050 | | −0.400 | | 0.300 | | −0.050 | |
| Q1, Q3 | −0.600, 0.000 | | −2.000, 0.300 | | −1.450, 2.450 | | −0.950, 3.700 | | −0.300, 2.150 | | −0.350, 0.200 | |
| Min, Max | −1.20, 0.00 | | −2.00, 0.30 | | −2.90, 2.80 | | −1.90, 8.80 | | −0.60, 3.70 | | −0.50, 0.30 | |
| Percent | | | | | | | | | | | | |
| Change from | | | | | | | | | | | | |
| Baseline | | | | | | | | | | | | |

| n | 1 | 3 | | 3 | | 7 | | 3 | | 4 | |
|---|---|---|---|---|---|---|---|---|---|---|---|
| Mean (SD) | −16.00 | −5.56 | (135.742) | −2.66 | (79.815) | 108.64 | (303.929) | 213.54 | (347.990) | 24.40 | (97.190) |

| Median | −16.00 | −66.67 | 29.58 | −26.67 | 115.63 | 7.14 |
|---|---|---|---|---|---|---|
| Q1, Q3 | −16.00, −16.00 | −100.00, 150.00 | −93.55, 56.00 | −73.08, 284.62 | −75.00, 600.00 | −51.19, 100.00 |
| Min, Max | −16.0, −16.0 | −100.0, 150.0 | −93.5, 56.0 | −100.0, 733.3 | −75.00, 600.0 | −66.7, 150.0 |

TABLE M
Change from baseline and Percent change from baseline-Modified ITT Population-Part A. Visual Analog Scale-Raynaud's.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 4 | |
| Mean (SD) | 1.600 | (1.9270) | 2.300 | (1.3115) | 5.775 | (4.0252) | 3.640 | (2.9057) | 2.325 | (3.9911) | 3.250 | (2.3274) |
| Median | 1.000 | | 2.500 | | 6.400 | | 2.400 | | 0.500 | | 2.550 | |
| Q1, Q3 | 0.450, 2.750 | | 0.900, 3.500 | | 3.350, 8.200 | | 1.400, 5.000 | | 0.200, 4.450 | | 1.750, 4.750 | |
| Min, Max | 0.00, 4.40 | | 0.90, 3.50 | | 0.30, 10.00 | | 1.30, 8.10 | | 0.00, 8.30 | | 1.30, 6.60 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 1.925 | (1.9207) | 1.033 | (0.4041) | 4.950 | (3.7749) | 3.400 | (2.0626) | 1.500 | (2.4752) | 2.750 | (3.6538) |
| Median | 1.700 | | 1.100 | | 5.200 | | 3.400 | | 0.400 | | 1.500 | |
| Q1, Q3 | 0.400, 3.450 | | 0.600, 1.400 | | 2.200, 7.700 | | 2.050, 4.450 | | 0.150, 2.850 | | 0.150, 5.350 | |
| Min, Max | 0.00, 4.30 | | 0.60, 1.40 | | 0.20, 9.20 | | 0.40, 7.00 | | 0.00, 5.20 | | 0.10, 7.90 | |
| Change from | | | | | | | | | | | | |
| Baseline | | | | | | | | | | | | |
| n | 4 | | 3 | | 4 | | 8 | | 4 | | 4 | |
| Mean (SD) | 0.325 | (1.0468) | −1.267 | (1.0599) | −0.825 | (0.9674) | −0.750 | (1.8150) | −0.825 | (1.5262) | −0.500 | (1.4306) |
| Median | 0.350 | | −1.100 | | −0.500 | | −0.500 | | −0.150 | | −0.650 | |
| Q1, Q3 | −0.500, 1.150 | | −2.400, −0.300 | | −1.500, −0.150 | | −1.600, 0.150 | | −1.700, 0.050 | | −1.600, 0.600 | |
| Min, Max | −0.90, 1.50 | | −2.40, −0.30 | | −2.20, −0.10 | | −4.10, 2.00 | | −3.10, 0.10 | | −2.00, 1.30 | |
| Percent | | | | | | | | | | | | |
| Change from | | | | | | | | | | | | |
| Baseline | | | | | | | | | | | | |
| n | 3 | | 3 | | 4 | | 8 | | 3 | | 4 | |
| Mean (SD) | 11.36 | (118.770) | −48.63 | (18.070) | −19.71 | (16.460) | −16.67 | (36.309) | −20.78 | (40.151) | −41.74 | (58.354) |

| Median | −2.27 | −44.00 | −20.67 | −11.00 | −37.35 | −47.18 |
|---|---|---|---|---|---|---|
| Q1, Q3 | −100.00, −136.36 | −68.57, −33.33 | −33.85, −5.56 | −47.31, 6.25 | −50.00, 25.00 | −91.61, 8.12 |
| Min, Max | −100.0, 136.4 | −68.6, −33.3 | −34.4, −3.1 | −69.2, 40.0 | −50.00, 25.0 | −92.3, 19.7 |

TABLE N
Change from baseline and Percent change from baseline-Modified ITT Population-Part A. Finger Ulcers.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 3 | |
| Mean (SD) | 0.700 | (0.8718) | 1.800 | (2.8618) | 2.075 | (4.0836) | 0.620 | (0.8319) | 2.125 | (4.1836) | 3.133 | (4.0612) |
| Median | 0.500 | | 0.300 | | 0.050 | | 0.200 | | 0.050 | | 1.200 | |
| Q1, Q3 | 0.000, 1.400 | | 0.000, 5.100 | | 0.000, 4.150 | | 0.100, 0.800 | | 0.000, 4.250 | | 0.400, 7.800 | |
| Min, Max | 0.00, 1.80 | | 0.00, 5.10 | | 0.00, 8.20 | | 0.00, 2.00 | | 0.00, 8.40 | | 0.40, 7.80 | |
| In Clinic Visit | | | | | | | | | | | | |
| (Day 12) | | | | | | | | | | | | |
| n | 4 | | 3 | | 4 | | 8 | | 3 | | 4 | |
| Mean (SD) | 0.275 | (0.5500) | 0.867 | (0.9609) | 2.325 | (4.5836) | 0.863 | (1.1070) | 1.867 | (3.2332) | 2.275 | (3.8922) |
| Median | 0.000 | | 0.700 | | 0.050 | | 0.450 | | 0.000 | | 0.450 | |
| Q1, Q3 | 0.000, 0.550 | | 0.000, 1.900 | | 0.000, 4.650 | | 0.000, 1.500 | | 0.000, 5.600 | | 0.150, 4.400 | |
| Min, Max | 0.00, 1.10 | | 0.00, 1.90 | | 0.00, 9.20 | | 0.00, 3.00 | | 0.00, 5.60 | | 0.10, 8.10 | |
| Change from | | | | | | | | | | | | |
| Baseline | | | | | | | | | | | | |
| n | 4 | | 3 | | 4 | | 8 | | 3 | | 3 | |
| Mean (SD) | −0.425 | (0.5058) | −0.933 | (1.9732) | 0.250 | (0.5000) | −0.275 | (0.8795) | −0.967 | (1.5885) | −0.167 | (0.8083) |
| Median | −0.350 | | 0.000 | | 0.000 | | −0.050 | | −0.100 | | 0.300 | |
| Q1, Q3 | −0.850, 0.000 | | −3.200, 0.400 | | 0.000, 0.500 | | −0.550, 0.000 | | −2.800, 0.000 | | −1.100, 0.300 | |
| Min, Max | −1.00, 0.00 | | −3.20, 0.40 | | 0.00, 1.00 | | −2.00, 1.00 | | −2.80, 0.00 | | −1.10, 0.30 | |
| Percent | | | | | | | | | | | | |
| Change from | | | | | | | | | | | | |
| Baseline | | | | | | | | | | | | |
| n | 2 | | 2 | | 2 | | 7 | | 2 | | 3 | |
| Mean (SD) | −69.44 | (43.212) | 35.29 | (138.648) | 6.10 | (8.623) | −30.36 | (55.835) | −66.67 | (47.140) | −4.27 | (83.629) |

| Median | −69.44 | 35.29 | 6.10 | −12.50 | −66.67 | 3.85 |
|---|---|---|---|---|---|---|
| Q1, Q3 | −100.00, −38.89 | −62.75, 133.33 | 0.00, 12.20 | −100.00, 0.00 | −100.00, −33.33 | −91.67, 75.00 |
| Min, Max | −100.0, −38.9 | −62.7, 133.3 | 0.0, 12.2 | −100.0, 50.0 | −100.0, −33.3 | −91.7, 75.0 |

TABLE O
Change from baseline and Percent change from baseline-Modified ITT Population-Part A. Overall Change in Severity.

| | | | | | | | Cilnidipine | | Cilnidipine | | | |
|---|---|---|---|---|---|---|---|---|---|---|---|---|
| | | | | | | | 10 mg + | | 20 mg + | | | |
| | Cilnidipine | | Cilnidipine | | Tadalafil | | Tadalafil | | Tadalafil | | | |
| | 10 mg | | 20 mg | | 5 mg | | 5 mg | | 5 mg | | Placebo | |
| Statistics | (N = 4) | | (N = 3) | | (N = 4) | | (N = 5) | | (N = 4) | | (N = 4) | |
| Baseline | | | | | | | | | | | | |
| n | 4 | | 3 | | 4 | | 5 | | 4 | | 4 | |
| Mean (SD) | 2.725 | (3.2877) | 2.667 | (0.4933) | 4.975 | (3.3837) | 3.300 | (2.3152) | 3.600 | (3.2321) | 2.000 | (1.7963) |
| Median | 1.900 | | 2.900 | | 6.000 | | 1.900 | | 2.550 | | 1.300 | |
| Q1, Q3 | 0.200, 5.250 | | 2.100, 3.000 | | 2.800, 7.150 | | 1.900, 4.700 | | 1.250, 5.950 | | 0.800, 3.200 | |
| Min, Max | 0.00, 7.10 | | 2.10, 3.00 | | 0.10, 7.80 | | 1.30, 6.70 | | 1.20, 8.10 | | 0.80, 4.60 | |
| In Clinic Visit | | | | | | | | | | | | |
| (Day 12) | | | | | | | | | | | | |
| n | 4 | | 3 | | 4 | | 8 | | 4 | | 4 | |
| Mean (SD) | 1.825 | (2.4061) | 1.033 | (0.4726) | 5.600 | (4.2528) | 2.975 | (1.8367) | 2.750 | (3.0957) | 2.700 | (1.9511) |
| Median | 1.050 | | 1.200 | | 6.550 | | 3.300 | | 1.500 | | 2.950 | |
| Q1, Q3 | 0.100, 3.550 | | 0.500,1.400 | | 2.200, 9.000 | | 1.150,4.050 | | 0.800, 4.700 | | 1.400, 4.000 | |
| Min, Max | 0.00, 5.20 | | 0.50, 1.40 | | 0.20, 9.10 | | 0.80, 6.00 | | 0.70, 7.30 | | 0.10, 4.80 | |
| Change from | | | | | | | | | | | | |
| Baseline | | | | | | | | | | | | |
| n | 4 | | 3 | | 4 | | 8 | | 4 | | 4 | |
| Mean (SD) | −0.900 | (0.9695) | −1.633, | (0.1528 | 0.625, | (1.5903) | −0.850 | (1.4948) | −0.850 | (0.5916) | 0.700 | (1.3089) |
| Median | −0.950 | | −1.600 | | 0.700 | | −0.700 | | −0.650 | | 0.550 | |
| Q1, Q3 | −1.700, −0.100 | | −1.800, −1.500 | | −0.600, 1.850 | | −1.500, −0.450 | | −1.250, −0.450 | | −0.250, 1.650 | |
| Min, Max | −1.90, 0.20 | | −1.80, −1.50 | | −1.30, 2.40 | | −3.40, 1.90 | | −1.70, −0.40 | | −0.70, 2.40 | |
| Percent | | | | | | | | | | | | |
| Change from | | | | | | | | | | | | |
| Baseline | | | | | | | | | | | | |
| n | 3 | | 3 | | 4 | | 8 | | 4 | | 4 | |
| Mean (SD) | −56.96 | (38.271) | −62.64 | (12.445) | 32.49 | (51.568) | −16.48 | (52.198) | −31.76 | (15.773) | 66.71 | (165.704) |

| Median | −44.12 | −60.00 | 26.790 | −18.99 | −36.22 | 27.17 |
|---|---|---|---|---|---|---|
| Q1, Q3 | −100.00, −26.76 | −76.19, −51.72 | −3.48, 68.46 | −49.16, −11.61 | −43.20, −20.32 | −41.58, 175.00 |
| Min, Max | −100.0, −26.8 | −76.2, −51.7 | −23.6, 100.0 | −72.3, 100.0 | −44.7, −9.9 | −87.5, 300.0 |

Table P shows the SHAQ parameter differences between Pooled cilnidipine (n) and placebo (n). Results with a (*) show a favorable treatment effect for cilnidipine.

| SHAQ PARAMETER | Pooled cilnidipine (n) | Placebo (n) |
|---|---|---|
| % Δ from baseline, | −25, −16.7 (4)* | −16.5, 0 (3) |
| standard disability | | |
| mean, median | | |
| % Δ from baseline, | −12.9, −22.2 (4)* | −13, 0 (3) |
| alternative disability | | |
| mean, median | | |
| % Δ from baseline, | −31.2, −45 (6)* | 7.3, 16.6 (3) |
| VAS Pain mean, median | | |
| % Δ from baseline, | −32.4, −44.1 (5)* | 57, −62 (3) |
| Intestinal Problems | | |
| mean, median | | |
| % Δ from baseline, | −8.2, −41.3 (4)* | 24.4, 7.1 (4) |
| VAS Breathing | | |
| mean, median | | |
| % Δ from baseline, | −18.6, −38.7 (6) | −41.8, −47.2 (4) |
| VAS Raynaud's | | |
| mean, median | | |
| % Δ from baseline, | −17.1, −50.9 (4)* | −4.3, 3.9 (3) |
| VAS Finger Ulcers | | |
| mean, median | | |
| % Δ from baseline, | −59.8, −55.9 (6)* | 66.7, 27 (4) |
| VAS Overall Disease | | |
| Severity mean, median | | |

Table Q shows difference for pooled cilnidipine patients compared to placebo on the SHAQ rating of overall disease severity is significant (p=0.01).

Scleroderma health assessment questionnaire (SHAQ)

| | | | Wilcoxon | T test |
|---|---|---|---|---|
| Parameter | 20 mg | placebo | p value | p value |
| VAS-Pain | −1.1 (0.79) | 0.78 (1.23) | 0.200 | 0.136 |
| VAS- Breathing | −0.83 (1.15) | −0.075 (0.35) | 0.943 | 0.540 |
| problems | | | | |
| Change | | | | |
| VAS- Raynaud's | −1.27 (1.06) | −0.5 (1.43) | 0.686 | 0.436 |
| Change | | | | |
| VAS- overall | −1.63 (0.15) | 0.7 (1.31) | 0.029 | 0.0115 |
| disease severity | | | | |
| Change | | | | |

Other results

| | | Cilnidipine (20 | Rirash 2017 |
|---|---|---|---|
| Parameter | Placebo(n = 4) | mg) **(n-3) | metanalysis^{†} |
| Mean weekly | 16.7 | 12.7 | 16.4 (n = 117 |
| attacks at | | | for 2° RP) |
| baseline | | | |
| Mean % | 18.9* | 43 | 24% (n = 117 |
| reduction of | | | for 2° RP) |
| weekly attacks | | | |
| at baseline | | | |
| Mean severity | 3.3 | 3.7 | Unknown |
| at baseline | | | |
| Mean % | −16.2 | 35.5 | 10.2(n = 138 |
| reduction in | | | for 2° RP) |
| severity | | | |
| Mean duration | 164.2 | 19.4 | Unknown |
| of attack at | | | |
| baseline | | | |
| Mean % | 12.3 | 20.0 | 6(n = 138) |
| reduction in | | | |
| duration | | | |
| Mean pain level | 2.3 | 2.3 | Unknown |
| at baseline | | | |
| Mean % | −3.9 | −11.8 | Ns |
| reduction in pain | | | |
| Mean RCS at | 2.6 | 3.5 | Unknown |
| baseline | | | |
| Mean % | 5.1 | 27.9 | 9(n = 192) |
| reduction | | | |
| in RCS | | | |

^{†}Rirash, F. et. al.; Cochrane Syst Rev. 2017 Dec. 13; 12 (12)

Table R shows additional results.

| | Cilnidipine 20 mg | Placebo |
|---|---|---|
| Parameter | (n = 3) | (n = 4) |
| Standard disability index | 50% improvement | 11% improvement |
| Alternative disability index | 42% improvement | 13% improvement |
| VAS- PAIN | 53% improvement* | 23% worse** |
| VAS - Breathing problems | 75% improvement* | 24% improvement |
| VAS - Raynaud's | 55% improvement | 16% improvemnt |
| VAS Overall Disease Severity | 63% improvement | 35% worse |

*Baseline was 2.17 on a 0-10 VAS
**Baseline was 3.3 on a 0-10 VAS
**Baseline was 1.13 on a 0-10 VAS

Table S provides a summary of data:

| | | Grade 1 Adverse | |
|---|---|---|---|
| Safety | No treatment D/Cs | Events (AEs) only | Efficacy Seen |
| Dose | 20 mg dose superior to | Worst AE in tadalafil | 20 mg dose group |
| response seen | all groups > Part B | monotherapy group | shows best overall |
| | | | response |
| Tadalafil 10 | 17% adverse effects in | Study conduct good | Attacks reduced |
| mg helps | any Profervia dose | | 43%_vs 23% with |
| | | | standard calcium |
| | | | channel blockers |

| | Pooled | Cilnidipine | |
|---|---|---|---|
| | Cilnidipine | 20 mg | Placebo |
| PARAMETER | (n = 7) | (n = 3) | (n = 4) |
| Weekly attack | −41% | −43% | −19% |
| frequency | | | |
| If baseline | −27% | −42% | −18% |
| RCS < 3.5 | | | |
| Duration | 20% | −20% | −12% |
| Severity | −16% | −36% | 16% |
| Daily RCS | 12% (n = 3) | −28% | −5% |
| Change | | | |
| Average | −27% | 12% | 4% |
| Raynaud's Pain | | | |
| Safety | 1AE (in combo with | 1AE (HA-mild; | 1AE (Increase |
| | T); no withdrawals | 1 withdrawal | in GERD) |
| | (n = 9) | (n = 8) | |
| Subgroup | <10 baseline −55% | <10 baseline −29% | <10 baseline 43% |
| attack | (n = 2) | (n = 2) | (n = 1) |
| frequency | >10 baseline −28% | >10 baseline −50% | >10 baseline −39% |
| | (n = 2) | (n = 2) | (n = 2) |
| Subgroup | <3.5 baseline −27% | <3.5 baseline −42% | <3.5 baseline −18% |
| Baseline | (n = 3) | (n = 1) | (n = 3) |
| RCS | 3.5-7.5 baseline −86% | >3.5-7.5 baseline −43% | 3.5-7.5 baseline −32% |
| | (n = 1) | (n = 2) | (n = 3) |

This evaluation study demonstrated successful implementation of the study processes, as indicated by the absence of Serious Adverse Events (SAEs), unblinding, or treatment discontinuations due to drug-related effects. Furthermore, only Grade 1 Adverse Events (AEs) were reported for patients treated with cilnidipine. Both cilnidipine doses, 10 mg and 20 mg, were well tolerated when administered alone or in combination with tadalafil. Notably, the 20 mg dose exhibited a greater overall effect compared to the 10 mg dose. The addition of tadalafil to cilnidipine demonstrated a more pronounced effect with the 10 mg dose compared to the 20 mg dose of cilnidipine alone. In comparison to an imputed historical comparator encompassing all Calcium Channel Blocker (CCB) trials for Systemic Sclerosis-Related Raynaud's Phenomenon (SSc-RP), cilnidipine exhibited improved safety with a lower incidence of Adverse Events (17% AE versus 4300 AE, p=0.024′7). Cilnidipine demonstrated an improvement in the overall severity of Systemic Sclerosis (SSc) as measured by the Scleroderma Health Assessment Questionnaire (SHAQ) when compared to the placebo group (p=0.01). Specifically, in the group receiving cilnidipine 20 mg (n=3), there was an improvement in the SHAQ scales of disability, pain, skin ulcers, Raynaud's phenomenon, and breathing difficulty compared to the placebo group (n=4). Furthermore, the addition of tadalafil, which enhances the brain concentration of cilnidipine by inhibiting plasma proteins that bind the drug, resulted in improved pain relief related to all causes of SSc when compared to cilnidipine alone.

### Conclusion:

In this study, cilnidipine demonstrated good tolerability among patients, with an overall adverse event rate of 17% observed in all patients who received any dose of cilnidipine (n=17). Importantly, only mild (Grade 1) adverse events occurred, and no treatment discontinuations due to intolerance were reported. A dose response was evident between cilnidipine 10 mg and 20 mg, with the higher dose showing a greater effect. Regarding the primary endpoint, cilnidipine 20 mg led to a significant reduction of 43% in weekly attack frequency, compared to 19% in patients treated with placebo. This reduction surpasses the clinically meaningful threshold of 25%. Comparatively, in this study, cilnidipine appeared to be safer and more effective than a large published meta-analysis of Calcium Channel Blocker (CCB) use in Raynaud's (both primary and secondary). Moreover, cilnidipine 20 mg exhibited a more substantial impact on alleviating the underlying burdens and manifestations of systemic sclerosis (SSc) when compared to placebo. In summary, cilnidipine holds promise as a well-tolerated and effective treatment option for patients with secondary Raynaud's primarily due to SSc.

The disclosures of all publications cited herein are expressly incorporated herein by reference, each in its entirety, to the same extent as if each were incorporated by reference individually.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating one or more symptoms associated with a comorbidity of secondary Raynaud's syndrome in a subject in need thereof, comprising:
determining that the subject has (i) secondary Raynaud's syndrome, and (ii) a comorbidity of secondary Raynaud's syndrome;
orally administering to the subject about 30 mg or about 20 mg of cilnidipine and optionally orally administering to the subject about 5 mg of tadalafil;
wherein the subject is cilnidipine-naïve;
wherein the cilnidipine and the optional tadalafil are administered to the subject once daily for at least five days; and
wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.

2. The method of claim 1, wherein after a first administration of cilnidipine or a pharmaceutically acceptable salt thereof and optional tadalafil or a pharmaceutically acceptable salt thereof, the severity and/or duration of the one or more symptoms is reduced in the subject.

3. The method of claim 1, wherein about 20 mg of cilnidipine or a pharmaceutically acceptable salt thereof on a free base basis of cilnidipine is administered to the subject.

4. The method of claim 1, wherein about 30 mg of cilnidipine or a pharmaceutically acceptable salt thereof on a free base basis of cilnidipine is administered to the subject.

5. The method of claim 1, wherein the tadalafil is not administered to the subject.

6. The method of claim 1, wherein the tadalafil is administered to the subject.

7. (canceled)

8. The method of claim 1, wherein the cilnidipine or a pharmaceutically acceptable salt thereof and the optional tadalafil or a pharmaceutically acceptable salt thereof are administered to the subject in the morning.

9. The method of claim 6, wherein the cilnidipine or a pharmaceutically acceptable salt thereof and tadalafil or a pharmaceutically acceptable salt thereof are administered as a fixed dosage form.

10. The method of claim 1, wherein the comorbidity of secondary Raynaud's syndrome is systemic sclerosis, lupus, rheumatoid arthritis, or any combination thereof.

11. The method of claim 1, wherein the comorbidity of secondary Raynaud's syndrome is systemic sclerosis.

12. The method of claim 1, wherein the comorbidity of secondary Raynaud's syndrome is lupus.

13. The method of claim 1, wherein the comorbidity of secondary Raynaud's syndrome is rheumatoid arthritis.

14. The method of claim 1, wherein the cilnidipine or a pharmaceutically acceptable salt thereof and optional tadalafil or a pharmaceutically acceptable salt thereof are formulated as a liquid suspension or as an oral thin film.

15. The method of claim 1, wherein the cilnidipine or a pharmaceutically acceptable salt thereof and optional tadalafil or a pharmaceutically acceptable salt thereof are formulated as a liquid suspension.

16. The method of claim 1, wherein the cilnidipine or a pharmaceutically acceptable salt thereof and optional tadalafil or a pharmaceutically acceptable salt thereof are formulated as an oral thin film.

17. The method of claim 1, wherein the one or more symptoms associated with the comorbidity of secondary Raynaud's syndrome is tooth pain.

18. The method of claim 1, wherein the one or more symptoms associated with the comorbidity of secondary Raynaud's syndrome is muscle pain.

19. The method of claim 1, wherein the one or more symptoms associated with the comorbidity of secondary Raynaud's syndrome is back pain.

20. The method of claim 1, wherein the one or more symptoms associated with the comorbidity of secondary Raynaud's syndrome is joint pain.

21. The method of claim 1, wherein the one or more symptoms associated with the comorbidity of secondary Raynaud's syndrome is headache.

22. The method of claim 1, wherein the one or more symptoms associated with the comorbidity of secondary Raynaud's syndrome is skin ulcer pain.

23. The method of claim 1, wherein the one or more symptoms associated with the comorbidity of secondary Raynaud's syndrome is neuropathic pain.

24. The method of claim 1, wherein the one or more symptoms associated with the comorbidity of secondary Raynaud's syndrome are gastrointestinal symptoms.

25. A method of treating one or more symptoms associated with a comorbidity of secondary Raynaud's syndrome in a subject in need thereof, comprising:
determining that the subject has (i) secondary Raynaud's syndrome, and (ii) a comorbidity of secondary Raynaud's syndrome;
orally administering to the subject about 30 mg or about 20 mg of cilnidipine and optionally orally administering to the subject about 5 mg of tadalafil;
measuring a reduction in the frequency of the one or more symptoms associated with Raynaud's syndrome of at least 25% after administering the cilnidipine or a pharmaceutically acceptable salt thereof and the optional tadalafil or a pharmaceutically acceptable salt thereof;
wherein the subject is cilnidipine-naïve; and
wherein the one or more symptoms associated with the comorbidity of secondary Raynaud's syndrome are selected from the group consisting of: tooth pain, muscle pain, back pain, joint pain, headache, skin ulcer pain, neuropathic pain, gastrointestinal symptoms, or any combination thereof.
